(19) United States
(12) Patent Application Publication
(10) Pub. No.: US 2024/0360411 A1
(43) Pub. Date: Oct. 31, 2024

(54) METHODS AND COMPOSITIONS FOR INDUCING FETAL HEMOGLOBIN, MODULATING ERYTHROID CELL LINEAGES, AND PERTURBING MEGAKARYOCYTE LINEAGES

(71) Applicant: FLAGSHIP PIONEERING INNOVATIONS VI, LLC, Cambridge, MA (US)

(72) Inventors: Mauricio CORTES, Wellesley, MA (US); Fabian WOLF, Cambridge, MA (US); Nicholas Mccartney PLUGIS, Cambridge, MA (US); Morag Helen STEWART, Cambridge, MA (US); Umut ESER, Cambridge, MA (US)

(21) Appl. No.: 18/687,661

(22) PCT Filed: Sep. 1, 2022
(86) PCT No.: PCT/US2022/042346

§ 371 Date: Feb. 28, 2024

Related U.S. Application Data
(60) Provisional application No. 63/239,816, filed on Sep. 1, 2021.
(60) Provisional application No. 63/242,233, filed on Sep. 9, 2021.
(60) Provisional application No. 63/244,022, filed on Sep. 14, 2021.

(51) Int. Cl. C12N 5/078; A61K 35/18; A61K 35/19; A61P 7/00

(52) U.S. Cl. CPC C12N 5/0641; A61K 35/18; A61K 35/19; A61P 7/00; C12N 5/0644; C12N 2501/999; C12N 2503/00; C12N 2506/11

(57) ABSTRACT

The present disclosure provides methods for increasing the quantity and/or the ratios of erythroblasts, reticulocytes, and/or erythrocytes, or progenitors thereof, in which any of these cells express HbF (e.g. HbF⁺ and/or HbF^{high} cells); increasing the quantity of erythrocytes and/or the ratios of erythrocytes to other related cells. The present disclosure relates, inter alia, to perturbagens and methods for directing a change in the cell state of a progenitor cell. The present disclosure further provides methods for treating diseases or disorders characterized by, for example, oxygen delivery deficiencies and/or reduced expression and/or activity of hemoglobin; abnormal erythron distribution and/or physiology or an erythrocyte deficiency; and diseases or disorders characterized by, at least, abnormal ratios and/or abnormal numbers of megakaryocytes, proplatelets, and/or platelets or immediate progenitors thereof.

### CROSS-REFERENCE TO RELATED APPLICATIONS

This application claims priority to and benefit of U.S. Provisional Patent Application No. 63/239,816 filed on Sep. 1, 2021, U.S. Provisional Patent Application No. 63/242,233 filed on Sep. 9, 2021, and U.S. Provisional Patent Application No. 63/244,022 filed on Sep. 14, 2021, the contents of all of which are hereby incorporated by reference in their entireties.

### BACKGROUND

An understanding of cellular mechanisms, including cellular mechanisms relating to development of erythroblasts, reticulocytes, and erythrocytes, and their lineages, and development of megakaryocyte, platelets, and their lineages, as well as methods and agents for directing changes in development of erythroblasts, reticulocytes, erythrocytes (including erythrocytes comprising fetal hemoglobin), megakaryocyte and platelets, may be useful for treating diseases or disorders, including those underscored by abnormal amounts or ratio of various cell types. Currently, there is an unmet need for such methods and agents that can be used for the treatment of such diseases and disorders.

### SUMMARY

In aspects and embodiments, there is provided a method for directing a change in cell state of a progenitor cell comprising, contacting a population of cells comprising a progenitor cell with at least one perturbagen selected from Table 4, Table 5, and/or Table 6, or a variant thereof, including combinations of the foregoing, wherein the at least one perturbagen is capable of altering a gene signature in the progenitor cell; and wherein the progenitor cell is a non-lineage committed CD34+ cell.

In aspects and embodiments, there is provided a method for directing a change in cell state of a progenitor cell, comprising, contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1, Table 2, and/or Table 3, including combinations of the foregoing, and/or a decrease in expression and/or activity in the progenitor cell of one or more genes selected from the genes designated as a “down” gene in the gene directionality column of Table 1, Table 2, and/or Table 3, including combinations of the foregoing, and wherein the progenitor cell is a non-lineage committed CD34+ cell.

In aspects and embodiments, there is provided a method for directing a change in cell state of a progenitor cell, comprising, contacting a population of cells comprising a progenitor cell with at least one perturbagen selected from Table 4, Table 5, and/or Table 6, or a variant thereof, including combinations of the foregoing, and capable of altering a gene signature in the progenitor cell, wherein altering the gene signature comprises an increase in expression and/or activity in the progenitor cell of one or more genes selected from the genes designated as an “up” gene in the gene directionality column of Table 1, Table 2, and/or Table 3, including combinations of the foregoing, and/or a decrease in expression and/or activity in the progenitor cell of one or more genes selected from the genes designated as a “down” gene in the gene directionality column of Table 1, Table 2, and/or Table 3, including combinations of the foregoing, and wherein the progenitor cell is a non-lineage committed CD34+ cell.

In embodiments, altering the gene signature comprises an increase in expression and/or activity in the progenitor cell of a network module designated in the network module column of Table 1, Table 2, and/or Table 3, including combinations of the foregoing.

In embodiments, the change in cell state provides an increase in the number of one or more of proerythroblasts, early erythroblasts, intermediate erythroblasts, late erythroblasts, reticulocytes, and erythrocytes, wherein one or more of the proerythroblasts, early erythroblasts, intermediate erythroblasts, late erythroblasts, reticulocytes, and erythrocytes expresses fetal hemoglobin (HbF).

In embodiments, the change in cell state provides an increase in F cells.

In embodiments, one or more of the proerythroblasts, early erythroblasts, intermediate erythroblasts, late erythroblasts, reticulocytes, and erythrocytes expresses HBG1 and/or HBG2 In embodiments, the increase in the number of erythrocytes comprising HbF is relative to the number of erythrocytes obtained from a population of progenitor cells that is not contacted with the at least one perturbagen, or relative to the population of progenitor cells prior to contacting with the at least one perturbagen.

In embodiments, the increase in the ratio of the number of F cells to non-F cells is increased relative to the ratio obtained from a population of progenitor cells that is not contacted with the at least one perturbagen or relative to the ratio in the population of progenitor cells prior to contacting with the at least one perturbagen.

In embodiments, the change in cell state provides an increase in the number of one or more of proerythroblasts, early erythroblasts, intermediate erythroblasts, late erythroblasts, reticulocytes, and/or erythrocytes.

In embodiments, the increase in the number of erythrocytes is relative to the number of erythrocytes obtained from a population of progenitor cells that is not contacted with the at least one perturbagen or relative to the population of progenitor cells prior to contacting with the at least one perturbagen.

In embodiments, the change in cell state provides an increase in the number of one or more of megakaryocyte/erythroid progenitor cells (MEP), committed megakaryocyte progenitor cells, promegakaryocytes, megakaryocytes, proplatelets, and platelets.

In embodiments, the change in cell state provides an increase in the number of megakaryocytes, proplatelets, and/or platelets relative to the number of megakaryocytes, proplatelets, and/or platelets obtained from a population of progenitor cells that is not contacted with the at least one perturbagen or relative to the population of progenitor cells prior to contacting with the at least one perturbagen.

In embodiments, the number of progenitor cells is decreased. In embodiments, the number of progenitor cells is increased.

In embodiments, the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.

In embodiments, the number of proerythroblasts, early erythroblasts, intermediate erythroblasts, late erythroblasts, reticulocytes, and/or erythrocytes is increased after contacting the population of cells comprising a CD34+ cell with the at least one perturbagen.

In embodiments, the number of MEP cells, committed megakaryocyte progenitor cells, promegakaryocytes, megakaryocytes, proplatelets, and/or platelets is increased after contacting the population of cells comprising a CD34+ cell with the at least one perturbagen.

In embodiments, at least one perturbagen is selected from Table 4, Table 5, and/or Table 6, or a variant thereof, including combinations of the foregoing, comprises at least 2, at least 3, at least 4, or at least 5 perturbagens selected from Table 4, Table 5, and/or Table 6, or variants thereof, including combinations of the foregoing.

In embodiments, one or more genes are selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, genes selected from the genes designated as an “up” gene in the gene directionality column of Table 1.

In embodiments, one or more genes are selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1.

In embodiments, one or more genes are selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1.

In embodiments, one or more genes are selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2.

In embodiments, one or more genes are selected from the genes designated as an “up” gene in the gene directionality column of Table 2, comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2.

In embodiments the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39.

In embodiments, one or more genes are selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2.

In embodiments, one or more genes are selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNA/2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1.

In embodiments, one or more genes are selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or 23 or more genes designated as an “up” gene in the gene directionality column of Table 3.

In embodiments, one or more genes are selected from Table 3 comprises at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK.

In embodiments, one or more genes are one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3.

In embodiments, one or more genes are selected from Table 3 comprises at least one of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.

In embodiments, contacting the population of progenitor cells occurs in vitro or ex vivo.

In aspects and embodiments, there is provided a perturbagen for use in any of the methods described herein.

In aspects and embodiments, there is provided a pharmaceutical composition comprising a perturbagen described herein.

In aspects and embodiments, there is provided a method for promoting the formation of a megakaryocyte cell, or an immediate progenitor thereof, comprising, exposing a starting population of stem/progenitor cells comprising a non-lineage committed CD34+ cell to a perturbation having a perturbation signature that promotes the transition of the starting population of stem/progenitor cells into a MEP cell, committed megakaryocyte progenitor cell, or a promegakaryocyte, wherein the perturbation signature comprises increased expression and/or activity of one or more of genes selected from Table 3 designated as an “up” gene in the gene directionality column of Table 3 and/or a decreased expression and/or activity in the non-lineage committed CD34+ cell of one or more genes selected from Table 3 designated as a “down” gene in the gene directionality column of Table 3.

In embodiments, the perturbation signature comprises an activation of a network module designated in the network module column of Table 3.

In aspects and embodiments, there is provided a method of increasing a quantity of megakaryocyte cell, or immediate progenitors thereof, comprising, exposing a starting population of stem/progenitor cells comprising a non-lineage committed CD34+ cell to a pharmaceutical composition that promotes the formation of lineage specific progenitor population selected from MEP cell, committed megakaryocyte progenitor cell, or a promegakaryocyte, the pharmaceutical composition promoting the transition of a primitive stem/progenitor population into the lineage specific progenitor population that has the capacity to differentiate into megakaryocytes, proplatelets, and/or platelets or immediate progenitors thereof, wherein the pharmaceutical composition comprises at least one perturbagen selected from Table 6, or a variant thereof.

In aspects and embodiments, there is provided a method for treating a disease or disorder disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, the method comprising, (a) administering to a patient in need thereof a therapeutically effective amount of at least one perturbagen selected from Table 6, or a variant thereof, wherein the at least one perturbagen is capable of changing a gene signature in a progenitor cell, or (b) administering to a patient in need thereof a cell, the cell having been contacted with at least one perturbagen selected from Table 6, or a variant thereof, wherein the at least one perturbagen is capable of changing a gene signature in a progenitor cell.

In aspects and embodiments, there is provided a method for treating a disease or disorder characterized by an abnormal number of megakaryocytes, proplatelets, and/or platelets, comprising, (a) administering to a patient in need thereof a therapeutically effective amount of at least one perturbagen selected from Table 6, or a variant thereof, wherein the at least one perturbagen is capable of changing a gene signature in a progenitor cell, or (b) administering to a patient in need thereof a cell, the cell having been contacted with at least one perturbagen selected from Table 6, or a variant thereof, wherein the at least one perturbagen is capable of changing a gene signature in a progenitor cell.

In embodiments, the abnormal ratio comprises a decreased number of megakaryocytes, proplatelets, and/or platelets and/or an increased number of progenitor cells.

In aspects and embodiments, there is provided a method for treating a disease or disorder characterized by an abnormal oxygen delivery, comprising: (a) administering to a subject in need thereof a therapeutically effective amount of at least one perturbagen selected from Table 4, or a variant thereof, wherein the at least one perturbagen is capable of changing a gene signature in a progenitor cell or (b) administering to a subject in need thereof a cell, the cell having been contacted with at least one perturbagen selected from Table 4, or a variant thereof, wherein the at least one perturbagen is capable of changing a gene signature in a progenitor cell.

In aspects and embodiments, there is provided a method for treating a disease or disorder characterized by a hemoglobin deficiency, comprising, (a) administering to a subject in need thereof a therapeutically effective amount of at least one perturbagen selected from Table 4, or a variant thereof, wherein the at least one perturbagen is capable of changing a gene signature in a progenitor cell or (b) administering to a subject in need thereof a cell, the cell having been contacted with at least one perturbagen selected from Table 4, or a variant thereof, wherein the at least one perturbagen is capable of changing a gene signature in a progenitor cell.

In aspects and embodiments, there is provided a method for treating or preventing an sickle cell disease or a thalassemia, comprising, (a) administering to a subject in need thereof a therapeutically effective amount of at least one perturbagen selected from Table 4, or a variant thereof, wherein the at least one perturbagen is capable of changing a gene signature in a progenitor cell or (b) administering to a subject in need thereof a cell, the cell having been contacted with at least one perturbagen selected from Table 4, or a variant thereof, wherein the at least one perturbagen is capable of changing a gene signature in a progenitor cell.

In aspects and embodiments, there is provided a method for treating a disease or disorder characterized by an abnormal erythron distribution and/or physiology, comprising, (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.

In aspects and embodiments, there is provided a method for treating a disease or disorder characterized by an erythrocyte deficiency, comprising, (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.

In aspects and embodiments, there is provided a method for treating or preventing an anemia, comprising, (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.

In embodiments, the sickle cell disease or a thalassemia is sickle cell anemia (SS), sickle hemoglobin-C disease (SC), sickle beta-plus thalassemia and sickle beta-zero thalassemia.

In embodiments, the anemia is selected from aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia.

In embodiments, the disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia.

In embodiments, the subject is selected by steps comprising, obtaining from the subject having the disease or disorder a sample of cells comprising a non-lineage committed CD34+ cell; and contacting the sample of cells with at least one perturbagen selected from Table 4, Table 5, and/or Table 6, or a variant thereof, including combinations of the foregoing, wherein the at least one perturbagen alters a gene signature in the sample of cells.

In embodiments, the subject is selected by steps comprising, obtaining from a subject having the disease or disorder a sample of cells comprising a non-lineage committed CD34+ cell, and contacting the sample of cells with at least one perturbagen capable of altering a gene signature in a non-lineage committed CD34+ cell, wherein the at least one perturbagen increases in the sample of cells the expression and/or activity of one or more genes selected from the genes designated as an “up” gene in the gene directionality column of Table 1, Table 2, and/or Table 3, including combinations of the foregoing, and/or decreases in the sample of cells the expression and/or activity of one or more genes selected from the genes designated as a “down” gene in the gene directionality column of Table 1, Table 2, and/or Table 3, including combinations of the foregoing.

In embodiments, altering the gene signature comprises an activation of a network module designated in the network module column of Table 3.

In embodiments, the subject is selected by steps comprising, obtaining from a subject having the disease or disorder a sample of cells comprising a non-lineage committed CD34+ cell, and contacting the sample of cells with at least one perturbagen selected from Table 4, Table 5, and/or Table 6, or a variant thereof, including combinations of the foregoing; wherein the at least one perturbagen increases in the sample of cells the expression and/or activity of one or more genes selected from the genes designated as an “up” gene in the gene directionality column of Table 1, Table 2, and/or Table 3, including combinations of the foregoing, and/or decreases in the sample of cells the expression and/or activity of one or more genes selected from the genes designated as a “down” gene in the gene directionality column of Table 1, Table 2, and/or Table 3, including combinations of the foregoing.

In embodiments, altering the gene signature comprises an activation of a network module designated in the network module column of Table 3.

In aspects and embodiments, there is provided a use of the perturbagen of Table 4, or a variant thereof in the manufacture of a medicament for treating a disease or disorder characterized an abnormal oxygen delivery or a hemoglobin deficiency.

In aspects and embodiments, there is provided a use of a perturbagen of Table 4, or a variant thereof in the manufacture of a medicament for treating sickle cell disease or a thalassemia.

In aspects and embodiments, there is provided a use of a perturbagen of Table 5, or a variant thereof in the manufacture of a medicament for treating a disease or disorder characterized by an abnormal ratio of erythrocytes to progenitor cells.

In aspects and embodiments, there is provided a use of a perturbagen of Table 6, or a variant thereof in the manufacture of a medicament for treating a disease or disorder characterized by an abnormal ratio of megakaryocytes, proplatelets, and/or platelets to progenitor cells.

In aspects and embodiments, there is provided a use of a perturbagen of Table 6, or a variant thereof in the manufacture of a medicament for treating a disease or disorder characterized by an abnormal ratio of megakaryocytes, proplatelets, and/or platelets to other committed blood cells, optionally erythrocytes.

In aspects and embodiments, there is provided a method of identifying a candidate perturbation for promoting the transition of a starting population of progenitor cells into erythrocytes comprising HbF or HbF-expressing progenitors thereof, the method comprising, exposing the starting population of progenitor cells to a perturbation, identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation; and identifying the perturbation as a candidate perturbation for promoting the transition of a population of progenitor cells into erythrocytes comprising HbF or HbF-expressing progenitors thereof based on the perturbation signature, wherein the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as a “down” gene in the gene directionality column of Table 1.

In aspects and embodiments, there is provided a method of identifying a candidate perturbation for promoting the transition of a starting population of progenitor cells into erythrocytes or immediate progenitors thereof, the method comprising, exposing the starting population of progenitor cells to a perturbation, identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes or immediate progenitors thereof following exposure of the population of cells to the perturbation, and identifying the perturbation as a candidate perturbation for promoting the transition of a population of progenitor cells into erythrocytes or immediate progenitors thereof based on the perturbation signature, wherein the perturbation signature is an increase in expression and/or activity in the progenitor cell of one or more genes selected from the genes designated as an “up” gene in the gene directionality column of Table 2, and/or a decrease in expression and/or activity in the progenitor cell of one or more genes selected from the genes designated as an “down” gene in the gene directionality column of Table 2.

In aspects and embodiments, there is provided a method of identifying a candidate perturbation for promoting the transition of a starting population of progenitor cells into megakaryocytes, proplatelets, and/or platelets or immediate progenitors thereof, the method comprising, exposing the starting population of progenitor cells to a perturbation, identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into megakaryocytes, proplatelets, and/or platelets or immediate progenitors thereof following exposure of the population of cells to the perturbation, and identifying the perturbation as a candidate perturbation for promoting the transition of a population of progenitor cells into megakaryocytes, proplatelets, and/or platelets or immediate progenitors thereof based on the perturbation signature, wherein the perturbation signature is an increase in expression and/or activity in the progenitor cell of one or more genes selected from Table 3 designated as an “up” gene in the gene directionality column of Table 3, and/or a decrease in expression and/or activity in the progenitor cell of one or more genes selected from Table 3 designated as a “down” gene in the gene directionality column of Table 3.

In embodiments, the perturbation signature is an increase in expression and/or activity in the progenitor cell of a network module designated in the network module column of Table 1, Table 2, and/or Table 3, including combinations of the foregoing.

In embodiments, there is provided a method for making a therapeutic agent for a disease or disorder selected from a sickle cell disease or a thalassemia or a disease or disorder characterized by an abnormal oxygen delivery or a hemoglobin deficiency, comprising, (a) identifying a candidate perturbation for therapy according to a method disclosed herein, and (b) formulating the candidate perturbation as a therapeutic agent for the treatment of the disease or disorder.

In embodiments, there is provided a method for making a therapeutic agent for a disease or disorder selected from a disease or disorder characterized by an abnormal erythron distribution and/or physiology or erythrocyte deficiency, comprising, (a) identifying a candidate perturbation for therapy and (b) formulating the candidate perturbation as a therapeutic agent for the treatment of the disease or disorder.

In embodiments, there is provided a method for making a therapeutic agent for a disease or disorder selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, comprising, (a) identifying a candidate perturbation according to a method described herein, and (b) formulating the candidate perturbation as a therapeutic agent for the treatment of the disease or disorder.

In aspects and embodiments, there is provided a method for directing a change in cell state of a plurality of progenitor cells comprising: contacting a population of cells comprising a plurality of progenitor cells with at least one perturbagen selected from Table 4, Table 5, and/or Table 6, or a variant thereof, including combinations of the foregoing, wherein the at least one perturbagen is capable of altering one or more gene signatures in the plurality of progenitor cells; and wherein the plurality of progenitor cells are non-lineage committed CD34+ cells.

In embodiments, the change in cell state provides an increase in the number of one or more of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets, and any combination thereof, optionally wherein one or more of the proerythroblasts, early erythroblasts, intermediate erythroblasts, late erythroblasts, reticulocytes, and erythrocytes expresses fetal hemoglobin (HbF).

In embodiments, the number of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets is increased after contacting the population of cells comprising a CD34+ cell with the at least one perturbagen.

In embodiments, the at least one perturbagen selected from Table 4, Table 5, and/or Table 6, or a variant thereof, including combinations of the foregoing, comprises at least 2, at least 3, at least 4, or at least 5 perturbagens selected from Table 4, Table 5, and/or Table 6, or variants thereof, including combinations of the foregoing.

In embodiments, the one or more gene signatures are selected from:
a) one or more genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, genes selected from the genes designated as an “up” gene in the gene directionality column of Table 1;
b) one or more genes selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1;
c) one or more genes selected from the genes designated as an “up” gene in the gene directionality column of Table 2, comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2:
d) one or more genes selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2;
e) one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or 23 or more genes designated as an “up” gene in the gene directionality column of Table 3; and
f) one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3.

In embodiments: the one or more genes comprise one or more of a)-f):
a) the one or more genes selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1;
b) the one or more genes selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2;
c) the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39;
d) the one or more genes selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNAI2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1;
e) the one or more genes selected from Table 3 comprises at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK; and/or
f) the one or more genes selected from Table 3 comprises at least one of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.

### BRIEF DESCRIPTION OF THE DRAWINGS

FIG. 1A is schematic showing lineage directions of human hematopoiesis. Each numbered cluster represents cells of a specific state/lineage. Cluster 10 represents non-lineage committed CD34+ cells and cluster 8 represents cells in early erythroid progenitor lineages, which ultimately differentiate into reticulocytes and erythrocytes. The arrow shows the effect of perturbagens that drive cells from the non-lineage committed CD34+ cells (of cluster 10) towards cells of the erythroid lineages (cluster 8). Cluster 9 represents cells of the granulocyte-monocyte progenitor (GMP) lineage, cluster 15 represents cells of the megakaryocyte lineage, and cluster 13 represents cells of the mast cell/basophil lineage.

FIG. 1B illustrates a process for identifying and characterizing perturbagens that drive non-lineage committed CD34+ cells towards the erythrocyte lineages.

FIG. 1C shows in vitro cell culture for testing perturbagens on promoting erythroid lineage differentiation from non-lineage committed CD34+ cells as measured by increase in erythroid progenitors: erythroid progenitor cells marked by (CD71+CD235a+) at day 7 with a perturbagen, tracking erythroid progenitor cell maturation by flow cytometry using a four-antibody panel (CD71, CD235a, CD233, CD49d) over 18 days post perturbagen addition by monitoring the increase of CD233 expression, with a concomitant loss of CD49d expression, and a shift in CD71^{HI} to CD71^{low} erythroid population (CD235a+). Perturbagens are listed in Table 4.

FIG. 2 shows a panel of 15 compounds promoting the induction of HbF in in vitro derived erythrocytes from sickle patients as measured by increase in % of F+ cells at day 18 post compound addition as compared with DMSO as negative control and hydroxyurea treatment as positive control. Perturbagens are listed in Table 4.

FIG. 3A shows a t-distributed stochastic neighbor embedding (t-SNE) plot illustrating the predictions that drive the transition of cells from CD34+ erythroid stem cells to erythroid lineage. Clusters are highlighted in different shades. Each numbered cluster represents cells of a specific state/lineage. Cluster 18 represents granulocyte progenitors, cluster 13 represents cells of the mast cell/basophil lineage, cluster 15 represents cells of the megakaryocyte lineage, and Cluster 0 represents erythroid progenitor lineages, which ultimately differentiate into reticulocytes and erythrocytes.

FIG. 3B is a schematic showing experimental procedure used to study the transition of cells from CD34+ erythroid stem cells to erythroid lineage.

FIG. 3C is a stacked bar graph showing experimental procedure used to study the transition of cells from CD34+ erythroid stem cells to the indicated cell types.

FIG. 4A is flow cytometry data demonstrating that treatment of cells with Perturbagen 26 induces early erythroid lineage commitment (CD34−CD41−CD71+CD36+CD235a−) compared to vehicle control. Perturbagen 26 is listed in Table 5.

FIG. 4B is a bar graph of experimental data demonstrating the identification of compounds that induce early erythroid lineage commitment (CD34−CD41−CD71+CD36+CD235a−). Statistical analysis: unpaired T-test compared to DMSO; *p<0.05, **p<0.01, ***p<0.001. The data bars (from left to right) represent the following: NT, DMSO, Erythroid, Megakaryocyte, Perturbagen 1 (10 μM), Perturbagen 2 (1 μM), Perturbagen 3 (10 μM), Perturbagen 4 (10 μM), Perturbagen 5 (3 μM), Perturbagen 6 (10 μM), Perturbagen 7 (0.003 μM), Perturbagen 8 (10 μM), Perturbagen 9 (10 μM), Perturbagen 10 (10 μM), Perturbagen 11 (10 μM), Perturbagen 12 (0.003 μM), Perturbagen 13 (0.003 μM), Perturbagen 14 (3 μM), Perturbagen 15 (10 μM), Perturbagen 16 (10 μM), Perturbagen 11 (3 μM), Perturbagen 18 (10 μM), Perturbagen 19 (10 μM), Perturbagen 20 (10 μM), Perturbagen 21 (10 μM), Perturbagen 22 (10 μM), Perturbagen 23 (1 μM), Perturbagen 24 (0.3 μM), Perturbagen 25 (0.3 μM), and Perturbagen 26 (3 μM). Perturbagens are listed in Table 5.

FIG. 5A is schematic showing lineage directions of human hematopoiesis. Each numbered cluster represents cells of a specific state/lineage. Cluster 10 represents non-lineage committed CD34+ cells and cluster 9 represents cells in the granulocyte-monocyte progenitor (GMP) lineages, which ultimately differentiate into monocytes and neutrophils. The arrow shows the effect of perturbagens that drive cells from the non-lineage committed CD34+ cells (of cluster 10) towards cells of the GMP lineages (cluster 9). Cluster 8 represent cells of the early erythroid lineage, cluster 15 represent cells of the megakaryocyte lineage, and cluster 13 represents cells of the mast cell/basophil lineage.

FIG. 5B illustrates a process for identifying and characterizing perturbagens that drive non-lineage committed CD34+ cells towards the megakaryocyte lineages (bottom left) or away from the megakaryocyte lineages (bottom right).

FIG. 5C illustrates an analysis in which progenitor cells are provided control treatments or a cocktail of cytokines (perturbagens) which drive specific cell lineage fates. NT control is the no treatment control. MK stands for megakaryocyte.

FIG. 6A and FIG. 6B show compounds promoting MkP lineage differentiation as measured by increase in Megakaryocyte progenitors (CD34+CD38+/−CD41+CD71+) at 48 h (FIG. 6A) and 5 days (CD34-CD38+/−CD41+CD71+) (FIG. 6B) post-compounds addition. Perturbagens are listed in Table 6.

FIG. 7A to FIG. 7C show results obtained when animals (C57Bl/6J mice) were dosed daily i.p. for 14 days with three concentrations of Perturbagen 3 (0.1 mg, 0.3, and 1 mg/Kg) or the FDA approved thrombopoietin mimetic (Nplate). Changes in bone marrow Mkps (FIG. 7B) and blood platelets (FIG. 7C) are shown. Perturbagens are listed in Table 6.

FIG. 8A and FIG. 8B show an in vivo mouse model of thrombocytopenia depicting the kinetics of loss and recovery of platelets by mild irradiation (FIG. 8A) and busulfan conditioning (FIG. 8B).

FIG. 9 is a bar graph of the fold change of early megakaryocytes progenitors in vitro human lineage differentiation assay of Example 9 showing several compounds were active at increasing megakaryocyte progenitors. Plot represent at least 3 biological replicates. Early MkP were defined at day 5 are defined as CD71+CD41+CD235a−. Perturbagens are listed in Table 6.

FIG. 10 is a bar graph of the fold change of early megakaryocytes in vitro human lineage differentiation assay of Example 9 showing that Perturbagen 6 and related compounds were active at increasing megakaryocyte progenitors. Early MkP were defined at day 5 are defined as CD71+CD41+CD235a−. Perturbagens are listed in Table 6.

FIG. 11 shows a schematic of healthy mouse study, depicting an 8-day study. Similarly study can be extended to 14 days. A daily dosing is shown. Dosing may be less frequent, e.g., every other day.

FIG. 12A and FIG. 12B provide flow cytometry scatter plots defining Linage-Sca1+cKit+LSK cells to further elucidate HSCs MPPs and short-term HSCs (FIG. 12A). Long term HSC's are delineated as CD34−CD135−, ST-HSCs and MPPs are further divided to CD34+CD135(Flk2)− or CD34+CD135(Flk2)+, respectively (FIG. 12B).

FIG. 13A and FIG. 13B provide flow cytometry scatter plots defining Linage−Sca1−cKit− population to further elucidate oligo and bipotent progenitor (FIG. 13A). Megakaryocyte/erythroid progenitors (MEPs) were defined by CD34− and CD16/32−, GMP is CD34+CD16/32 high and Common myeloid progenitor (CMP) is CD34+CD16/32 low (FIG. 13B).

FIG. 14A to FIG. 14C provide flow cytometry scatter plots defining megakaryocyte progenitors and mature megakaryocytes are gated from lineage negative population (FIG. 14A). Early MkP are Lin−CD41+Cd42−, where late MkPs and MK are Lin−CD41+CD42+(FIG. 14B). Megakaryocytes and late Mkps are further distinguished based on size using FSC vs SCC (FIG. 14C).

FIG. 15 is a bar graph summarizing the results of bone marrow analysis of vehicle- and Perturbagen 3-treated animals (1 mg/kg every-other-day (EOD) for 7 days), demonstrating an increase in the early megakaryocyte compartment. Vehicle (N=2), Perturbagen 3 (N=3).

FIG. 16A shows the model design (FIG. 16A) for CIT and FIG. 16B shows a line graph of a time course of platelet depletion in a dose dependent manner. Mice that were treated received a single dose of carboplatin showed a nadir at day 9 with full recovery by day 21.

FIG. 17A and FIG. 17B show a line graphs of a time course analysis of early Mkps (FIG. 17A) and mature megakaryocytes (FIG. 17B) after treatment with a single dose of carboplatin.

### DETAILED DESCRIPTION

The present disclosure is based, in part, on the discovery that cells of hematopoietic lineages comprising erythroblasts, reticulocytes, and erythrocytes, and their progenitors can be characterized by specific gene signatures. Additionally, the present disclosure is based on the discovery that certain active agents (i.e., perturbagens) can alter these specific gene signatures. Such alteration is associated with the acquisition of specific cell states by cells of erythrocyte and/or erythroid lineages. These alterations are also associated with the acquisition of specific cell states by cells of megakaryocyte and/or platelet lineages. These perturbagens are, in some instance, useful as therapeutics and derive benefit by directing the reactivation of fetal hemoglobin (HbF). These perturbagens are, in some instance, useful as therapeutics and derive benefit by promoting the production of erythrocytes. These perturbagens are, in some instance, useful as therapeutics and derive benefit by directing the progenitor cells towards megakaryocyte and/or platelet states.

### Fetal Hemoglobin

### Gene Signature

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins (i.e., antigen expression) that are characteristic of the cell type or cells of a specific lineage.

The erythroid progenitor cells at different maturation stages may be characterized by its antigen expression. The erythroblasts express transferrin receptor (also known as CD71 in human) and glycophorin A (GlyA, also known as CD235a in human) (Hattangadiet. al., Blood, 2011, 118 (24):6258-68.), but express little or no hemoglobin (Hb). The erythroblasts have the capacity to mature into hemoglobinized erythrocytes and reticulocytes. During maturation, CD71 expression decreases but remains detectable on most cells, GlyA expression remains high or increases further, and cell pellets become visibly red due to the accumulation of Hb.

Genome-wide association studies (GWAS) demonstrated that about half of the heritable variation in HbF level is due to polymorphism in three loci including β-globin cluster, an intergenic interval between the HBS1L and MYB genes, and the BCL11A gene. BCL11A gene is a zinc-finger transcriptional factor that functions as a developmental stage-specific repressor of HbF expression. (Sankaran et al. Human fetal hemoglobin expression is regulated by the developmental stage-specific repressor BCL11A, Science. 2008; 322:1839-1842; Sankaran et al. Developmental and species-divergent globin switching are driven by BCL11A, Nature. 2009; 460:1093-10). KLF1 gene is reported as a DNA-binding transcription factor that activates BCL11A expression by associating with the BCL11A promoter, suggesting a dual role of KLF1 gene in globin gene regulation by both functioning as a direct activator of adult-stage β-globin and indirect repressor of fetal-stage γ-globin (Zhou et al. KLF1 regulates BCL11A expression and γ- to β-globin gene switching, Nat Genet, 2010; vol. 42, pp. 742-744).

Fetal hemoglobin also known as hemoglobin F, HbF, or α2γ2) is as a hemoglobin tetramer composed of gamma globin subunit encoded by the HBG1 or HBG2 gene. Fetal hemoglobin is the main oxygen transport protein in the human fetus during the last seven months of development in the uterus and persists in the newborn until roughly 2-4 months old. Fetal hemoglobin differs most from adult hemoglobin in that it is able to bind oxygen with greater affinity than the adult form the (the p₅₀ of fetal hemoglobin is roughly 19 mmHg, whereas adult hemoglobin is approximately 26.8 mmHg). Like most types of normal hemoglobin, fetal hemoglobin is a tetramer composed of four protein subunits and four heme prosthetic groups. However, adult hemoglobin is composed of two α (alpha) and two β (beta) subunits, while fetal hemoglobin is composed of two a subunits and two γ (gamma) subunits (α2γ2).

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

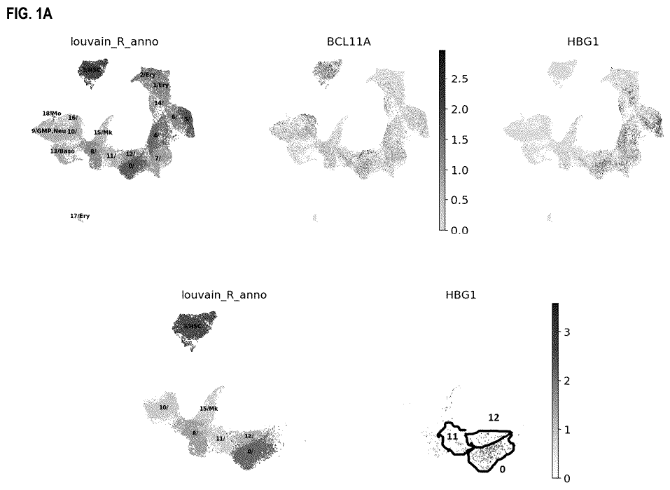

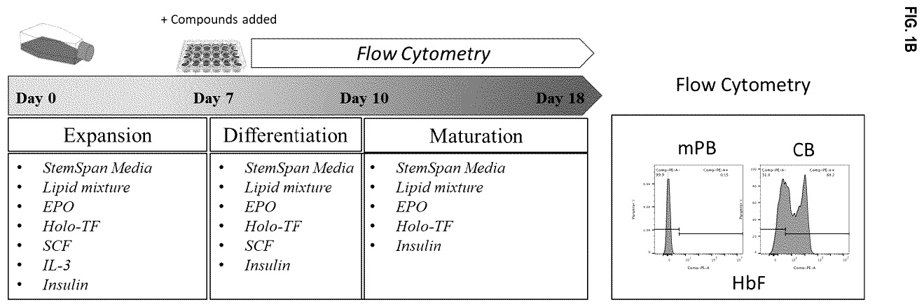

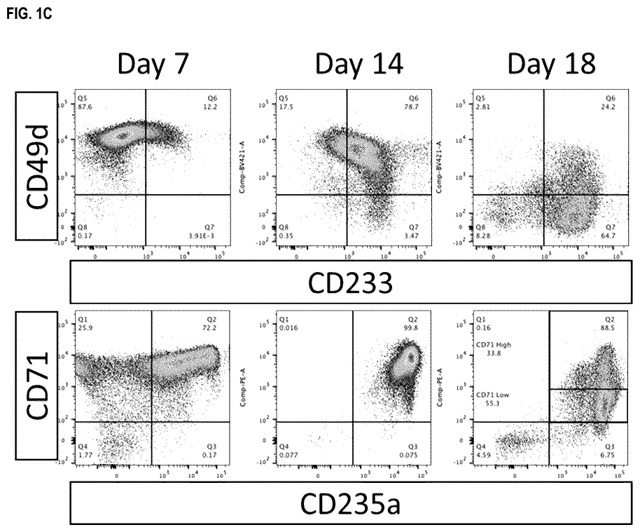

FIG. 1A shows annotated clusters that associate gene signature with cell types or cells of a specific lineage. Differential gene signatures for the 11 to 0 transition, i.e., from a non-lineage committed CD34⁺ progenitor cell to cells of the erythroid lineage expressing HBG1, were used to predict perturbations that would promote the transition

Genes that are differentially expressed and positively associated with the promotion of erythroid lineage progression and/or erythrocyte differentiation are listed in Table 1.

TABLE 1
Genes showing an change in expression.

| | | Gene | Gene | Network |
|---|---|---|---|---|
| | Gene | EntrezID | Directionality | Module |
| 0 | KIT | 3815 | up | 0 |
| 1 | APOE | 348 | up | 0 |
| 2 | CDK6 | 1021 | down | 0 |
| 3 | PLP2 | 5355 | down | 0 |
| 4 | MAP7 | 9053 | down | 0 |
| 5 | TRAPPC6A | 79090 | down | 0 |
| 6 | BID | 637 | down | 0 |
| 7 | SYK | 3735 | down | 0 |
| 8 | FAIM | 55179 | down | 0 |
| 9 | BTK | 695 | down | 0 |
| 10 | TBXA2R | 6915 | down | 0 |
| 11 | RNH1 | 6050 | up | 1 |
| 12 | LYPLA1 | 10434 | down | 1 |
| 13 | MAPKAPK3 | 7867 | down | 1 |
| 14 | SLC35F2 | 54733 | down | 1 |
| 15 | ANXA7 | 310 | down | 1 |
| 16 | ATP6V0B | 533 | down | 1 |
| 17 | SYPL1 | 6856 | down | 1 |
| 18 | BCL7B | 9275 | down | 1 |
| 19 | INPP1 | 3628 | down | 1 |
| 20 | ADI1 | 55256 | down | 1 |
| 21 | MACF1 | 23499 | down | 1 |
| 22 | ID2 | 3398 | up | 2 |
| 23 | BLVRA | 644 | up | 2 |
| 24 | TSKU | 25987 | up | 2 |
| 25 | MLLT11 | 10962 | down | 2 |
| 26 | FHL2 | 2274 | down | 2 |
| 27 | RNPS1 | 10921 | down | 2 |
| 28 | TPM1 | 7168 | down | 2 |
| 29 | THAP11 | 57215 | down | 2 |
| 30 | DUSP14 | 11072 | down | 2 |
| 31 | HEBP1 | 50865 | up | 3 |
| 32 | TRAK2 | 66008 | up | 3 |
| 33 | HK1 | 3098 | up | 3 |
| 34 | PSMB8 | 5696 | down | 3 |
| 35 | EIF4EBP1 | 1978 | down | 3 |
| 36 | MFSD10 | 10227 | down | 3 |
| 37 | PSMD2 | 5708 | down | 3 |
| 38 | SPTLC2 | 9517 | down | 3 |
| 39 | CORO1A | 11151 | down | 3 |
| 40 | GAPDH | 2597 | up | 4 |
| 41 | MPC2 | 25874 | up | 4 |
| 42 | CTNNAL1 | 8727 | up | 4 |
| 43 | PDLIM1 | 9124 | down | 4 |
| 44 | CCDC85B | 11007 | down | 4 |
| 45 | ITGAE | 3682 | down | 4 |
| 46 | CCDC86 | 79080 | down | 4 |
| 47 | SLC5A6 | 8884 | down | 4 |
| 48 | GRWD1 | 83743 | down | 4 |
| 49 | CAST | 831 | up | 6 |
| 50 | CALM3 | 808 | up | 6 |
| 51 | SNCA | 6622 | down | 6 |
| 52 | IL1B | 3553 | down | 6 |
| 53 | MEST | 4232 | down | 6 |
| 54 | DAXX | 1616 | down | 6 |
| 55 | UBE2L6 | 9246 | down | 6 |
| 56 | PTPRC | 5788 | down | 6 |
| 57 | GADD45A | 1647 | down | 6 |
| 58 | RPA3 | 6119 | up | 7 |
| 59 | ELOVL6 | 79071 | up | 7 |
| 60 | NENF | 29937 | down | 7 |
| 61 | PTPN6 | 5777 | down | 7 |
| 62 | RHOA | 387 | down | 7 |
| 63 | EVL | 51466 | down | 7 |
| 64 | VDAC1 | 7416 | down | 7 |
| 65 | TIMM17B | 10245 | down | 7 |
| 66 | BNIP3 | 664 | up | 8 |
| 67 | SPAG4 | 6676 | up | 8 |
| 68 | MTHFD2 | 10797 | down | 8 |
| 69 | XBP1 | 7494 | down | 8 |
| 70 | EBNA1BP2 | 10969 | down | 8 |
| 71 | CYCS | 54205 | down | 8 |
| 72 | TCEAL4 | 79921 | down | 8 |
| 73 | S100A4 | 6275 | up | 9 |
| 74 | RALB | 5899 | up | 9 |
| 75 | TMEM109 | 79073 | down | 9 |
| 76 | MLEC | 9761 | down | 9 |
| 77 | HDAC2 | 3066 | down | 9 |
| 78 | SKP1 | 6500 | down | 9 |
| 79 | RAP1GAP | 5909 | up | 10 |
| 80 | DENND2D | 79961 | up | 10 |
| 81 | MEF2C | 4208 | down | 10 |
| 82 | SPAG7 | 9552 | down | 10 |
| 83 | ICAM3 | 3385 | down | 10 |
| 84 | RPL39L | 116832 | down | 10 |
| 85 | SOX4 | 6659 | down | 11 |
| 86 | MYC | 4609 | down | 11 |
| 87 | IL4R | 3566 | down | 11 |
| 88 | TES | 26136 | down | 11 |
| 89 | CASP3 | 836 | down | 11 |
| 90 | CTSL | 1514 | up | 12 |
| 91 | DDIT4 | 54541 | up | 12 |
| 92 | BNIP3L | 665 | up | 12 |
| 93 | VAT1 | 10493 | up | 12 |
| 94 | PHGDH | 26227 | down | 12 |
| 95 | DRAP1 | 10589 | down | 13 |
| 96 | RPS6 | 6194 | down | 13 |
| 97 | RNF167 | 26001 | down | 13 |
| 98 | PSME2 | 5721 | down | 13 |

In Table 1 and associated embodiments:
“Gene ID”: at the time of filing the present disclosure, the World Wide Web at ncbi.nlm.nih.gov/gene provides a description of and the nucleic acid sequence for each GeneID listed in Table 1; the contents of each of which is incorporated herein by reference in its entirety.
“Up” indicates a gene for which an increase in expression and/or activity in the progenitor cell is associated with the gene signature.
“Down” indicates a gene for which an decrease in expression and/or activity in the progenitor cell is associated with the gene signature.

A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition—such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 1, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1. In certain embodiments, a perturbation activates multiple network modules for the desired cell state transition, such as 2, 3, 4, 5, 6, 7, 8, 9, 10, 11, or 12 modules. In certain embodiments, a perturbation activates at least one network modules for the desired cell state transition selected from network modules 0, 1, 2, 3, 4, 6, 7, 8, 9, 10, 11, 12, and 13 described in Table 1. In certain embodiments, a perturbation activates at least, as 2, 3, 4, 5, 6, 7, 8, 9, 10, 11 network modules for the desired cell state transition selected from network modules 0, 1, 2, 3, 4, 6, 7, 8, 9, 10, 11, 12, and 13 described in Table 1. In certain embodiments, a perturbation activates each of the network modules 0, 1, 2, 3, 4, 6, 7, 8, 9, 10, 11, 12, and 13 described in Table 1.

In some embodiments, one or more genes of network module 0 are modulated. In some embodiments, the perturbation activates network module 1, e.g., modulation of the expression and/or activity of one or more of (inclusive of all of) KIT, APOE, CDK6, PLP2, MAP7, TRAPPC6A, BID, SYK, FAIM, BTK, and TBXA2R genes. In some embodiments, the modulation is upmodulation or downmodulation as described in Gene Directionality column of Table 1.

In some embodiments, one or more genes of network module 1 are modulated. In some embodiments, the perturbation activates network module 1, e.g., modulation of the expression and/or activity of one or more of (inclusive of all of) RNH1, LYPLA1, MAPKAPK3, SLC35F2, ANXA7, ATP6VOB, SYPL1, BCL7B, INPP1, ADI1, and MACF1 genes. In some embodiments, the modulation is upmodulation or downmodulation as described in Gene Directionality column of Table 1.

In some embodiments, one or more genes of network module 2 are modulated. In some embodiments, the perturbation activates network module 2, e.g., modulation of the expression and/or activity of one or more of (inclusive of all of) ID2, BLVRA, TSKU, MLLT11, FHL2, RNPS1, TPM1, THAP11, and DUSP14 genes. In some embodiments, the modulation is upmodulation or downmodulation as described in Gene Directionality column of Table 1.

In some embodiments, one or more genes of network module 3 are modulated. In some embodiments, the perturbation activates network module 3, e.g., modulation of the expression and/or activity of one or more of (inclusive of all of) HEBP1, TRAK2, HK1, PSMB8, EIF4EBP1, MFSD10, PSMD2, SPTLC2, and CORO1A genes. In some embodiments, the modulation is upmodulation or downmodulation as described in Gene Directionality column of Table 1.

In some embodiments, one or more genes of network module 4 are modulated. In some embodiments, the perturbation activates network module 4, e.g., modulation of the expression and/or activity of one or more of (inclusive of all of) GAPDH, MPC2, CTNNAL1, PDLIM1, CCDC85B, ITGAE, CCDC86, SLC5A6, and GRWD1 genes. In some embodiments, the modulation is upmodulation or downmodulation as described in Gene Directionality column of Table 1.

In some embodiments, one or more genes of network module 6 are modulated. In some embodiments, the perturbation activates network module 6, e.g., modulation of the expression and/or activity of one or more of (inclusive of all of) CAST, CALM3, SNCA, IL1B, MEST, DAXX, UBE2L6, PTPRC, and GADD45A genes. In some embodiments, the modulation is upmodulation or downmodulation as described in Gene Directionality column of Table 1.

In some embodiments, one or more genes of network module 7 are modulated. In some embodiments, the perturbation activates network module 7, e.g., modulation of the expression and/or activity of one or more of (inclusive of all of) RPA3, ELOVL6, NENF, PTPN6, RHOA, EVL, VDAC1, and TIMM17B genes. In some embodiments, the modulation is upmodulation or downmodulation as described in Gene Directionality column of Table 1.

In some embodiments, one or more genes of network module 8 are modulated. In some embodiments, the perturbation activates network module 8, e.g., modulation of the expression and/or activity of one or more of (inclusive of all of) BNIP3, SPAG4, MTHFD2, XBP1, EBNA1BP2, CYCS, and TCEAL4 genes. In some embodiments, the modulation is upmodulation or downmodulation as described in Gene Directionality column of Table 1.

In some embodiments, one or more genes of network module 9 are modulated. In some embodiments, the perturbation activates network module 9, e.g., modulation of the expression and/or activity of one or more of (inclusive of all of) S100A4, RALB, TMEM109, MLEC, HDAC2, and SKP1 genes. In some embodiments, the modulation is upmodulation or downmodulation as described in Gene Directionality column of Table 1.

In some embodiments, one or more genes of network module 10 are modulated. In some embodiments, the perturbation activates network module 10, e.g., modulation of the expression and/or activity of one or more of (inclusive of all of) RAP1GAP, DENND2D, MEF2C, SPAG7, ICAM3, and RPL39L genes. In some embodiments, the modulation is upmodulation or downmodulation as described in Gene Directionality column of Table 1.

In some embodiments, one or more genes of network module 11 are modulated. In some embodiments, the perturbation activates network module 11, e.g., modulation of the expression and/or activity of one or more of (inclusive of all of) SOX4, MYC, IL4R, TES, and CASP3 genes. In some embodiments, the modulation is upmodulation or downmodulation as described in Gene Directionality column of Table 1.

In some embodiments, one or more genes of network module 12 are modulated. In some embodiments, the perturbation activates network module 12, e.g., modulation of the expression and/or activity of one or more of (inclusive of all of) CTSL, DDIT4, BNIP3L, VAT1, and PHGDH genes. In some embodiments, the modulation is upmodulation or downmodulation as described in Gene Directionality column of Table 1.

In some embodiments, one or more genes of network module 13 are modulated. In some embodiments, the perturbation activates network module 13, e.g., modulation of the expression and/or activity of one or more of (inclusive of all of) DRAP1, RPS6, RNF167, and PSME2 genes. In some embodiments, the modulation is upmodulation or downmodulation as described in Gene Directionality column of Table 1.

In some embodiments, the present methods alter a gene signature in the sample of cells, comprising an activation of a network module designated in the network module column of Table 1.

In some embodiments, the activation of the network module designated in the network module column of Table 1 comprises modulating expression and/or activity of 2 or more genes within a network module.

In some embodiments, the activation of the network module designated in the network module column of Table 1 comprises modulating expression and/or activity of all of the genes within a network module.

In some embodiments, the activation of the network module designated in the network module column of Table 1 comprises modulating expression and/or activity of 2 or more genes within 2 or more network modules. In some embodiments, the activation of the network module designated in the network module column of Table 1 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more, 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, or 67 or more, or 68 or more, or 69 or more, 70 or more, or 71 or more, or 72 or more, or 73 or more, or 74 or more, or 75 or more, or 76 or more, or 77 or more, or 78 or more, or 79 or more, 80 or more, or 81 or more, or 82 or more, or 83 or more, or 84 or more, or 85 or more, or 86 or more, or 87 or more, or 88 or more, or 89 or more, 90 or more, or 91 or more, or 92 or more, or 93 or more, or 94 or more, or 95 or more, or 96 or more, or 97 or more genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 network modules selected from the network modules 0, 1, 2, 3, 4, 6, 7, 8, 9, 10, 11, 12, and 13 described in Table 1).

At the time of filing the present disclosure, the World Wide Web at ncbi.nlm.nih.gov/gene provides a description of and the nucleic acid sequence for each Gene designated as an “up” gene in the gene directionality column of Table 1; the contents of each of which is incorporated herein by reference in its entirety. At the time of filing the present disclosure, the World Wide Web at ncbi.nlm.nih.gov/gene provides a description of and the nucleic acid sequence for each Gene listed in the genes designated as a “down” gene in the gene directionality column of Table 1; the contents of each of which is incorporated herein by reference in its entirety.

### Perturbagens

### Fetal Hemaglobin Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting erythrocyte lineage differentiation are listed in Table 4.

TABLE 4
Perturbagens

| Perturbagen | Molecular | MW | Effective in |
|---|---|---|---|
| No. | Formula | (g/mol) | vitro conc. |
| 1 | C₁₇H₂₂N₂O₃ | 302.37 | 10 nM |
| 2 | C₁₉H₂₇BN₄O₄ | 386.3 | 1 nM |
| 3 | C₃₅H₄₅N₇O₈S | 723.8 | 10.0 μM |
| 4 | C₂₀H₁₆N₂O₄ | 348.4 | 10 nM |
| 5 | C₂₈H₂₇ClF₅NO | 524.0 | 30 nM |
| 6 | C₂₃H₂₄O₈ | 428.4 | 10 nM |
| 7 | C₂₀H₂₁N₃O₇S | 447.5 | 10 nM |
| 8 | C₃₅H₄₉NO₉ | 627.8 | 10 nM |
| 9 | C₂₄H₂₈N₂O₃ | 392.5 | 0.12 μM |
| 10 | C₁₅H₁₂F₂N₆O₃S | 394.4 | 1 μM |
| 11 | C₂₀H₂₃ClN₂O₅ | 406.9 | 10.0 μM |
| 12 | C₂₀H₁₉N₄O₃ | 363.4 | 10 nM |
| 13 | C₂₆H₄₁N₃O₅ | 475.6 | 10.0 μM |
| 14 | C₆H₇N₃O | 137.14 | 0.12 μM |
| 15 | C₂₁H₂₁ClN₂O | 352.9 | 100 nM |

In various embodiments herein, a perturbagen of Table 4 encompasses the perturbagens and/or other perturbagens identified in Table 4. Thus, the perturbagens of Table 4 represent examples of perturbagens of the present disclosure.

In Table 4, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In some embodiments, a perturbagen used in the present disclosure is a variant of a perturbagen of Table 4.

A variant may be a derivative, analog, enantiomer or a mixture of enantiomers thereof or a pharmaceutically acceptable salt, solvate, hydrate, co-crystal, clathrate, or polymorph of the perturbagen of Table 4. A variant of a perturbagen of Table 4 retains the biological activity of the perturbagen of Table 4.

### Methods and Perturbagens for Directing a Change in Cell State

### Fetal Hemaglobin

Particular cellular changes in cell state can be matched to differential gene expression (which collectively define a gene signature), caused by exposure of a cell to a perturbagen. In some embodiments, a change in cell state may be from one progenitor cell type to another progenitor cell type. For example, a megakaryocyte-erythroid progenitor cell (MEP) may give rise to an erythrocyte. In some embodiments, a change in cell state may be from an upstream progenitor cell (e.g. proerythroblasts) to a downstream progenitor cell (e.g., late erythroblasts). Lastly, in some embodiments, a change in cell state may be from the final non-differentiated cell into a differentiated cell.

An aspect of the present disclosure is related to a method for directing a change in cell state of a progenitor cell. This method includes a step of contacting (e.g. in vitro or in vivo) a population of cells comprising a progenitor cell with at least one perturbagen selected from Table 4, or a variant of perturbagens described in Table 4. In this aspect, the at least one perturbagen is capable of altering a gene signature in the progenitor cell. In one embodiment, the progenitor cell is a non-lineage committed CD34+ cell. In one embodiment, the progenitor cell is a non-lineage committed CD34+ cell. In one embodiment, the progenitor cell is a non-lineage committed CD34+ cells selected from a hematopoietic stem cell (an HSC; e.g., a CD34+HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), a orthochromatic erythroblast (also known as a late erythroblasts).

Another aspect of the present disclosure is related to a method for directing a change in cell state of a progenitor cell. This method includes a step of contacting a population of cells comprising a progenitor cell with at least one perturbagen capable of altering a gene signature in the progenitor cell. In this aspect, altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as a “down” gene in the gene directionality column of Table 1. In one embodiment, the progenitor cell is a non-lineage committed CD34+ cell.

Yet another aspect of the present disclosure is related to a method for directing a change in cell state of a progenitor cell. This method includes a step of contacting a population of cells comprising a progenitor cell with at least one perturbagen selected from Table 4, or a variant of perturbagens described in Table 4, and capable of altering a gene signature in the progenitor cell. In this aspect, altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as a “down” gene in the gene directionality column of Table 1. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), a orthochromatic erythroblast (also known as a late erythroblasts).

In some embodiments, the non-lineage committed CD34+ cell is a hematopoietic stem and progenitor cell (HSPC). In some embodiments, the step of contacting a population of cells comprising a progenitor cell with a perturbagen causes a change in the cell state. In some embodiments, the erythrocytes are marked by antigen expression CD34+CD38+CD71^{low}+CD235a⁺+CD41⁻. (See Example 2 infra).

In some embodiments, the erythrocytes can be derived from the canonical MEP developmental pathway. In other embodiments, the erythrocytes can be derived from a developmental pathway that does not include the canonical MEP cell. In embodiments, the erythrocytes may be produced from erythropoietin-independent pathway, for example, signal through gp130 and c-kit dramatically promote erythropoiesis from human CD34+ cells (Sui et al., Erythropoietin-independent erythrocyte production: signals through gp130 and c-kit dramatically promote erythropoiesis from human CD34⁺ cells, J. Exp. Med., 1996, vol. 183, pp. 837-845).

In some embodiment, the change in the number of proerythroblasts, erythroblasts, reticulocytes, and/or erythrocytes is relative to a control population of cells. For example, in some embodiments, the increase in the number of proerythroblasts, erythroblasts, reticulocytes, and/or erythrocytes upon contacting the cells with a perturbagen-is relative to the population of progenitor cells that is not contacted with the perturbagen. In other embodiments, the increase in the number of proerythroblasts, erythroblasts, reticulocytes, and/or erythrocytes upon contacting the cells with a perturbagen- is relative to the population of progenitor cells prior to contacting it with the perturbagen. In some embodiments, a change in the number of proerythroblasts, erythroblasts, reticulocytes, and/or erythrocytes is caused by change in the state of the cells of a population of progenitor cells. For example, an increase in the number of proerythroblasts, erythroblasts, reticulocytes, and/or erythrocytes within a population of progenitor cell can be due to a change in the state of the cells.

Methods for determining the extension of the lifespan of a specific cell type or a reduction of cell death is well known in the art. For examples, markers for dying cells, e.g., caspases can be detected, or dyes for dead cells, e.g., methylene blue, may be used.

In some embodiments, the number of progenitor cells is decreased. In some embodiments, the decrease in the number of progenitor cells is due in part to decreased cell proliferation of the progenitor cells. In some embodiments, the decrease in the number of progenitor cells is due in part to a decreased lifespan of the progenitor cells. In some embodiments, the decrease in the number of progenitor cells is due in part to increased cell death among the progenitor cells. In some embodiments, the decrease in the number of progenitor cells is relative to the number of progenitor cells in a population of progenitor cells that is not contacted with the at least one perturbagen. In some embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into the erythrocyte lineage.

In some embodiments, the number of progenitor cells is increased. In some embodiments, the increase in the number of progenitor cells is due in part to increased cell proliferation of the progenitor cells. In some embodiments, the increase in the number of progenitor cells is due in part to an increased lifespan of the progenitor cells. In some embodiments, the increase in the number of progenitor cells is due in part to decreased cell death among the progenitor cells. In some embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In some embodiments, the number of proerythroblasts, BFU-E cells, CFU-E cells, early erythroblasts, intermediate erythroblasts, late erythroblasts, reticulocytes, and/or erythrocytes is increased after contacting the population of cells comprising a CD34⁺ cell with the at least one perturbagen. In some embodiments, the number of proerythroblasts, early erythroblasts, intermediate erythroblasts, and/or late erythroblasts is increased after contacting the population of cells comprising a CD34⁺ cell with the at least one perturbagen. In some embodiments, the number of proerythroblasts is increased after contacting the population of cells comprising a CD34⁺ cell with the at least one perturbagen. In other embodiments, the number of proerythroblasts, early erythroblasts, intermediate erythroblasts, and/or late erythroblasts is increased after contacting the population of cells comprising a CD34⁺ cell with the at least one perturbagen. In other embodiments, the number of early erythroblasts, intermediate erythroblasts, and/or late erythroblasts is increased after contacting the population of cells comprising a CD34⁺ cell with the at least one perturbagen. In some embodiments, the number of reticulocytes, and/or erythrocytes is increased after contacting the population of cells comprising a CD34⁺ cell with the at least one perturbagen. In some embodiments, the number of proerythroblasts is increased after contacting the population of cells comprising a CD34⁺ cell with the at least one perturbagen. In some embodiments, the number of proerythroblasts is increased after contacting the population of cells comprising a CD34⁺ cell with the at least one perturbagen. In some embodiments, the number of reticulocytes is increased after contacting the population of cells comprising a CD34⁺ cell with the at least one perturbagen. In some embodiments, the number of erythrocytes is increased after contacting the population of cells comprising a CD34⁺ cell with the at least one perturbagen.

In some embodiments, the ratio of the number of proerythroblasts, erythroblasts, reticulocytes, and/or erythrocytes to the number of progenitor cells is increased relative to the ratio obtained from a population of progenitor cells that is not contacted with the at least one perturbagen. In some embodiments, the ratio of the number of proerythroblasts, erythroblasts, reticulocytes, and/or erythrocytes to the number of progenitor cells is increased relative to the ratio in the population of progenitor cells prior to contacting with the at least one perturbagen.

In some embodiments, the ratio of the number of proerythroblasts, early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of proerythroblasts, early erythroblasts, intermediate erythroblasts, and/or late erythroblasts to the number of proerythroblasts is increased relative to the ratio in the population of progenitor cells prior to contacting with the at least one perturbagen.

In some embodiments, the ratio of the number of early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of early erythroblasts, intermediate erythroblasts, and/or late erythroblasts to the number of proerythroblasts is increased relative to the ratio in the population of progenitor cells prior to contacting with the at least one perturbagen.

In yet other embodiments, for the methods described herein, the ratio of the number of reticulocytes, and/or erythrocytes to the number of proerythroblasts is increased relative to the ratio obtained from a population of progenitor cells that is not contacted with the at least one perturbagen. In other embodiments, for the methods described here, the ratio of the number of reticulocytes, and/or erythrocytes to the number of proerythroblasts is increased relative to the ratio in the population of progenitor cells prior to contacting with the at least one perturbagen. In yet other embodiments, for the methods described herein, the ratio of the number of reticulocytes to the number of proerythroblasts is increased relative to the ratio obtained from a population of progenitor cells that is not contacted with the at least one perturbagen. In other embodiments, for the methods described here, the ratio of the number of reticulocytes to the number of proerythroblasts is increased relative to the ratio in the population of progenitor cells prior to contacting with the at least one perturbagen. In yet other embodiments, for the methods described herein, the ratio of the number of erythrocytes to the number of proerythroblasts is increased relative to the ratio obtained from a population of progenitor cells that is not contacted with the at least one perturbagen. In other embodiments, for the methods described here, the ratio of the number of erythrocytes to the number of proerythroblasts is increased relative to the ratio in the population of progenitor cells prior to contacting with the at least one perturbagen.

In some embodiments, for the methods described herein, the ratio of the number of reticulocytes and/or erythrocytes to the number of proerythroblasts, early erythroblasts, intermediate erythroblasts, and/or late erythroblasts is increased relative to the ratio obtained from a population of progenitor cells that is not contacted with the at least one perturbagen. In other embodiments, for the methods described herein, the ratio of the number of reticulocytes and/or erythrocytes to the number of proerythroblasts, early erythroblasts, intermediate erythroblasts, and/or late erythroblasts is increased relative to the ratio in the population of progenitor cells prior to contacting with the at least one perturbagen. In some embodiments, for the methods described herein, the ratio of the number of erythrocytes to the number of proerythroblasts, early erythroblasts, intermediate erythroblasts, and/or late erythroblasts is increased relative to the ratio obtained from a population of progenitor cells that is not contacted with the at least one perturbagen. In other embodiments, for the methods described herein, the ratio of the number of erythrocytes to the number of proerythroblasts, early erythroblasts, intermediate erythroblasts, and/or late erythroblasts is increased relative to the ratio in the population of progenitor cells prior to contacting with the at least one perturbagen. In some embodiments, for the methods described herein, the ratio of the number of reticulocytes to the number of proerythroblasts, early erythroblasts, intermediate erythroblasts, and/or late erythroblasts is increased relative to the ratio obtained from a population of progenitor cells that is not contacted with the at least one perturbagen. In other embodiments, for the methods described herein, the ratio of the number of reticulocytes to the number of proerythroblasts, early erythroblasts, intermediate erythroblasts, and/or late erythroblasts is increased relative to the ratio in the population of progenitor cells prior to contacting with the at least one perturbagen.

In some embodiments, for the methods described herein, the ratio of the number of reticulocytes, and/or erythrocytes to the number of early erythroblasts, intermediate erythroblasts, and/or late erythroblasts is increased relative to the ratio obtained from a population of progenitor cells that is not contacted with the at least one perturbagen. In other embodiments, for the methods described here, the ratio of the number of reticulocytes, and/or erythrocytes to the number of early erythroblasts, intermediate erythroblasts, and/or late erythroblasts is increased relative to the ratio in the population of progenitor cells prior to contacting with the at least one perturbagen. In some embodiments, for the methods described herein, the ratio of the number of reticulocytes to the number of early erythroblasts, intermediate erythroblasts, and/or late erythroblasts is increased relative to the ratio obtained from a population of progenitor cells that is not contacted with the at least one perturbagen. In other embodiments, for the methods described here, the ratio of the number of reticulocytes to the number of early erythroblasts, intermediate erythroblasts, and/or late erythroblasts is increased relative to the ratio in the population of progenitor cells prior to contacting with the at least one perturbagen. In some embodiments, the ratio of the number erythrocytes to the number of early erythroblasts, intermediate erythroblasts, and/or late erythroblasts is increased relative to the ratio obtained from a population of progenitor cells that is not contacted with the at least one perturbagen. In other embodiments, the ratio of the number of erythrocytes to the number of early erythroblasts, intermediate erythroblasts, and/or late erythroblasts is increased relative to the ratio in the population of progenitor cells prior to contacting with the at least one perturbagen.

In some embodiments, the ratio of the number of proerythroblasts, early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of proerythroblasts, early erythroblasts, intermediate erythroblasts, and/or late erythroblasts to the number of proerythroblasts is increased relative to the ratio in the population of progenitor cells prior to contacting with the at least one perturbagen. In some embodiments, the ratio of the number of proerythroblasts to the number of proerythroblasts is increased relative to the ratio obtained from a population of progenitor cells that is not contacted with the at least one perturbagen. In other embodiments, the ratio of the number of proerythroblasts to the number of proerythroblasts is increased relative to the ratio in the population of progenitor cells prior to contacting with the at least one perturbagen. In some embodiments, the ratio of the number of early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of early erythroblasts, intermediate erythroblasts, and/or late erythroblasts to the number of proerythroblasts is increased relative to the ratio in the population of progenitor cells prior to contacting with the at least one perturbagen.

In some embodiments, the ratio of the number of early erythroblasts, intermediate erythroblasts, late erythroblasts, reticulocytes, and/or erythrocytes to the number of proerythroblasts is increased relative to the ratio obtained from a population of progenitor cells that is not contacted with the at least one perturbagen. In other embodiments, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen.

In some embodiments, the ratio of the number of early erythroblasts, intermediate erythroblasts, late erythroblasts, reticulocytes, and/or erythrocytes to the number of proerythroblasts is increased relative to the ratio obtained from a population of progenitor cells that is not contacted with the at least one perturbagen. In other embodiments, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen.

In some embodiments, the ratio of the number of reticulocytes, and/or erythrocytes to the number of proerythroblasts is increased relative to the ratio obtained from a population of progenitor cells that is not contacted with the at least one perturbagen. In other embodiments, the ratio of the number of reticulocytes, and/or erythrocytes to the number of proerythroblasts is increased relative to the ratio in the population of progenitor cells prior to contacting with the at least one perturbagen. In some embodiments, the ratio of the number of reticulocytes to the number of proerythroblasts is increased relative to the ratio obtained from a population of progenitor cells that is not contacted with the at least one perturbagen. In other embodiments, the ratio of the number of reticulocytes to the number of proerythroblasts is increased relative to the ratio in the population of progenitor cells prior to contacting with the at least one perturbagen. In some embodiments, the ratio of the number of erythrocytes to the number of proerythroblasts is increased relative to the ratio obtained from a population of progenitor cells that is not contacted with the at least one perturbagen. In other embodiments, the ratio of the number of erythrocytes to the number of proerythroblasts is increased relative to the ratio in the population of progenitor cells prior to contacting with the at least one perturbagen.

In some embodiments of the methods described herein, the ratio of the number of erythrocytes to the number of erythroblasts and/or reticulocytes is increased relative to the ratio obtained from a population of progenitor cells that is not contacted with the at least one perturbagen. In other embodiments, the ratio of the number of erythrocytes to the number of erythroblasts and/or reticulocytes is increased relative to the ratio in the population of progenitor cells prior to contacting with the at least one perturbagen.

In some embodiments, the number of proerythroblasts is decreased. In some embodiments, the number of erythroblasts is decreased. In some embodiments, the number of proerythroblasts is decreased. In some embodiments, the number of proerythroblasts is increased. In some embodiments, the number of early erythroblasts, intermediate erythroblasts, and/or late erythroblasts is increased. In some embodiments, the number of reticulocytes is increased. In some embodiments, the number of proerythroblasts is increased.

Methods for counting cells are well known in the art. Non-limiting examples include hemocytometry, flow cytometry, and cell sorting techniques, e.g., fluorescence activated cell sorting (FACS). The maturation of the erythrocytes is, in some embodiments, determined by loss of CD71 expression. Erythroid maturation is determined by, in some embodiments, flow cytometry using a four-antibody panel (See Example 2 infra) (CD71, CD235a, CD233, CD49d) with increased CD233 expression, with a concomitant loss of CD49d expression, and a shift in CD71^{Hi} to CD7^{low} erythroid population (CD235a+).

In some embodiments, the change in cell state of a progenitor cell provides an increase in the number of one or more of proerythroblasts, early erythroblasts, intermediate erythroblasts, late erythroblasts, reticulocytes, and erythrocytes. In some embodiments, one or more of the proerythroblasts, early erythroblasts, intermediate erythroblasts, late erythroblasts, reticulocytes, and erythrocytes comprise fetal hemoglobin (HbF).

In one embodiment, the change in cell state of a progenitor cell provides an increase in F cells. See Boyer et al. 1975. Fetal hemoglobin restriction to a few erythrocytes (F cells) in normal human adults. Science 188: 361-363

In some embodiments, one or more of the proerythroblasts, early erythroblasts, intermediate erythroblasts, late erythroblasts, reticulocytes, and erythrocytes expresses HBG1 and/or HBG2. In embodiments, the increase in the number of erythrocytes comprising HbF is relative to the number of erythrocytes obtained from a population of progenitor cells that is not contacted with the at least one perturbagen. In other embodiments, the increase in the number of erythrocytes comprising HbF is relative to the population of progenitor cells prior to contacting with the at least one perturbagen.

Methods for counting cells are well known in the art. Non-limiting examples include hemocytometry, flow cytometry, and cell sorting techniques, e.g., fluorescence activated cell sorting (FACS). The maturation of the erythrocytes is, in some embodiments, determined by loss of CD71 expression. Erythroid maturation is determined by, in some embodiments, flow cytometry using a four-antibody panel (See Example 2 infra) (CD71, CD235a, CD233, CD49d) with increased CD233 expression, with a concomitant loss of CD49d expression, and a shift in CD71^{Hi} to CD71^{low} erythroid population (CD235a+).

In some embodiments, the change in cell state of a progenitor cell provides an increase in the number of one or more of proerythroblasts, early erythroblasts, intermediate erythroblasts, late erythroblasts, reticulocytes, and erythrocytes. In this aspect, one or more of the proerythroblasts, early erythroblasts, intermediate erythroblasts, late erythroblasts, reticulocytes, and erythrocytes comprise fetal hemoglobin (HbF). In some embodiments, one or more of the proerythroblasts, early erythroblasts, intermediate erythroblasts, late erythroblasts, reticulocytes, and erythrocytes expresses HBG1 and/or HBG2.

In an embodiment, the change in cell state of a progenitor cell provides an increase in F cells. See Boyer et al. 1975. Fetal hemoglobin restriction to a few erythrocytes (F cells) in normal human adults. Science 188: 361-363

In some embodiments, the increase in the number of erythrocytes comprising HbF is relative to the number of erythrocytes obtained from a population of progenitor cells that is not contacted with the at least one perturbagen. In other embodiments, the increase in the number of erythrocytes comprising HbF is relative to the population of progenitor cells prior to contacting with the at least one perturbagen.

In some embodiments, the change in cell state of a progenitor cell provides an increase in the number of erythrocytes comprising HbF.

In an embodiment, the change in cell state provides an increase in the ratio of the number of erythrocytes comprising HbF to the number of progenitor cells relative to the ratio obtained from a population of progenitor cells that is not contacted with the at least one perturbagen. In an embodiment, the change in cell state provides an increase in the ratio of the number of erythrocytes comprising HbF to the number of progenitor cells relative to the ratio in the population of progenitor cells prior to contacting with the at least one perturbagen.

In an embodiment, the increase in the number of erythrocytes comprising HbF, is due in part to increased cell proliferation of the erythrocytes comprising HbF. In other embodiments, the increase in the number of erythrocytes comprising HbF, is due in part to an increased lifespan of the erythrocytes comprising HbF. In other embodiments, the increase in the number of erythrocytes comprising HbF, is due in part to reduced cell death among the erythrocytes comprising HbF. In other embodiments, the increase in the number of erythrocytes comprising HbF, is due in part to a change of cell state from progenitor cells into the erythrocyte lineage.

In an embodiment, the change in cell state provides a decrease in the number of progenitor cells. In embodiments, the decrease in the number of progenitor cells is due in part to decreased cell proliferation of the progenitor cells. In other embodiments, the decrease in the number of progenitor cells is due in part to a decreased lifespan of the progenitor cells. In other embodiments, the decrease in the number of progenitor cells is due in part to increased cell death among the progenitor cells. In some embodiments, the decrease in the number of progenitor cells is relative to the number of progenitor cells in a population of progenitor cells that is not contacted with the at least one perturbagen. In some embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into the erythrocyte lineage.

In an embodiment, the change in cell state provides an increase in the number of progenitor cells. In embodiments, the increase in the number of progenitor cells is due in part to increased cell proliferation of the progenitor cells. In other embodiments, the increase in the number of progenitor cells is due in part to an increased lifespan of the progenitor cells. In other embodiments, the increase in the number of progenitor cells is due in part to decreased cell death among the progenitor cells. In some embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In an embodiment, the change in cell state provides an increase in the number of proerythroblasts, BFU-E cells, CFU-E cells, early erythroblasts, intermediate erythroblasts, late erythroblasts, reticulocytes, and/or erythrocytes after contacting the population of cells comprising a CD34⁺ cell with the at least one perturbagen. In embodiments, one or more of the proerythroblasts, early erythroblasts, intermediate erythroblasts, late erythroblasts, reticulocytes, and erythrocytes comprise HbF.

In an embodiment, the change in cell state provides an increase in the ratio of the number of other committed blood cells to the number of progenitor cells relative to the ratio obtained from a population of progenitor cells that is not contacted with the at least one perturbagen.

In an embodiment, the change in cell state provides an increase in the ratio of the number of proerythroblasts, BFU-E cells, CFU-E cells, early erythroblasts, intermediate erythroblasts, late erythroblasts, reticulocytes, and/or erythrocytes to the number of progenitor cells relative to the ratio obtained from a population of progenitor cells that is not contacted with the at least one perturbagen. In embodiments, one or more of the proerythroblasts, early erythroblasts, intermediate erythroblasts, late erythroblasts, reticulocytes, and erythrocytes comprise HbF. In other embodiments, the ratio of the number proerythroblasts, BFU-E cells, CFU-E cells, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen. In embodiments, one or more of the proerythroblasts, early erythroblasts, intermediate erythroblasts, late erythroblasts, reticulocytes, and erythrocytes comprise HbF.

In some embodiments, the change in cell state provides an increase in the number of proerythroblasts and/or erythrocytes comprising HbF after contacting the population of cells comprising a CD34+ cell with the at least one perturbagen. In other embodiments, the number of reticulocytes comprising HbF, and/or erythrocytes comprising HbF is increased after contacting the population of cells comprising a CD34+ cell with the at least one perturbagen.

In some embodiments, the change in cell state provides an increase in the ratio of the number of early erythroblasts, intermediate erythroblasts, late erythroblasts, reticulocytes, and/or erythrocytes to the number of proerythroblasts relative to the ratio obtained from a population of progenitor cells that is not contacted with the at least one perturbagen. In embodiments, one or more of the proerythroblasts, early erythroblasts, intermediate erythroblasts, late erythroblasts, reticulocytes, and erythrocytes comprise HbF.

In some embodiments, the change in cell state provides an increase in the ratio of the number of HbF-expressing early erythroblasts, intermediate erythroblasts, and/or late erythroblasts to the number of HbF-expressing proerythroblasts relative to the ratio obtained from a population of progenitor cells that is not contacted with the at least one perturbagen. In other embodiments, the ratio of the number of HbF-expressing early erythroblasts, intermediate erythroblasts, and/or late erythroblasts to the number of HbF-expressing proerythroblasts is increased relative to the ratio in the population of progenitor cells prior to contacting with the at least one perturbagen.

In other embodiments, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen. In embodiments, one or more of the proerythroblasts, early erythroblasts, intermediate erythroblasts, late erythroblasts, reticulocytes, and erythrocytes comprise HbF.

In some embodiments, the change in cell state provides an increase in the ratio of the number of HbF-expressing early erythroblasts to the number of HbF-expressing proerythroblasts relative to the ratio obtained from a population of progenitor cells that is not contacted with the at least one perturbagen. In other embodiments, the ratio of the number of HbF-expressing early erythroblasts to the number of HbF-expressing proerythroblasts is increased relative to the ratio in the population of progenitor cells prior to contacting with the at least one perturbagen.

In some embodiments, the change in cell state provides an increase in the ratio of the number of HbF-expressing intermediate erythroblasts to the number of HbF-expressing early erythroblasts relative to the ratio obtained from a population of progenitor cells that is not contacted with the at least one perturbagen. In other embodiments, the ratio of the number of HbF-expressing intermediate erythroblasts to the number of HbF-expressing early erythroblasts is increased relative to the ratio in the population of progenitor cells prior to contacting with the at least one perturbagen.

In some embodiments, the change in cell state provides an increase in the ratio of the number of HbF-expressing late erythroblasts to the number of HbF-expressing intermediate erythroblasts relative to the ratio obtained from a population of progenitor cells that is not contacted with the at least one perturbagen. In other embodiments, the ratio of the number of HbF-expressing late erythroblasts to the number of HbF-expressing intermediate erythroblasts is increased relative to the ratio in the population of progenitor cells prior to contacting with the at least one perturbagen.

In some embodiments, the change in cell state provides an increase in the ratio of the number of reticulocytes comprising HbF to the number of HbF-expressing late erythroblasts relative to the ratio obtained from a population of progenitor cells that is not contacted with the at least one perturbagen. In other embodiments, the ratio of the number of reticulocytes comprising HbF to the number of HbF-expressing late erythroblasts is increased relative to the ratio in the population of progenitor cells prior to contacting with the at least one perturbagen.

In some embodiments, the change in cell state provides an increase in the ratio of the number of erythrocytes comprising HbF to the number of reticulocytes comprising HbF relative to the ratio obtained from a population of progenitor cells that is not contacted with the at least one perturbagen. In other embodiments, the ratio of the number of erythrocytes comprising HbF to the number of reticulocytes comprising HbF is increased relative to the ratio in the population of progenitor cells prior to contacting with the at least one perturbagen.

In some embodiments, the change in cell state provides an increase in the ratio of the number of erythrocytes comprising HbF to HbF-expressing late erythroblasts relative to the ratio obtained from a population of progenitor cells that is not contacted with the at least one perturbagen. In other embodiments, the ratio of the number of erythrocytes comprising HbF to HbF-expressing late erythroblasts is increased relative to the ratio in the population of progenitor cells prior to contacting with the at least one perturbagen.

In some embodiments, the change in cell state provides an increase in the ratio of the number of erythrocytes comprising HbF to HbF-expressing intermediate erythroblasts relative to the ratio obtained from a population of progenitor cells that is not contacted with the at least one perturbagen. In other embodiments, the ratio of the number of erythrocytes comprising HbF to HbF-expressing intermediate erythroblasts is increased relative to the ratio in the population of progenitor cells prior to contacting with the at least one perturbagen.

In some embodiments, the change in cell state provides an increase in the ratio of the number of erythrocytes comprising HbF to HbF-expressing early erythroblasts relative to the ratio obtained from a population of progenitor cells that is not contacted with the at least one perturbagen. In other embodiments, the ratio of the number of erythrocytes comprising HbF to HbF-expressing early erythroblasts is increased relative to the ratio in the population of progenitor cells prior to contacting with the at least one perturbagen.

In some embodiments, the change in cell state provides an increase in the ratio of the number of erythrocytes comprising HbF to HbF-expressing proerythroblasts relative to the ratio obtained from a population of progenitor cells that is not contacted with the at least one perturbagen. In other embodiments, the ratio of the number of erythrocytes comprising HbF to HbF-expressing proerythroblasts is increased relative to the ratio in the population of progenitor cells prior to contacting with the at least one perturbagen.

In some embodiments, the change in cell state provides an increase in the ratio of the number of erythrocytes comprising HbF to proerythroblasts relative to the ratio obtained from a population of progenitor cells that is not contacted with the at least one perturbagen. In other embodiments, the ratio of the number of erythrocytes comprising HbF to proerythroblasts is increased relative to the ratio in the population of progenitor cells prior to contacting with the at least one perturbagen.

In some embodiments, the change in cell state provides an increase in the ratio of the number of erythrocytes comprising HbF and/or reticulocytes to the number of HbF-expressing early erythroblasts, intermediate erythroblasts, and/or late erythroblasts relative to the ratio obtained from a population of progenitor cells that is not contacted with the at least one perturbagen. In other embodiments, the ratio of the number of erythrocytes comprising HbF and/or reticulocytes to the number of HbF-expressing early erythroblasts, intermediate erythroblasts, and/or late erythroblasts is increased relative to the ratio in the population of progenitor cells prior to contacting with the at least one perturbagen.

In some embodiments, the change in cell state provides an increase in the ratio of the number of erythrocytes comprising HbF to the number of HbF-expressing early erythroblasts, intermediate erythroblasts, and/or late erythroblasts relative to the ratio obtained from a population of progenitor cells that is not contacted with the at least one perturbagen. In other embodiments, the ratio of the number of erythrocytes comprising HbF to the number of HbF-expressing early erythroblasts, intermediate erythroblasts, and/or late erythroblasts is increased relative to the ratio in the population of progenitor cells prior to contacting with the at least one perturbagen. In some embodiments, the change in cell state provides an increase in the ratio of the number of reticulocytes comprising HbF to the number of HbF-expressing early erythroblasts, intermediate erythroblasts, and/or late erythroblasts relative to the ratio obtained from a population of progenitor cells that is not contacted with the at least one perturbagen. In other embodiments, the ratio of the number of reticulocytes comprising HbF to the number of HbF-expressing early erythroblasts, intermediate erythroblasts, and/or late erythroblasts is increased relative to the ratio in the population of progenitor cells prior to contacting with the at least one perturbagen.

In some embodiments, the change in cell state provides an increase in the ratio of the number of erythrocytes comprising HbF to reticulocytes comprising HbF relative to the ratio obtained from a population of progenitor cells that is not contacted with the at least one perturbagen. In other embodiments, the ratio of the number of erythrocytes comprising HbF to reticulocytes comprising HbF is increased relative to the ratio in the population of progenitor cells prior to contacting with the at least one perturbagen.

In one embodiment, the change in cell state provides a decrease in the number of proerythroblasts. In another embodiment, the change in cell state provides a decrease in the number of HbF-negative or HbF-low proerythroblasts. In another embodiment, the number of HbF-negative or HbF-low early erythroblasts is decreased. In another embodiment, the number of HbF-negative or HbF-low intermediate erythroblasts is decreased. In another embodiment, the number of HbF-negative or HbF-low late erythroblasts is decreased. In another embodiment, the number of HbF-negative or HbF-low reticulocytes is decreased.

In one embodiment, the change in cell state provides an increase in the number of proerythroblasts. In another embodiment, the number of HbF-positive or HbF-high proerythroblasts is increased. In another embodiment, the number of HbF-positive or HbF-high early erythroblasts is increased. In another embodiment, the number of HbF-positive or HbF-high intermediate erythroblasts is increased. In another embodiment, the number of HbF-positive or HbF-high late erythroblasts is increased. In another embodiment, the number of HbF-positive or HbF-high reticulocytes is increased. In another embodiment, the number of HbF-positive or HbF-high erythrocytes is increased.

In an embodiment, the change in cell state provides an increase in the number of F cells. In another embodiment, the ratio of the number of F cells to non-F cells is increased relative to the ratio obtained from a population of progenitor cells that is not contacted with the at least one perturbagen. In another embodiment, the ratio of the number of F cells to non-F cells is increased relative to the ratio in the population of progenitor cells prior to contacting with the at least one perturbagen.

In some embodiments, contacting the population of progenitor cells with the at least one perturbagen occurs in vitro or ex vivo. In an embodiment, contacting the population of progenitor cells with the at least one perturbagen occurs in vivo in a subject. In an embodiment, the subject is a human. In some embodiments, the human is an adult human. In some embodiments, the adult human has an abnormal number of one or more of erythroblasts, or erythrocytes, or a disease or disorder characterized thereby. In some embodiments, the adult human has a disease or disorder characterized by abnormal oxygen delivery, or hemoglobin deficiency. In some embodiments, the adult human suffers from a sickle cell disease or a thalassemia.

Percentage of fetal hemoglobin (% HbF) and fetal hemoglobin containing red blood cells (% F+ cells) are important parameters for determining the efficacy of the perturbagens as described above at promoting a fetal hemoglobin (HbF) cell state. % HbF is the proportion of HbF in the total Hb in hemolysate, which ignores the numbers of red blood cells. % F⁺ cells is the proportion of the HbF containing red blood cells in total red blood cells. (See Mundee et al., Cytometry (Communication in Clinical Cytometry), 2000, vol. 42, p. 389-393).

Methods for assaying % HbF and % F⁺ cells are well known in the art. Non-limiting examples include high-performance liquid chromatography (HPLC), flow cytometry, or ion-exchange chromatography. The HbF % is usually measured by HPLC. The flow cytometry assay, the standard clinical method, may be used for assaying % F⁺ cells by immunofluorescent techniques. In addition to flow cytometry, ion-exchange chromatography may be used to measure the fraction HbF relative to all other hemoglobin (HbF/HbA+HbF) (See Example 2 below).

The baseline level of HbF and F cells in the total blood may serve as control for determining the efficacy of the perturbagens upon induction of HbF in red blood cells. Non-limiting examples of HbF level in a subject may serve as baseline % HbF include DMSO negative control, a normal individual, a normal individual of specific ethnicity, an individual having sickle cell disease, an individual having sickle cell disease with hydroxyurea treatment, or a population of erythrocytes having specific % HbF etc.

The mean % HbF and % F⁺ cells in normal adults is 1.0%±0.1% and 3.0%±0.4%. The normal adult is considered to have very low % HbF and % F⁺ cells. The mean % HbF of about 3.0% or less is considered as low % HbF. The mean % HbF ranging from about 4.0% to 12.0% is considered as medium % HbF, for example, mean % HbF and % F⁺ cells in sickle cell patient is 4.1%±0.8% and 14.8%±1.8%. The mean % HbF of about 13.0% or higher is considered as high % HbF, e.g., the mean % HbF and % F⁺ cells in sickle cell patient treated with hydroxyurea is 15.8%±2.0% and 54.1%±8.5%. (See Mundee et al. supra). In embodiments, % HbF is measured by HPLC. In embodiments, % F+ cells are measured by flow cytometry.

In various embodiments, the baseline % HbF and induced % HbF is gated by vehicle control and % HbF induced by 50 μM hydroxyurea (Example 2 infra). It is to be understood that the HbF-low and HbF-high F+ cells characterization described above are classified by the mean % HbF values that typically presents in normal adult, patient having sickle cell disease and sickle cell disease patent with hydroxyurea treatment. It is to be understood that the absolute value ranges for mean % HbF that characterized as low and high % HbF may vary depends on the assay techniques and detection limits thereof.

In some embodiments, the at least one perturbagen selected from Table 4, or a variant thereof, comprises at least 2, at least 3, at least 4, or at least 5 perturbagens selected from Table 4, or variants thereof.

In some embodiments, altering the gene signature comprises increased expression and/or increased activity in the progenitor cell of one or more genes selected from the genes designated as an “up” gene in the gene directionality column of Table 1. In some embodiments, the one or more genes selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, genes. In another embodiment, the one or more genes selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1.

In some embodiments, altering the gene signature comprises decreased expression and/or decreased activity in the progenitor cell of one or more genes selected from the genes designated as a “down” gene in the gene directionality column of Table 1. In some embodiments, the one or more genes selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1. In some embodiments, the one or more genes selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2.

In some embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In an aspect, the present disclosure provides a method for promoting the formation of a erythrocytes or an immediate progenitor thereof. The method includes a step of exposing a starting population of stem/progenitor cells comprising a non-lineage committed CD34⁺ cell to a perturbation having a perturbation signature that promotes the transition of the starting population of stem/progenitor cells into a HbF-expressing proerythroblasts, early erythroblasts, intermediate erythroblasts, and/or late erythroblasts. In embodiments, the perturbation signature comprises increased expression and/or activity of one or more of genes selected from the genes designated as an “up” gene in the gene directionality column of Table 1 and/or a decreased expression and/or activity in the non-lineage committed CD34⁺ cell of one or more genes selected from the genes designated as a “down” gene in the gene directionality column of Table 1. Embodiments associated with the above aspects are likewise relevant to the present aspect. In other words, each of the embodiment mentioned above for the above aspects may be revised/adapted to be applicable to the present aspect.

In another aspect, the present disclosure provides a method of increasing a quantity of reticulocytes comprising HbF and/or erythrocytes or progenitors thereof. The method includes a step of exposing a starting population of stem/progenitor cells comprising a non-lineage committed CD34⁺ cell to a pharmaceutical composition that promotes the formation of lineage specific progenitor population selected from proerythroblasts, early erythroblasts, intermediate erythroblasts, and/or late erythroblasts. The pharmaceutical composition promotes the transition of a primitive stem/progenitor population into the lineage specific progenitor population that has the capacity to differentiate into reticulocytes comprising HbF and/or erythrocytes or progenitors thereof. In embodiments, the pharmaceutical composition comprises at least one perturbagen selected from Table 4, or a variant thereof. Embodiments associated with the above aspects are likewise relevant to the present aspect. In other words, each of the embodiment mentioned above for the above aspects may be revised/adapted to be applicable to the present aspect.

In yet another aspect, the present disclosure provides a perturbagen for use in any herein disclosed method.

In a further aspect, the present disclosure provides a pharmaceutical composition comprising perturbagen for use in any herein disclosed method.

### Methods and Perturbagens for Treating a Disease or Disorder

### Fetal Hemaglobin

The major β-globin disorders, for example, sickle cell disease (SCD) and β-thalassemia, are classical Mendelian anemias caused by mutations in the adult β-globin gene. β-thalassemia results from mutations that decrease or ablate β-globin production. A single amino acid alteration, glutamine-valine substitution at codon 6 in the β-globin protein, leads to SCD and polymerization of deoxy sickle hemoglobin (HbS). Patients with elevated levels of fetal hemoglobin (also known as hemoglobin F, HbF, γ-globin) as adults exhibit reduced symptoms and enhanced survival.

HbF is the major hemoglobin produced during fetal life but is largely replaced by adult hemoglobin (HbA) following a “switch” around birth in normal individuals. In individuals having SCD, HbF is replaced by HbS. In most adults, HbF production is restricted to a small number of erythroid precursors and their progeny in the blood are F-cells. The γ-globin genes of fetal hemoglobin can be reactivated in adult individuals by several pharmacologic means and physiological manipulations.

Hydroxyurea (HU), a FDA-approved drug for SCD, is a potent inducer for HbF (γ-globin gene expression). While HU is effective for many patients, its utility is limited due to unpredictable induction of HbF and marginal benefit for patients with β-thalassemia. There exists a need for more effective therapies based on HbF reactivation. HU induces the expression of HbF via the mechanism of action as a potent inhibitor of ribonucleotide reductase.

In some embodiments, the therapeutic induction of HbF is through the transcriptional regulation of the human globin genes, for example, regulating chromatin modifying enzymes such as histone deacetylase (HDACs) with epigenetic regulators including selective and/or non-selective HDAC inhibitors (Brander et al., Chemical genetic strategy identifies histone deacetylase 1 (HDAC1) and HDAC 2 as therapeutic targets in sickle cell disease, PNAS, 2010, vol. 107, pp. 12617-12622). In some embodiments, the therapeutic induction of HbF is through the inhibition of BCL11A and KLF1 gene expressions (Steinberg et al., Blood, 2014, vol. 123, pp. 481-485).

The complexity of the HbF regulation suggests that combination therapy of different agents (e.g. one or more perturbagens, and/or another agent described herein, each optionally with a different mechanism of action, is an effective strategy for the induction of very high level of HbF while optionally limiting adverse effects. For example, combined therapy with hydroxyurea and recombinant erythropoietin elevates HbF level more than hydroxyurea alone in SCD patients (Roger et al., N Engl. J. Med. 1993, vol. 328, pp. 73-80).

In some embodiments, the present methods of treatment further comprise administration of one or more of recombinant erythropoietin and hydroxyurea. In some embodiments, the present methods of treatment involve a subject undergoing treatment with one or more of recombinant erythropoietin and hydroxyurea In some embodiments, the present methods of treatment further comprise administration of a P-selectin antibody, e.g. crizanlizumab. In some embodiments, the present methods of treatment involve a subject undergoing treatment with a P-selectin antibody, e.g. crizanlizumab.

An aspect of the present disclosure is a method for treating a disease or disorder characterized by an abnormal oxygen delivery. In this aspect, the method comprises the step of administering to a subject in need thereof a therapeutically effective amount of at least one perturbagen capable of altering a gene associated with at least one functionality in a progenitor cell.

An aspect of the present disclosure is a method for treating a disease or disorder characterized by an abnormal oxygen delivery. In this aspect, the method comprises the step of administering to a subject in need thereof a cell, the cell having been contacted with at least one perturbagen capable of altering a gene associated with at least one functionality in a progenitor cell.

An aspect of the present disclosure is a method for treating a disease or disorder characterized by an abnormal oxygen delivery. In this aspect, the method comprises the step of administering to a subject in need thereof a therapeutically effective amount of a combination therapy having hydroxyurea and at least one perturbagen, wherein the combination therapy is capable of altering a gene associated with at least one functionality in a progenitor cell. Another aspect of the present disclosure is a method for treating a disease or disorder characterized by an abnormal oxygen delivery. In this aspect, the method comprises the step of administering to a subject in need thereof a cell, the cell having been contacted with a combination therapy having hydroxyurea and at least one perturbagen, wherein the combination therapy is capable of altering a gene associated with at least one functionality in a progenitor cell.

An aspect of the present disclosure is a method for treating a disease or disorder characterized by a hemoglobin deficiency. In this aspect, the method comprises the step of administering to a subject in need thereof a therapeutically effective amount of at least one perturbagen capable of altering a gene associated with at least one functionality in a progenitor cell.

Another aspect of the present disclosure is a method for treating a disease or disorder characterized by a hemoglobin deficiency. In this aspect, the method comprises the step of administering to a subject in need thereof a cell, the cell having been contacted with at least one perturbagen capable of altering a gene associated with at least one functionality in a progenitor cell.

An aspect of the present disclosure is a method for treating a disease or disorder characterized by a hemoglobin deficiency. In this aspect, the method comprises the step of administering to a subject in need thereof a therapeutically effective amount of a combination therapy having hydroxyurea and at least one perturbagen, wherein the combination therapy is capable of altering a gene associated with at least one functionality in a progenitor cell.

Another aspect of the present disclosure is a method for treating a disease or disorder characterized by a hemoglobin deficiency. In this aspect, the method comprises the step of administering to a subject in need thereof a cell, the cell having been contacted with a combination therapy having hydroxyurea and at least one perturbagen, wherein the combination therapy is capable of altering a gene associated with at least one functionality in a progenitor cell.

An aspect of the present disclosure is a method for treating or preventing a sickle cell disease or a thalassemia. In this aspect, the method comprises the step of administering to a subject in need thereof a therapeutically effective amount of at least one perturbagen capable of altering a gene associated with at least one functionality in a progenitor cell.

Another aspect of the present disclosure is a method for treating or preventing a sickle cell disease or a thalassemia. In this aspect, the method comprises the step of administering to a subject in need thereof a cell, the cell having been contacted with at least one perturbagen capable of altering a gene associated with at least one functionality in a progenitor cell.

An aspect of the present disclosure is a method for treating or preventing a sickle cell disease or a thalassemia. In this aspect, the method comprises the step of administering to a subject in need thereof a therapeutically effective amount of a combination therapy of hydroxyurea and at least one perturbagen, wherein the combination therapy is capable of altering a gene associated with at least one functionality in a progenitor cell. Another aspect of the present disclosure is a method for treating or preventing a sickle cell disease or a thalassemia. In this aspect, the method comprises the step of administering to a subject in need thereof a cell, the cell having been contacted with a combination therapy of hydroxyurea and at least one perturbagen, wherein the combination therapy is capable of altering a gene associated with at least one functionality in a progenitor cell.

An aspect of the present disclosure is a method for treating a disease or disorder characterized by an abnormal oxygen delivery. The method comprises the steps of: (a) administering to a subject in need thereof a therapeutically effective amount of at least one perturbagen selected from Table 4, or a variant thereof, wherein the at least one perturbagen is capable of altering a gene associated with at least one functionality in a progenitor cell or (b) administering to a subject in need thereof a cell, the cell having been contacted with at least one perturbagen selected from Table 4, or a variant thereof.

In another aspect, the present disclosure provides a method for treating a disease or disorder characterized by a hemoglobin deficiency. The method comprises the steps of: (a) administering to a subject in need thereof a therapeutically effective amount of at least one perturbagen selected from Table 4, or a variant thereof, wherein the at least one perturbagen is capable of altering a gene associated with at least one functionality in a progenitor cell or (b) administering to a subject in need thereof a cell, the cell having been contacted with at least one perturbagen selected from Table 4, or a variant thereof. In some embodiments, the hemoglobin deficiency is an abnormal and/or reduced oxygen delivery functionality of hemoglobin, optionally resultant from mutations in one or more beta-like hemoglobin subunit genes, an exemplary mutation being in the adult HBB gene.

In some embodiments, for any herein disclosed method, the disease or disorder characterized by an abnormal oxygen delivery and/or a hemoglobin deficiency is an anemia.

In some embodiments, for any herein disclosed method, the administering is directed to the bone marrow of the subject. In some embodiments, for any herein disclosed method, the administering is via intraosseous injection or intraosseous infusion. In some embodiments, for any herein disclosed method, the administering the cell is via intravenous injection or intravenous infusion. In some embodiments, the administering is simultaneously or sequentially to one or more mobilization agents.

In another aspect, the present disclosure provides a method for treating or preventing a sickle cell disease or a thalassemia. The method comprises the steps of: (a) administering to a subject in need thereof a therapeutically effective amount of at least one perturbagen selected from Table 4, or a variant thereof, where the at least one perturbagen is capable of altering a gene associated with at least one functionality in a progenitor cell or (b) administering to a subject in need thereof a cell, the cell having been contacted with at least one perturbagen selected from Table 4, or a variant thereof.

In some embodiments, for any herein disclosed method, the sickle cell disease or a thalassemia is beta-thalassemia (transfusion dependent). In some embodiments, for any herein disclosed method, the sickle cell disease or a thalassemia is beta-thalassemia major. In some embodiments, for any herein disclosed method, the sickle cell disease or a thalassemia is beta-thalassemia intermedia. In some embodiments, for any herein disclosed method, the sickle cell disease or a thalassemia is beta-thalassemia minor. In a further embodiment, for any herein disclosed method, the sickle cell disease or a thalassemia is sickle cell anemia (SS), sickle hemoglobin-C disease (SC), or sickle beta-plus thalassemia and sickle beta-zero thalassemia.

In some embodiments, for any herein disclosed method, the at least one perturbagen is capable of altering a gene associated with at least one functionality selected from the functionality of the genes designated as an “up” gene in the gene directionality column of Table 1 and/or the genes designated as a “down” gene in the gene directionality column of Table 1.

In an embodiment, the subject is selected by steps including: obtaining from the subject having the disease or disorder a sample of cells comprising a non-lineage committed CD34⁺ cell; and contacting the sample of cells with least one perturbagen selected from Table 4, or a variant thereof. In this aspect, the at least one perturbagen alters a gene associated with at least one functionality in the sample of cells. In some embodiments, the subject is selected by steps including: obtaining from a subject having the disease or disorder a sample of cells comprising a non-lineage committed CD34⁺ cell; and contacting the sample of cells with at least one perturbagen capable of altering a gene associated with at least one functionality in a non-lineage committed CD34⁺ cell. In this aspect, the at least one perturbagen increases in the sample of cells the expression and/or activity of a gene associated with at least one functionality selected from the functionality of the genes designated as an “up” gene in the gene directionality column of Table 1 and/or decreases in the sample of cells the expression and/or activity of one or more genes associated with at least one functionality selected from the functionality of the genes designated as a “down” gene in the gene directionality column of Table 1.

In an embodiment, the subject is selected by steps including: obtaining from the subject having the disease or disorder a sample of cells comprising a non-lineage committed CD34⁺ cell; and contacting the sample of cells with least one perturbagen selected from Table 4, or a variant thereof. In this aspect, the at least one perturbagen increases in the sample of cells the expression and/or activity of a gene associated with at least one functionality selected from the functionality of the genes designated as an “up” gene in the gene directionality column of Table 1 and/or decreases in the sample of cells the expression and/or activity of one or more genes associated with at least one functionality selected from the functionality of the genes designated as a “down” gene in the gene directionality column of Table 1.

In an embodiment, the subject is selected by steps including: obtaining from the subject having the disease or disorder a sample of cells comprising a non-lineage committed CD34⁺ cell; and contacting the sample of cells with least one perturbagen selected from Table 4, or a variant thereof. In this aspect, when the at least one perturbagen alters a gene associated with at least one functionality in the sample of cells, the subject is selected as a subject.

In some embodiments, the present disclosure provides a method for selecting the subject for the treatment described above, the method including the steps of: obtaining from a subject having the disease or disorder a sample of cells comprising a non-lineage committed CD34⁺ cell; and contacting the sample of cells with at least one perturbagen capable of altering a gene associated with at least one functionality in a non-lineage committed CD34⁺ cell. In this aspect, when the at least one perturbagen causes the increases in the sample of cells the expression and/or activity of a gene associated with at least one functionality selected from the functionality of the genes designated as an “up” gene in the gene directionality column of Table 1 and/or decreases in the sample of cells the expression and/or activity of one or more genes associated with at least one functionality selected from the functionality of the genes designated as a “down” gene in the gene directionality column of Table 1, the subject is selected as a subject.

In an embodiment, the subject is selected by steps including: obtaining from the subject having the disease or disorder a sample of cells comprising a non-lineage committed CD34⁺ cell; and contacting the sample of cells with least one perturbagen selected from Table 4, or a variant thereof. In this aspect, when the at least one perturbagen causes the increases in the sample of cells the expression and/or activity of a gene associated with at least one functionality selected from the functionality of the genes designated as an “up” gene in the gene directionality column of Table 1 and/or decreases in the sample of cells the expression and/or activity of one or more genes associated with at least one functionality selected from the functionality of the genes designated as a “down” gene in the gene directionality column of Table 1, the subject is selected as a subject.

In some embodiments, the present disclosure provides a method for selecting the subject for the treatment described above, the method including the steps of: obtaining from a subject having the disease or disorder a sample of cells comprising a non-lineage committed CD34⁺ cell; and contacting the sample of cells with at least one perturbagen selected from Table 4, or a variant thereof. In this aspect, when the at least one perturbagen increases in the sample of cells the expression and/or activity of one or more genes selected from the genes designated as an “up” gene in the gene directionality column of Table 1 and/or decreases in the sample of cells the expression and/or activity of one or more genes selected from the genes designated as a “down” gene in the gene directionality column of Table 1, the subject is selected as a subject.

An aspect of the present disclosure provides use of the perturbagen of Table 4, or a variant thereof in the manufacture of a medicament for treating a disease or disorder characterized by an abnormal oxygen delivery or a hemoglobin deficiency. In another aspect, the present disclosure provides use of the perturbagen of Table 4, or a variant thereof in the manufacture of a medicament for treating sickle cell disease or a thalassemia.

An aspect of the present disclosure is related to a method of identifying a candidate perturbation for promoting the transition of a starting population of progenitor cells into erythrocytes comprising HbF or HbF-expressing progenitors thereof. The method include the steps of: exposing the starting population of progenitor cells to a perturbation identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation, identifying the perturbation as a candidate perturbation for promoting the transition of a population of progenitor cells into erythrocytes comprising HbF or HbF-expressing progenitors thereof based on the perturbation signature. In this aspect, the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as a “down” gene in the gene directionality column of Table 1.

Further, in this aspect, in some embodiments, altering the gene signature comprises an increase in expression and/or activity in the progenitor cell of a network module designated in the network module column of Table 1. Further, in this aspect, in some embodiments, the activation of one or more genes of the network module designated in the network module column of Table 1 comprises modulating expression and/or activity of 2 or more genes within a network module. Further, in some embodiments of this aspect, altering the gene signature comprises an increase in expression and/or activity in the progenitor cell of two or more genes designated as an “up” gene in the gene directionality column of Table 1. Further, in this aspect, in some embodiments, altering the gene signature comprises a decrease in expression and/or activity in the progenitor cell of two or more genes designated as a “down” gene in the gene directionality column of Table 1.

In another aspect, the present disclosure provides a method for making a therapeutic agent for a disease or disorder selected from a sickle cell disease, or a thalassemia, or a disease or disorder characterized by an abnormal oxygen delivery or a hemoglobin deficiency. In this aspect, the method comprises the steps of: (a) identifying a candidate perturbation for therapy as described above and (b) formulating the candidate perturbation as a therapeutic agent for the treatment of the disease or disorder.

In some embodiments, the promoting the transition of a starting population of progenitor cells into erythrocytes comprising HbF or HbF-expressing progenitors thereof occurs in vitro or ex vivo. In an embodiment, promoting the transition of a starting population of progenitor cells into erythrocytes comprising HbF or HbF-expressing progenitors thereof occurs in vivo in a subject. In an embodiment, the subject is a human. In an embodiment, the human is an adult human.

The efficacy of the treatment with at least one perturbagen in the subject may be measured by an absolute increase or relative increase of HbF %, and or the F-cell percentage increase. % HbF is the proportion of HbF in the total Hb in hemolysate, which ignores the numbers of red blood cells. % F⁺ cells is the proportion of the HbF containing red blood cells in total red blood cells. (See Mundee et al., Cytometry (Communication in Clinical Cytometry), 2000, vol. 42, p. 389-393).

In some embodiments, the at least one perturbagen induces at least 50% absolute increase or a 100% relative increase in HbF percentage levels (HbF %). In some embodiments, the mean % HbF and % F⁺ cells in the subject treated with at least one perturbagen is about 13.0% or greater as measured by, e.g., HPLC and about 45.0% or greater as measured by, e.g., immunofluorescence technique respectively. In some embodiments, the mean % HbF in the subject treated with at least one perturbagen ranges from about 25.0% to about 30.0% as measured by, e.g., HPLC.

In some embodiments, the mean % HbF in the subject treated with at least one perturbagen is selected from about 4.0%, about 5.0%, about 6.0%, about 7.0%, about 8.0%, about 9.0%, about 10.0%, about 11.0%, about 12.0%, about 13.0%, about 13.1%, about 13.2%, about 13.3%, about 13.4%, about 13.5%, about 13.6%, about 13.7%, about 13.8%, about 13.9%, about 14.0%, about 14.1%, about 14.2%, about 14.3%, about 14.4%, about 14.5%, about 14.6%, about 14.7%, about 14.8%, about 14.9%, about 15.0%, about 15.1%, about 15.2%, about 15.3%, about 15.4%, about 15.5%, about 15.6%, about 15.7%, about 15.8%, about 15.9%, about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 18.1%, about 18.2%, about 18.3%, about 18.4%, about 18.5%, about 18.6%, about 18.7%, about 18.8%, about 18.9%, about 20.0%, about 20.1%, about 20.2%, about 20.3%, about 20.4%, about 20.5%, about 20.6%, about 20.7%, about 20.8%, about 20.9%, about 21.0%, about 21.1%, about 21.2%, about 21.3%, about 21.4%, about 21.5%, about 21.6%, about 21.7%, about 21.8%, about 21.9%, about 22.0%, about 22.1%, about 22.2%, about 22.3%, about 22.4%, about 22.5%, about 22.6%, about 22.7%, about 22.8%, about 22.9%, about 23.0%, about 23.1%, about 23.2%, about 23.3%, about 23.4%, about 23.5%, about 23.6%, about 23.7%, about 23.8%, about 23.9%, about 24.0%, about 24.1%, about 24.2%, about 24.3%, about 24.4%, about 24.5%, about 24.6%, about 24.7%, about 24.8%, about 24.9%, about 25.0%, about 25.1%, about 25.2%, about 25.3%, about 25.4%, about 25.5%, about 25.6%, about 250.7%, about 25.8%, about 25.9%, about 26.0%, about 26.1%, about 26.2%, about 26.3%, about 26.4%, about 26.5%, about 26.6%, about 26.7%, about 26.8%, about 26.9%, about 27.0%, about 27.1%, about 27.2%, about 27.3%, about 27.4%, about 27.5%, about 27.6%, about 27.7%, about 27.8%, about 27.9%, about 28.0%, about 28.1%, about 28.2%, about 28.3%, about 28.4%, about 28.5%, about 28.6%, about 28.7%, about 28.8%, about 28.9%, about 29.0%, about 29.1%, about 29.2%, about 29.3%, about 29.4%, about 29.5%, about 29.6%, about 29.7%, about 29.8%, about 29.9%, and about 30.0% as measured by e.g., HPLC.

In some embodiments, the mean % HbF in the subject treated with at least one perturbagen is selected from about 25.0%, about 25.1%, about 25.2%, about 25.3%, about 25.4%, about 25.5%, about 25.6%, about 250.7%, about 25.8%, about 25.9%, about 26.0%, about 26.1%, about 26.2%, about 26.3%, about 26.4%, about 26.5%, about 26.6%, about 26.7%, about 26.8%, about 26.9%, about 27.0%, about 27.1%, about 27.2%, about 27.3%, about 27.4%, about 27.5%, about 27.6%, about 27.7%, about 27.8%, about 27.9%, about 28.0%, about 28.1%, about 28.2%, about 28.3%, about 28.4%, about 28.5%, about 28.6%, about 28.7%, about 28.8%, about 28.9%, about 29.0%, about 29.1%, about 29.2%, about 29.3%, about 29.4%, about 29.5%, about 29.6%, about 29.7%, about 29.8%, about 29.9%, and about 30.0% as measured by e.g., HPLC.

In some embodiments, the treatment with at least one perturbagen causes a median % F+ cells increase ranges about 0.1% to about 50% above the baseline % F+ cells as measured by immunofluorescence technique. In some embodiments, the treatment with at least one perturbagen causes a median % F+ cells increase selected from the group consisting of about 0.1%, about 0.5%, about 1.0%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 21.0%, about 22.0%, about 23.0%, about 24.0%, about 25.0%, about 26.0%, about 27.0%, about 28.0%, about 29.0%, about 30.0%, about 31.0%, about 32.0%, about 33.0%, about 34.0%, about 35.0%, about 36.0%, about 37.0%, about 38.0%, about 39.0%, about 40.0%, about 41.0%, about 42.0%, about 43.0%, about 44.0%, about 45.0%, about 46.0%, about 47.0%, about 48.0%, about 49.0%, and about 50.0% as measured by e.g., HPLC.

Any aspect or embodiment disclosed herein can be combined with any other aspect or embodiment as disclosed herein.

Administration, Dosing, and Treatment Regimens As examples, administration results in the delivery of one or more perturbagens disclosed herein into the bloodstream (via enteral or parenteral administration), or alternatively, the one or more perturbagens is administered directly to the site of hematopoietic cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11(1): 78-88; Hopman and DiPersio “Advances in Stem Cell Mobilization.” Blood Review, 2014, 28(1): 31-40; and Kim “Hematopoietic stem cell mobilization: current status and future perspective.” Blood Res. 2017 June; 52(2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed.

Any perturbagen disclosed herein can be administered in a single daily dose (also known as QD, qd or q.d.), or the total daily dosage can be administered in divided doses of twice daily (also known as BID, bid, or b.i.d.), three times daily (also known as TID, tid, or tid.), or four times daily (also known as QID, qid, or q.i.d.). Furthermore, any perturbagen disclosed herein can be administered continuously rather than intermittently throughout the dosage regimen.

### Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In some embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In some embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

In some embodiments, the present disclosure provides a pharmaceutical composition comprising the combination of two or more perturbagens selected from Table 4 for the treatment of a disease or disorder selected from the group consisting of sickle cell disease, thalassemia, disease or disorder characterized by an abnormal oxygen delivery, and hemoglobin deficiency.

In some embodiments, the present disclosure provides a pharmaceutical composition comprising the combination of two or more perturbagens, each with a different mechanism of action.

In some embodiments, the present disclosure provides a pharmaceutical composition comprising the combination of two or more perturbagens, each with a different mechanism of action, selected from Table 4 for the treatment of a disease or disorder selected from the group consisting of sickle cell disease, thalassemia, disease or disorder characterized by an abnormal oxygen delivery, and hemoglobin deficiency. In some embodiments, the present disclosure provides a pharmaceutical composition comprising the combination of two or more perturbagens, each with a different mechanism of action, selected from Table 4 for the treatment a disease or disorder selected from the group consisting of anemia, beta-thalassemia, sickle cell anemia (SS), sickle hemoglobin-C disease (SC), sickle beta-plus thalassemia, and sickle beta-zero thalassemia. In some embodiments, the present disclosure provides a pharmaceutical composition comprising the combination of two or more perturbagens, each with a different mechanism of action, selected from Table 4 for the treatment beta-thalassemia, or sickle cell anemia.

In some embodiments, the present disclosure provides a pharmaceutical composition comprising the combination of hydroxyurea and one or more perturbagens selected from Table 4 for the treatment of a disease or disorder selected from the group consisting of sickle cell disease, thalassemia, disease or disorder characterized by an abnormal oxygen delivery, and hemoglobin deficiency. In some embodiments, the present disclosure provides a pharmaceutical composition comprising the combination of hydroxyurea and one or more perturbagens selected from Table 4 for the treatment a disease or disorder selected from the group consisting of anemia, beta-thalassemia, sickle cell anemia (SS), sickle hemoglobin-C disease (SC), sickle beta-plus thalassemia, and sickle beta-zero thalassemia. In some embodiments, the present disclosure provides a pharmaceutical composition comprising the combination of hydroxyurea and one or more perturbagens selected from Table 4 for the treatment beta-thalassemia, or sickle cell anemia.

In some embodiments, the present disclosure provides a pharmaceutical composition comprising the combination of hydroxyurea and one or more HDAC inhibitors selected from Table 4.

In some embodiments, two or more perturbagens selected from Table 4 may be mixed into a single preparation or two or more perturbagens of the combination may be formulated into separate preparations for use in combination separately or at the same time. In some embodiments, the present disclosure provides a kit containing the two or more perturbagens selected from Table 4 of the combination, formulated into separate preparations. In some embodiments, the combination therapies, comprising more than one perturbagen, can be co-delivered in a single delivery vehicle or delivery device.

As used herein, the term “combination” or “pharmaceutical combination” refers to the combined administration of the perturbagens. The combination of two or more perturbagens may be formulated as fixed dose combination or co-packaged discrete perturbagen dosages. In some embodiments, the fixed dose combination therapy of perturbagens comprises bilayer tablet, triple layer tablet, multilayered tablet, or capsule having plurality populations of particles of perturbagens. In some embodiments, the combination of two or more perturbagens may be administered to a subject in need thereof, e.g., concurrently or sequentially.

In some embodiments, the combination therapies of perturbagens as described above give synergistic effects on induction of HbF in a subject. The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination (Chou, Cancer Res. 2010, vol. 70, pp. 440-446). When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In some embodiments, any perturbagens disclosed herein is formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiment mentioned above for the above aspects may be revised/adapted to be applicable to the below aspects.

Yet another aspect of the present disclosure is a use of the perturbagen of Table 4, or a variant thereof in the manufacture of a medicament for treating a disease or disorder characterized by an abnormal oxygen delivery, hemoglobin deficiency, major β-globin, sickle cell disease, and thalassemia.

In another aspect, the present disclosure provides a method of identifying a candidate perturbation for promoting the transition of a starting population of progenitor cells into erythrocytes and/or reticulocytes or progenitors thereof. The method includes the steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of cells in the population of progenitor cells into erythrocytes and/or reticulocytes or progenitors thereof following exposure of the population of cells to the perturbation; and identifying the perturbation as a candidate perturbation for promoting the transition of a population of progenitor cells into erythrocytes and/or reticulocytes or progenitors thereof based on the perturbation signature. In this aspect, the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as a “down” gene in the gene directionality column of Table 1.

In another aspect, the present disclosure provides a method for making a therapeutic agent for a disease or disorder selected from sickle cell disease, thalassemia, disease or disorder characterized by an abnormal oxygen delivery, and hemoglobin deficiency. In another aspect, the present disclosure provides a method for making a therapeutic agent for a disease or disorder selected from the group consisting of anemia, beta-thalassemia, sickle cell anemia (SS), sickle hemoglobin-C disease (SC), sickle beta-plus thalassemia, and sickle beta-zero thalassemia. In another aspect, the present disclosure provides a method for making a therapeutic agent for sickle cell disease, or thalassemia. In embodiments, the method includes the steps of: (a) identifying a candidate perturbation for therapy; and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into erythrocytes and/or reticulocytes or progenitors thereof following exposure of the population of cells to the perturbation; and identifying the perturbation as a candidate perturbation for promoting the transition of a population of progenitor cells into erythrocytes and/or reticulocytes or progenitors thereof based on the perturbation signature. Further, in some embodiments of this aspect, the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as a “down” gene in the gene directionality column of Table 1.

In various embodiments, the present methods reduce the amount of cells having sickling hemoglobin (HbS). In various embodiments, the present methods increase the amount of cells anti-sickling hemoglobin (HbF).

In various embodiments, the present methods involving the monitoring of cell sickling, e.g. with one or more in vitro sickling assays (see Example 3 and Smith et al. Variable deformability of irreversibly sickled erythrocytes. Blood. 1981; 58(1):71-78; van Beers et al. Imaging flow cytometry for automated detection of hypoxia-induced erythrocyte shape change in sickle cell disease Am J Hematol. 2014 June; 89(6): 598-603; and Rab, et al. Rapid and reproducible characterization of sickling during automated deoxygenation in sickle cell disease patients Am J Hematol. 2019 May; 94(5): 575-584.

To further evaluate the impact of the perturbagens in reducing disease burden, are performed on sickle derived erythrocytes cells by enrichment of enucleated erythrocytes followed by incubation of cells at low oxygen or incubation in 2% sodium metabisulfite. Cell sickling is monitored using time lapse imaging.

Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows:
Embodiment 1: A method for directing a change in cell state of a progenitor cell comprising: contacting a population of cells comprising a progenitor cell with at least one perturbagen selected from Table 4, or a variant thereof, wherein the at least one perturbagen is capable of altering a gene signature in the progenitor cell; and wherein the progenitor cell is a non-lineage committed CD34+ cell.
Embodiment 2: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as a “down” gene in the gene directionality column of Table 1 and wherein the progenitor cell is a non-lineage committed CD34+ cell.
Embodiment 3: A method for directing a change in cell state of a progenitor cell, comprising: contacting a population of cells comprising a progenitor cell with at least one perturbagen selected from Table 4, or a variant thereof, and capable of altering a gene signature in the progenitor cell, wherein altering the gene signature comprises an increase in expression and/or activity in the progenitor cell of one or more genes selected from the genes designated as an “up” gene in the gene directionality column of Table 1 and/or a decrease in expression and/or activity in the progenitor cell of one or more genes selected from the genes designated as a “down” gene in the gene directionality column of Table 1 and wherein the progenitor cell is a non-lineage committed CD34+ cell.
Embodiment 4: The method of Embodiment 2 or 3, wherein altering the gene signature comprises an increase in expression and/or activity in the progenitor cell of a network module designated in the network module column of Table 1.
Embodiment 5: The method of Embodiment 4, wherein the activation of one or more genes of the network module designated in the network module column of Table 1 comprises modulating expression and/or activity of 2 or more genes within a network module.
Embodiment 6: The method of Embodiment 2 or 3, wherein altering the gene signature comprises an increase in expression and/or activity in the progenitor cell of two or more genes designated as an “up” gene in the gene directionality column of Table 1.
Embodiment 7: The method of Embodiment 6, wherein altering the gene signature comprises a decrease in expression and/or activity in the progenitor cell of two or more genes designated as a “down” gene in the gene directionality column of Table 1.
Embodiment 8: The method of any one of Embodiments 1 to 7, wherein the change in cell state provides an increase in the number of one or more of proerythroblasts, early erythroblasts, intermediate erythroblasts, late erythroblasts, reticulocytes, and erythrocytes, wherein one or more of the proerythroblasts, early erythroblasts, intermediate erythroblasts, late erythroblasts, reticulocytes, and erythrocytes expresses fetal hemoglobin (HbF).
Embodiment 9: The method of any one of Embodiments 1 to 8, wherein the change in cell state provides an increase in F cells.
Embodiment 10: The method of Embodiment 8 or 9, wherein one or more of the proerythroblasts, early erythroblasts, intermediate erythroblasts, late erythroblasts, reticulocytes, and erythrocytes expresses HBG1 and/or HBG2
Embodiment 11: The method of Embodiment 10, wherein the increase in the number of erythrocytes comprising HbF is relative to the number of erythrocytes obtained from a population of progenitor cells that is not contacted with the at least one perturbagen.
Embodiment 12: The method of Embodiment 10, wherein the increase in the number of erythrocytes comprising HbF is relative to the population of progenitor cells prior to contacting with the at least one perturbagen.
Embodiment 13: The method of Embodiment 11 or Embodiment 12, wherein the change in cell state provides an increase in the number of erythrocytes comprising HbF.
Embodiment 14: The method of Embodiment 8, wherein the ratio of the number of erythrocytes comprising HbF to the number of progenitor cells is increased relative to the ratio obtained from a population of progenitor cells that is not contacted with the at least one perturbagen.
Embodiment 15: The method of Embodiment 8, wherein the ratio of the number of erythrocytes comprising HbF to the number of progenitor cells is increased relative to the ratio in the population of progenitor cells prior to contacting with the at least one perturbagen.
Embodiment 16: The method of any one of Embodiments 8 to 15, wherein the increase in the number of erythrocytes comprising HbF, is due in part to increased cell proliferation of the erythrocytes comprising HbF.
Embodiment 17: The method of any one of Embodiments 8 to 16, wherein the increase in the number of erythrocytes comprising HbF, is due in part to an increased lifespan of the erythrocytes comprising HbF.
Embodiment 18: The method of any one of Embodiments 8 to 17, wherein the increase in the number of erythrocytes comprising HbF, is due in part to reduced cell death among the erythrocytes comprising HbF.
Embodiment 19: The method of any one of Embodiments 8 to 18, wherein the increase in the number of erythrocytes comprising HbF, is due in part to a change of cell state from progenitor cells into the erythrocyte lineage.
Embodiment 20: The method of any one of Embodiments 1 to 19, wherein the number of progenitor cells is decreased.
Embodiment 21: The method of Embodiment 20, wherein the decrease in the number of progenitor cells is due in part to decreased cell proliferation of the progenitor cells.
Embodiment 22: The method of Embodiment 20 or Embodiment 21, wherein the decrease in the number of progenitor cells is due in part to a decreased lifespan of the progenitor cells.
Embodiment 23: The method of any one of Embodiments 20 to 22, wherein the decrease in the number of progenitor cells is due in part to increased cell death among the progenitor cells.
Embodiment 24: The method of any one of Embodiments 20 to 23, wherein the decrease in the number of progenitor cells is relative to the number of progenitor cells in a population of progenitor cells that is not contacted with the at least one perturbagen.
Embodiment 25: The method of any one of Embodiments 20 to 24, wherein the decrease in the number of progenitor cells is relative to the number of progenitor cells in the population prior to contacting with the at least one perturbagen.
Embodiment 26: The method of any one of Embodiments 20 to 25, wherein the decrease in the number of progenitor cells is due to a change of cell state from a progenitor cell into the erythrocyte lineage.
Embodiment 27: The method of any one of Embodiments 1 to 19, wherein the number of progenitor cells is increased.
Embodiment 28: The method of Embodiment 27, wherein the increase in the number of progenitor cells is due in part to increased cell proliferation of the progenitor cells.
Embodiment 29: The method of Embodiment 27 or Embodiment 28, wherein the increase in the number of progenitor cells is due in part to an increased lifespan of the progenitor cells.
Embodiment 30: The method of any one of Embodiments 27 to 29, wherein the increase in the number of progenitor cells is due in part to decreased cell death among the progenitor cells.
Embodiment 31: The method of any one of Embodiments 27 to 30, wherein the increase in the number of progenitor cells is relative to the number of progenitor cells in a population of progenitor cells that is not contacted with the at least one perturbagen.
Embodiment 32: The method of any one of Embodiments 27 to 31, wherein the increase in the number of progenitor cells is relative to the number of progenitor cells in the population prior to contacting with the at least one perturbagen.
Embodiment 33: The method of any one of Embodiments 1 to 19, wherein the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.
Embodiment 34: The method of any one of Embodiments 1 to 19, wherein the number of erythrocytes comprising HbF is increased after contacting the population of cells comprising a CD34+ cell with the at least one perturbagen.
Embodiment 35: The method of any one of Embodiments 1 to 19, wherein the number of reticulocytes comprising HbF, and/or erythrocytes comprising HbF is increased after contacting the population of cells comprising a CD34+ cell with the at least one perturbagen.
Embodiment 36: The method of Embodiment 33, wherein the ratio of the number of proerythroblasts, BFU-E cells, CFU-E cells, early erythroblasts, intermediate erythroblasts, late erythroblasts, reticulocytes, and/or erythrocytes to the number of progenitor cells is increased relative to the ratio obtained from a population of progenitor cells that is not contacted with the at least one perturbagen, wherein one or more of the proerythroblasts, early erythroblasts, intermediate erythroblasts, late erythroblasts, reticulocytes, and erythrocytes comprise HbF.
Embodiment 37: The method of Embodiment 33, wherein the ratio of the number proerythroblasts, BFU-E cells, CFU-E cells, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen, wherein one or more of the proerythroblasts, early erythroblasts, intermediate erythroblasts, late erythroblasts, reticulocytes, and erythrocytes comprise HbF.
Embodiment 38: The method of any one of Embodiments 1 to 19, wherein the ratio of the number of early erythroblasts, intermediate erythroblasts, late erythroblasts, reticulocytes, and/or erythrocytes to the number of proerythroblasts cells is increased relative to the ratio obtained from a population of progenitor cells that is not contacted with the at least one perturbagen, wherein one or more of the proerythroblasts, early erythroblasts, intermediate erythroblasts, late erythroblasts, reticulocytes, and erythrocytes comprise HbF.
Embodiment 39: The method of any one of Embodiments 1 to 19, wherein the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen, wherein one or more of the proerythroblasts, early erythroblasts, intermediate erythroblasts, late erythroblasts, reticulocytes, and erythrocytes comprise HbF.
Embodiment 40: The method of any one of Embodiments 1 to 19, wherein the ratio of the number of HBF-expressing early erythroblasts to the number of HBF-expressing proerythroblasts is increased relative to the ratio obtained from a population of progenitor cells that is not contacted with the at least one perturbagen.
Embodiment 41: The method of any one of Embodiments 1 to 19, wherein the ratio of the number of HBF-expressing early erythroblasts to the number of HBF-expressing proerythroblasts is increased relative to the ratio in the population of progenitor cells prior to contacting with the at least one perturbagen.
Embodiment 42: The method of any one of Embodiments 1 to 19, wherein the ratio of the number of HBF-expressing intermediate erythroblasts to the number of HBF-expressing early erythroblasts is increased relative to the ratio obtained from a population of progenitor cells that is not contacted with the at least one perturbagen.
Embodiment 43: The method of any one of Embodiments 1 to 19, wherein the ratio of the number of HBF-expressing intermediate erythroblasts to the number of HBF-expressing early erythroblasts is increased relative to the ratio in the population of progenitor cells prior to contacting with the at least one perturbagen.
Embodiment 44: The method of any one of Embodiments 1 to 19, wherein the ratio of the number of HBF-expressing late erythroblasts to the number of HBF-expressing intermediate erythroblasts is increased relative to the ratio obtained from a population of progenitor cells that is not contacted with the at least one perturbagen.
Embodiment 45: The method of any one of Embodiments 1 to 19, wherein the ratio of the number of HBF-expressing late erythroblasts to the number of HBF-expressing intermediate erythroblasts is increased relative to the ratio in the population of progenitor cells prior to contacting with the at least one perturbagen.
Embodiment 46: The method of any one of Embodiments 1 to 19, wherein the ratio of the number of r reticulocytes comprising HbF to the number of HBF-expressing late erythroblasts is increased relative to the ratio obtained from a population of progenitor cells that is not contacted with the at least one perturbagen.
Embodiment 47: The method of any one of Embodiments 1 to 19, wherein the ratio of the number of reticulocytes comprising HbF to the number of HBF-expressing late erythroblasts is increased relative to the ratio in the population of progenitor cells prior to contacting with the at least one perturbagen.
Embodiment 48: The method of any one of Embodiments 1 to 19, wherein the ratio of the number of erythrocytes comprising HbF to the number of reticulocytes comprising HbF is increased relative to the ratio obtained from a population of progenitor cells that is not contacted with the at least one perturbagen.
Embodiment 49: The method of any one of Embodiments 1 to 19, wherein the ratio of the number of erythrocytes comprising HbF to the number of reticulocytes comprising HbF is increased relative to the ratio in the population of progenitor cells prior to contacting with the at least one perturbagen.
Embodiment 50: The method of any one of Embodiments 1 to 19, wherein the ratio of the number of erythrocytes comprising HbF to HBF-expressing late erythroblasts is increased relative to the ratio obtained from a population of progenitor cells that is not contacted with the at least one perturbagen.
Embodiment 51: The method of any one of Embodiments 1 to 19, wherein the ratio of the number of erythrocytes comprising HbF to HBF-expressing late erythroblasts is increased relative to the ratio in the population of progenitor cells prior to contacting with the at least one perturbagen.
Embodiment 52: The method of any one of Embodiments 1 to 19, wherein the ratio of the number of erythrocytes comprising HbF to HBF-expressing intermediate erythroblasts is increased relative to the ratio obtained from a population of progenitor cells that is not contacted with the at least one perturbagen.
Embodiment 53: The method of any one of Embodiments 1 to 19, wherein the ratio of the number of erythrocytes comprising HbF to HBF-expressing intermediate erythroblasts is increased relative to the ratio in the population of progenitor cells prior to contacting with the at least one perturbagen.
Embodiment 54: The method of any one of Embodiments 1 to 19, wherein the ratio of the number of erythrocytes comprising HbF to HBF-expressing early erythroblasts is increased relative to the ratio obtained from a population of progenitor cells that is not contacted with the at least one perturbagen.
Embodiment 55: The method of any one of Embodiments 1 to 19, wherein the ratio of the number of erythrocytes comprising HbF to HBF-expressing early erythroblasts is increased relative to the ratio in the population of progenitor cells prior to contacting with the at least one perturbagen.
Embodiment 56: The method of any one of Embodiments 1 to 19, wherein the ratio of the number of erythrocytes comprising HbF to HBF-expressing proerythroblasts is increased relative to the ratio obtained from a population of progenitor cells that is not contacted with the at least one perturbagen.
Embodiment 57: The method of any one of Embodiments 1 to 19, wherein the ratio of the number of erythrocytes comprising HbF to HBF-expressing proerythroblasts is increased relative to the ratio in the population of progenitor cells prior to contacting with the at least one perturbagen.
Embodiment 58: The method of any one of Embodiments 1 to 19, wherein the ratio of the number of erythrocytes comprising HbF to proerythroblasts is increased relative to the ratio obtained from a population of progenitor cells that is not contacted with the at least one perturbagen.
Embodiment 59: The method of any one of Embodiments 1 to 19, wherein the ratio of the number of erythrocytes comprising HbF to proerythroblasts is increased relative to the ratio in the population of progenitor cells prior to contacting with the at least one perturbagen.
Embodiment 60: The method of any of Embodiments 1 to 59, wherein the number of proerythroblasts is decreased.
Embodiment 61: The method of any of Embodiments 1 to 59, wherein the number of HBF-negative or HBF-low proerythroblasts is decreased.
Embodiment 62: The method of any of Embodiments 1 to 59, wherein the number of HBF-negative or HBF-low early erythroblasts is decreased.
Embodiment 63: The method of any of Embodiments 1 to 59, wherein the number of HBF-negative or HBF-low intermediate erythroblasts is decreased.
Embodiment 64: The method of any of Embodiments 1 to 59, wherein the number of HBF-negative or HBF-low late erythroblasts is decreased.
Embodiment 65: The method of any of Embodiments 1 to 59, wherein the number of HBF-negative or HBF-low reticulocytes is decreased.
Embodiment 66: The method of any of Embodiments 1 to 59, wherein the number of proerythroblasts is increased.
Embodiment 67: The method of any of Embodiments 1 to 59, wherein the number of HBF-positive or HBF-high proerythroblasts is increased.
Embodiment 68: The method of any of Embodiments 1 to 59, wherein the number of HBF-positive or HBF-high early erythroblasts is increased.
Embodiment 69: The method of any of Embodiments 1 to 59, wherein the number of HBF-positive or HBF-high intermediate erythroblasts is increased.
Embodiment 70: The method of any of Embodiments 1 to 59, wherein the number of HBF-positive or HBF-high late erythroblasts is increased.
Embodiment 71: The method of any of Embodiments 1 to 59, wherein the number of HBF-positive or HBF-high reticulocytes is increased.
Embodiment 72: The method of any of Embodiments 1 to 59, wherein the number of HBF-positive or HBF-high erythrocytes is increased.
Embodiment 73: The method of any of Embodiments 1 to 59, wherein the number of F cells is increased.
Embodiment 74: The method of any of Embodiments 1 to 59, wherein the ratio of the number of F cells to non-F cells is increased relative to the ratio obtained from a population of progenitor cells that is not contacted with the at least one perturbagen.
Embodiment 75: The method of any of Embodiments 1 to 59, wherein the ratio of the number of F cells to non-F cells is increased relative to the ratio in the population of progenitor cells prior to contacting with the at least one perturbagen.
Embodiment 76: The method of any one of Embodiments 1 to 75, wherein the at least one perturbagen selected from Table 4, or a variant thereof, comprises at least 2, at least 3, at least 4, or at least 5 perturbagens selected from Table 4, or variants thereof.
Embodiment 77: The method of Embodiment 2 or 3, wherein the one or more genes selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 19 or more, genes selected from the genes designated as an “up” gene in the gene directionality column of Table 1.
Embodiment 78: The method of Embodiment 77, wherein the one or more genes selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1.
Embodiment 79: The method of Embodiment 2 or 3, wherein the one or more genes selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1.
Embodiment 80: The method of Embodiment 79, wherein the one or more genes selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2.
Embodiment 81: The method of any one of Embodiments 1 to 80, wherein contacting the population of progenitor cells occurs in vitro or ex vivo.
Embodiment 82: The method of any one of Embodiments 1 to 80, wherein contacting the population of progenitor cells occurs in vivo in a subject.
Embodiment 83: The method of Embodiment 82, wherein the subject is a human.
Embodiment 84: The method of Embodiment 83, wherein the human is an adult human.
Embodiment 85: A perturbagen for use in the method of any one of Embodiments 1 to 84.
Embodiment 86: A pharmaceutical composition comprising the perturbagen of Embodiment 85.
Embodiment 87: A method for treating a disease or disorder characterized by an abnormal oxygen delivery, comprising: (a) administering to a subject in need thereof a therapeutically effective amount of at least one perturbagen selected from Table 4, or a variant thereof, wherein the at least one perturbagen is capable of changing a gene signature in a progenitor cell or (b) administering to a subject in need thereof a cell, the cell having been contacted with at least one perturbagen selected from Table 4, or a variant thereof, wherein the at least one perturbagen is capable of changing a gene signature in a progenitor cell.
Embodiment 88: A method for treating a disease or disorder characterized by a hemoglobin deficiency, comprising: (a) administering to a subject in need thereof a therapeutically effective amount of at least one perturbagen selected from Table 4, or a variant thereof, wherein the at least one perturbagen is capable of changing a gene signature in a progenitor cell or (b) administering to a subject in need thereof a cell, the cell having been contacted with at least one perturbagen selected from Table 4, or a variant thereof, wherein the at least one perturbagen is capable of changing a gene signature in a progenitor cell.
Embodiment 89: A method for treating or preventing an sickle cell disease or a thalassemia, comprising: (a) administering to a subject in need thereof a therapeutically effective amount of at least one perturbagen selected from Table 4, or a variant thereof, wherein the at least one perturbagen is capable of changing a gene signature in a progenitor cell or (b) administering to a subject in need thereof a cell, the cell having been contacted with at least one perturbagen selected from Table 4, or a variant thereof, wherein the at least one perturbagen is capable of changing a gene signature in a progenitor cell.
Embodiment 90: The method of Embodiment 88, wherein the hemoglobin deficiency is an abnormal and/or reduced oxygen delivery functionality of hemoglobin, optionally resultant from mutations in one or more hemoglobin genes, the mutation optionally being in a HBB gene.
Embodiment 91: The method of any one of Embodiments 87 to 89, wherein the administering is directed to the bone marrow of the subject.
Embodiment 92: The method of Embodiment 91, wherein the administering is via intraosseous injection or intraosseous infusion.
Embodiment 93: The method of any one of Embodiments 87 to 92, wherein the administering the cell is via intravenous injection or intravenous infusion.
Embodiment 94: The method of any one of Embodiments 87 to 93, wherein the administering is simultaneously or sequentially to one or more mobilization agents.
Embodiment 95: The method of any one of Embodiments 87 to 94 wherein the disease or disorder characterized by an abnormal oxygen delivery and/or a hemoglobin deficiency is an anemia.
Embodiment 96: The method of any one of Embodiments 87 to 95, wherein the sickle cell disease or a thalassemia is beta-thalassemia (transfusion dependent).
Embodiment 97: The method of any one of Embodiments 87 to 95, wherein the sickle cell disease or a thalassemia is beta-thalassemia major.
Embodiment 98: The method of any one of Embodiments 87 to 95, wherein the sickle cell disease or a thalassemia is beta-thalassemia intermedia.
Embodiment 99: The method of any one of Embodiments 87 to 95, wherein the sickle cell disease or a thalassemia is beta-thalassemia minor.
Embodiment 100: The method of any one of Embodiments 87 to 95, wherein the sickle cell disease or a thalassemia is sickle cell anemia (SS), sickle hemoglobin-C disease (SC), sickle beta-plus thalassemia and sickle beta-zero thalassemia.
Embodiment 101: The method of any one of Embodiments 87 to 100, wherein the at least one perturbagen is capable of changing a gene signature in a progenitor cell.
Embodiment 102: The method of any one of Embodiments 87 to 100, wherein the subject is selected by steps comprising: obtaining from the subject having the disease or disorder a sample of cells comprising a non-lineage committed CD34+ cell; and contacting the sample of cells with least one perturbagen selected from Table 4, or a variant thereof, wherein the at least one perturbagen alters a gene signature in the sample of cells.
Embodiment 103: The method of any one of Embodiments 87 to 100, wherein the subject is selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from the genes designated as an “up” gene in the gene directionality column of Table 1 and/or decreases in the sample of cells the expression and/or activity of one or more genes selected from the genes designated as a “down” gene in the gene directionality column of Table 1.
Embodiment 104: The method of any one of Embodiments 87 to 100, wherein the subject is selected by steps comprising: obtaining from a subject having the disease or disorder a sample of cells comprising a non-lineage committed CD34+ cell; and contacting the sample of cells with at least one perturbagen selected from Table 4, or a variant thereof; wherein the at least one perturbagen increases in the sample of cells the expression and/or activity of one or more genes selected from the genes designated as an “up” gene in the gene directionality column of Table 1 and/or decreases in the sample of cells the expression and/or activity of one or more genes selected from the genes designated as a “down” gene in the gene directionality column of Table 1.
Embodiment 105: A method for selecting the subject of any one of Embodiments 87 to 100, comprising: obtaining from a subject having the disease or disorder a sample of cells comprising a non-lineage committed CD34+ cell; and contacting the sample of cells with least one perturbagen selected from Table 4, or a variant thereof, wherein when the at least one perturbagen alters a gene signature in the sample of cells, the subject is selected as a subject.
Embodiment 106: A method for selecting the subject of any one of Embodiments 87 to 100, comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from the genes designated as an “up” gene in the gene directionality column of Table 1 and/or decreases in the sample of cells the expression and/or activity of one or more genes selected from the genes designated as a “down” gene in the gene directionality column of Table 1, the subject is selected as a subject.
Embodiment 107: A method for selecting the subject of any one of Embodiments 87 to 100, comprising: obtaining from a subject having the disease or disorder a sample of cells comprising a non-lineage committed CD34+ cell; and contacting the sample of cells with at least one perturbagen selected from Table 4, or a variant thereof; wherein when the at least one perturbagen increases in the sample of cells the expression and/or activity of one or more genes selected from the genes designated as an “up” gene in the gene directionality column of Table 1 and/or decreases in the sample of cells the expression and/or activity of one or more genes selected from the genes designated as a “down” gene in the gene directionality column of Table 1, the subject is selected as a subject.
Embodiment 108: Use of the perturbagen of Table 4, or a variant thereof in the manufacture of a medicament for treating a disease or disorder characterized an abnormal oxygen delivery or a hemoglobin deficiency.
Embodiment 109: Use of the perturbagen of Table 4, or a variant thereof in the manufacture of a medicament for treating sickle cell disease or a thalassemia.
Embodiment 110: A method of identifying a candidate perturbation for promoting the transition of a starting population of progenitor cells into erythrocytes comprising HbF or HbF-expressing progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation; and identifying the perturbation as a candidate perturbation for promoting the transition of a population of progenitor cells into erythrocytes comprising HbF or HbF-expressing progenitors thereof based on the perturbation signature, wherein the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as a “down” gene in the gene directionality column of Table 1.
Embodiment 111: The method of Embodiment 110, wherein the perturbation signature is an increase in expression and/or activity in the progenitor cell of a network module designated in the network module column of Table 1.
Embodiment 112: The method of Embodiment 111, wherein the perturbation signature comprises, the activation of one or more genes of the network module designated in the network module column of Table 1 comprises modulating expression and/or activity of 2 or more genes within a network module.
Embodiment 113: The method of Embodiment 110, wherein the perturbation signature is an increase in expression and/or activity in the progenitor cell of two or more genes designated as an “up” gene in the gene directionality column of Table 1.
Embodiment 114: The method of Embodiment 113, wherein the perturbation signature is a decrease in expression and/or activity in the progenitor cell of two or more genes designated as a “down” gene in the gene directionality column of Table 1.
Embodiment 115: A method for making a therapeutic agent for a disease or disorder selected from a sickle cell disease or a thalassemia or a disease or disorder characterized by an abnormal oxygen delivery or a hemoglobin deficiency, comprising: (a) identifying a candidate perturbation for therapy according to the method of Embodiment 110 and (b) formulating the candidate perturbation as a therapeutic agent for the treatment of the disease or disorder.

### Erythrocytes

### Gene Signature

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins (i.e., antigen expression) that are characteristic of the cell type or cells of a specific lineage.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

The erythroid progenitor cells at different maturation stages may be characterized by its antigen expression. The erythroblasts express transferrin receptor (also known as CD71 in human) and glycophorin A (GlyA, also known as CD235a in human) (Hattangadiet. al., Blood, 2011, 118 (24):6258-68.), but express little or no hemoglobin (Hb). The erythroblasts have the capacity to mature into hemoglobinized erythrocytes and reticulocytes. During maturation, CD71 expression decreases but remains detectable on most cells, GlyA expression remains high or increases further, and cell pellets become visibly red due to the accumulation of Hb.

Genes that are differentially expressed and positively associated with the promotion of erythroid lineage progression and/or erythrocyte differentiation are listed in Table 2.

TABLE 2
Genes showing an increase in expression in at
least one perturbagen.

| | | Gene | Gene | Network |
|---|---|---|---|---|
| | Gene | EntrezID | Directionality | Module |
| 0 | DNAJC15 | 29103 | down | 0 |
| 1 | SNCA | 6622 | down | 0 |
| 2 | CEP57 | 9702 | down | 0 |
| 3 | BZW2 | 28969 | down | 0 |
| 4 | BID | 637 | down | 0 |
| 5 | SMC3 | 9126 | down | 0 |
| 6 | VDAC1 | 7416 | down | 1 |
| 7 | RNPS1 | 10921 | down | 1 |
| 8 | PSMB8 | 5696 | down | 1 |
| 9 | MLEC | 9761 | down | 1 |
| 10 | SNX6 | 58533 | down | 1 |
| 11 | SMARCA4 | 6597 | down | 1 |
| 12 | TSC22D3 | 1831 | up | 2 |
| 13 | DDIT4 | 54541 | up | 2 |
| 14 | HSPD1 | 3329 | down | 2 |
| 15 | NUCB2 | 4925 | down | 2 |
| 16 | PHGDH | 26227 | down | 2 |
| 17 | GABPB1 | 2553 | down | 2 |
| 18 | TNIP1 | 10318 | up | 3 |
| 19 | FHL2 | 2274 | up | 3 |
| 20 | HMGCS1 | 3157 | up | 3 |
| 21 | CYCS | 54205 | up | 3 |
| 22 | CCNH | 902 | down | 3 |
| 23 | RBM6 | 10180 | down | 3 |
| 24 | HK1 | 3098 | up | 4 |
| 25 | ACLY | 47 | up | 4 |
| 26 | JADE2 | 23338 | up | 4 |
| 27 | MAT2A | 4144 | down | 4 |
| 28 | RAB4A | 5867 | down | 4 |
| 29 | HEBP1 | 50865 | down | 4 |
| 30 | PIH1D1 | 55011 | up | 5 |
| 31 | BAX | 581 | up | 5 |
| 32 | CORO1A | 11151 | down | 5 |
| 33 | ACAA1 | 30 | down | 5 |
| 34 | PPOX | 5498 | down | 5 |
| 35 | RPA2 | 84172 | up | 6 |
| 36 | CCND3 | 896 | up | 6 |
| 37 | MEST | 4232 | down | 6 |
| 38 | STX4 | 6810 | down | 6 |
| 39 | FKBP4 | 2288 | down | 6 |
| 40 | UBE2A | 7319 | down | 7 |
| 41 | DERA | 51071 | down | 7 |
| 42 | ATG3 | 64422 | down | 7 |
| 43 | NUSAP1 | 51203 | down | 7 |
| 44 | NUP88 | 4927 | down | 7 |
| 45 | KIT | 3815 | up | 8 |
| 46 | CYB561 | 1534 | up | 8 |
| 47 | H2AFV | 94239 | down | 8 |
| 48 | PLP2 | 5355 | down | 8 |
| 49 | UBE2L6 | 9246 | down | 8 |
| 50 | S100A4 | 6275 | up | 9 |
| 51 | HLA-DRA | 3122 | down | 9 |
| 52 | MLLT11 | 10962 | down | 9 |
| 53 | SCP2 | 10388 | down | 9 |
| 54 | OXA1L | 5018 | down | 10 |
| 55 | KTN1 | 3895 | down | 10 |
| 56 | GNAI2 | 2771 | down | 10 |
| 57 | DECR1 | 1666 | down | 10 |
| 58 | LSM6 | 11157 | down | 11 |
| 59 | HADH | 3033 | down | 11 |
| 60 | WDR61 | 80349 | down | 11 |
| 61 | DCK | 1633 | down | 11 |
| 62 | KLHDC2 | 23588 | down | 12 |
| 63 | CAT | 10249 | down | 12 |
| 64 | CBR3 | 874 | down | 12 |
| 65 | DHRS7 | 51635 | down | 12 |
| 66 | PIN1 | 5300 | up | 13 |
| 67 | NT5DC2 | 64943 | up | 13 |
| 68 | CD320 | 51293 | up | 13 |
| 69 | BAD | 572 | down | 13 |
| 70 | GAPDH | 2597 | down | 14 |
| 71 | CDK4 | 1019 | down | 14 |
| 72 | MAPKAPK3 | 7867 | down | 14 |
| 73 | PSIP1 | 11168 | down | 15 |
| 74 | PCM1 | 4152 | down | 15 |
| 75 | PSMD4 | 5710 | down | 15 |
| 76 | APOE | 348 | up | 16 |
| 77 | HSPA8 | 3312 | down | 16 |
| 78 | SPTLC2 | 9517 | down | 16 |
| 79 | ID2 | 3398 | up | 17 |
| 80 | DAXX | 1616 | up | 17 |
| 81 | SOX4 | 6659 | down | 17 |
| 82 | HLA-DMA | 3108 | down | 18 |
| 83 | SCCPDH | 51097 | down | 18 |
| 84 | LAGE3 | 8270 | down | 18 |
| 85 | CTTN | 2017 | up | 19 |
| 86 | PDLIM1 | 9124 | down | 19 |
| 87 | EAPP | 55837 | down | 19 |
| 88 | IFRD2 | 7866 | up | 20 |
| 89 | MRPS16 | 51021 | down | 20 |
| 90 | VPS28 | 51160 | down | 20 |
| 91 | FAH | 2184 | down | 21 |
| 92 | PSMB10 | 5699 | down | 21 |
| 93 | ICAM3 | 3385 | down | 21 |
| 94 | CAB39 | 51719 | up | 22 |
| 95 | HSD17B11 | 51170 | down | 22 |
| 96 | MIF | 4282 | down | 23 |
| 97 | NENF | 29937 | down | 23 |
| 98 | RPA3 | 6119 | down | 24 |
| 99 | ADI1 | 55256 | down | 24 |
| 100 | AKR7A2 | 8574 | down | 25 |
| 101 | KDELR2 | 11014 | down | 25 |
| 102 | PGAM1 | 5223 | down | 26 |
| 103 | CREG1 | 8804 | down | 26 |

In Table 2 and associated embodiments:
“Gene ID”: at the time of filing the present disclosure, the World Wide Web at ncbi.nlm.nih.gov/gene provides a description of and the nucleic acid sequence for each GeneID listed in Table 2; the contents of each of which is incorporated herein by reference in its entirety.
“Up” indicates a gene for which an increase in expression and/or activity in the progenitor cell is associated with the gene signature.
“Down” indicates a gene for which an decrease in expression and/or activity in the progenitor cell is associated with the gene signature.

A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition—such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 2, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 2. In certain embodiments, a perturbation activates multiple network modules for the desired cell state transition, such as 2, 3, 4, 5, 6, 7, 8, 9, 10, 11, 12, 13, 14, 15, 16, 17, 18, 19, 20, 21, 22, 23, 24, 25, 26, or 27 modules.

In some embodiments, one or more genes of network module 0 are modulated. In some embodiments, the present technology relates to the activation of network module 0, e.g., one or more of (inclusive of all of) DNAJC15, SNCA, CEP57, BZW2, BID, and SMC3. In some embodiments, the modulation is upmodulation or downmodulation as described in Gene Directionality column of Table 2.

In some embodiments, one or more genes of network module 1 are modulated. In some embodiments, the present technology relates to the activation of network module 1, e.g., one or more of (inclusive of all of) VDAC1, RNPS1, PSMB8, MLEC, SNX6, and SMARCA4. In some embodiments, the modulation is upmodulation or downmodulation as described in Gene Directionality column of Table 2.

In some embodiments, one or more genes of network module 2 are modulated. In some embodiments, the present technology relates to the activation of network module 2, e.g., one or more of (inclusive of all of) TSC22D3, DDIT4, HSPD1, NUCB2, PHGDH, and GABPB1. In some embodiments, the modulation is upmodulation or downmodulation as described in Gene Directionality column of Table 2.

In some embodiments, one or more genes of network module 3 are modulated. In some embodiments, the present technology relates to the activation of network module 3, e.g., one or more of (inclusive of all of) TNIP1, FHL2, HMGCS1, CYCS, CCNH, and RBM6. In some embodiments, the modulation is upmodulation or downmodulation as described in Gene Directionality column of Table 2.

In some embodiments, one or more genes of network module 4 are modulated. In some embodiments, the present technology relates to the activation of network module 4, e.g., one or more of (inclusive of all of) HK1, ACLY, JADE2, MAT2A, RAB4A, and HEBP1. In some embodiments, the modulation is upmodulation or downmodulation as described in Gene Directionality column of Table 2.

In some embodiments, one or more genes of network module 5 are modulated. In some embodiments, the present technology relates to the activation of network module 5, e.g., one or more of (inclusive of all of) PIH1D1, BAX, CORO1A, ACAA1, and PPOX. In some embodiments, the modulation is upmodulation or downmodulation as described in Gene Directionality column of Table 2.

In some embodiments, one or more genes of network module 6 are modulated. In some embodiments, the present technology relates to the activation of network module 6, e.g., one or more of (inclusive of all of) RPA2, CCND3, MEST, STX4, and FKBP4. In some embodiments, the modulation is upmodulation or downmodulation as described in Gene Directionality column of Table 2.

In some embodiments, one or more genes of network module 7 are modulated. In some embodiments, the present technology relates to the activation of network module 7, e.g., one or more of (inclusive of all of) UBE2A, DERA, ATG3, NUSAP1, and NUP88. In some embodiments, the modulation is upmodulation or downmodulation as described in Gene Directionality column of Table 2.

In some embodiments, one or more genes of network module 8 are modulated. In some embodiments, the present technology relates to the activation of network module 8, e.g., one or more of (inclusive of all of) KIT, CYB561, H2AFV, PLP2, and UBE2L6. In some embodiments, the modulation is upmodulation or downmodulation as described in Gene Directionality column of Table 2.

In some embodiments, one or more genes of network module 9 are modulated. In some embodiments, the present technology relates to the activation of network module 9, e.g., one or more of (inclusive of all of) S100A4, HLA-DRA, MLLT11, and SCP2.

In some embodiments, one or more genes of network module 10 are modulated. In some embodiments, the present technology relates to the activation of network module 10, e.g., one or more of (inclusive of all of) OXA1L, KTN1, GNAI2, and DECR1. In some embodiments, the modulation is upmodulation or downmodulation as described in Gene Directionality column of Table 2.

In some embodiments, one or more genes of network module 11 are modulated. In some embodiments, the present technology relates to the activation of network module 11, e.g., one or more of (inclusive of all of) LSM6, HADH, WDR61, and DCK. In some embodiments, the modulation is upmodulation or downmodulation as described in Gene Directionality column of Table 2.

In some embodiments, one or more genes of network module 12 are modulated. In some embodiments, the present technology relates to the activation of network module 12, e.g., one or more of (inclusive of all of) KLHDC2, CAT, CBR3, and DHRS7. In some embodiments, the modulation is upmodulation or downmodulation as described in Gene Directionality column of Table 2.

In some embodiments, one or more genes of network module 13 are modulated. In some embodiments, the present technology relates to the activation of network module 13, e.g., one or more of (inclusive of all of) PIN1, NT5DC2, CD320, and BAD. In some embodiments, the modulation is upmodulation or downmodulation as described in Gene Directionality column of Table 2.

In some embodiments, one or more genes of network module 14 are modulated. In some embodiments, the present technology relates to the activation of network module 14, e.g., one or more of (inclusive of all of) GAPDH, CDK4, and MAPKAPK3. In some embodiments, the modulation is upmodulation or downmodulation as described in Gene Directionality column of Table 2.

In some embodiments, one or more genes of network module 15 are modulated. In some embodiments, the present technology relates to the activation of network module 15, e.g., one or more of (inclusive of all of) PSIP1, PCM1, and PSMD4. In some embodiments, the modulation is upmodulation or downmodulation as described in Gene Directionality column of Table 2.

In some embodiments, one or more genes of network module 16 are modulated. In some embodiments, the present technology relates to the activation of network module 16, e.g., one or more of (inclusive of all of) APOE, HSPA8, SPTLC2. In some embodiments, the modulation is upmodulation or downmodulation as described in Gene Directionality column of Table 2.

In some embodiments, one or more genes of network module 17 are modulated. In some embodiments, the present technology relates to the activation of network module 17, e.g., one or more of (inclusive of all of) ID2, DAXX, and SOX4. In some embodiments, the modulation is upmodulation or downmodulation as described in Gene Directionality column of Table 2.

In some embodiments, one or more genes of network module 18 are modulated. In some embodiments, the present technology relates to the activation of network module 18, e.g., one or more of (inclusive of all of) HLA-DMA, SCCPDH, and LAGE3. In some embodiments, the modulation is upmodulation or downmodulation as described in Gene Directionality column of Table 2.

In some embodiments, one or more genes of network module 19 are modulated. In some embodiments, the present technology relates to the activation of network module 19, e.g., one or more of (inclusive of all of) CTTN, PDLIM1, and EAPP. In some embodiments, the modulation is upmodulation or downmodulation as described in Gene Directionality column of Table 2.

In some embodiments, one or more genes of network module 20 are modulated. In some embodiments, the present technology relates to the activation of network module 20, e.g., one or more of (inclusive of all of) IFRD2, MRPS16, and VPS28. In some embodiments, the modulation is upmodulation or downmodulation as described in Gene Directionality column of Table 2.

In some embodiments, one or more genes of network module 21 are modulated. In some embodiments, the present technology relates to the activation of network module 21, e.g., one or more of (inclusive of all of) FAH, PSMB10, and ICAM3. In some embodiments, the modulation is upmodulation or downmodulation as described in Gene Directionality column of Table 2.

In some embodiments, one or more genes of network module 22 are modulated. In some embodiments, the present technology relates to the activation of network module 22, e.g., one or both of CAB39 and HSD17B11. In some embodiments, the modulation is upmodulation or downmodulation as described in Gene Directionality column of Table 2.

In some embodiments, one or more genes of network module 23 are modulated. In some embodiments, the present technology relates to the activation of network module 23, e.g., one or both of MIF and NENF. In some embodiments, the modulation is upmodulation or downmodulation as described in Gene Directionality column of Table 2.

In some embodiments, one or more genes of network module 24 are modulated. In some embodiments, the present technology relates to the activation of network module 24, e.g., one or both of RPA3 and ADI1. In some embodiments, the modulation is upmodulation or downmodulation as described in Gene Directionality column of Table 2.

In some embodiments, one or more genes of network module 25 are modulated. In some embodiments, the present technology relates to the activation of network module 25, e.g., one or both of AKR7A2 and KDELR2. In some embodiments, the modulation is upmodulation or downmodulation as described in Gene Directionality column of Table 2.

In some embodiments, one or more genes of network module 26 are modulated. In some embodiments, the present technology relates to the activation of network module 26, e.g., one or both of PGAM1 and CREG1. In some embodiments, the modulation is upmodulation or downmodulation as described in Gene Directionality column of Table 2.

In some embodiments, the present methods alter a gene signature in the sample of cells, comprising an activation of a network module designated in the network module column of Table 2.

In some embodiments, the activation of the network module designated in the network module column of Table 2 comprises modulating expression and/or activity of 2 or more genes within a network module.

In some embodiments, the activation of the network module designated in the network module column of Table 2 comprises modulating expression and/or activity of all of the genes within a network module.

In some embodiments, the activation of the network module designated in the network module column of Table 2 comprises modulating expression and/or activity of 2 or more genes within 2 or more network modules. In some embodiments, the activation of the network module designated in the network module column of Table 2 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more, or 51 or more, or 52 or more, or 53 or more, or 54 or more, or 55 or more, or 56 or more, or 57 or more, or 58 or more, or 59 or more, or 60 or more, or 61 or more, or 62 or more, or 63 or more, or 64 or more, or 65 or more, or 66 or more, or 67 or more, or 68 or more, or 69 or more, or 70 or more, or 71 or more, or 72 or more, or 73 or more, or 74 or more, or 75 or more, or 76 or more, or 77 or more, or 78 or more, or 79 or more, or 80 or more, or 81 or more, or 82 or more, or 83 or more, or 84 or more, or 85 or more, or 86 or more, or 87 or more, or 88 or more, or 89 or more, or 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 network modules).

At the time of filing the present disclosure, the World Wide Web at ncbi.nlm.nih.gov/gene provides a description of and the nucleic acid sequence for each Gene designated as an “up” gene in the gene directionality column of Table 2; the contents of each of which is incorporated herein by reference in its entirety. At the time of filing the present disclosure, the World Wide Web at ncbi.nlm.nih.gov/gene provides a description of and the nucleic acid sequence for each Gene listed in the genes designated as an “down” gene in the gene directionality column of Table 2; the contents of each of which is incorporated herein by reference in its entirety.

### Perturbagens

A perturbagen useful in the present technology can be a small molecule, a biologic, a protein, a nucleic acid, such as a cDNA over-expressing a wild-type gene or an mRNA encoding a wild-type gene, or any combination of any of the foregoing. Illustrative perturbagens useful in the present technology and capable of promoting erythrocyte lineage differentiation are listed in Table 5.

TABLE 5
Perturbagens

| | | Molecular | |
|---|---|---|---|
| Perturbagen | Molecular | weight | |
| No. | formula | (g/mol) | Dose |
| 1 | C₃₃H₃₂N₄O₄ | 548.6 | 10 μM |
| 2 | C₁₂H₁₀FN₃O₂S | 279.29 | 10 μM |
| 3 | C₃₆H₃₃N₅O₂ | 567.7 | 10 μM |
| 4 | C₈H₁₆O₂ | 144.21 | 3.33 μM |
| 5 | C₃₄H₅₉NO₁₅ | 721.8 | 100 μM |
| 6 | CgH₁₅N₄O₈P | 338.21 | 10 μM |
| 7 | C₁₇H₁₉FN₄O₂S | 362.4 | 1.11 μM |
| 8 | C₂₁H₂₄FN₃O₄ | 401.4 | 10 μM |
| 9 | C₂₄H₂₇NO₅S | 441.5 | 10 μM |
| 10 | C₃₄H₃₅ClN₂O₃S | 587.2 | 80 μM |
| 11 | C₈H₇NO₂S | 181.21 | 10 μM |
| 12 | C₁₄H₁₃NO₇ | 307.25 | 10 μM |
| 13 | C₁₈H₂₁N₅O₂S | 371.5 | 3.33 μM |
| 14 | C₁₇H₂₃ClO₄ | 326.8 | 10 μM |
| 15 | C₂₆H₂₆F₃N₃O₃ | 485.5 | 10 μM |
| 16 | C₃₁H₃₈N₂O₇ | 550.6 | 10 μM |
| 17 | C₃₃H₃₅FN₂O₅ | 558.6 | 3.33 μM |
| 18 | C₅₁H₇₉NO₁₃ | 914.2 | 10 μM |
| 19 | C₂₅H₁₉NO₃S | 413.5 | 3.33 μM |
| 20 | C₁₈H₂₂F₂N₄O | 348.4 | 10 μM |
| 21 | C₁₄H₁₁F₃N₂OS | 312.31 | 10 μM |
| 22 | C₁₇H₁₁CIN₄O | 322.7 | 10.0 μM |
| 23 | C₂₂H₂₀Cl₂N₄O₄ | 475.3 | 10 μM |
| 24 | C₁₀H₉NO₂S | 207.25 | 10 μM |
| 25 | C₂₄H₂₅N₅O | 399.5 | 10 μM |
| 26 | C₁₉H₁₈FN₃O | 323.4 | 10 μM |

In various embodiments herein, a perturbagen encompasses the perturbagens named in Table 5. Thus, the named perturbagens of Table 5 represent examples of perturbagens of the present technology.

The “dose” described in Table 5 is non-limiting. The doses of perturbagens of Table 5 represent examples of doses used in certain embodiments of the present technology.

In Table 5, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In some embodiments, a perturbagen used in the present technology is a variant of a perturbagen of Table 5. A variant may be a derivative, analog, enantiomer or a mixture of enantiomers thereof or a pharmaceutically acceptable salt, solvate, hydrate, co-crystal, clathrate, or polymorph of the perturbagen of Table 5. A variant of a perturbagen of Table 5 retains the biological activity of the perturbagen of Table 5.

### Methods and Perturbagens for Directing a Change in Cell State

Particular cellular changes in cell state can be matched to differential gene expression (which collectively define a gene signature), caused by exposure of a cell to a perturbagen. In some embodiments, a change in cell state may be from one progenitor cell type to another progenitor cell type. For example, a megakaryocyte/erythroid progenitor (MEP) may give rise to an erythroblast. In some embodiments, a change in cell state may be from an upstream progenitor cell (e.g. proerythroblasts) to a downstream progenitor cell (e.g., late erythroblasts). Lastly, in some embodiments, a change in cell state may be from the final non-differentiated cell into a differentiated cell.

Erythrocytes (also called red blood cells, RBC) are the most abundant cells in the blood and are essential for oxygen transport around the body. RBC is the principal means of delivering oxygen (O₂) to the body tissue via the blood flow through the circulatory system. The cytoplasm of RBCs is rich in hemoglobin, an iron-containing protein that binds oxygen and is responsible for the red color of the blood.

In human, the bone marrow (BM) is the major site for steady-state erythropoiesis. Hematopoietic stem cells (HSCs) reside in BM niches and the cytokines or signals generated by stromal cells regulate the differentiation of various blood lineage, including erythroid cells. HSCs first differentiate into megakaryocyte-erythroid progenitors (MEP cells), subsequently into the burst-forming unit-erythroid (BFU-Es), and finally into the colony-forming unit-erythroid (CFU-Es). CFU-Es are more mature than BFU-Es (Dzierzak et al., Erythropoiesis: development and differentiation. Cold Spring Harb Perspect Med., 2013, vol. 3, p. a011601).

Erythropoietin (EPO) is the main regulator of erythroid cell proliferation, differentiation, and survival (Fisher, Erythropoietin: physiology and pharmacology update. Exp. Biol. Med., 2003, vol. 228, pp. 1-14). EPO production is upregulated under hypoxic conditions through the activity of the hypoxia-inducible transcriptional factor (HIF) (Stockmann et al., Hypoxia-induced erythropoietin production: a paradigm for oxygen-regulated gene expression. Clin Exp Pharmacol. Physiol. 2006, vol. 33, pp. 968-79). The EPO receptor (EPOR) is expressed dominantly on CFU-Es and gradually downregulated during erythroid differentiation. Upon stimulation initiated by EPO binding to the EPORs, CFU-Es develop into proerythroblasts (stage R1), subsequently into basophilic erythroblasts (stage R2), then polychromatic erythroblasts (stage R3), and finally orthochromatic erythroblasts (Stage R4) (Elliott et al., Erythropoietin: a common mechanism of action, Exp. Hematol., 2008, vol. 36, pp. 1573-84). The final stage of erythroid differentiation involves the enucleation and maturation of reticulocytes into circulating erythrocytes.

Hypoxia causes induction of hypoxia-inducible factor (HIF), which stimulates erythropoietin (EPO) synthesis. Prolyl hydroxylase domain (PHD) enzyme inhibition can stabilize hypoxia-inducible factor (HIF). HIF stabilization also decreases hepcidin, a hormone of hepatic origin, which regulates iron homeostasis.

Granulocyte colony stimulating factor (G-CSF) has been reported to have the ability to mobilize newly synthetized erythrocytes to the peripheral blood and promote erythrocytic differentiation and proliferation in vitro and ex vivo. G-CSF promotes progression of erythropoiesis through transition of early stage R2 (basophilic erythroblasts) to late stage R4 (orthochromatophilic erythroblasts). G-CSF induces more orthochromatophilic erythroblast production than does EPO in the BM and spleen (Chen et al., Stem cell Research & Therapy, 2018, vol. 9, pp. 119).

An aspect of the present technology relates to a method for directing a change in cell state of a progenitor cell. This method includes a step of contacting a population of cells comprising a progenitor cell with at least one perturbagen selected from Table 5, or a variant of perturbagens described in Table 5. In some embodiments, the at least one perturbagen is capable of altering a gene signature in the progenitor cell. In this aspect, the at least one perturbagen is capable of altering a gene signature in the progenitor cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), a orthochromatic erythroblast (also known as a late erythroblasts). In one embodiment, the progenitor cell is selected from a proerythroblast, early erythroblast, intermediate erythroblast, late erythroblast, and reticulocyte. In some embodiments, the progenitor cell is selected from a proerythroblast, early erythroblast, intermediate erythroblast, late erythroblast, and reticulocyte.

Another aspect of the present technology relates to a method for directing a change in cell state of a progenitor cell. This method includes a step of contacting a population of cells comprising a progenitor cell with at least one perturbagen capable of altering a gene signature in the progenitor cell. In this aspect, altering the gene signature comprises an increase in expression and/or activity in the progenitor cell of one or more genes selected from the genes designated as an “up” gene in the gene directionality column of Table 2 and/or a decrease in expression and/or activity in the progenitor cell of one or more genes selected from the genes designated as an “down” gene in the gene directionality column of Table 2. In some embodiments, the at least one perturbagen is capable of altering a gene signature in the progenitor cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), a orthochromatic erythroblast (also known as a late erythroblasts). In some embodiments, the progenitor cell is selected from a proerythroblast, early erythroblast, intermediate erythroblast, late erythroblast, and reticulocyte.

Yet another aspect of the present technology relates to a method for directing a change in cell state of a progenitor cell. This method includes a step of contacting a population of cells comprising a progenitor cell with at least one perturbagen selected from Table 5, or a variant of perturbagens described in Table 5, and capable of altering a gene signature in the progenitor cell. In this aspect, altering the gene signature comprises an increase in expression and/or activity in the progenitor cell of one or more genes selected from the genes designated as an “up” gene in the gene directionality column of Table 2 and/or a decrease in expression and/or activity in the progenitor cell of one or more genes selected from the genes designated as an “down” gene in the gene directionality column of Table 2. In some embodiments, the at least one perturbagen is capable of altering a gene signature in the progenitor cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), a orthochromatic erythroblast (also known as a late erythroblasts). In some embodiments, the progenitor cell is selected from a proerythroblast, early erythroblast, intermediate erythroblast, late erythroblast, and reticulocyte. In some embodiments, altering the gene signature comprises an increase in expression and/or activity in the progenitor cell of a network module designated in the network module column of Table 2. In other embodiments, the activation of one or more genes of the network module designated in the network module column of Table 2 comprises modulating expression and/or activity of 2 or more genes within a network module. In some embodiments, altering the gene signature comprises an increase in expression and/or activity in the progenitor cell of two or more genes designated as an “up” gene in the gene directionality column of Table 2. In other embodiments, altering the gene signature comprises a decrease in expression and/or activity in the progenitor cell of two or more genes designated as a “down” gene in the gene directionality column of Table 2.

In some embodiments, the non-lineage committed CD34⁺ cell is a hematopoietic stem and progenitor cell (HSPC). In some embodiments, the step of contacting a population of cells comprising a progenitor cell with a perturbagen causes a change in the cell state.

In some embodiments, the change in cell state causes an increase in the number of one or more of proerythroblasts, early erythroblasts, intermediate erythroblasts, late erythroblasts, reticulocytes, and erythrocytes. In some embodiments, the change in the number of proerythroblasts, erythroblasts, reticulocytes, and/or erythrocytes is relative to a control population of cells. For example, in some embodiments, the increase in the number of proerythroblasts, erythroblasts, reticulocytes, and/or erythrocytes upon contacting the cells with a perturbagen- is relative to the population of progenitor cells that is not contacted with the perturbagen. In other embodiments, the increase in the number of proerythroblasts, erythroblasts, reticulocytes, and/or erythrocytes upon contacting the cells with a perturbagen- is relative to the population of progenitor cells prior to contacting it with the perturbagen. In some embodiments, the change in the number of proerythroblasts, erythroblasts, reticulocytes, and/or erythrocytes is caused by change in the state of the cells of a population of progenitor cells. For example, an increase in the number of proerythroblasts, erythroblasts, reticulocytes, and/or erythrocytes within a population of progenitor cell can be due to a change in the state of the cells.

In some embodiments, the number of proerythroblasts, early erythroblasts, intermediate erythroblasts, and/or late erythroblasts is increased after contacting the population of cells comprising a CD34⁺ cell with the at least one perturbagen. In other embodiments, the number of early erythroblasts, intermediate erythroblasts, and/or late erythroblasts is increased after contacting the population of cells comprising a CD34⁺ cell with the at least one perturbagen. In some embodiments, the number of reticulocytes, and/or erythrocytes is increased after contacting the population of cells comprising a CD34⁺ cell with the at least one perturbagen. In some embodiments, the ratio of the number of proerythroblasts, early erythroblasts, intermediate erythroblasts, late erythroblasts, reticulocytes, and/or erythrocytes to the number of progenitor cells is increased relative to the ratio obtained from a population of progenitor cells that is not contacted with the at least one perturbagen. In some embodiments, the ratio of the number proerythroblasts, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen. In some embodiments, the number of proerythroblasts is increased after contacting the population of cells comprising a CD34⁺ cell with the at least one perturbagen. In some embodiments, the number of proerythroblasts is decreased after contacting the population of cells comprising a CD34⁺ cell with the at least one perturbagen. In some embodiments, the number of reticulocytes is increased after contacting the population of cells comprising a CD34⁺ cell with the at least one perturbagen. In some embodiments, the number of erythrocytes is increased after contacting the population of cells comprising a CD34⁺ cell with the at least one perturbagen.

In some embodiments of any of the methods disclosed herein, the ratio of the number of early erythroblasts, intermediate erythroblasts, late erythroblasts, reticulocytes, and/or erythrocytes to the number of proerythroblasts is increased relative to the ratio obtained from a population of progenitor cells that is not contacted with the at least one perturbagen. In some embodiments of any of the methods disclosed herein, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen. In some embodiments of any of the methods disclosed herein, the ratio of the number of early erythroblasts to the number of proerythroblasts is increased relative to the ratio obtained from a population of progenitor cells that is not contacted with the at least one perturbagen. In some embodiments of any of the methods disclosed herein, the ratio of the number of early erythroblasts to the number of proerythroblasts is increased relative to the ratio in the population of progenitor cells prior to contacting with the at least one perturbagen. In some embodiments of any of the methods disclosed herein, the ratio of the number of intermediate erythroblasts to the number of early erythroblasts is increased relative to the ratio obtained from a population of progenitor cells that is not contacted with the at least one perturbagen. In some embodiments of any of the methods disclosed herein, the ratio of the number of intermediate erythroblasts to the number of early erythroblasts is increased relative to the ratio in the population of progenitor cells prior to contacting with the at least one perturbagen. In some embodiments of any of the methods disclosed herein, the ratio of the number of late erythroblasts to the number of intermediate erythroblasts is increased relative to the ratio obtained from a population of progenitor cells that is not contacted with the at least one perturbagen. In some embodiments of any of the methods disclosed herein, the ratio of the number of late erythroblasts to the number of intermediate erythroblasts is increased relative to the ratio in the population of progenitor cells prior to contacting with the at least one perturbagen. In some embodiments of any of the methods disclosed herein, the ratio of the number of reticulocytes to the number of late erythroblasts is increased relative to the ratio obtained from a population of progenitor cells that is not contacted with the at least one perturbagen. In some embodiments of any of the methods disclosed herein, the ratio of the number of reticulocytes to the number of late erythroblasts is increased relative to the ratio in the population of progenitor cells prior to contacting with the at least one perturbagen. In some embodiments of any of the methods disclosed herein, the ratio of the number of erythrocytes to the number of reticulocytes is increased relative to the ratio obtained from a population of progenitor cells that is not contacted with the at least one perturbagen. In some embodiments of any of the methods disclosed herein, the ratio of the number of erythrocytes to the number of reticulocytes is increased relative to the ratio in the population of progenitor cells prior to contacting with the at least one perturbagen. In some embodiments of any of the methods disclosed herein, the ratio of the number of erythrocytes to late erythroblasts is increased relative to the ratio obtained from a population of progenitor cells that is not contacted with the at least one perturbagen. In some embodiments of any of the methods disclosed herein, the ratio of the number of erythrocytes to late erythroblasts is increased relative to the ratio in the population of progenitor cells prior to contacting with the at least one perturbagen. In some embodiments of any of the methods disclosed herein, the ratio of the number of erythrocytes to intermediate erythroblasts is increased relative to the ratio obtained from a population of progenitor cells that is not contacted with the at least one perturbagen. In some embodiments of any of the methods disclosed herein, the ratio of the number of erythrocytes to intermediate erythroblasts is increased relative to the ratio in the population of progenitor cells prior to contacting with the at least one perturbagen. In some embodiments of any of the methods disclosed herein, the ratio of the number of erythrocytes to early erythroblasts is increased relative to the ratio obtained from a population of progenitor cells that is not contacted with the at least one perturbagen. In some embodiments of any of the methods disclosed herein, the ratio of the number of erythrocytes to early erythroblasts is increased relative to the ratio in the population of progenitor cells prior to contacting with the at least one perturbagen. In some embodiments of any of the methods disclosed herein, the ratio of the number of erythrocytes to proerythroblasts is increased relative to the ratio obtained from a population of progenitor cells that is not contacted with the at least one perturbagen. In some embodiments of any of the methods disclosed herein, the ratio of the number of erythrocytes to proerythroblasts is increased relative to the ratio in the population of progenitor cells prior to contacting with the at least one perturbagen. In some embodiments of any of the methods disclosed herein, the ratio of the number of erythrocytes to early erythroblasts, intermediate erythroblasts, late erythroblasts, reticulocytes, and/or proerythroblasts is increased relative to the ratio obtained from a population of progenitor cells that is not contacted with the at least one perturbagen. In some embodiments of any of the methods disclosed herein, the ratio of the number of erythrocytes to early erythroblasts, intermediate erythroblasts, late erythroblasts, reticulocytes, and/or proerythroblasts is increased relative to the ratio in the population of progenitor cells prior to contacting with the at least one perturbagen.

In some embodiments, the increase in the number of proerythroblasts, erythroblasts, reticulocytes, and/or erythrocytes is due in part to 1) increased cell proliferation, 2) an increased lifespan, or 3) reduced cell death of the erythroblasts, reticulocytes, and/or erythrocytes. In some embodiments, the increase in the number of erythroblasts, reticulocytes, and/or erythrocytes is due in part to a change of cell state from progenitor cells into the erythrocyte lineage.

In some embodiments, the change in cell state provides an increase in the number of one or more of proerythroblasts, early erythroblasts, intermediate erythroblasts, late erythroblasts, reticulocytes, and erythrocytes. In some embodiments, the change in the state of the cells of a population of progenitor cells provides an increase in the number of committed blood cells, e.g., erythrocytes. In other embodiments, the change in cell state provide a substantial increase in the number of committed blood cells, e.g., erythrocytes. In some embodiments, the increase in the number of erythrocytes is relative to the number of erythrocytes obtained from a population of progenitor cells that is not contacted with the at least one perturbagen. In other embodiments, the increase in the number of erythrocytes is relative to the population of progenitor cells prior to contacting with the at least one perturbagen.

In some embodiments, the number of proerythroblasts, early erythroblasts, intermediate erythroblasts, late erythroblasts, reticulocytes, and/or erythrocytes is increased after contacting the population of cells comprising a CD34⁺ cell with the at least one perturbagen. In some embodiments, the number of erythrocytes is increased after contacting the population of cells comprising a CD34⁺ cell with the at least one perturbagen.

In some embodiments, the ratio of the number of erythrocytes to the number of progenitor cells is increased relative to the ratio obtained from a population of progenitor cells that is not contacted with the at least one perturbagen. In some embodiments, the ratio of the number of erythrocytes to the number of progenitor cells is increased relative to the ratio in the population of progenitor cells prior to contacting with the at least one perturbagen. In some embodiments, the increase in the number of erythrocytes is due in part to an increased lifespan of the erythrocytes. In some embodiments, the increase in the number of erythrocytes is due in part to reduced cell death among the erythrocytes. In some embodiments, the increase in the number of erythrocytes is due in part to a change of cell state from progenitor cells into the erythrocyte.

In some embodiments, the increase in the number of erythrocytes is due in part to increased cell proliferation of the erythrocytes. In some embodiments, the increase in the number of erythrocytes, is due in part to an increased lifespan of the erythrocytes. In some embodiments, the increase in the number of erythrocytes is due in part to reduced cell death among the erythrocytes. In some embodiments, the increase in the number of erythrocytes, is due in part to a change of cell state from progenitor cells into the erythrocyte lineage.

In some embodiments, the number of progenitor cells is decreased. In some embodiments, the decrease in the number of progenitor cells is due in part to decreased cell proliferation of the progenitor cells. In some embodiments, the decrease in the number of progenitor cells is due in part to a decreased lifespan of the progenitor cells. In some embodiments, the decrease in the number of progenitor cells is due in part to increased cell death among the progenitor cells. In some embodiments, the decrease in the number of progenitor cells is relative to the number of progenitor cells in a population of progenitor cells that is not contacted with the at least one perturbagen. In some embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into the erythrocyte lineage.

In some embodiments, the number of proerythroblasts is decreased. In some embodiments, the number of early erythroblasts is decreased. In some embodiments, the number of intermediate erythroblasts is decreased. In some embodiments, the number of late erythroblasts is decreased. In some embodiments, the number of reticulocytes is decreased. In some embodiments, proerythroblasts, early erythroblasts, intermediate erythroblasts, late erythroblasts, and/or reticulocytes is decreased.

In some embodiments, the number of progenitor cells is increased. In some embodiments, the increase in the number of progenitor cells is due in part to increased cell proliferation of the progenitor cells. In some embodiments, the increase in the number of progenitor cells is due in part to an increased lifespan of the progenitor cells. In some embodiments, the increase in the number of progenitor cells is due in part to decreased cell death among the progenitor cells. In some embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In some embodiments, the number of proerythroblasts is increased. In some embodiments, the number of proerythroblasts is decreased. In some embodiments, the number of early erythroblasts is increased. In some embodiments, the number of intermediate erythroblasts is increased. In some embodiments, the number of late erythroblasts is increased. In some embodiments, the number of reticulocytes is increased. In some embodiments, the number of erythrocytes is increased. In some embodiments, proerythroblasts, early erythroblasts, intermediate erythroblasts, late erythroblasts, and/or reticulocytes is increased.

In some embodiments, the erythrocytes can be derived from the canonical MEP developmental pathway. In other embodiments, the erythrocytes can be derived from a developmental pathway that does not include the canonical MEP cell. In embodiments, the erythrocytes may be produced from erythropoietin-independent pathway, for example, signal through gp130 and c-kit dramatically promote erythropoiesis from human CD34⁺ cells (Sui et al., Erythropoietin-independent erythrocyte production: signals through gp130 and c-kit dramatically promote erythropoiesis from human CD34⁺ cells, J. Exp. Med., 1996, vol. 183, pp. 837-845).

Methods for determining the extension of the lifespan of a specific cell type or a reduction of cell death is well known in the art. For examples, markers for dying cells, e.g., caspases can be detected, or dyes for dead cells, e.g., methylene blue, may be used.

In some embodiments, the expansion of erythroid progenitors was measured by positive expression of the surface marker CD235a⁺ (GYPA) with co-expression of CD71⁺ (transferrin receptor) with the CD34⁻CD38^{+/-} gate. In some embodiments, the megakaryocyte/erythroid progenitor cells are marked by antigen expression CD34⁺CD38⁺CD71^{high}CD41⁻CD235a⁻, in other embodiments, the early erythroid progenitor cells are marked by antigen expression CD34⁺CD38⁺CD71^{high}CD41⁻CD238⁺. In some embodiments, the late erythroid progenitors are marked by antigen expression CD34⁺CD38⁺CD71^{high}CD41-CD235a+. In some embodiments, the erythroid progenitor cells are marked by antigen expression CD34⁺CD38⁺CD71^{High}CD41⁻CD235a⁻. In some embodiments, the erythrocytes are marked by antigen expression CD34⁺CD38⁺CD71^{low}CD235a⁺CD41⁻. In other embodiments, the megakaryocytes are marked by antigen expression CD34⁺CD38⁺CD71⁺CD235a⁻CD41⁺. (See Example 2 infra).

In some embodiments, the expansion of erythroid progenitors was measured by positive expression of the surface marker CD235a⁺ (GYPA) with co-expression of CD71⁺ (transferrin receptor) with the CD34⁻CD38^{+/-} gate. In some embodiments, the megakaryocyte/erythroid progenitor cells are marked by antigen expression CD34⁺CD38⁺CD71⁺CD41a⁻CD36⁻CD235a⁻, in other embodiments, the early erythroid progenitor cells are marked by antigen expression CD34⁺CD38⁺CD71^{high}CD41a⁻CD36⁺CD235a⁻. In some embodiments, the late erythroid progenitors are marked by antigen expression CD34⁺CD38⁺CD71^{high}CD41a⁻CD36⁺CD235a⁺. In some embodiments, the erythroid progenitor cells are marked by antigen expression CD34⁻CD38⁺CD71^{High}CD41a⁻CD235a⁻. In some embodiments, the erythrocytes are marked by antigen expression CD34⁻CD38⁺CD71^{low} CD41a⁻CD235a⁺. (See Example 2 infra).

In some embodiments, the change in cell state causes an increase in the ratio of the number of erythrocytes to the number of progenitor cells relative to the ratio obtained from a population of progenitor cells that is not contacted with the at least one perturbagen. In other embodiments, the change in cell state causes an increase in the ratio of the number of erythrocytes to the number of progenitor cells relative to the ratio in the population of progenitor cells prior to contacting with the at least one perturbagen.

In some embodiments, the change in cell state causes an increase in the ratio of the number of early erythroblasts to the number of proerythroblasts relative to the ratio obtained from a population of progenitor cells that is not contacted with the at least one perturbagen. In other embodiments, the change in cell state causes an increase in the ratio of the number of early erythroblasts to the number of proerythroblasts relative to the ratio in the population of progenitor cells prior to contacting with the at least one perturbagen.

In some embodiments, the change in cell state causes an increase in the ratio of the number of intermediate erythroblasts to the number of early erythroblasts relative to the ratio obtained from a population of progenitor cells that is not contacted with the at least one perturbagen. In other embodiments, the change in cell state causes an increase in the ratio of the number of intermediate erythroblasts to the number of early erythroblasts relative to the ratio in the population of progenitor cells prior to contacting with the at least one perturbagen.

In some embodiments, the change in cell state causes an increase in the ratio of the number of late erythroblasts to the number of intermediate erythroblasts relative to the ratio obtained from a population of progenitor cells that is not contacted with the at least one perturbagen. In other embodiments, the change in cell state causes an increase in the ratio of the number of late erythroblasts to the number of intermediate erythroblasts relative to the ratio in the population of progenitor cells prior to contacting with the at least one perturbagen.

In some embodiments, the change in cell state causes an increase in the ratio of the number of reticulocytes to the number of late erythroblasts relative to the ratio obtained from a population of progenitor cells that is not contacted with the at least one perturbagen. In other embodiments, the change in cell state causes an increase in the ratio of the number of reticulocytes to the number of late erythroblasts relative to the ratio in the population of progenitor cells prior to contacting with the at least one perturbagen.

In some embodiments, the change in cell state causes an increase in the ratio of the number of erythrocytes to the number of reticulocytes relative to the ratio obtained from a population of progenitor cells that is not contacted with the at least one perturbagen. In other embodiments, the change in cell state causes an increase in the ratio of the number of erythrocytes to the number of reticulocytes relative to the ratio in the population of progenitor cells prior to contacting with the at least one perturbagen.

In some embodiments, the change in cell state causes an increase in the ratio of the number of erythrocytes to late erythroblasts relative to the ratio obtained from a population of progenitor cells that is not contacted with the at least one perturbagen. In other embodiments, the change in cell state causes an increase in the ratio of the number of erythrocytes to late erythroblasts relative to the ratio in the population of progenitor cells prior to contacting with the at least one perturbagen.

In some embodiments, the change in cell state causes an increase in the ratio of the number of erythrocytes to intermediate erythroblasts relative to the ratio obtained from a population of progenitor cells that is not contacted with the at least one perturbagen. In other embodiments, the change in cell state causes an increase in the ratio of the number of erythrocytes to intermediate erythroblasts relative to the ratio in the population of progenitor cells prior to contacting with the at least one perturbagen.

In some embodiments, the change in cell state causes an increase in the ratio of the number of erythrocytes to early erythroblasts relative to the ratio obtained from a population of progenitor cells that is not contacted with the at least one perturbagen. In other embodiments, the change in cell state causes an increase in the ratio of the number of erythrocytes to early erythroblasts relative to the ratio in the population of progenitor cells prior to contacting with the at least one perturbagen.

In some embodiments, the change in cell state causes an increase in the ratio of the number of erythrocytes to proerythroblasts relative to the ratio obtained from a population of progenitor cells that is not contacted with the at least one perturbagen. In other embodiments, the change in cell state causes an increase in the ratio of the number of erythrocytes to proerythroblasts relative to the ratio in the population of progenitor cells prior to contacting with the at least one perturbagen.

In some embodiments, the change in cell state causes an increase in the ratio of the number of erythrocytes to proerythroblasts relative to the ratio obtained from a population of progenitor cells that is not contacted with the at least one perturbagen. In other embodiments, the change in cell state causes an increase in the ratio of the number of erythrocytes to proerythroblasts relative to the ratio in the population of progenitor cells prior to contacting with the at least one perturbagen.

In some embodiments, the change in cell state causes an increase in the ratio of the number of proerythroblasts, erythroblasts, reticulocytes, and/or erythrocytes to the number of progenitor cells relative to the ratio obtained from a population of progenitor cells that is not contacted with the at least one perturbagen. In other embodiments, the change in cell state causes an increase in the ratio of the number of proerythroblasts, erythroblasts, reticulocytes, and/or erythrocytes to the number of progenitor cells relative to the ratio in the population of progenitor cells prior to contacting with the at least one perturbagen.

In some embodiments, the change in cell state causes an increase in the ratio of the number of proerythroblasts, erythroblasts, reticulocytes, and/or erythrocytes to the number of progenitor cells relative to the ratio obtained from a population of progenitor cells that is not contacted with the at least one perturbagen. In other embodiments, the change in cell state causes an increase in the ratio of the number of proerythroblasts, erythroblasts, reticulocytes, and/or erythrocytes to the number of progenitor cells relative to the ratio in the population of progenitor cells prior to contacting with the at least one perturbagen.

In some embodiments, the change in cell state causes an increase in the ratio of the number of early erythroblasts, intermediate erythroblasts, and/or late erythroblasts to the number of proerythroblasts relative to the ratio obtained from a population of progenitor cells that is not contacted with the at least one perturbagen. In other embodiments, the change in cell state causes an increase in the ratio of the number of early erythroblasts, intermediate erythroblasts, and/or late erythroblasts to the number of proerythroblasts relative to the ratio in the population of progenitor cells prior to contacting with the at least one perturbagen.

In some embodiments, the change in cell state causes an increase in the ratio of the number of early erythroblasts, intermediate erythroblasts, and/or late erythroblasts to the number of proerythroblasts relative to the ratio obtained from a population of progenitor cells that is not contacted with the at least one perturbagen. In other embodiments, the change in cell state causes an increase in the ratio of the number of early erythroblasts, intermediate erythroblasts, and/or late erythroblasts to the number of proerythroblasts relative to the ratio in the population of progenitor cells prior to contacting with the at least one perturbagen.

In some embodiments, the change in cell state causes an decrease in the ratio of the number of early erythroblasts, intermediate erythroblasts, and/or late erythroblasts to the number of proerythroblasts relative to the ratio obtained from a population of progenitor cells that is not contacted with the at least one perturbagen. In other embodiments, the change in cell state causes an increase in the ratio of the number of early erythroblasts, intermediate erythroblasts, and/or late erythroblasts to the number of proerythroblasts relative to the ratio in the population of progenitor cells prior to contacting with the at least one perturbagen.

In yet other embodiments, the change in cell state causes an increase in the ratio of the number of reticulocytes, and/or erythrocytes to the number of proerythroblasts relative to the ratio obtained from a population of progenitor cells that is not contacted with the at least one perturbagen. In other embodiments, the change in cell state causes an increase in the ratio of the number of reticulocytes, and/or erythrocytes to the number of proerythroblasts relative to the ratio in the population of progenitor cells prior to contacting with the at least one perturbagen. In yet other embodiments, the change in cell state causes an increase in the ratio of the number of reticulocytes to the number of proerythroblasts relative to the ratio obtained from a population of progenitor cells that is not contacted with the at least one perturbagen. In other embodiments, the change in cell state causes an increase in the ratio of the number of reticulocytes to the number of proerythroblasts relative to the ratio in the population of progenitor cells prior to contacting with the at least one perturbagen. In yet other embodiments, the change in cell state causes an increase in the ratio of the number of erythrocytes to the number of proerythroblasts relative to the ratio obtained from a population of progenitor cells that is not contacted with the at least one perturbagen. In other embodiments, the change in cell state causes an increase in the ratio of the number of erythrocytes to the number of proerythroblasts relative to the ratio in the population of progenitor cells prior to contacting with the at least one perturbagen.

In some embodiments, the change in cell state causes an increase in the ratio of the number of reticulocytes and/or erythrocytes to the number of proerythroblasts, early erythroblasts, intermediate erythroblasts, and/or late erythroblasts relative to the ratio obtained from a population of progenitor cells that is not contacted with the at least one perturbagen. In other embodiments, the change in cell state causes an increase in the ratio of the number of reticulocytes and/or erythrocytes to the number of proerythroblasts, early erythroblasts, intermediate erythroblasts, and/or late erythroblasts relative to the ratio in the population of progenitor cells prior to contacting with the at least one perturbagen. In some embodiments, the change in cell state causes an increase in the ratio of the number of erythrocytes to the number of proerythroblasts, early erythroblasts, intermediate erythroblasts, and/or late erythroblasts relative to the ratio obtained from a population of progenitor cells that is not contacted with the at least one perturbagen. In other embodiments, the change in cell state causes an increase in the ratio of the number of erythrocytes to the number of proerythroblasts, early erythroblasts, intermediate erythroblasts, and/or late erythroblasts relative to the ratio in the population of progenitor cells prior to contacting with the at least one perturbagen. In some embodiments, the change in cell state causes an increase in the ratio of the number of reticulocytes to the number of proerythroblasts, early erythroblasts, intermediate erythroblasts, and/or late erythroblasts relative to the ratio obtained from a population of progenitor cells that is not contacted with the at least one perturbagen. In other embodiments, the change in cell state causes an increase in the ratio of the number of reticulocytes to the number of proerythroblasts, early erythroblasts, intermediate erythroblasts, and/or late erythroblasts relative to the ratio in the population of progenitor cells prior to contacting with the at least one perturbagen.

In some embodiments, the change in cell state causes an increase in the ratio of the number of reticulocytes, and/or erythrocytes to the number of early erythroblasts, intermediate erythroblasts, and/or late erythroblasts relative to the ratio obtained from a population of progenitor cells that is not contacted with the at least one perturbagen. In other embodiments, the change in cell state causes an increase in the ratio of the number of reticulocytes, and/or erythrocytes to the number of early erythroblasts, intermediate erythroblasts, and/or late erythroblasts relative to the ratio in the population of progenitor cells prior to contacting with the at least one perturbagen. In some embodiments, the change in cell state causes an increase in the ratio of the number of reticulocytes to the number of early erythroblasts, intermediate erythroblasts, and/or late erythroblasts relative to the ratio obtained from a population of progenitor cells that is not contacted with the at least one perturbagen. In other embodiments, the change in cell state causes an increase in the ratio of the number of reticulocytes to the number of early erythroblasts, intermediate erythroblasts, and/or late erythroblasts relative to the ratio in the population of progenitor cells prior to contacting with the at least one perturbagen. In some embodiments, the change in cell state causes an increase in the ratio of the number erythrocytes to the number of early erythroblasts, intermediate erythroblasts, and/or late erythroblasts relative to the ratio obtained from a population of progenitor cells that is not contacted with the at least one perturbagen. In other embodiments, the change in cell state causes an increase in the ratio of the number of erythrocytes to the number of early erythroblasts, intermediate erythroblasts, and/or late erythroblasts relative to the ratio in the population of progenitor cells prior to contacting with the at least one perturbagen.

In some embodiments, the change in cell state causes an increase in the ratio of the number of early erythroblasts, intermediate erythroblasts, and/or late erythroblasts to the number of proerythroblasts relative to the ratio obtained from a population of progenitor cells that is not contacted with the at least one perturbagen. In other embodiments, the change in cell state causes an increase in the ratio of the number of early erythroblasts, intermediate erythroblasts, and/or late erythroblasts to the number of proerythroblasts relative to the ratio in the population of progenitor cells prior to contacting with the at least one perturbagen. In some embodiments, the change in cell state causes an increase in the ratio of the number of early erythroblasts to the number of proerythroblasts relative to the ratio obtained from a population of progenitor cells that is not contacted with the at least one perturbagen. In other embodiments, the change in cell state causes an increase in the ratio of the number of early erythroblasts to the number of proerythroblasts relative to the ratio in the population of progenitor cells prior to contacting with the at least one perturbagen. In some embodiments, the change in cell state causes an increase in the ratio of the number of early erythroblasts, intermediate erythroblasts, and/or late erythroblasts to the number of proerythroblasts relative to the ratio obtained from a population of progenitor cells that is not contacted with the at least one perturbagen. In other embodiments, the change in cell state causes an increase in the ratio of the number of early erythroblasts, intermediate erythroblasts, and/or late erythroblasts to the number of proerythroblasts relative to the ratio in the population of progenitor cells prior to contacting with the at least one perturbagen.

In some embodiments, the change in cell state causes an increase in the ratio of the number of early erythroblasts, intermediate erythroblasts, late erythroblasts, reticulocytes, and/or erythrocytes to the number of proerythroblasts relative to the ratio obtained from a population of progenitor cells that is not contacted with the at least one perturbagen. In other embodiments, the change in cell state causes an increase in the ratio of the number of early erythroblasts, intermediate erythroblasts, late erythroblasts, reticulocytes, and/or erythrocytes to the number of proerythroblasts relative to the ratio in the population of progenitor cells prior to contacting with the at least one perturbagen.

In some embodiments, the change in cell state causes an increase in the ratio of the number of intermediate erythroblasts, late erythroblasts, reticulocytes, and/or erythrocytes to the number of proerythroblasts relative to the ratio obtained from a population of progenitor cells that is not contacted with the at least one perturbagen. In other embodiments, the change in cell state causes an increase in the ratio of the number of intermediate erythroblasts, late erythroblasts, reticulocytes, and/or erythrocytes to the number of proerythroblasts relative to the ratio in the population of progenitor cells prior to contacting with the at least one perturbagen.

In some embodiments, the change in cell state causes an increase in the ratio of the number of reticulocytes, and/or erythrocytes to the number of proerythroblasts relative to the ratio obtained from a population of progenitor cells that is not contacted with the at least one perturbagen. In other embodiments, the change in cell state causes an increase in the ratio of the number of reticulocytes, and/or erythrocytes to the number of proerythroblasts relative to the ratio in the population of progenitor cells prior to contacting with the at least one perturbagen. In some embodiments, the change in cell state causes an increase in the ratio of the number of reticulocytes to the number of proerythroblasts relative to the ratio obtained from a population of progenitor cells that is not contacted with the at least one perturbagen. In other embodiments, the change in cell state causes an increase in the ratio of the number of reticulocytes to the number of proerythroblasts relative to the ratio in the population of progenitor cells prior to contacting with the at least one perturbagen. In some embodiments, the change in cell state causes an increase in the ratio of the number of erythrocytes to the number of proerythroblasts relative to the ratio obtained from a population of progenitor cells that is not contacted with the at least one perturbagen. In other embodiments, the change in cell state causes an increase in the ratio of the number of erythrocytes to the number of proerythroblasts relative to the ratio in the population of progenitor cells prior to contacting with the at least one perturbagen.

In some embodiments, the change in cell state causes an increase in the ratio of the number of erythrocytes to the number of erythroblasts and/or reticulocytes relative to the ratio obtained from a population of progenitor cells that is not contacted with the at least one perturbagen. In other embodiments, the change in cell state causes an increase in the ratio of the number of erythrocytes to the number of erythroblasts and/or reticulocytes relative to the ratio in the population of progenitor cells prior to contacting with the at least one perturbagen.

Methods for counting cells are well known in the art. Non-limiting examples include hemocytometry, flow cytometry, and cell sorting techniques, e.g., fluorescence activated cell sorting (FACS). The maturation of the erythrocytes is, in some embodiments, determined by loss of CD71 expression. Erythroid maturation is determined by, in some embodiments, flow cytometry using a four-antibody panel (See Example 2 infra) (CD71, CD235a, CD233, CD49d) with increased CD233 expression, with a concomitant loss of CD49d expression, and a shift in CD71^{Hi} to CD71^{low} erythroid population (CD235a+).

In some embodiments, the at least one perturbagen selected from Table 5, or a variant thereof, comprises at least 2, at least 3, at least 4, or at least 5 perturbagens selected from Table 5, or variants thereof.

In some embodiments, altering the gene signature comprises increased expression and/or increased activity in the progenitor cell of one or more genes selected from the genes designated as an “up” gene in the gene directionality column of Table 2. In this aspect, the one or more genes selected from the genes designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2. In other embodiments, the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39.

In some embodiments, altering the gene signature comprises decreased expression and/or decreased activity in the progenitor cell of one or more genes selected from the genes designated as an “down” gene in the gene directionality column of Table 2. In this aspect, the one or more genes selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2. In some embodiments, the one or more genes selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNA/2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1.

In some embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In an aspect, the present technology provides a method for promoting the formation of a erythrocytes or an immediate progenitor thereof. The method includes a step of exposing a starting population of stem/progenitor cells comprising a non-lineage committed CD34⁺ cell to a perturbation having a perturbation signature that promotes the transition of the starting population of stem/progenitor cells into proerythroblasts, early erythroblasts, intermediate erythroblasts, and/or late erythroblasts. In some embodiments, the perturbation signature comprises increased expression and/or activity of one or more of genes associated with at least one functionality selected from the genes designated as an “up” gene in the gene directionality column of Table 2 and/or a decreased expression and/or activity in the non-lineage committed CD34⁺ cell of one or more genes selected from the genes designated as an “down” gene in the gene directionality column of Table 2. Embodiments associated with the above aspects are likewise relevant to the present aspect. In other words, each of the embodiment mentioned above for the above aspects may be revised/adapted to be applicable to the present aspect.

In an aspect, the present technology provides methods of preparing cultured erythroblasts having at least one perturbation signature selected from Table 2 using hematopoietic stem cells. In some embodiments, the hematopoietic stem cells comprise CD34⁺ cells derived from mobilized peripheral blood (mPB), cord blood (CB), or bone marrow (BM). As demonstrated in the Examples 1-2 below, methods and protocols have been established for differentiating non-lineage committed CD34+ cells into a CD71⁺CD235a⁺ erythroblasts in culture.

In some embodiments, the method the present technology provides methods of preparing cultured erythroblasts having at least one perturbation signature selected from Table 2. In this aspect, the method comprises the steps including: (1) providing non-lineage committed CD34⁺ cells, and (2) culturing the CD34⁺ cells in a culture medium containing factors sufficient to make the erythroblasts (e.g., StemSpan™ serum free expansion medium supplemented with StemSpan™ Erythroid Expansion Supplement, Stem Cell Technologies Cat #09650). In this aspect, the CD34⁺ cells are derived from a sample of cells comprising a non-lineage committed CD34⁺ cell from a subject exhibiting an abnormal number of erythrocyte, or a disease or disorder characterized thereby. In some embodiments, the CD34⁺ cells are derived from a sample of cells comprising a non-lineage committed CD34⁺ cell from a healthy subject. In some embodiments, the CD34⁺ cells are freshly isolated or cryopreserved from cord blood or bone marrow sample. In some embodiments, the CD34⁺ cells are freshly isolated or cryopreserved from peripheral blood sample. In some embodiments, the non-lineage committed CD34⁺ cells comprises autologous CD34⁺ cells. In some embodiments, the non-lineage committed CD34⁺ cells comprises allogenic CD34⁺ cells.

In some embodiments, the culture medium comprises a combination of one or more factors selected from IL-3, IL-6, glucocorticoids, or SCF which together promote the proliferation and differentiation of the progenitor cells to generate thousands of erythroblasts per input CD34⁺ cell after 1-3 weeks of culture.

In some embodiments, the culture-expanded erythroblasts as described herein express transferrin receptor (CD71) and glycophorin (GlyA, CD235a), but express little or no hemoglobin (Hb). In some embodiments, the cell culture produces at least 1000 CD71⁺CD235a⁺ erythroblasts per original CD34⁺ cell after two weeks of culture. In some embodiments, the culture produces about 1×10⁵ CD71⁺CD235a⁺ erythroblasts per original CD34⁺ cell after two weeks of culture.

In some embodiments, the present technology provides a method for promoting the formation of erythroblasts in the presence of a perturbagen. In this aspect, the method includes a step of contacting a starting population of non-lineage committed CD34⁺ cells to a perturbation having a perturbation signature that promotes the transition of the starting population of stem/progenitor cells into a erythroblasts. In some embodiments, the perturbation signature comprises increased expression and/or activity of one or more of genes selected from the genes designated as an “up” gene in the gene directionality column of Table 2 and/or a decreased expression and/or activity in the non-lineage committed CD34⁺ cell of one or more genes selected from the genes designated as an “down” gene in the gene directionality column of Table 2. Embodiments associated with the above aspects are likewise relevant to the present aspect. In other words, each of the embodiment mentioned above for the above aspects may be revised/adapted to be applicable to the present aspect.

In some embodiments, the present technology provides a method of promoting the formation of erythroblasts. The method includes a step of exposing a starting population of non-lineage committed CD34⁺ cells to a pharmaceutical composition that promotes the formation of lineage specific progenitor population of erythroblasts. In embodiments, the pharmaceutical composition comprises at least one perturbagen selected from Table 5, or a variant thereof. Embodiments associated with the above aspects are likewise relevant to the present aspect. In other words, each of the embodiment mentioned above for the above aspects may be revised/adapted to be applicable to the present aspect.

In some embodiments, the non-lineage committed CD34⁺ cells are differentiated into erythroblasts in the presence of at least one perturbagen selected from Table 5, or a variant thereof. In some embodiments, the presence of the at least one perturbagen selected from Table 5, or a variant thereof causes substantial increases in the number of erythroblasts in the culture as compared with the prior art methods. Differentiation of the non-lineage committed CD34⁺ cells into erythroblasts is a multi-step process as the CD34⁺ cells undergo a series of cell fate determinations as they differentiate from a starting pluripotent state, to a hematopoietic-endothelial state, to a multipotent HSC state, and to an erythroblast. In some embodiments, the perturbagen is present in the culture media for at least part of the total cell culture period (sub-period). In some embodiments, the sub-period with the presence of the at least one perturbagen is selected from 1 to 12 hours, from 3 to 12 hours, from 6 to 12 hours, from 6 to 24 hours, from 12 to 24 hours, from 1 day to 2 days, from 2 days to 4 days, from 3 days to 6 days, from 1 to 2 weeks, or from 2 to 4 weeks. In some embodiments, the sub-period with the presence of the at least one perturbagen is selected from at least 1 hour, at least 2 hours, at least 3 hours, at least 4 hours, at least 5 hours, at least 7 hours, at least 8 hours, at least 9 hours, at least 10 hours, at least 11 hours, at least 12 hours, at least 18 hours, at least 24 hours, at least 36 hours, at least 2 days, at least 3 days, at least 4 days, at least 5 days, at least 6 days, at least 7 day, at least two weeks, or at least 3 weeks.

In some embodiments, the at least one perturbagen selected from Table 5, or a variant thereof is present for the entire cell culture period. In some embodiments, the culture of the non-lineage committed CD34⁺ cells is initiated in the absence of the at least one perturbagen selected from Table 5, or a variant thereof and the at least one perturbagen selected from Table 5, or a variant thereof is added after a period of time selected from at least 1 day, at least 2 days, at least 3 days, at least 4 days, at least 5 days, at least 6 days, at least 1 week, or at least 2 weeks. In some embodiments, the culture of the non-lineage committed CD34⁺ cells is initiated in the absence of the at least one perturbagen selected from Table 5, or a variant thereof and the at least one perturbagen selected from Table 5, or a variant thereof is added after a period of time selected from: from 12 hours to 24 hours, from 1 day to 2 days, from 1 day to 3 days, from 2 days to 4 days, from 3 days to 6 days, from 4 days to 7 days, from 5 days to 10 days, from 6 days to 10 days, or from 7 days to 10 days. In some embodiments, the culture of the non-lineage committed CD34⁺ cells begin to differentiate erythroblasts within 7 to 10 days.

In some embodiments, the non-lineage committed CD34⁺ cells are differentiated into erythroblasts in a culture medium comprising a combination of factors selected from SCF, Flt3L, IL-3, and IL-6, which promotes expansion and non-directed lineage differentiation.

In some embodiments, the non-lineage committed CD34⁺ cells are differentiated into erythroblasts in the presence of at least one perturbagen selected from Table 5.

In some embodiments, the cultured erythroblasts made by these or other methods described herein are isolated, e.g., by centrifugation. In a some embodiments, the present technology provides a composition comprising the cultured erythroblasts prepared according to the herein described methods suspended in physiological saline or red blood cell additive solution. In some embodiments, the composition of the cultured erythroblasts has a concentration of about 5×10⁵ to 10×10⁵ cells per mL. In some embodiments, the frozen stocks of the composition containing the cultured erythroblasts are prepared using appropriate cryoprotectant (e.g., glycerol). Such stocks may be thawed periodically to provide an indefinite supply of cell culture that differentiated into erythrocytes.

In an aspect, the present technology provides a method for preparing cultured erythrocytes having at least on gene signature selected from Table 2 using hematopoietic stem cells. In some embodiments, the hematopoietic stem cells are CD34⁺ cells derived from mobilized peripheral blood (mPB), cord blood (CB), or bone marrow (BM). In this aspect, the method comprises a multi-phase process including: (1) a first phase of preparing cultured erythroblasts by providing progenitor cells selected from non-lineage committed CD34⁺ cells, and culturing the progenitor cells in a culture medium containing factors sufficient to make the erythroblasts (e.g., StemSpan™ serum free expansion medium supplemented with StemSpan™ Erythroid Expansion Supplement, Stem Cell Technologies Cat #09650); and a second phase of promoting the differentiation of expanded erythroblasts into erythrocytes in the presence of erythropoietin stimulating agent (ESA, e.g., erythropoietin (EPO)). In a further aspect, the CD34⁺ cells are derived from a sample of cells comprising a non-lineage committed CD34⁺ cell from a subject exhibiting an abnormal number of erythrocyte, or a disease or disorder characterized thereby. In some embodiments, the CD34⁺ cells are derived from a sample of cells comprising a non-lineage committed CD34⁺ cell from a healthy subject. In some embodiments, the CD34⁺ cells are freshly isolated or cryopreserved from cord blood or bone marrow sample. In some embodiments, the CD34⁺ cells are freshly isolated or cryopreserved from peripheral blood sample. In some embodiments, the non-lineage committed CD34⁺ cells comprises autologous CD34⁺ cells. In some embodiments, the non-lineage committed CD34⁺ cells comprises allogenic CD34⁺ cells.

In some embodiments, the culture medium comprises a combination of one or more selected from IL-3, IL-6, glucocorticoids, or SCF which together promote the proliferation and differentiation of the progenitor cells to generate thousands of erythroblasts per input CD34⁺ cell after 1-3 weeks of culture. The culture-expanded erythroblasts as described herein express transferrin receptor (CD71) and glycophorin (GlyA, CD235a), but express little or no hemoglobin (Hb). In some embodiments, the culture produces at least 1×10⁵ CD71⁺CD235a⁺ erythroblasts per mL.

In some embodiments, the method comprises culturing the progenitor cells in the presence of at least one perturbagen selected from Table 5, or a variant thereof. In some embodiments, the culture media comprise erythropoietin (EPO). EPO has its primary effect on red blood cell progenitors and precursors by promoting their survival through protecting these cells from apoptosis to promote definitive erythropoiesis. Erythropoietin is the primary erythropoietic factor that cooperates with various other growth factors (interleukin 3 (IL-3), interleukin 6 (IL-6), glucocorticoids, cytokine stem cell factor (SCF) as in StemSpan™ Erythroid Expansion Supplement) involved in the development of erythroid lineage from non-lineage committed CD34⁺ cells as described herein. In some embodiments, the culture-expanded erythroblasts as described herein are isolated and combined with culture media containing EPO and one or more selected from IL-3, IL-6, glucocorticoids, or SCF which together promote the proliferation and differentiation of the erythroblasts derived from CD34⁺ cells to yield hemoglobin expressing reticulocytes and/or erythrocytes.

In some embodiments, the present technology provides the use of CD34⁺ cells derived from mobilized peripheral blood (mPB), cord blood (CB) or bone marrow (BM) to prepare the cultured erythrocytes for use in transfusion therapy. In some embodiments, the present technology utilizes a three-phase process. to expand and cause cellular maturation: In Phase 1, CD34+ cells are thawed and cultured for five days in expansion media comprising StemSpan Serum Free media (Stem Cell Technologies Cat #09650) and CC100 cocktail (Stem Cell Technologies Cat #02690), which contains SCF, Flt3L, IL-3, and IL-6, which promotes expansion and non-directed lineage differentiation. In Phase 1 (days 0-7), the StemSpan media is supplemented with lipid mixture, SCF, IL-3, holotransferrin, insulin and EPO. Following Phase 1 in expansion media, cells were transferred to differentiation media, containing EPO, to drive erythroid differentiation. In Phase 2 (days 7-11), the StemSpan expansion media is supplemented with lipid mixture, holotransferrin, insulin and EPO which promotes differentiation of progenitor cells into committed blood cells. On day 11 the cells were once again harvested and resuspended for Phase 3 (days 11-18) with StemSpan expansion media supplemented with lipid mixture, holotransferrin, insulin and EPO. This methodology allows for cell expansion and maturation of CD34+ cells into erythrocytes.

In some embodiments, the method comprises culturing the CD34⁺ cells in the presence of at least one perturbagen selected from Table 5, or a variant thereof to produce cultured red blood cells (cRBCs) from CD34⁺ cells. In some embodiments, the at least one perturbagen selected from Table 5, or a variant thereof is present in the cell culture media during the entire culture period of 18 days. In some embodiments, the at least one perturbagen selected from Table 5, or a variant thereof is added to the cell culture during days 7-11. In some embodiments, the at least one perturbagen selected from Table 5, or a variant thereof is added to the cell culture at day 7. In some embodiments, the at least one perturbagen selected from Table 5, or a variant thereof is present in the cell culture media during Phase 2 and Phase 3 period.

In some embodiments, the non-lineage committed CD34⁺ cells are differentiated into erythrocytes (cRBCs) in a culture medium comprising a combination of factors selected from EPO, SCF, Flt3L, IL-3, and IL-6, which promotes the differentiation of non-lineage committed CD34⁺ cells in to matured erythrocytes.

In some embodiments, the non-lineage committed CD34⁺ cells are differentiated into matured erythrocytes in the presence of at least one perturbagen selected from Table 5.

In some embodiments, the cRBCs prepared according to the methods above have a biconcave shape.

In some embodiments, the cRBCs are isolated. In some embodiments, the present technology provides a composition comprising cRBCs as described above suspended in physiological saline or red blood cell additive solution for administering to a mammal (cRBC stock). In some embodiments, the stock of cRBCs can be stored for normal time frame (4 weeks) without loss of RBC characteristics.

In some embodiments, the frozen stocks of composition of the cRBC are created using appropriate cryoprotectant (e.g., glycerol). In some embodiments, the frozen stock of cRBC comprises autologous red blood cells. In some embodiments, the frozen stock of cRBC comprises autologous red blood cells of rare groups and phenotypes. In some embodiments, the frozen cRBC stock is thawed, washed, deglycerolized, resuspended in physiological saline or red blood cell additive solution before being used.

In a further aspect, the present technology provides compositions containing the cultured RBCs as described herein for use in transfusion therapy (e.g., as supportive care for anemia patient). In some embodiments, cRBCs are capable of complete maturation after transfusion to an animal or a human as determined by loss of CD71 expression, organelles, and surface area. In some embodiments, the administering the cRBC cell is via intravenous infusion.

In yet another aspect, the present technology provides a perturbagen for use in any herein disclosed method.

In a further aspect, the present technology provides a pharmaceutical composition comprising perturbagen for use in any herein disclosed method. In some embodiments, the at least one perturbagen is administered on the basis of previously determining the subject exhibits an abnormal number of erythrocyte, or a disease or disorder characterized thereby.

### Methods and Perturbagens for Treating a Disease or Disorder

Blood flows to every cell in the body and is important to the health and function of all of the body's organs. The main components of blood include plasma, red blood cells, white blood cells and platelets. Blood cells and blood proteins provide the following functions: red blood cells carry oxygen to every part of the body, white blood cells and antibodies fight infection and cancers, and platelets and blood clotting factors makes bleeding stop or prevent bleeding from occurring. The normal daily production of red blood cells (RBC) in a healthy adult is about 0.25 mL/kg and the average lifespan of the cells is about 120 days, whereas that of transfused RBCs is about 50-60 days and can be significantly shorter in the presence of factors reducing their survival.

There are many blood disorders, and they can affect the quantity as well as the function of the cells in the blood (blood cells) or proteins in the blood clotting system or immune system. Some blood disorders cause the number of cells in the blood to decrease: e.g., anemia (the number of red blood cells being too low), leukopenia (the number of white blood cells being too low), and thrombocytopenia (the number of platelets being too low). Other blood disorders cause the numbers of blood cells to increase: e.g., erythrocytosis (the number of red blood cells being too high, e.g., polycythemia vera), leukocytosis (the number of white blood cells being too high), and thrombocythemia (the number of platelets being too high). Other blood disorders affect proteins within the blood cells or blood plasma: e.g., hemoglobin (Hb), immune system proteins, such as antibodies, and blood clotting factors.

Anemia is defined as a reduction of the hemoglobin (Hb) concentration, red blood cell count or packed cell volume below normal level. Anemia can develop from loss of red blood cells (RBCs), a reduction in RBC production, increased destruction of RBCs, or a shorter RBC lifespan. The World health Organization defines anemia as a hemoglobin level lower than 12 g/dL in women and lower than 13 g/dL in men. Mild anemia has an Hb of ranging from 10 g/dL to 11.9 g/dL, moderate anemia has an Hb ranging from 8.0 g/dL to 9.9 g/dL, and severe anemia has an Hb less than 8.0 g/dL.

Decreased erythropoietin (EPO) production, shortened erythrocyte survival, and other factors reducing the response to EPO contribute to anemia in patients who have a variety of underlying pathologies such as myelodysplastic syndrome, aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia.

Hematopoietic drugs (e.g., iron, vitamin B12, folic acid, recombinant erythropoietin) have been used to treat anemia. Treating anemia with recombinant human EPO (rHuEPO, an erythropoietin stimulating agent (ESA)) at supraphysiologic concentrations has proven to be efficacious. However, it does not ameliorate the condition in all patients, and it presents its own risks, including cardiovascular complications. New ESAs (e.g., short peptide based ESA), activation of endogenous EPO production through HIF stabilization and GATA1 inhibition, and EPO gene therapy have been developed.

The transcription factors hypoxia-inducible factor (HIF) family has three hypoxia responsive proteins: HIF-1α, HIF-2α and HIF-3α. HIF-1α and HIF-2α control the physiologic response to hypoxia and invoke a program of increased erythropoiesis. Under normoxia, HIFα is continually ubiquitinated and degraded by the proteasome. E3 ubiquitin ligase-mediated degradation of HIFα depends upon interaction with von Hippel-Lindau tumor suppressor protein. The oxygen-sensing mechanism controlling HIFα stabilization involves a family of HIF-prolyl hydroxylases (PHDs), which regulate the hydroxylation of conserved proline residues in HIFα, furnishing the essential recognition element for the HIFα-VHL interaction. Levels of HIFα are modulated by oxygen tension via the action of a family of HIF-prolyl hydroxylases (PHDs), which tag HIFα for proteasomal degradation (Schödel et al., High-resolution genome-wide mapping of HIF-binding sites by ChIP-seq. Blood, 2011, vol. 117, pp. e207-e217; Semenza, Hypoxia-inducible factors in physiology and medicine, Cell, 2012, vol. 148, pp. 399-408).

Activin receptor ligands are members of the TGF-β superfamily which negatively regulate erythropoiesis by induction of apoptosis and cell-cycle arrest in erythroblasts resulting in inhibition of erythroid differentiation. The compounds inhibit the TGF-β pathway by binding to select TGF-β superfamily ligands to reduce aberrant Smad 2/3 signaling. After inhibition of Smad-signaling, late stage erythropoiesis such as differentiation of erythroblastst to RBC is promoted (Kubasch et al., Setting Fire to ESA and EMA Resistance: New Targeted Treatment Options in Lower Risk Myelodysplastic Syndromes, Int. J. Mol. Sci., 2019, vol. 20, pp. 3853-3862).

Myelodysplastic syndrome are clonal hematopoietic stem/progenitor cell (HSPC) disorders characterized by ineffective hematopoiesis, peripheral cytopenia, and abnormal marrow cell morphology. Erythroid differentiation in DBA is arrested at the earliest progenitor stage, the erythroid burst-forming unit (BFU-E). Current standard treatment options for anemia in MDS include supportive care with regular red blood cell transfusions and ESAs. However, RBC transfusions and chronic anemia are independent risk factors associated with iron overload, resulting increased cardiovascular risk affecting survival. Response rate to ESAs are low and mostly only transient. Erythroid maturation agents (EMAs), ligand of activing receptor II promoting later stage erythropoiesis, are promising alternative treatments. Other new treatment alternatives include immune modulatory drug (ImiDs) (e.g., lenalidomide), PHD inhibitor (e.g., roxadustat), telomerase inhibitor (e.g., imetelstat), and immunosuppressive therapy.

EPO refractory anemia (RA) is a clonal disorder originating from a totipotent stem cell or from a multipotent myeloid progenitor cell, characterized by ineffective hemopoiesis and diserythropoiesis. In the WHO classification RA shows anemia, no or rare blasts in the peripheral blood, isolated erythroid dysplasia with <5% blasts and <15% ringed sideroblasts in the BM. The conventional RA treatment is to get the number and type of blood cells in the bloodstream back to normal, and treat symptoms with supportive treatment (blood transfusions, injections of growth factor drugs and antibiotics), immunosuppression treatment (anti-thymocyte immunoglobulin (ATG) and ciclosporin) chemotherapy, and stem-cell bone marrow transplant.

Aplastic anemia (AA) is a bone marrow failure disorder caused by lymphocyte destruction of early hematopoietic cells. A trigger-related abnormal T cell response facilitated by some genetic predisposition has been postulated as the pathogenic mechanism leading to the overproduction of bone marrow-inhibiting cytokines. The frontline therapy for AA include bone marrow transplantation.

Diamond-Blackfan anemia (DBA) is a congenital disorder characterized by the failure of erythroid progenitor differentiation, severely curtailing red blood cell production. Diamond-Blackfan anemia can be caused by mutations on one of many genes, including the RPL5, RPL11, RPL35A, RPS10, RPS17, RPS19, RPS24, and RPS26. Current standard treatment options for anemia in DBA include red blood cell transfusions, stem cell/bone marrow transplantation, and corticosteroid therapy (e.g., prednisone). However, many DBA patients fail to respond to corticosteroid therapy, there is considerable need for therapeutics for this disorder.

However, none of these prior art methods have shown efficacy superior to that of existing ESAs. There exists a need for new anemia therapies with satisfactory levels of efficacy and safety and faster action.

An aspect of the present technology is a method for treating a disease or disorder characterized by an abnormal erythron distribution and/or physiology. In this aspect, the method comprises the step of administering to a subject in need thereof a therapeutically effective amount of at least one perturbagen capable of altering a gene signature in a progenitor cell.

Another aspect of the present technology is a method for treating a disease or disorder characterized by an abnormal erythron distribution and/or physiology. In this aspect, the method comprises the step of administering to a subject in need thereof a cell, the cell having been contacted with at least one perturbagen capable of altering a gene signature in a progenitor cell.

An aspect of the present technology is a method for treating a disease or disorder characterized an abnormal erythron distribution and/or physiology. In this aspect, the method comprises the step of administering to a subject in need thereof a therapeutically effective amount of a combination therapy having an ESA and at least one perturbagen, wherein the combination therapy is capable of altering a gene signature in a progenitor cell.

In some embodiments, for any herein disclosed method, the ESA is selected from the group consisting of rHuEPO, darbeportin alpha (Aranesp®), epoetin alpha (Epogen®, Procrit®), epoetin alpha-epbx (Retacrit®), and methoxy polyethylene glycol-epoetin beta (Mircera®).

An aspect of the present technology is a method for treating a disease or disorder characterized by an erythrocyte deficiency. In this aspect, the method comprises the step of administering to a subject in need thereof a therapeutically effective amount of at least one perturbagen capable of altering a gene signature in a progenitor cell.

Another aspect of the present technology is a method for treating a disease or disorder characterized by an erythrocyte deficiency. In this aspect, the method comprises the step of administering to a subject in need thereof a cell, the cell having been contacted with at least one perturbagen capable of altering a gene signature in a progenitor cell.

An aspect of the present technology is a method for treating a disease or disorder characterized an erythrocyte deficiency. In this aspect, the method comprises the step of administering to a subject in need thereof a therapeutically effective amount of a combination therapy having an ESA and at least one perturbagen, wherein the combination therapy is capable of altering a gene signature in a progenitor cell.

In some embodiments, for any herein disclosed method, the ESA is selected from the group consisting of rHuEPO, darbeportin alpha (Aranesp®), epoetin alpha (Epogen®, Procrit®), epoetin alpha-epbx (Retacrit®), and methoxy polyethylene glycol-epoetin beta (Mircera®).

An aspect of the present technology is a method for treating or preventing anemia. In this aspect, the method comprises the step of administering to a subject in need thereof a therapeutically effective amount of at least one perturbagen capable of altering a gene signature in a progenitor cell.

Another aspect of the present technology is a method for treating or preventing anemia. In this aspect, the method comprises the step of administering to a subject in need thereof a cell, the cell having been contacted with at least one perturbagen capable of altering a gene signature in a progenitor cell.

An aspect of the present technology is a method for treating or preventing anemia. In this aspect, the method comprises the step of administering to a subject in need thereof a therapeutically effective amount of a combination therapy having an ESA and at least one perturbagen, wherein the combination therapy is capable of altering a gene signature in a progenitor cell.

In some embodiments, for any herein disclosed method, the ESA is selected from the group consisting of rHuEPO, darbeportin alpha (Aranesp®), epoetin alpha (Epogen®, Procrit®), epoetin alpha-epbx (Retacrit®), and methoxy polyethylene glycol-epoetin beta (Mircera®).

In some embodiments, for any herein disclosed method, anemia is selected from the group consisting of aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia.

In some embodiments, any herein disclosed methods of treatment further comprise administration of one or more of recombinant erythropoietin and hydroxyurea. In some embodiments, the present methods of treatment involve a subject undergoing treatment with one or more of recombinant erythropoietin and hydroxyurea.

An aspect of the present technology is a method for treating a disease or disorder characterized by an abnormal erythron distribution and/or physiology. The method comprises the steps of: (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.

In another aspect, the present technology provides a method for treating a disease or disorder characterized by an abnormal erythron distribution and/or physiology. The method comprises the steps of: (a) administering to a subject in need thereof a therapeutically effective amount of at least one perturbagen selected from Table 5, or a variant thereof, wherein the at least one perturbagen is capable of altering a gene signature in a progenitor cell or (b) administering to a subject in need thereof a cell, the cell having been contacted with at least one perturbagen selected from Table 5, or a variant thereof. In this aspect, the erythrocyte deficiency is due partly to decreased erythropoietin (EPO) production, shortened erythrocyte survival, other factors reducing the response to EPO, and optionally resultant from mutations in one or more hemoglobin genes, the mutation optionally being in a HBB gene.

An aspect of the present technology is a method for treating a disease or disorder characterized by an erythrocyte deficiency. The method comprises the steps of: (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.

In another aspect, the present technology provides a method for treating a disease or disorder characterized by an erythrocyte deficiency. The method comprises the steps of: (a) administering to a subject in need thereof a therapeutically effective amount of at least one perturbagen selected from Table 5, or a variant thereof, wherein the at least one perturbagen is capable of altering a gene signature in a progenitor cell or (b) administering to a subject in need thereof a cell, the cell having been contacted with at least one perturbagen selected from Table 5, or a variant thereof. In this aspect, the erythrocyte deficiency is due partly to decreased erythropoietin (EPO) production, shortened erythrocyte survival, other factors reducing the response to EPO, and optionally resultant from mutations in one or more hemoglobin genes, the mutation optionally being in a HBB gene.

In another aspect, the present technology provides a method for treating or preventing an anemia. The method comprises the steps of: (a) administering to a subject in need thereof a therapeutically effective amount of at least one perturbagen selected from Table 5, or a variant thereof, where the at least one perturbagen is capable of changing a gene signature in a progenitor cell or (b) administering to a subject in need thereof a cell, the cell having been contacted with at least one perturbagen selected from Table 5, or a variant thereof, where the at least one perturbagen is capable of changing a gene signature in a progenitor cell.

In another aspect, the present technology provides a method for treating or preventing an anemia. The method comprises the steps of: (a) administering to a subject in need thereof a therapeutically effective amount of at least one perturbagen selected from Table 5, or a variant thereof, where the at least one perturbagen is capable of changing a gene signature in a progenitor cell or (b) administering to a subject in need thereof a cell, the cell having been contacted with at least one perturbagen selected from Table 5, or a variant thereof, where the at least one perturbagen is capable of changing a gene signature in a progenitor cell. In this aspect, the erythrocyte deficiency is due partly to decreased erythropoietin (EPO) production, shortened erythrocyte survival, other factors reducing the response to EPO, and optionally resultant from mutations in one or more hemoglobin genes, the mutation optionally being in a HBB gene.

In one embodiment, the administering is directed to the bone marrow of the subject. In other embodiments, the administering is via intraosseous injection or intraosseous infusion. In other embodiments, the administering the cell is via intravenous injection or intravenous infusion. In other embodiments, the administering is simultaneously or sequentially to one or more mobilization agents.

An aspect of the present technology is a method for treating a disease or disorder characterized by an abnormal erythron distribution and/or physiology. In this aspect, the method comprises the steps of administering to a subject in need thereof a therapeutically effective amount of at least one perturbagen capable of altering a gene signature in a progenitor cell. Another aspect of the present technology is a method for treating a disease or disorder characterized by an abnormal erythron distribution and/or physiology. In this aspect, the method comprises the steps of administering to a subject in need thereof a cell, the cell having been contacted with at least one perturbagen capable of altering a gene signature in a progenitor cell.

An aspect of the present technology is a method for treating a disease or disorder characterized by an erythrocyte deficiency. In this aspect, the method comprises the steps of administering to a subject in need thereof a therapeutically effective amount of at least one perturbagen capable of altering a gene signature in a progenitor cell. In other embodiments, the method comprises the steps of administering to a subject in need thereof a cell, the cell having been contacted with at least one perturbagen capable of altering a gene signature in a progenitor cell.

An aspect of the present technology is a method for treating or preventing anemia. In this aspect, the method comprises the steps of administering to a subject in need thereof a therapeutically effective amount of at least one perturbagen capable of altering a gene signature in a progenitor cell. In other embodiments, the method comprises the steps of administering to a subject in need thereof a cell, the cell having been contacted with at least one perturbagen capable of altering a gene signature in a progenitor cell.

An aspect of the present technology is a method for treating a disease or disorder characterized by an abnormal erythron distribution and/or physiology. In this aspect, the method comprises the steps of administering to a subject in need thereof a therapeutically effective amount of a combination therapy of rhuEPO and at least one perturbagen selected from Table 5, wherein the combination therapy is capable of altering a gene signature in a progenitor cell. In other embodiments, the method comprises the steps of administering to a subject in need thereof a cell, the cell having been contacted with a combination therapy of rhuEPO and at least one perturbagen selected from Table 5, wherein the combination therapy is capable of altering a gene signature in a progenitor cell.

An aspect of the present technology is a method for treating a disease or disorder characterized by an erythrocyte deficiency. In this aspect, the method comprises the steps of administering to a subject in need thereof a therapeutically effective amount of a combination therapy of rhuEPO and at least one perturbagen selected from Table 5, wherein the combination therapy is capable of altering a gene signature in a progenitor cell. In other embodiments, the method comprises the steps of administering to a subject in need thereof a cell, the cell having been contacted with a combination therapy of rhuEPO and at least one perturbagen selected from Table 5, wherein the combination therapy is capable of altering a gene signature in a progenitor.

An aspect of the present technology is a method for treating or preventing anemia. In this aspect, the method comprises the steps of administering to a subject in need thereof a therapeutically effective amount of a combination therapy of rhuEPO and at least one perturbagen selected from Table 5, wherein the combination therapy is capable of altering a gene signature in a progenitor cell. In other embodiments, the method comprises the steps of administering to a subject in need thereof a cell, the cell having been contacted with a combination therapy of rhuEPO and at least one perturbagen selected from Table 5, wherein the combination therapy is capable of altering a gene signature in a progenitor.

In some embodiments, for any herein disclosed method, the disease or disorder characterized by an abnormal erythron distribution and/or physiology or an erythrocyte deficiency is an anemia. In some embodiments, the erythrocyte deficiency is an abnormal count or activity of erythrocytes. In other embodiments, for any herein disclosed method, the anemia is selected from aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia. In some embodiments, for any herein disclosed method, the disease or disorder characterized by an abnormal erythron distribution and/or physiology or erythrocyte deficiency is an erythropoietin deficiency. In some embodiments, for any herein disclosed method, the disease or disorder characterized by an abnormal erythron distribution and/or physiology or erythrocyte deficiency is a hemolytic disease or disorder.

In some embodiments, for any herein disclosed method, the disease or disorder is characterized by an abnormal ratio of erythrocytes to proerythroblasts, early erythroblasts, intermediate erythroblasts, late erythroblasts, and/or reticulocytes. In this aspect, the abnormal ratio comprises a decreased number of erythrocytes and/or an increased number of progenitor cells. In some embodiments, the abnormal ratio comprises an increased number of progenitor cells. In some embodiments, for any herein disclosed method, the abnormal ratio comprises an increased number of proerythroblasts, early erythroblasts, intermediate erythroblasts, late erythroblasts, and/or reticulocytes.

In some embodiments, for any herein disclosed method, the administering is directed to the bone marrow of the subject. In some embodiments, for any herein disclosed method, the administering is via intraosseous injection or intraosseous infusion. In some embodiments, for any herein disclosed method, the administering the cell is via intravenous injection or intravenous infusion. In some embodiments, the administering is simultaneously or sequentially to one or more mobilization agents. In some embodiments, the subject is a human. In some embodiments, the subject is an adult human.

In one embodiment, the subject is selected by steps including: obtaining from the subject having the disease or disorder a sample of cells comprising a non-lineage committed CD34⁺ cell; and contacting the sample of cells with least one perturbagen selected from Table 5, or a variant thereof. In this aspect, the at least one perturbagen alters a gene signature in the sample of cells. In some embodiments, the subject is selected by steps including: obtaining from a subject having the disease or disorder a sample of cells comprising a non-lineage committed CD34⁺ cell; and contacting the sample of cells with at least one perturbagen capable of altering a gene signature in a non-lineage committed CD34⁺ cell. In this aspect, the at least one perturbagen increases in the sample of cells the expression and/or activity of a gene selected from the genes designated as an “up” gene in the gene directionality column of Table 2 and/or decreases in the sample of cells the expression and/or activity of one or more genes selected from the genes designated as an “down” gene in the gene directionality column of Table 2.

In one embodiment, the subject is selected by steps including: obtaining from the subject having the disease or disorder a sample of cells comprising a non-lineage committed CD34⁺ cell; and contacting the sample of cells with least one perturbagen selected from Table 5, or a variant thereof. In this aspect, the at least one perturbagen increases in the sample of cells the expression and/or activity of a gene selected from the genes designated as an “up” gene in the gene directionality column of Table 2 and/or decreases in the sample of cells the expression and/or activity of one or more genes selected from the genes designated as an “down” gene in the gene directionality column of Table 2.

In one embodiment, the subject is selected by steps including: obtaining from the subject having the disease or disorder a sample of cells comprising a non-lineage committed CD34⁺ cell; and contacting the sample of cells with least one perturbagen selected from Table 5, or a variant thereof. In this aspect, when the at least one perturbagen alters a gene signature in the sample of cells, the subject is selected as a subject.

In some embodiments, the present technology provides a method for selecting the subject as described above, the method including the steps of: obtaining from a subject having the disease or disorder a sample of cells comprising a non-lineage committed CD34⁺ cell; and contacting the sample of cells with at least one perturbagen capable of altering a gene signature in a non-lineage committed CD34⁺ cell. In this aspect, when the at least one perturbagen causes the increases in the sample of cells the expression and/or activity of a gene selected from the genes designated as an “up” gene in the gene directionality column of Table 2 and/or decreases in the sample of cells the expression and/or activity of one or more genes selected from the genes designated as an “down” gene in the gene directionality column of Table 2, the subject is selected as a subject.

In one embodiment, the subject is selected by steps including: obtaining from the subject having the disease or disorder a sample of cells comprising a non-lineage committed CD34⁺ cell; and contacting the sample of cells with least one perturbagen selected from Table 5, or a variant thereof. In this aspect, when the at least one perturbagen causes the increases in the sample of cells the expression and/or activity of a gene selected from the genes designated as an “up” gene in the gene directionality column of Table 2 and/or decreases in the sample of cells the expression and/or activity of one or more genes selected from the genes designated as an “down” gene in the gene directionality column of Table 2, the subject is selected as a subject.

In some embodiments, the present technology provides a method for selecting the subject as described above, the method including the steps of: obtaining from a subject having the disease or disorder a sample of cells comprising a non-lineage committed CD34⁺ cell; and contacting the sample of cells with at least one perturbagen selected from Table 5, or a variant thereof. In this aspect, when the at least one perturbagen increases in the sample of cells the expression and/or activity of one or more genes selected from the genes designated as an “up” gene in the gene directionality column of Table 2 and/or decreases in the sample of cells the expression and/or activity of one or more genes selected from the genes designated as an “down” gene in the gene directionality column of Table 2, the subject is selected as a subject.

An aspect of the present technology provides use of the perturbagen of Table 5, or a variant thereof in the manufacture of a medicament for treating a disease or disorder characterized by disease or disorder characterized by an abnormal erythron distribution and/or physiology and/or erythrocyte deficiency is an anemia. In another aspect, the present technology provides use of the perturbagen of Table 5, or a variant thereof in the manufacture of a medicament for treating sickle cell disease or a thalassemia.

In another aspect, the present technology provides a method for making a therapeutic agent for a disease or disorder selected from anemia, or a disease or disorder characterized by an abnormal erythron distribution and/or physiology, or an erythrocyte deficiency. In this aspect, the method comprises the steps of: (a) identifying a candidate perturbation for therapy and (b) formulating the candidate perturbation as a therapeutic agent for the treatment of the disease or disorder.

In some embodiments, the promoting the transition of a starting population of progenitor cells into erythrocytes or immediate progenitors thereof occurs in vitro or ex vivo. In one embodiment, promoting the transition of a starting population of progenitor cells into erythrocytes or progenitors thereof occurs in vivo in a subject. In one embodiment, the subject is a human. In one embodiment, the human is an adult human.

Any aspect or embodiment disclosed herein can be combined with any other aspect or embodiment as disclosed herein.

### Administration, Dosing, and Treatment Regimens

As examples, administration results in the delivery of cultured erythroblasts or erythrocytes as disclosed herein into the bloodstream via intravenous infusion.

As examples, administration results in the delivery of one or more perturbagens disclosed herein into the bloodstream (via enteral or parenteral administration), or alternatively, the one or more perturbagens is administered directly to the site of hematopoietic cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11(1): 78-88; Hopman and DiPersio “Advances in Stem Cell Mobilization.” Blood Review, 2014, 28(1): 31-40; and Kim “Hematopoietic stem cell mobilization: current status and future perspective.” Blood Res. 2017 June; 52(2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In other embodiments, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in teMEPrature, stimulation by an appropriate wavelength of light, concentration or availability of enzymes, concentration or availability of water, or other physiological conditions or compounds.

In other embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In other embodiments, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed.

Any perturbagen disclosed herein can be administered in a single daily dose (also known as QD, qd or q.d.), or the total daily dosage can be administered in divided doses of twice daily (also known as BID, bid, or b.i.d.), three times daily (also known as TID, tid, or t.i.d.), or four times daily (also known as QID, qid, or q.i.d.). Furthermore, any perturbagen disclosed herein can be administered continuously rather than intermittently throughout the dosage regimen.

### Pharmaceutical Compositions and Formulations

Aspects of the present technology include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In some embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In some embodiments, the compositions, e.g., pharmaceutical compositions, disclosed herein are suspended in a saline buffer (including, without limitation TBS, PBS, and the like).

The present technology includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

In some embodiments, the present technology provides a pharmaceutical composition comprising the combination of two or more perturbagens selected from Table 5 for the treatment of a disease or disorder selected from the group consisting of anemia, an abnormal erythron distribution and/or physiology, or an erythrocyte deficiency.

In some embodiments, the present technology provides a pharmaceutical composition comprising the combination of two or more perturbagens selected from Table 5.

In some embodiments, the present technology provides a pharmaceutical composition comprising the combination of two or more perturbagens, each with a different mechanism of action, selected from Table 5, or a variant thereof for the treatment of a disease or disorder selected from the group consisting of disease or disorder characterized by an abnormal erythron distribution and/or physiology, or erythrocyte deficiency. In some embodiments, the present technology provides a pharmaceutical composition comprising the combination of two or more perturbagens, each with a different mechanism of action, selected from Table 5, or a variant thereof for the treatment or prevention of anemia. In some embodiments, the present technology provides a pharmaceutical composition comprising the combination of two or more perturbagens, each with a different mechanism of action, selected from Table 5, or a variant thereof for the treatment a disease or disorder selected from the group consisting of aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia. In some embodiments, the present technology provides a pharmaceutical composition comprising the combination of two or more perturbagens, each with a different mechanism of action, selected from Table 5, or a variant thereof for the treatment thalassemia, or sickle cell anemia. In some embodiments, the present technology provides a pharmaceutical composition comprising the combination of two or more perturbagens, each with a different mechanism of action, selected from Table 5, or a variant thereof for the treatment of chemotherapy induced anemia.

In some embodiments, the present technology provides a pharmaceutical composition comprising the combination of rhuEPO and one or more perturbagens selected from Table 5, or a variant thereof for the treatment anemia. In some embodiments, the present technology provides a pharmaceutical composition comprising the combination of rhuEPO and one or more perturbagens selected from Table 5, or a variant thereof for the treatment of a disease or disorder selected from the group consisting of aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia. In some embodiments, the present technology provides a pharmaceutical composition comprising the combination of rhuEPO and one or more perturbagens selected from Table 5, or a variant thereof for the treatment a disease or disorder selected from the group consisting of thalassemia, sickle cell anemia (SS). In some embodiments, the present technology provides a pharmaceutical composition comprising the combination of rhuEPO and one or more perturbagens selected from Table 5, or a variant thereof for the treatment of chemotherapy induced anemia.

In some embodiments, two or more perturbagens selected from Table 5, or a variant thereof may be mixed into a single preparation or two or more perturbagens of the combination may be formulated into separate preparations for use in combination separately or at the same time. In some embodiments, the present technology provides a kit containing the two or more perturbagens selected from Table 5, or a variant thereof, formulated into separate preparations. In some embodiments, the combination therapies, comprising more than one perturbagen, can be co-delivered in a single delivery vehicle or delivery device.

As used herein, the term “combination” or “pharmaceutical combination” refers to the combined administration of the perturbagens. The combination of two or more perturbagen may be formulated as fixed dose combination or co-packaged discrete perturbagen dosages. In some embodiments, the fixed dose combination therapy of perturbagens comprises bilayer tablet, triple layer tablet, multilayered tablet, or capsule having plurality populations of particles of perturbagens. In some embodiments, the combination of two or more perturbagens may be administered to a subject in need thereof, e.g., concurrently or sequentially.

In some embodiments, the combination therapies of perturbagens as described above give synergistic effects on promoting the proliferation of erythrocytes in a subject. The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination (Chou, Cancer Res. 2010, vol. 70, pp. 440-446). When the Cl value is less than 1, there is synergy between the compounds used in the combination; when the Cl value is equal to 1, there is an additive effect between the compounds used in the combination and when Cl value is more than 1, there is an antagonistic effect. The synergistic effect may be attained by co-formulating the agents of the pharmaceutical combination. The synergistic effect may be attained by administering two or more agents as separate formulations administered simultaneously or sequentially.

Compositions for administration can optionally include a local anesthetic such as, for example, lignocaine to lessen pain at the site of the injection.

The pharmaceutical compositions comprising the perturbagens of the present technology may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In some embodiments, the present technology provides compositions comprising the cultured blood cells produced by any herein disclosed methods suspended in red blood cell additive solution, or physiological saline. In some embodiments, the red blood cell additive solution is selected from the group consisting of saline-adenine-glucose solution (SAG), saline-adenine-glucose (45)-mannitol (30) solution (SAGM), saline-adenine-glucose (111)-mannitol (41) solution (AS-1), Saline-adenine-glucose (55)-NaH₂PO₄-citric acid-Na-citrate solution (AS-3), saline-adenine-glucose (45)-mannitol (45.5) solution (AS-5), saline-adenine-glucose (40)-mannitol (80)-NaH₂PO₄-citric acid-Na-citrate solution, and saline-adenine-guanosine-glucose-mannitol-Na₂HPO₄—NaH₂PO₄ (PAGGSM).

In some embodiments, any perturbagens disclosed herein is formulated in accordance with routine procedures as a pharmaceutical composition adapted for a mode of administration disclosed herein.

### Other Aspects of the Present Technology

Embodiments associated with any of the above-disclosed aspects are likewise relevant to the below-mentioned aspects. In other words, each of the embodiment mentioned above for the above aspects may be revised/adapted to be applicable to the below aspects.

In an aspect, the present technology provides a use of the perturbagen of Table 5, or a variant thereof in the manufacture of a medicament for treating a disease or disorder characterized by an abnormal erythron distribution and/or physiology, or an erythrocyte deficiency.

Yet another aspect of the present technology is a use of the perturbagen of Table 5, or a variant thereof in the manufacture of a medicament for the treating a disease or disorder selected from the group consisting of aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia.

In another aspect, the present technology provides a method of identifying a candidate perturbation for promoting the transition of a starting population of progenitor cells into erythrocytes and/or reticulocytes or immediate progenitors thereof. The method includes the steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of cells in the population of progenitor cells into erythrocytes and/or reticulocytes or immediate progenitors thereof following exposure of the population of cells to the perturbation; and identifying the perturbation as a candidate perturbation for promoting the transition of a population of progenitor cells into erythrocytes and/or reticulocytes or immediate progenitors thereof based on the perturbation signature. In this aspect, the perturbation signature is an increase in expression and/or activity in the progenitor cell of one or more genes selected from the genes designated as an “up” gene in the gene directionality column of Table 2, and/or a decrease in expression and/or activity in the progenitor cell of one or more genes selected from the genes designated as an “down” gene in the gene directionality column of Table 2.

In another aspect, the present technology provides a method for making a therapeutic agent for use in treating a disease or disorder characterized by an abnormal erythron distribution and/or physiology, or an erythrocyte deficiency. In another aspect, the present technology provides a method for making a therapeutic agent for used in treating a disease or disorder selected from the group consisting of aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia. In another aspect, the present technology provides a method for making a therapeutic agent for use in treating anemia. In some embodiments, the method includes the steps of: (a) identifying a candidate perturbation for therapy and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into erythrocytes and/or reticulocytes or immediate progenitors thereof following exposure of the population of cells to the perturbation; and identifying the perturbation as a candidate perturbation for promoting the transition of a population of progenitor cells into erythrocytes and/or reticulocytes or immediate progenitors thereof based on the perturbation signature. Further, in this aspect, the perturbation signature is an increase in expression and/or activity in the progenitor cell of one or more genes selected from the genes designated as an “up” gene in the gene directionality column of Table 2, and/or a decrease in expression and/or activity in the progenitor cell of one or more genes selected from the genes designated as an “down” gene in the gene directionality column of Table 2.

In various embodiments, the present methods involving the monitoring of cell sickling, e.g. with one or more in vitro sickling assays (see Example 3 and Smith et al. Variable deformability of irreversibly sickled erythrocytes. Blood. 1981; 58(1):71-78; van Beers et al. Imaging flow cytometry for automated detection of hypoxia-induced erythrocyte shape change in sickle cell disease Am J Hematol. 2014 June; 89(6): 598-603; and Rab, et al. Rapid and reproducible characterization of sickling during automated deoxygenation in sickle cell disease patients Am J Hematol. 2019 May; 94(5): 575-584.

To further evaluate the impact of the perturbagens in reducing disease burden, assays are performed on sickle derived erythrocytes cells by enrichment of enucleated erythrocytes followed by incubation of cells at low oxygen or incubation in 2% sodium metabisulfite. Cell sickling is monitored using time lapse imaging.

In various embodiments, the present methods involving the monitoring of hemoglobin concentration, or red blood cell counts (Liumbruno et al., Blood Transfus. 2009, vol. 7, pp. 49-64) after administering the therapeutic composition containing at least one perturbagen selected from Table 5, or cells after being contacted with at least one perturbagen selected from Table 5.

Yet another aspect of the present technology is a perturbagen capable of causing a change in a gene signature.

In an aspect, the present technology provides a perturbagen capable of causing a change in cell fate.

In another aspect, the present technology provides a perturbagen capable of causing a change in a gene signature and a change in cell fate.

In yet another aspect, the present technology provides a pharmaceutical composition comprising any herein disclosed perturbagen.

In a further aspect, the present technology provides a unit dosage form comprising an effective amount of the pharmaceutical composition comprising any herein disclosed perturbagen.

The instant disclosure also provides certain embodiments as follows:

Embodiment 116: A method for directing a change in cell state of a progenitor cell comprising: contacting a population of cells comprising a progenitor cell with at least one perturbagen selected from Table 5, or a variant thereof, wherein the at least one perturbagen is capable of altering a gene signature in the progenitor cell; and wherein the progenitor cell is a non-lineage committed CD34+ cell.
Embodiment 117: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 2 and/or a decrease in expression and/or activity in the progenitor cell of one or more genes selected from the genes designated as an “down” gene in the gene directionality column of Table 2 and wherein the progenitor cell is a non-lineage committed CD34+ cell.
Embodiment 118: A method for directing a change in cell state of a progenitor cell, comprising: contacting a population of cells comprising a progenitor cell with at least one perturbagen selected from Table 5, or a variant thereof, and capable of altering a gene signature in the progenitor cell, wherein altering the gene signature comprises an increase in expression and/or activity in the progenitor cell of one or more genes selected from the genes designated as an “up” gene in the gene directionality column of Table 2 and/or a decrease in expression and/or activity in the progenitor cell of one or more genes selected from the genes designated as an “down” gene in the gene directionality column of Table 2 and wherein the progenitor cell is a non-lineage committed CD34+ cell.
Embodiment 119: The method of Embodiment 117 or 118, wherein altering the gene signature comprises an increase in expression and/or activity in the progenitor cell of a network module designated in the network module column of Table 2.
Embodiment 120: The method of Embodiment 119, wherein the activation of one or more genes of the network module designated in the network module column of Table 2 comprises modulating expression and/or activity of 2 or more genes within a network module.
Embodiment 121: The method of Embodiment 117 or 118, wherein altering the gene signature comprises an increase in expression and/or activity in the progenitor cell of two or more genes designated as an “up” gene in the gene directionality column of Table 2.
Embodiment 122: The method of Embodiment 121, wherein altering the gene signature comprises a decrease in expression and/or activity in the progenitor cell of two or more genes designated as a “down” gene in the gene directionality column of Table 2.
Embodiment 123: The method of any one of Embodiments 116 to 122, wherein the change in cell state provides an increase in the number of one or more of proerythroblasts, early erythroblasts, intermediate erythroblasts, late erythroblasts, reticulocytes, and/or erythrocytes.
Embodiment 124: The method of Embodiment 123, wherein the increase in the number of erythrocytes is relative to the number of erythrocytes obtained from a population of progenitor cells that is not contacted with the at least one perturbagen.
Embodiment 125: The method of Embodiment 123, wherein the increase in the number of erythrocytes is relative to the population of progenitor cells prior to contacting with the at least one perturbagen.
Embodiment 126: The method of Embodiment 124 or Embodiment 125, wherein the change in cell state provides an increase in the number of erythrocytes.
Embodiment 127: The method of Embodiment 123, wherein the ratio of the number of erythrocytes to the number of progenitor cells is increased relative to the ratio obtained from a population of progenitor cells that is not contacted with the at least one perturbagen.
Embodiment 128: The method of Embodiment 123, wherein the ratio of the number of erythrocytes to the number of progenitor cells is increased relative to the ratio in the population of progenitor cells prior to contacting with the at least one perturbagen.
Embodiment 129: The method of any one of Embodiments 123 to 128, wherein the increase in the number of erythrocytes is due in part to an increased lifespan of the erythrocytes.
Embodiment 130: The method of any one of Embodiments 123 to 129, wherein the increase in the number of erythrocytes is due in part to reduced cell death among the erythrocytes.
Embodiment 131: The method of any one of Embodiments 123 to 130, wherein the increase in the number of erythrocytes is due in part to a change of cell state from progenitor cells into the erythrocyte.
Embodiment 132: The method of any one of Embodiments 116 to 131, wherein the number of progenitor cells is decreased.
Embodiment 133: The method of Embodiment 132, wherein the decrease in the number of progenitor cells is due in part to decreased cell proliferation of the progenitor cells.
Embodiment 134: The method of Embodiment 132 or Embodiment 133, wherein the decrease in the number of progenitor cells is due in part to a decreased lifespan of the progenitor cells.
Embodiment 135: The method of any one of Embodiments 132 to 134, wherein the decrease in the number of progenitor cells is due in part to increased cell death among the progenitor cells.
Embodiment 136: The method of any one of Embodiments 132 to 135, wherein the decrease in the number of progenitor cells is relative to the number of progenitor cells in a population of progenitor cells that is not contacted with the at least one perturbagen.
Embodiment 137: The method of any one of Embodiments 132 to 136, wherein the decrease in the number of progenitor cells is relative to the number of progenitor cells in the population prior to contacting with the at least one perturbagen.
Embodiment 138: The method of any one of Embodiments 132 to 137, wherein the decrease in the number of progenitor cells is due to a change of cell state from a progenitor cell into the erythrocyte lineage and/or megakaryocyte lineage.
Embodiment 139: The method of any one of Embodiments 116 to 131, wherein the number of progenitor cells is increased.
Embodiment 140: The method of Embodiment 139, wherein the increase in the number of progenitor cells is due in part to increased cell proliferation of the progenitor cells.
Embodiment 141: The method of Embodiment 139 or Embodiment 140, wherein the increase in the number of progenitor cells is due in part to an increased lifespan of the progenitor cells.
Embodiment 142: The method of any one of Embodiments 139 to 141, wherein the increase in the number of progenitor cells is due in part to decreased cell death among the progenitor cells.
Embodiment 143: The method of any one of Embodiments 139 to 142, wherein the increase in the number of progenitor cells is relative to the number of progenitor cells in a population of progenitor cells that is not contacted with the at least one perturbagen.
Embodiment 144: The method of any one of Embodiments 139 to 142, wherein the increase in the number of progenitor cells is relative to the number of progenitor cells in the population prior to contacting with the at least one perturbagen.
Embodiment 145: The method of any one of Embodiments 116 to 131, wherein the number of proerythroblasts, early erythroblasts, intermediate erythroblasts, late erythroblasts, reticulocytes, and/or erythrocytes is increased after contacting the population of cells comprising a CD34+ cell with the at least one perturbagen.
Embodiment 146: The method of any one of Embodiments 116 to 131, wherein the number of erythrocytes is increased after contacting the population of cells comprising a CD34+ cell with the at least one perturbagen.
Embodiment 147: The method of any one of Embodiments 116 to 131, wherein the number of reticulocytes, and/or erythrocytes is increased after contacting the population of cells comprising a CD34+ cell with the at least one perturbagen.
Embodiment 148: The method of Embodiment 145, wherein the ratio of the number of proerythroblasts, early erythroblasts, intermediate erythroblasts, late erythroblasts, reticulocytes, and/or erythrocytes to the number of progenitor cells is increased relative to the ratio obtained from a population of progenitor cells that is not contacted with the at least one perturbagen.
Embodiment 149: The method of Embodiment 145, wherein the ratio of the number proerythroblasts, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen.
Embodiment 150: The method of any one of Embodiments 116 to 131, wherein the ratio of the number of early erythroblasts, intermediate erythroblasts, late erythroblasts, reticulocytes, and/or erythrocytes to the number of proerythroblasts is increased relative to the ratio obtained from a population of progenitor cells that is not contacted with the at least one perturbagen.
Embodiment 151: The method of any one of Embodiments 116 to 131, wherein the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen.
Embodiment 152: The method of any one of Embodiments 116 to 131, wherein the ratio of the number of early erythroblasts to the number of proerythroblasts is increased relative to the ratio obtained from a population of progenitor cells that is not contacted with the at least one perturbagen.
Embodiment 153: The method of any one of Embodiments 116 to 131, wherein the ratio of the number of early erythroblasts to the number of proerythroblasts is increased relative to the ratio in the population of progenitor cells prior to contacting with the at least one perturbagen.
Embodiment 154: The method of any one of Embodiments 116 to 131, wherein the ratio of the number of intermediate erythroblasts to the number of early erythroblasts is increased relative to the ratio obtained from a population of progenitor cells that is not contacted with the at least one perturbagen.
Embodiment 155: The method of any one of Embodiments 116 to 131, wherein the ratio of the number of intermediate erythroblasts to the number of early erythroblasts is increased relative to the ratio in the population of progenitor cells prior to contacting with the at least one perturbagen.
Embodiment 156: The method of any one of Embodiments 116 to 131, wherein the ratio of the number of late erythroblasts to the number of intermediate erythroblasts is increased relative to the ratio obtained from a population of progenitor cells that is not contacted with the at least one perturbagen.
Embodiment 157: The method of any one of Embodiments 116 to 131, wherein the ratio of the number of late erythroblasts to the number of intermediate erythroblasts is increased relative to the ratio in the population of progenitor cells prior to contacting with the at least one perturbagen.
Embodiment 158: The method of any one of Embodiments 116 to 131, wherein the ratio of the number of reticulocytes to the number of late erythroblasts is increased relative to the ratio obtained from a population of progenitor cells that is not contacted with the at least one perturbagen.
Embodiment 159: The method of any one of Embodiments 116 to 131, wherein the ratio of the number of reticulocytes to the number of late erythroblasts is increased relative to the ratio in the population of progenitor cells prior to contacting with the at least one perturbagen.
Embodiment 160: The method of any one of Embodiments 116 to 131, wherein the ratio of the number of erythrocytes to the number of reticulocytes is increased relative to the ratio obtained from a population of progenitor cells that is not contacted with the at least one perturbagen.
Embodiment 161: The method of any one of Embodiments 116 to 131, wherein the ratio of the number of erythrocytes to the number of reticulocytes is increased relative to the ratio in the population of progenitor cells prior to contacting with the at least one perturbagen.
Embodiment 162: The method of any one of Embodiments 116 to 131, wherein the ratio of the number of erythrocytes to late erythroblasts is increased relative to the ratio obtained from a population of progenitor cells that is not contacted with the at least one perturbagen.
Embodiment 163: The method of any one of Embodiments 116 to 131, wherein the ratio of the number of erythrocytes to late erythroblasts is increased relative to the ratio in the population of progenitor cells prior to contacting with the at least one perturbagen.
Embodiment 164: The method of any one of Embodiments 116 to 131, wherein the ratio of the number of erythrocytes to intermediate erythroblasts is increased relative to the ratio obtained from a population of progenitor cells that is not contacted with the at least one perturbagen.
Embodiment 165: The method of any one of Embodiments 116 to 131, wherein the ratio of the number of erythrocytes to intermediate erythroblasts is increased relative to the ratio in the population of progenitor cells prior to contacting with the at least one perturbagen.
Embodiment 166: The method of any one of Embodiments 116 to 131, wherein the ratio of the number of erythrocytes to early erythroblasts is increased relative to the ratio obtained from a population of progenitor cells that is not contacted with the at least one perturbagen.
Embodiment 167: The method of any one of Embodiments 116 to 131, wherein the ratio of the number of erythrocytes to early erythroblasts is increased relative to the ratio in the population of progenitor cells prior to contacting with the at least one perturbagen.
Embodiment 168: The method of any one of Embodiments 116 to 131, wherein the ratio of the number of erythrocytes to proerythroblasts is increased relative to the ratio obtained from a population of progenitor cells that is not contacted with the at least one perturbagen.
Embodiment 169: The method of any one of Embodiments 116 to 131, wherein the ratio of the number of erythrocytes to proerythroblasts is increased relative to the ratio in the population of progenitor cells prior to contacting with the at least one perturbagen.
Embodiment 170: The method of any one of Embodiments 116 to 131, wherein the ratio of the number of erythrocytes to early erythroblasts, intermediate erythroblasts, late erythroblasts, reticulocytes, and/or proerythroblasts is increased relative to the ratio obtained from a population of progenitor cells that is not contacted with the at least one perturbagen.
Embodiment 171: The method of any one of Embodiments 116 to 131, wherein the ratio of the number of erythrocytes to early erythroblasts, intermediate erythroblasts, late erythroblasts, reticulocytes, and/or proerythroblasts is increased relative to the ratio in the population of progenitor cells prior to contacting with the at least one perturbagen.
Embodiment 172: The method of any of Embodiments 116 to 144, wherein the number of proerythroblasts, early erythroblasts, intermediate erythroblasts, late erythroblasts, and/or reticulocytes is decreased.
Embodiment 173: The method of any of Embodiments 116 to 144, wherein the number of proerythroblasts is decreased.
Embodiment 174: The method of any of Embodiments 116 to 144, wherein the number of early erythroblasts is decreased.
Embodiment 175: The method of any of Embodiments 116 to 144, wherein the number of intermediate erythroblasts is decreased.
Embodiment 176: The method of any of Embodiments 116 to 144, wherein the number of late erythroblasts is decreased.
Embodiment 177: The method of any of Embodiments 116 to 144, wherein the number of reticulocytes is decreased.
Embodiment 178: The method of any of Embodiments 116 to 144, wherein the number of proerythroblasts is increased.
Embodiment 179: The method of any of Embodiments 116 to 144, wherein the number of proerythroblasts, early erythroblasts, intermediate erythroblasts, late erythroblasts, and/or reticulocytes is increased.
Embodiment 180: The method of any of Embodiments 116 to 144, wherein the number of early erythroblasts is increased.
Embodiment 181: The method of any of Embodiments 116 to 144, wherein the number of intermediate erythroblasts is increased.
Embodiment 182: The method of any of Embodiments 116 to 144, wherein the number of late erythroblasts is increased.
Embodiment 183: The method of any of Embodiments 116 to 144, wherein the number of reticulocytes is increased.
Embodiment 184: The method of any of Embodiments 116 to 144, wherein the number of erythrocytes is increased.
Embodiment 185: The method of any one of Embodiments 116 to 184, wherein the at least one perturbagen selected from Table 5, or a variant thereof, comprises at least 2, at least 3, at least 4, or at least 5 perturbagens selected from Table 5, or variants thereof.
Embodiment 186: The method of Embodiment 117 or 118, wherein the one or more genes selected from the genes designated as an “up” gene in the gene directionality column of Table 2, comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2.
Embodiment 187: The method of Embodiment 186, wherein the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39.
Embodiment 188: The method of Embodiment 117 or 118, wherein the one or more genes selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2.
Embodiment 189: The method of Embodiment 188, wherein the one or more genes selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNA/2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1.
Embodiment 190: The method of any one of Embodiments 116 to 189, wherein contacting the population of progenitor cells occurs in vitro or ex vivo.
Embodiment 191: The method of any one of Embodiments 116 to 190, wherein contacting the population of progenitor cells occurs in vivo in a subject.
Embodiment 192: The method of Embodiment 191, wherein the subject is a human.
Embodiment 193: The method of Embodiment 192, wherein the human is an adult human.
Embodiment 194: A perturbagen for use in the method of any one of Embodiments 116 to 193.
Embodiment 195: A pharmaceutical composition comprising the perturbagen of Embodiment 194.
Embodiment 196: A method for treating a disease or disorder characterized by an abnormal erythron distribution and/or physiology, comprising: (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.
Embodiment 197: A method for treating a disease or disorder characterized by an erythrocyte deficiency, comprising: (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.
Embodiment 198: A method for treating or preventing an anemia, comprising: (a) administering to a subject in need thereof a therapeutically effective amount of at least one perturbagen selected from Table 5, or a variant thereof, wherein the at least one perturbagen is capable of changing a gene signature in a progenitor cell or (b) administering to a subject in need thereof a cell, the cell having been contacted with at least one perturbagen selected from Table 5, or a variant thereof, wherein the at least one perturbagen is capable of changing a gene signature in a progenitor cell.
Embodiment 199: The method of Embodiment 197, wherein the erythrocyte deficiency is an abnormal count or activity of erythrocytes.
Embodiment 200: The method of any one of Embodiments 196 to 198, wherein the administering is directed to the bone marrow of the subject.
Embodiment 201: The method of Embodiment 200, wherein the administering is via intraosseous injection or intraosseous infusion.
Embodiment 202: The method of any one of Embodiments 196 to 201, wherein the administering the cell is via intravenous injection or intravenous infusion.
Embodiment 203: The method of any one of Embodiments 196 to 202, wherein the administering is simultaneously or sequentially to one or more mobilization agents.
Embodiment 204: The method of any one of Embodiments 196 to 203, wherein the disease or disorder characterized by an abnormal erythron distribution and/or physiology or erythrocyte deficiency is an anemia.
Embodiment 205: The method of Embodiment 198 or 204, wherein the anemia is selected from aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia.
Embodiment 206: The method of any one of Embodiment 196 or 197, wherein the disease or disorder characterized by an abnormal erythron distribution and/or physiology or erythrocyte deficiency is an erythropoietin deficiency.
Embodiment 207: The method of any one of Embodiments 196 or 197, wherein the disease or disorder characterized by an abnormal erythron distribution and/or physiology or erythrocyte deficiency is a hemolytic disease or disorder.
Embodiment 208: The method of any one of Embodiments 196 to 207, wherein at least one perturbagen is administered on the basis of previously determining the subject exhibits an abnormal number of erythrocyte, or a disease or disorder characterized thereby.
Embodiment 209: The method of any one of Embodiments 196 to 208, wherein the disease or disorder is characterized by an abnormal ratio of erythrocytes to proerythroblasts, early erythroblasts, intermediate erythroblasts, late erythroblasts, and/or reticulocytes.
Embodiment 210: The method of Embodiment 209, wherein the abnormal ratio comprises a decreased number of erythrocytes and/or an increased number of progenitor cells.
Embodiment 211: The method of Embodiment 210, wherein the abnormal ratio comprises an increased number of progenitor cells.
Embodiment 212: The method of Embodiment 211, wherein the abnormal ratio comprises an increased number of proerythroblasts, early erythroblasts, intermediate erythroblasts, late erythroblasts, and/or reticulocytes.
Embodiment 213: The method of any one of Embodiments 196 to 212, wherein the at least one perturbagen is capable of changing a gene signature in a progenitor cell.
Embodiment 214: The method of any one of Embodiments 196 to 212, wherein the subject is selected by steps comprising: obtaining from the subject having the disease or disorder a sample of cells comprising a non-lineage committed CD34+ cell; and contacting the sample of cells with least one perturbagen selected from Table 5, or a variant thereof, wherein the at least one perturbagen alters a gene signature in the sample of cells.
Embodiment 215: The method of any one of Embodiments 196 to 212, wherein the subject is selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from the genes designated as an “up” gene in the gene directionality column of Table 2 and/or decreases in the sample of cells the expression and/or activity of one or more genes selected from the genes designated as an “down” gene in the gene directionality column of Table 2.
Embodiment 216: The method of any one of Embodiments 196 to 212, wherein the subject is selected by steps comprising: obtaining from a subject having the disease or disorder a sample of cells comprising a non-lineage committed CD34+ cell; and contacting the sample of cells with at least one perturbagen selected from Table 5, or a variant thereof; wherein the at least one perturbagen increases in the sample of cells the expression and/or activity of one or more genes selected from the genes designated as an “up” gene in the gene directionality column of Table 2 and/or decreases in the sample of cells the expression and/or activity of one or more genes selected from the genes designated as an “down” gene in the gene directionality column of Table 2.
Embodiment 217: A method for selecting the subject of any one of Embodiments 196 to 212, comprising: obtaining from a subject having the disease or disorder a sample of cells comprising a non-lineage committed CD34+ cell; and contacting the sample of cells with least one perturbagen selected from Table 5, or a variant thereof, wherein when the at least one perturbagen alters a gene signature in the sample of cells, the subject is selected as a subject.
Embodiment 218: A method for selecting the subject of any one of Embodiments 196 to 212, comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from the genes designated as an “up” gene in the gene directionality column of Table 2 and/or decreases in the sample of cells the expression and/or activity of one or more genes selected from the genes designated as an “down” gene in the gene directionality column of Table 2, the subject is selected as a subject.
Embodiment 219: A method for selecting the subject of any one of Embodiments 196 to 212, comprising: obtaining from a subject having the disease or disorder a sample of cells comprising a non-lineage committed CD34+ cell; and contacting the sample of cells with at least one perturbagen selected from Table 5, or a variant thereof; wherein when the at least one perturbagen increases in the sample of cells the expression and/or activity of one or more genes selected from the genes designated as an “up” gene in the gene directionality column of Table 2 and/or decreases in the sample of cells the expression and/or activity of one or more genes selected from the genes designated as an “down” gene in the gene directionality column of Table 2, the subject is selected as a subject.
Embodiment 220: Use of the perturbagen of Table 5, or a variant thereof in the manufacture of a medicament for treating a disease or disorder characterized by an abnormal ratio of erythrocytes to progenitor cells.
Embodiment 221: A method of identifying a candidate perturbation for promoting the transition of a starting population of progenitor cells into erythrocytes or immediate progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes or immediate progenitors thereof following exposure of the population of cells to the perturbation; and identifying the perturbation as a candidate perturbation for promoting the transition of a population of progenitor cells into erythrocytes or immediate progenitors thereof based on the perturbation signature, wherein the perturbation signature is an increase in expression and/or activity in the progenitor cell of one or more genes selected from the genes designated as an “up” gene in the gene directionality column of Table 2, and/or a decrease in expression and/or activity in the progenitor cell of one or more genes selected from the genes designated as an “down” gene in the gene directionality column of Table 2.
Embodiment 222: The method of Embodiment 221, wherein the perturbation signature is an increase in expression and/or activity in the progenitor cell of a network module designated in the network module column of Table 2.
Embodiment 223: The method of Embodiment 222, wherein the perturbation signature is an increase in expression and/or activity in the progenitor cell of one or more genes selected from the genes designated as an “up” gene in the gene directionality column of Table 2.
Embodiment 224: The method of Embodiment 221, wherein the perturbation signature is an increase in expression and/or activity in the progenitor cell of two or more genes designated as an “up” gene in the gene directionality column of Table 2.
Embodiment 225: The method of Embodiment 222, wherein the perturbation signature is a decrease in expression and/or activity in the progenitor cell of two or more genes designated as a “down” gene in the gene directionality column of Table 2.
Embodiment 226: A method for making a therapeutic agent for a disease or disorder selected from a disease or disorder characterized by an abnormal erythron distribution and/or physiology or erythrocyte deficiency, comprising: (a) identifying a candidate perturbation for therapy according to the method of Embodiment 222 and (b) formulating the candidate perturbation as a therapeutic agent for the treatment of the disease or disorder.

### Megakaryocyte Lineages

### Genes Signatures

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins that are characteristic of the cell type or cells of a specific lineage. Illustrative surface proteins are listed in Table 15 and Table 17.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

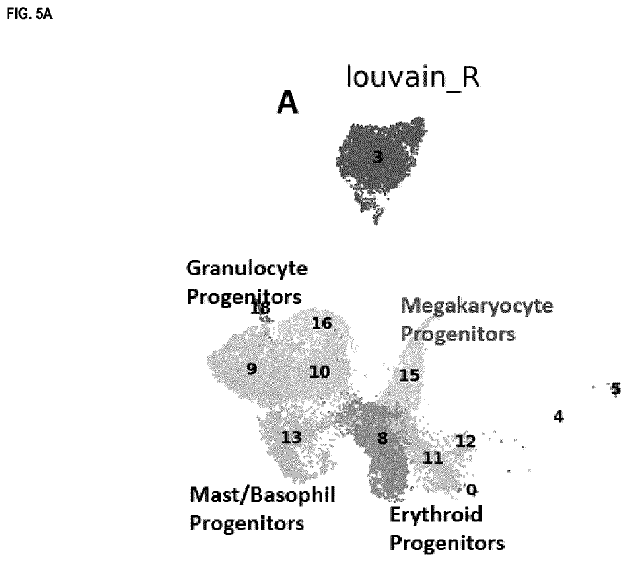

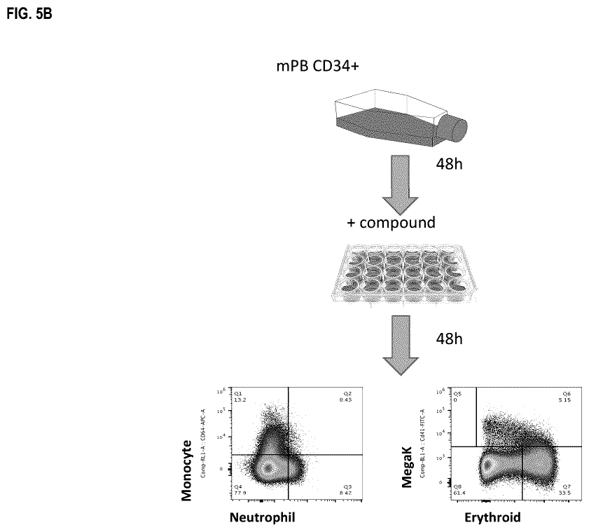

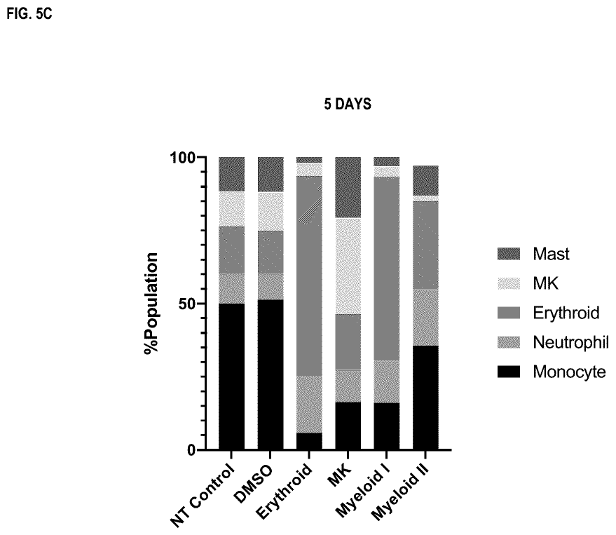

FIG. 5A, shows annotated clusters that associate gene signature with cell types or cells of a specific lineage. Differential gene signatures for the 8 to 15 transition, i.e., from a non-lineage committed CD34⁺ progenitor cell to cells of the megakaryocyte lineage, were used to predict perturbations that would promote the transition.

Genes that are differentially expressed and positively associated with the promotion of megakaryocyte lineage progression and/or megakaryocyte differentiation are listed in Table 3. The genes listed in Table 3 show an increase or decrease in expression and/or activity in the cell state change.

TABLE 3
Genes Showing an Increase or Decreases in Expression
in the Cell State Change.

| | | Gene | Gene | Network |
|---|---|---|---|---|
| | Gene | EntrezID | Directionality | Module |
| 0 | CCND3 | 896 | up | 1 |
| 1 | RSU1 | 6251 | up | 1 |
| 2 | CD320 | 51293 | down | 1 |
| 3 | PAFAH1B3 | 5050 | down | 1 |
| 4 | TRAP1 | 7190 | down | 1 |
| 5 | RRP1B | 23076 | down | 1 |
| 6 | PDLIM1 | 9124 | up | 2 |
| 7 | DNM1L | 10059 | up | 2 |
| 8 | HLA-DRA | 3122 | down | 2 |
| 9 | EIF4EBP1 | 1978 | down | 2 |
| 10 | TFDP1 | 7027 | down | 2 |
| 11 | PTPN12 | 5782 | up | 3 |
| 12 | CDK6 | 1021 | down | 3 |
| 13 | CDK4 | 1019 | down | 3 |
| 14 | MIF | 268 | down | 3 |
| 15 | GADD45A | 1647 | up | 4 |
| 16 | SH3BP5 | 9467 | up | 4 |
| 17 | TSC22D3 | 1831 | up | 4 |
| 18 | MYC | 4609 | down | 4 |
| 19 | CXCL2 | 2920 | up | 5 |
| 20 | RPL39L | 116832 | down | 5 |
| 21 | PAICS | 10606 | down | 5 |
| 22 | FBXO7 | 25793 | down | 5 |
| 23 | TPM1 | 7168 | up | 6 |
| 24 | PTPN6 | 5777 | up | 6 |
| 25 | ABHD4 | 63874 | up | 6 |
| 26 | IFRD2 | 7866 | down | 6 |
| 27 | SNCA | 6622 | up | 7 |
| 28 | CD44 | 960 | down | 7 |
| 29 | APOE | 348 | down | 7 |
| 30 | MAT2A | 4144 | down | 7 |
| 31 | INSIG1 | 3638 | up | 8 |
| 32 | STXBP2 | 6813 | up | 8 |
| 33 | LRRC16A | 55604 | up | 8 |
| 34 | MPC2 | 25874 | down | 8 |
| 35 | ZFP36 | 55552 | up | 9 |
| 36 | RPS5 | 6193 | down | 9 |
| 37 | ICAM3 | 3385 | down | 9 |
| 38 | RPS6 | 6194 | down | 9 |
| 39 | NFKBIA | 4792 | up | 10 |
| 40 | CISD1 | 55847 | down | 10 |
| 41 | GAPDH | 2597 | down | 10 |
| 42 | CXCR4 | 7852 | up | 11 |
| 43 | BTK | 695 | up | 11 |
| 44 | HSPA8 | 3312 | down | 11 |
| 45 | GNB5 | 10681 | up | 12 |
| 46 | PROS1 | 5627 | up | 12 |
| 47 | HSPB1 | 3315 | up | 12 |
| 48 | MYLK | 4638 | up | 13 |
| 49 | HSPD1 | 3329 | down | 13 |

In Table 3 and associated embodiments:
“Gene ID”: at the time of filing the present disclosure, the World Wide Web at ncbi.nlm.nih.gov/gene provides a description of and the nucleic acid sequence for each GeneID listed in Table 3; the contents of each of which is incorporated herein by reference in its entirety.
“Up” indicates a gene for which an increase in expression and/or activity in the progenitor cell is associated with the gene signature.
“Down” indicates a gene for which a decrease in expression and/or activity in the progenitor cell is associated with the gene signature.
A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition—such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 3, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 3. In certain embodiments, a perturbation activates multiple network modules for the desired cell state transition, such as 2, 3, 4, 5, 6, 7, 8, 9, 10, 11, 12, or all 13 modules.

In some embodiments, one or more genes of network module 1 are modulated. In some embodiments, the presents relate to the activation of network module 1, e.g., one or more of (inclusive of all of) CCND3, RSU1, CD320, PAFAH1B3, TRAP1, and RRP1B.

In some embodiments, one or more genes of network module 2 are modulated. In some embodiments, the presents relate to the activation of network module 2, e.g., one or more of (inclusive of all of) PDLIM1, DNM1L, HLA-DRA, EIF4EBP1, and TFDP1.

In some embodiments, one or more genes of network module 3 are modulated. In some embodiments, the presents relate to the activation of network module 3, e.g., one or more of (inclusive of all of) PTPN12, CDK6, CDK4, and MIF.

In some embodiments, one or more genes of network module 4 are modulated. In some embodiments, the presents relate to the activation of network module 4, e.g., one or more of (inclusive of all of) GADD45A, SH3BP5, TSC22D3, and MYC.

In some embodiments, one or more genes of network module 5 are modulated. In some embodiments, the presents relate to the activation of network module 5, e.g., one or more of (inclusive of all of) CXCL2, RPL39L, PAICS, and FBXO7.

In some embodiments, one or more genes of network module 6 are modulated. In some embodiments, the presents relate to the activation of network module 6, e.g., one or more of (inclusive of all of) TPM1, PTPN6, ABHD4, and IFRD2.

In some embodiments, one or more genes of network module 7 are modulated. In some embodiments, the presents relate to the activation of network module 7, e.g., one or more of (inclusive of all of) SNCA, CD44, APOE, and MAT2A.

In some embodiments, one or more genes of network module 8 are modulated. In some embodiments, the presents relate to the activation of network module 8, e.g., one or more of (inclusive of all of) INSIG1, STXBP2, LRRC16A, and MPC2.

In some embodiments, one or more genes of network module 9 are modulated. In some embodiments, the presents relate to the activation of network module 9, e.g., one or more of (inclusive of all of) ZFP36, RPS5, ICAM3, and RPS6.

In some embodiments, one or more genes of network module 10 are modulated. In some embodiments, the presents relate to the activation of network module 10, e.g., one or more of (inclusive of all of) NFKBIA, CISD1, and GAPDH.

In some embodiments, one or more genes of network module 11 are modulated. In some embodiments, the presents relate to the activation of network module 11, e.g., one or more of (inclusive of all of) CXCR4, BTK, and HSPA8.

In some embodiments, one or more genes of network module 12 are modulated. In some embodiments, the presents relate to the activation of network module 12, e.g., one or more of (inclusive of all of) GNB5, PROS1, and HSPB1.

In some embodiments, one or more genes of network module 13 are modulated. In some embodiments, the presents relate to the activation of network module 13, e.g., one or more of (inclusive of all of) MYLK and HSPD1.

In some embodiments, the present methods alter a gene signature in the sample of cells, comprising an activation of a network module designated in the network module column of Table 3.

In some embodiments, the activation of the network module designated in the network module column of Table 3 comprises modulating expression and/or activity of 2 or more genes within a network module.

In some embodiments, the activation of the network module designated in the network module column of Table 3 comprises modulating expression and/or activity of all of the genes within a network module.

In some embodiments, the activation of the network module designated in the network module column of Table 3 comprises modulating expression and/or activity of 2 or more genes within 2 or more network modules. In some embodiments, the activation of the network module designated in the network module column of Table 3 comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, or 50 or more genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 or more, or 13 or more network modules).

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting megakaryocyte lineage differentiation are listed in Table 6.

TABLE 6

| Perturbagen | | Molecular weight | Effective in vitro | |
|---|---|---|---|---|
| No. | Molecular formula | (g/mol) | concentration | |
| 1 | C₃₆H₅₆O₈ | 616.8 | 1 | μM |
| 2 | C₂₀H₃₀N₄O₂ | 358.5 | 1 | μM |
| 3 | C₂₄H₃₆N₄O₆S₂ | 540.7 | 5 | nm |
| 4 | C₂₁H₂₃N₃O₅ | 397.4 | 0.10 | μM |
| 5 | C₁₇H₁₅BrClFN₄O₃ | 457.7 | 10 | μM |
| 6 | C₂₁H₂₆N₆O₂ | 394.5 | 0.01 | μM |
| 7 | C₁₀H₁₆N₂O | 180.25 | 10 | μM |
| 8 | C₂₂H₂₇FN₄O₂ | 398.5 | 0.1 | μM |
| 9 | C₁₅H₁₄Cl₃N₃O₄ | 406.6 | 10 | μM |
| 10 | C₂₃H₁₅Cl₄FN₂O₃S₂ | 592.3 | 10 | μM |
| 11 | C₁₂H₆Cl₄O₂S | 356.0 | 10 | μM |
| 12 | C₄₃H₆₃NO₁₁ | 770 | 1 | μM |
| 13 | C₂₇H₂₆O₃ | 398.5 | 0.1 | μM |
| 14 | C₁₅H₁₄N₂O | 238.28 | 10 | μM |
| 15 | C₁₀H₅F₃N₄O | 254.17 | 20 | μM |
| 16 | C₁₃H₈Cl₂N₂O₄ | 327.12 | 5 | μM |
| 17 | C₃₀H₂₈O₈ | 516.5 | 5 | μM |
| 18 | C₂₁H₂₅N₅O₄S | 443.5 | 0.1 | μM |
| 19 | C₂₉H₄₁F₂N₅O | 513.7 | 10 | μM |
| 20 | C₁₂H₉N₃O | 211.22 | 10 | μM |

In various embodiments herein, a perturbagen encompasses the perturbagens named in Table 6. Thus, the named perturbagens of Table 6 represent examples of perturbagens of the present disclosure.

In Table 6, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In embodiments, a perturbagen used in the present disclosure is a variant of a perturbagen of Table 6. A variant may be a derivative, analog, enantiomer or a mixture of enantiomers thereof, or a pharmaceutically acceptable salt, solvate, hydrate, co-crystal, clathrate, prodrug, or polymorph of the perturbagen of Table 6. A variant of a perturbagen of Table 6 retains the biological activity of the perturbagen of Table 6.

### Methods and Perturbagens for Directing a Change in Cell State

Particular cellular changes in cell state can be matched to differential gene expression (which collectively define a gene signature), caused by exposure of a cell to a perturbagen. In embodiments, a change in cell state may be from one progenitor cell type to another progenitor cell type. For example, a megakaryocyte/erythroid progenitor (MEP) may change to a promegakaryocyte. In embodiments, a change in cell state may be from an upstream progenitor cell (e.g. early common myeloid progenitor) to a downstream progenitor cell. Lastly, in embodiments, a change in cell state may be from the final non-differentiated cell into a differentiated cell.

An aspect of the present disclosure is a method for directing a change in cell state of a progenitor cell. The method includes a step of contacting a population of cells comprising a progenitor cell with at least one perturbagen selected from Table 6, or a variant thereof. In this aspect, the at least one perturbagen is capable of altering a gene signature in the progenitor cell. In one embodiment the progenitor cell is a non-lineage committed CD34⁺ cell.

Another aspect of the present disclosure is a method for directing a change in cell state of a progenitor cell. This method includes a step of contacting a population of cells comprising a progenitor cell with at least one perturbagen capable of altering a gene signature in the progenitor cell. In this aspect, altering the gene signature comprises an increase in expression and/or activity in the progenitor cell of one or more genes selected from Table 3 designated as an “up” gene in the gene directionality column of Table 3 and/or a decrease in expression and/or activity in the progenitor cell of one or more genes selected from Table 3 designated as a “down” gene in the gene directionality column of Table 3. In one embodiment, the progenitor cell is a non-lineage committed CD34⁺ cell.

Yet another aspect of the present disclosure is a method for directing a change in cell state of a progenitor cell. The method includes a step of contacting a population of cells comprising a progenitor cell with at least one perturbagen selected from Table 6, or a variant thereof, and capable of altering a gene signature in the progenitor cell. In this aspect, altering the gene signature comprises an increase in expression and/or activity in the progenitor cell of one or more genes selected from Table 3 designated as an “up” gene in the gene directionality column of Table 3 and/or a decrease in expression and/or activity in the progenitor cell of one or more genes selected from Table 3 designated as a “down” gene in the gene directionality column of Table 3. In one embodiment, the progenitor cell is a non-lineage committed CD34+ cell.

In some embodiments, the non-lineage committed CD34+ cell is a hematopoietic stem and progenitor cell (HSPC). In some embodiments, the step of contacting a population of cells comprising a progenitor cell with a perturbagen causes a change in the cell state. Such change in cell state can provide an increase in the number of one or more of megakaryocyte/erythroid progenitor cells (MEP), committed megakaryocyte progenitor cells, promegakaryocytes, megakaryocytes, proplatelets, and platelets. In some embodiments, the MEP cells are marked by CD34+CD71hi; in other embodiments, the MEP cells are marked by CD34+CD71HighCD41−, CD235−. In some embodiments, the committed megakaryocyte progenitor cells are marked by CD34+CD71Low CD41+CD235a− and in other embodiments, the committed megakaryocyte progenitor cells are marked by CD34+CD71+CD41+CD235−. In some embodiments, the promegakaryocytes are marked by CD34−CD41+CD42+, and increase ploidy. In some embodiments, the megakaryocytes can be derived from the canonical MEP developmental pathway. In some embodiments, the megakaryocytes can be derived from a developmental pathway that does not include the canonical MEP cell.

In embodiments, the change in cell state (by contacting a population of cells comprising a progenitor cell with a perturbagen) provides an increase in the number of megakaryocytes, proplatelets, and/or platelets.

In some embodiment, the change in the number of megakaryocytes, proplatelets, and/or platelets is relative to a control population of cells. For example, in some embodiments, the increase in the number of megakaryocytes, proplatelets, and/or platelets—upon contacting the cells with a perturbagen—is relative to the number of megakaryocytes, proplatelets, and/or platelets obtained from a population of progenitor cells that is not contacted with the perturbagen. In other embodiments, the increase in the number of megakaryocytes, proplatelets, and/or platelets—upon contacting the cells with a perturbagen—is relative to the population of progenitor cells prior to contacting it with the perturbagen. In some embodiments, a change in the number of megakaryocytes, proplatelets, and/or platelets is caused by change in the state of the cells of a population of progenitor cells. For example, an increase in the number of megakaryocytes, proplatelets, and/or platelets within a population of progenitor cell can be due to a change in the state of the cells.

In some embodiments, the change in the state of the cells of a population of progenitor cells provides an increase in the number of megakaryocyte/erythroid progenitor cells, erythroid progenitor cells, or megakaryocyte progenitor cells. In some embodiments, the change in the state of the cells of a population of progenitor cells provides an increase in the number of other committed blood cells, e.g., erythrocytes. In other embodiments, the change in cell state does not provide a substantial increase in the number of other committed blood cells, e.g., erythrocytes. In some embodiments, the change in cell state provides a decrease in the number of other committed blood cells, e.g., erythrocytes.

In some embodiments, the change in cell state causes an increase in the ratio of the number of megakaryocytes, proplatelets, and/or platelets to the number of other committed blood cells, e.g., erythrocytes relative to the ratio obtained from a population of progenitor cells that is not contacted with the at least one perturbagen. In other embodiments, the change in cell state causes an increase in the ratio of the number of megakaryocytes, proplatelets, and/or platelets to the number of other committed blood cells, e.g., erythrocytes relative to the ratio obtained in a population of progenitor cells prior to contacting with the at least one perturbagen.

In some embodiments, the ratio of the number of other committed blood cells, e.g., erythrocytes to the number of progenitor cells is increased relative to the ratio obtained from a population of progenitor cells that is not contacted with the at least one perturbagen. In other embodiments, the ratio of the number of other committed blood cells, e.g., erythrocytes to the number of progenitor cells is increased relative to the ratio in the population of progenitor cells prior to contacting with the at least one perturbagen. In embodiments, the ratio of the number of megakaryocytes, proplatelets, and/or platelets to the number of progenitor cells is increased relative to the ratio obtained from a population of progenitor cells that is not contacted with the at least one perturbagen. In some embodiments, the ratio of the number of megakaryocytes, proplatelets, and/or platelets to the number of progenitor cells is increased relative to the ratio in the population of progenitor cells prior to contacting with the at least one perturbagen.

In embodiments, the increase in the number of megakaryocytes, proplatelets, platelets and/or the number of other committed blood cells, e.g., erythrocytes is due in part to 1) increased cell proliferation, 2) an increased lifespan, or 3) reduced cell death of the megakaryocytes, proplatelets, platelets and/or the other committed blood cells, e.g., erythrocytes. In some embodiments, the increase in the number of megakaryocytes, proplatelets, platelets and/or the number of other committed blood cells, e.g., erythrocytes is due in part to a change of cell state from progenitor cells into the megakaryocyte and/or erythrocyte lineage.

Methods for determining the extension of the lifespan of a specific cell type or a reduction of cell death is well known in the art. As examples, markers for dying cells, e.g., caspases can be detected, or dyes for dead cells, e.g., methylene blue, may be used.

In embodiments, the number of progenitor cells is decreased. In embodiments, the decrease in the number of progenitor cells is due in part to decreased cell proliferation of the progenitor cells. In embodiments, the decrease in the number of progenitor cells is due in part to a decreased lifespan of the progenitor cells. In embodiments, the decrease in the number of progenitor cells is due in part to increased cell death among the progenitor cells. In embodiments, the decrease in the number of progenitor cells is relative to the number of progenitor cells in a population of progenitor cells that is not contacted with the at least one perturbagen. In embodiments, the decrease in the number of progenitor cells is relative to the number of progenitor cells in the population prior to contacting with the at least one perturbagen. In embodiments, the decrease in the number of progenitor cells is due to a change of cell state from a progenitor cell into the megakaryocyte lineage and/or erythrocyte lineage.

In embodiments, the number of progenitor cells is increased. In embodiments, the increase in the number of progenitor cells is due in part to increased cell proliferation of the progenitor cells. In embodiments, the increase in the number of progenitor cells is due in part to an increased lifespan of the progenitor cells. In embodiments, the increase in the number of progenitor cells is due in part to decreased cell death among the progenitor cells. In embodiments, the increase in the number of progenitor cells is relative to the number of progenitor cells in a population of progenitor cells that is not contacted with the at least one perturbagen. In embodiments, the increase in the number of progenitor cells is relative to the number of progenitor cells in the population prior to contacting with the at least one perturbagen.

In some embodiments, the number of MEP cells, committed megakaryocyte progenitor cells, promegakaryocytes, megakaryocytes, proplatelets, and/or platelets is increased after contacting the population of cells comprising a CD34+ cell with the at least one perturbagen. In some embodiments, the number of MEP cells and megakaryocytes, proplatelets, and/or platelets is increased after contacting the population of cells comprising a CD34+ cell with the at least one perturbagen. In other embodiments, the number of promegakaryocytes and megakaryocytes, proplatelets, and/or platelets is increased after contacting the population of cells comprising a CD34+ cell with the at least one perturbagen. In some embodiments, the ratio of the number of MEP cells, committed megakaryocyte progenitor cells, promegakaryocyte cells, megakaryocytes, proplatelets, and/or platelets to the number of progenitor cells is increased relative to the ratio obtained from a population of progenitor cells that is not contacted with the at least one perturbagen. In other embodiments, the ratio of the number MEP cells, committed megakaryocyte progenitor cells, promegakaryocytes, megakaryocytes, proplatelets, and/or platelets to the number of progenitor cells is increased relative to the ratio in the population of progenitor cells prior to contacting with the at least one perturbagen.

In some embodiments, for the methods described herein, the ratio of the number of committed megakaryocyte progenitor cells to the number of MEP cells is increased relative to the ratio obtained from a population of progenitor cells that is not contacted with the at least one perturbagen. In embodiments, for the methods described herein, the ratio of the number of committed megakaryocyte progenitor cells to the number of MEP cells is increased relative to the ratio in the population of progenitor cells prior to contacting with the at least one perturbagen. In other embodiments, for the methods described herein, the ratio of the number of promegakaryocytes to the number of committed megakaryocyte progenitor cells is increased relative to the ratio obtained from a population of progenitor cells that is not contacted with the at least one perturbagen. In other embodiments, for the methods described herein, the ratio of the number of promegakaryocytes to the number of committed megakaryocyte progenitor cells is increased relative to the ratio in the population of progenitor cells prior to contacting with the at least one perturbagen. In yet other embodiments, for the methods described herein, the ratio of the number of megakaryocytes, proplatelets, and/or platelets to promegakaryocytes is increased relative to the ratio obtained from a population of progenitor cells that is not contacted with the at least one perturbagen. In other embodiments, for the methods described here, the ratio of the number of megakaryocytes, proplatelets, and/or platelets to promegakaryocytes is increased relative to the ratio in the population of progenitor cells prior to contacting with the at least one perturbagen.

In some embodiments of the methods described herein, the ratio of the number of megakaryocytes, proplatelets, and/or platelets to committed megakaryocyte progenitor cells is increased relative to the ratio obtained from a population of progenitor cells that is not contacted with the at least one perturbagen. In other embodiments, the ratio of the number of megakaryocytes, proplatelets, and/or platelets to committed megakaryocyte progenitor cells is increased relative to the ratio in the population of progenitor cells prior to contacting with the at least one perturbagen. In embodiments, the ratio of the number of megakaryocytes, proplatelets, and/or platelets to MEP cells is increased relative to the ratio obtained from a population of progenitor cells that is not contacted with the at least one perturbagen. In some other embodiments of the methods described herein, the ratio of the number of megakaryocytes, proplatelets, and/or platelets to MEP cells is increased relative to the ratio in the population of progenitor cells prior to contacting with the at least one perturbagen.

Methods for counting cells are well known in the art. Non-limiting examples include hemocytometry, flow cytometry, and cell sorting techniques, e.g., fluorescence activated cell sorting (FACS).

In some embodiments, the number of MEP cells is decreased. In embodiments, the number of committed megakaryocyte progenitor cells is decreased. In some embodiments, the number of promegakaryocytes is decreased. In some embodiments, the number of MEP cells is increased. In some embodiments, the number of committed megakaryocyte progenitor cells is increased. In embodiments, the number of promegakaryocytes is increased.

In embodiments, the at least one perturbagen selected from Table 6, or a variant thereof, comprises at least 2, at least 3, at least 4, or at least 5 perturbagens selected from Table 6, or variants thereof.

In any of the aspects or embodiments disclosed herein, the at least one perturbagen is selected from Table 6, or acceptable salt, solvate, hydrate, co-crystal, clathrate, prodrug, or polymorph of thereof.

In embodiments, altering the gene signature comprises increased expression and/or increased activity in the progenitor cell of one or more genes selected from Table 3. In embodiments, the one or more genes selected from Table 3 designated as an “up” gene in the gene directionality column of Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more genes. In some embodiments, the genes selected from Table 3 designated as an “up” gene in the gene directionality column of Table 3 comprise at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK.

In embodiments, altering the gene signature comprises decreased expression and/or decreased activity in the progenitor cell of one or more genes selected from Table 3 designated as a “down” gene in the gene directionality column of Table 3. In embodiments, the one or more genes selected from Table 3 designated as a “down” gene in the gene directionality column of Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3. In embodiments, the one or more genes selected from Table 3 designated as a “down” gene in the gene directionality column of Table 3 comprise at least one of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a progenitor cell occurs in vitro or ex vivo.

In embodiments, contacting the population of cells comprising a progenitor cell occurs in vivo in a subject. In embodiments, the subject is a human. In embodiments, the human is an adult human. In some embodiments, the adult human has an abnormal number of one or more of megakaryocytes, protoplatelets, or platelets, or a disease or disorder characterized thereby.

In an aspect, the present disclosure provides a method for promoting the formation of a megakaryocyte cell, or an immediate progenitor thereof. The method includes a step of exposing a starting population of stem/progenitor cells comprising a non-lineage committed CD34+ cell to a perturbation having a perturbation signature that promotes the transition of the starting population of stem/progenitor cells into a MEP cell, committed megakaryocyte progenitor cell, or a promegakaryocyte. In this aspect, the perturbation signature comprises increased expression and/or activity of one or more of genes selected from Table 3 designated as an “up” gene in the gene directionality column of Table 3 and/or a decreased expression and/or activity in the non-lineage committed CD34+ cell of one or more genes selected from Table 3 designated as a “down” gene in the gene directionality column of Table 3. Embodiments associated with the above aspects are likewise relevant to the present aspect. In other words, each of the embodiments mentioned above for the above aspects may be revised/adapted to be applicable to the present aspect.

In another aspect, the present disclosure provides a method of increasing a quantity of megakaryocyte cell, or immediate progenitors thereof. The method includes a step of exposing a starting population of stem/progenitor cells comprising a non-lineage committed CD34+ cell to a pharmaceutical composition that promotes the formation of lineage specific progenitor population selected from MEP cell, committed megakaryocyte progenitor cell, or a promegakaryocyte. The pharmaceutical composition promotes the transition of a primitive stem/progenitor population into the lineage specific progenitor population that has the capacity to differentiate into megakaryocytes, proplatelets, and/or platelets or immediate progenitors thereof. In this aspect, the pharmaceutical composition comprises at least one perturbagen selected from Table 6, or a variant thereof. Embodiments associated with the above aspects are likewise relevant to the present aspect. In other words, each of the embodiments mentioned above for the above aspects may be revised/adapted to be applicable to the present aspect.

In yet another aspect, the present disclosure provides a perturbagen for use in any herein disclosed method.

In a further aspect, the present disclosure provides a pharmaceutical composition comprising perturbagen for use in any herein disclosed method.

### Methods and Perturbagens for Treating a Disease or Disorder

The ability of a perturbagen to specifically promote megakaryocyte, proplatelets, and/or platelets lineages would be valuable in designing a therapeutic composition. As examples, for a disease characterized by a reduced number of megakaryocytes, a therapeutic composition comprising a perturbagen that increases the number of megakaryocytes could be beneficial and/or a disease (including the same disease) that would benefit from increased numbers of proplatelets or platelets could be treated by a therapeutic composition comprising a perturbagen that increases the number of proplatelets or platelets.

An aspect of the present disclosure is a method for treating a disease or disorder characterized by an abnormal number of megakaryocytes, proplatelets, and/or platelets. The method includes a step of: (a) administering to a patient in need thereof a therapeutically effective amount of at least one perturbagen selected from Table 6, or a variant thereof, in which the at least one perturbagen is capable of changing a gene signature in a progenitor cell; or (b) administering to a patient in need thereof a cell, the cell having been contacted with at least one perturbagen selected from Table 6, or a variant thereof, in which the at least one perturbagen is capable of changing a gene signature in a progenitor cell.

In embodiments, the abnormal number of megakaryocytes, proplatelets, and/or platelets is a megakaryocyte deficiency.

In embodiments, the administering is directed to the bone marrow of the patient. In embodiments, the administering is via intraosseous injection or intraosseous infusion. In embodiments, the administering the cell is via intravenous injection or intravenous infusion. In other embodiments, the administering of the cell is via intravenous injection or intravenous infusion. In some embodiments, the administering is simultaneously or sequentially to one or more mobilization agents.

In some embodiments, the methods described herein are useful for treatment of a disease or disorder characterized by an abnormal number of megakaryocytes, proplatelets, and/or platelets, e.g., thrombocytopenia. In embodiments, the disease or disorder characterized by impaired megakaryopoiesis. In some embodiments, the disease or disorder is characterized by an abnormal number of megakaryocytes, proplatelets, and/or platelets and is a bleeding disorder. In other embodiments, the disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia.

In some embodiments, the methods described herein are where at least one perturbagen is administered on the basis of previously determining the patient exhibits an abnormal number of one or more of megakaryocytes, protoplatelets, or platelets, or a disease or disorder characterized thereby. In some embodiments, the present disclosure is related to a method of treating a disease or disorder characterized by an abnormal number of megakaryocytes, proplatelets, and/or platelets and includes administering an effective amount of a perturbagen selected from Table 6 or a variant thereof to a subject in need thereof. In some embodiments, the present disclosure is related to a method of treating a disease or disorder characterized by an abnormal number of megakaryocytes, proplatelets, and/or platelets, e.g., a bleeding disorder and includes administering an effective amount of a perturbagen selected from Table 6 or a variant thereof to a subject in need thereof. In other embodiments, the present disclosure is related to a method of treating a disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, and includes administering an effective amount of a perturbagen selected from Table 6 or a variant thereof to a subject in need thereof.

Another aspect of the present disclosure is a method for treating disease or disorder selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia. The method comprising: (a) administering to a patient in need thereof a therapeutically effective amount of at least one perturbagen selected from Table 6, or a variant thereof, or (b) administering to a patient in need thereof a cell, the cell having been contacted with at least one perturbagen selected from Table 6, or a variant thereof.

Another aspect of the present disclosure is a method for treating thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In an aspect, the present disclosure provides a method for treating a bleeding disorder. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In another aspect, the present disclosure provides a method for treating congenital amegakaryocytic thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In yet another aspect, the present disclosure provides a method for treating thrombocytopenia with absent radii. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In a further aspect, the present disclosure provides a method for treating radio ulnar synostosis with congenital thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

An aspect of the present disclosure is a method for treating X-linked macrothrombocytopenia with thalassemia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Another aspect of the present disclosure is a method for treating GB11b-related thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Yet another aspect of the present disclosure is a method for treating Von Willebrand diseases Type 2B. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Another aspect of the present disclosure is a method for treating platelet-type Von Willebrand disease. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Yet another aspect of the present disclosure is a method for treating CYCS-Related thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Another aspect of the present disclosure is a method for treating immune thrombocytopenia (idiopathic thrombocytopenic purpura). The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Yet another aspect of the present disclosure is a method for treating myeloablation/chemotherapy induced thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating thrombocytopenia resulting from liver disease. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating thrombocytosis. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating myelofribrosis. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating radiation-induced thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

In embodiments, the administering is simultaneously or sequentially to one or more mobilization agents. In one aspect, the present disclosure is related to a method for treating a disease or disorder characterized by an abnormal ratio of megakaryocytes, proplatelets, and/or platelets to non-lineage committed CD34⁺ cells, MEP cells, committed megakaryocyte progenitor cells, and/or promegakaryocytes. This method includes (a) administering to a patient in need thereof at least one perturbagen, or a variant thereof, wherein the at least one perturbagen is capable of changing a gene signature in a progenitor cell, or (b) administering to a patient in need thereof a cell, the cell having been contacted with at least one perturbagen, or a variant thereof, wherein the at least one perturbagen is capable of changing a gene signature in a progenitor cell.

Another aspect of the present disclosure is a method for treating a disease or disorder characterized by an abnormal ratio of megakaryocytes, proplatelets, and/or platelets to non-lineage committed CD34⁺ cells, MEP cells, committed megakaryocyte progenitor cells, and/or promegakaryocytes. The method includes a step of: (a) administering to a patient in need thereof at least one perturbagen selected from Table 6, or a variant thereof, in which the at least one perturbagen is capable of changing a gene signature in a progenitor cell; or (b) administering to a patient in need thereof a cell, the cell having been contacted with at least one perturbagen selected from Table 6, or a variant thereof, in which the at least one perturbagen is capable of changing a gene signature in a progenitor cell.

In embodiments, the abnormal ratio comprises a decreased number of megakaryocytes, proplatelets, and/or platelets and/or increased number of progenitor cells. In embodiments, the abnormal ratio comprises an increased number of progenitor cells. In embodiments, the abnormal ratio comprises an increased number of non-lineage committed CD34+ cells, MEP cells, committed megakaryocyte progenitor cells, and/or promegakaryocytes.

In embodiments related to this aspect of the disclosure, the administering is directed to the bone marrow of the patient. In other embodiments, the administering is via intraosseous injection or intraosseous infusion.

In embodiments, the administering the cell is via intravenous injection or intravenous infusion.

In embodiments, the administering is simultaneously or sequentially to one or more mobilization agents.

In embodiments, the administering occurs about once per day for one or more days. In embodiments, the administering occurs more than once per day for one or more days. In embodiments, the administering occurs at most once per day for one or more days. In embodiments, the administering occurs substantially continuously per administration period.

In embodiments, the disease or disorder characterized by an abnormal ratio of megakaryocytes, proplatelets, and/or platelets to non-lineage committed CD34+ cells, MEP cells, committed megakaryocyte progenitor cells, and/or promegakaryocytes is a thrombocytopenia. In embodiments, the disease or disorder characterized by an abnormal ratio of megakaryocytes, proplatelets, and/or platelets to non-lineage committed CD34+ cells, MEP cells, committed megakaryocyte progenitor cells, and/or promegakaryocytes is a characterized by impaired megakaryopoiesis. In some embodiments, the disease or disorder characterized by an abnormal ratio of megakaryocytes, proplatelets, and/or platelets to non-lineage committed CD34+ cells, MEP cells, committed megakaryocyte progenitor cells, and/or promegakaryocytes is a bleeding disorder. In other embodiments, the disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia.

In some embodiments, the present disclosure is related to a method of treating a disease or disorder characterized by an abnormal number of megakaryocytes, proplatelets, and/or platelets and includes administering an effective amount of a perturbagen selected from Table 6 or a variant thereof to a subject in need thereof. In some embodiments, the present disclosure is related to a method of treating a disease or disorder characterized by an abnormal number of megakaryocytes, proplatelets, and/or platelets, e.g., a bleeding disorder and includes administering an effective amount of a perturbagen selected from Table 6 or a variant thereof to a subject in need thereof. In other embodiments, the present disclosure is related to a method of treating a disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, and includes administering an effective amount of a perturbagen selected from Table 6 or a variant thereof to a subject in need thereof.

Another aspect of the present disclosure is a method for treating thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In an aspect, the present disclosure provides a method for treating a bleeding disorder. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In another aspect, the present disclosure provides a method for treating congenital amegakaryocytic thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In yet another aspect, the present disclosure provides a method for treating thrombocytopenia with absent radii. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

In a further aspect, the present disclosure provides a method for treating radio ulnar synostosis with congenital thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

An aspect of the present disclosure is a method for treating X-linked macrothrombocytopenia with thalassemia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Another aspect of the present disclosure is a method for treating GB11b-related thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Yet another aspect of the present disclosure is a method for treating Von Willebrand diseases Type 2B. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Another aspect of the present disclosure is a method for treating platelet-type Von Willebrand disease. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Yet another aspect of the present disclosure is a method for treating CYCS-Related thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Another aspect of the present disclosure is a method for treating immune thrombocytopenia (idiopathic thrombocytopenic purpura). The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof.

Yet another aspect of the present disclosure is a method for treating myeloablation/chemotherapy induced thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating thrombocytopenia resulting from liver disease. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating thrombocytosis. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating myelofribrosis. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

Yet another aspect of the present disclosure is a method for treating radiation-induced thrombocytopenia. The method comprising a step of: (a) administering to a patient in need thereof a therapeutically effective amount of at least one perturbagen selected from Table 6, or a variant thereof; or (b) administering to a patient in need thereof a cell, the cell having been contacted with at least one perturbagen selected from Table 6, or a variant thereof. In some embodiments, the at least one perturbagen is capable of changing a gene signature in a progenitor cell.

In some embodiments, a suitable patient for the methods of treatment described herein is selected by steps comprising obtaining from the patient having the disease or disorder a sample of cells comprising a non-lineage committed CD34+ cell; and contacting the sample of cells with least one perturbagen selected from Table 6, or a variant thereof, wherein the at least one perturbagen alters a gene signature in the sample of cells. In embodiments, the patient is selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3.

In other embodiments, the patient is selected by steps including obtaining from a subject having the disease or disorder a sample of cells comprising a non-lineage committed CD34+ cell; and contacting the sample of cells with at least one perturbagen selected from Table 6, or a variant thereof; wherein the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3. In embodiments, the method for selecting the patient includes obtaining from a subject having the disease or disorder a sample of cells comprising a non-lineage committed CD34+ cell; and contacting the sample of cells with least one perturbagen selected from Table 6, or a variant thereof, wherein when the at least one perturbagen alters a gene signature in the sample of cells, the subject is selected as a patient. In yet other embodiments, the method for selecting the patient includes obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3, the subject is selected as a patient. In other embodiments, the method for selecting the patient includes obtaining from a subject having the disease or disorder a sample of cells comprising a non-lineage committed CD34+ cell; and contacting the sample of cells with at least one perturbagen selected from Table 6, or a variant thereof; wherein when the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3, the subject is selected as a patient.

### Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of hematopoietic cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11(1): 78-88; Hopman and DiPersio “Advances in Stem Cell Mobilization.” Blood Review, 2014, 28(1): 31-40; and Kim “Hematopoietic stem cell mobilization: current status and future perspective.” Blood Res. 2017 June; 52(2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

### Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

Combination therapies, comprising more than one perturbagen, can be co-delivered in a single delivery vehicle or delivery device.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any perturbagens disclosed herein is formulated in accordance with routine procedures as a pharmaceutical composition adapted for a mode of administration disclosed herein.

### Other Aspects of the Present Disclosure

Embodiments associated with any of the above-disclosed aspects are likewise relevant to the below-mentioned aspects. In other words, each of the embodiments mentioned above for the above aspects may be revised/adapted to be applicable to the below aspects.

Yet another aspect of the present disclosure is a use of the perturbagen of Table 6, or a variant thereof in the manufacture of a medicament for treating a disease or disorder characterized by an abnormal ratio of megakaryocytes, proplatelets, and/or platelets to progenitor cells.

In an aspect, the present disclosure provides a use of the perturbagen of Table 6, or a variant thereof in the manufacture of a medicament for treating a disease or disorder characterized by an abnormal ratio of megakaryocytes, proplatelets, and/or platelets to other committed blood cells, optionally erythrocytes.

In another aspect, the present disclosure provides a method of identifying a candidate perturbation for promoting the transition of a starting population of progenitor cells into megakaryocytes, proplatelets, and/or platelets or immediate progenitors thereof. The method includes the steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of cells in the population of progenitor cells into megakaryocytes, proplatelets, and/or platelets or immediate progenitors thereof following exposure of the population of cells to the perturbation; and identifying the perturbation as a candidate perturbation for promoting the transition of a population of progenitor cells into megakaryocytes, proplatelets, and/or platelets or immediate progenitors thereof based on the perturbation signature. In this aspect, the perturbation signature is an increase in expression and/or activity in the progenitor cell of one or more genes selected from Table 3 designated as an “up” gene in the gene directionality column of Table 3, and/or a decrease in expression and/or activity in the progenitor cell of one or more genes selected from Table 3 designated as a “down” gene in the gene directionality column of Table 3.

In another aspect, the present disclosure provides a method for making a therapeutic agent for a disease or disorder selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia. The method includes the steps of: (a) identifying a therapeutic agent for therapy; and (b) formulating the therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into megakaryocytes, proplatelets, and/or platelets or immediate progenitors thereof following exposure of the population of cells to the perturbation; and identifying the perturbation as a candidate perturbation for promoting the transition of a population of progenitor cells into megakaryocytes, proplatelets, and/or platelets or immediate progenitors thereof based on the perturbation signature. Further, in this aspect, the perturbation signature is an increase in expression and/or activity in the progenitor cell of one or more genes selected from Table 3 designated as an “up” gene in the gene directionality column of Table 3, and/or a decrease in expression and/or activity in the progenitor cell of one or more genes selected from Table 3 designated as a “down” gene in the gene directionality column of Table 3.

Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows:

Embodiment 227: A method for directing a change in cell state of a progenitor cell comprising: contacting a population of cells comprising a progenitor cell with at least one perturbagen selected from Table 6, or a variant thereof, wherein the at least one perturbagen is capable of altering a gene signature in the progenitor cell; and wherein the progenitor cell is a non-lineage committed CD34+ cell.
Embodiment 228: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from Table 3 designated as an “up” gene in the gene directionality column of Table 3 and/or a decrease in expression and/or activity in the progenitor cell of one or more genes selected from Table 3 designated as a “down” gene in the gene directionality column of Table 3 and wherein the progenitor cell is a non-lineage committed CD34+ cell.
Embodiment 229: The method of Embodiment 228, wherein altering the gene signature comprises an activation of a network module designated in the network module column of Table 3.
Embodiment 230: The method of Embodiment 229, wherein the activation of the network module designated in the network module column of Table 3 comprises modulating expression and/or activity of 2 or more genes within a network module.
Embodiment 231: The method of Embodiment 230, wherein the activation of the network module designated in the network module column of Table 3 comprises modulating expression and/or activity of all of the genes within a network module.
Embodiment 232: The method of Embodiment 228, wherein the activation of the network module designated in the network module column of Table 3 comprises modulating expression and/or activity of 2 or more genes within 2 or more network modules.
Embodiment 233: The method of Embodiment 228, wherein altering the gene signature comprises an increase in expression and/or activity in the progenitor cell of two or more genes designated as an “up” gene in the gene directionality column of Table 3.
Embodiment 234: The method of Embodiment 233, wherein altering the gene signature comprises a decrease in expression and/or activity in the progenitor cell of two or more genes selected from Table 3 designated as a “down” gene in the gene directionality column of Table 3.
Embodiment 235: A method for directing a change in cell state of a progenitor cell, comprising: contacting a population of cells comprising a progenitor cell with at least one perturbagen selected from Table 6, or a variant thereof, and capable of altering a gene signature in the progenitor cell, wherein altering the gene signature comprises an increase in expression and/or activity in the progenitor cell of one or more genes selected from Table 3 designated as an “up” gene in the gene directionality column of Table 3 and/or a decrease in expression and/or activity in the progenitor cell of one or more genes selected from Table 3 designated as a “down” gene in the gene directionality column of Table 3 and wherein the progenitor cell is a non-lineage committed CD34+ cell.
Embodiment 236: The method of Embodiment 235, wherein altering the gene signature comprises an activation of a network module designated in the network module column of Table 3.
Embodiment 237: The method of Embodiment 236, wherein the activation of the network module designated in the network module column of Table 3 comprises modulating expression and/or activity of 2 or more genes within a network module.
Embodiment 238: The method of Embodiment 237, wherein the activation of the network module designated in the network module column of Table 3 comprises modulating expression and/or activity of all of the genes within a network module.
Embodiment 239: The method of Embodiment 235, wherein the activation of the network module designated in the network module column of Table 3 comprises modulating expression and/or activity of 2 or more genes within 2 or more network modules.
Embodiment 240: The method of Embodiment 235, wherein altering the gene signature comprises an increase in expression and/or activity in the progenitor cell of two or more genes designated as an “up” gene in the gene directionality column of Table 3.
Embodiment 241: The method of Embodiment 240, wherein altering the gene signature comprises a decrease in expression and/or activity in the progenitor cell of two or more genes designated as a “down” gene in the gene directionality column of Table 3.
Embodiment 242: The method of any one of Embodiments 227 to 229, wherein the change in cell state provides an increase in the number of one or more of megakaryocyte/erythroid progenitor cells (MEP), committed megakaryocyte progenitor cells, promegakaryocytes, megakaryocytes, proplatelets, and platelets.
Embodiment 243: The method of Embodiment 242, wherein the increase in the number of megakaryocytes, proplatelets, and/or platelets is relative to the number of megakaryocytes, proplatelets, and/or platelets obtained from a population of progenitor cells that is not contacted with the at least one perturbagen.
Embodiment 244: The method of Embodiment 242, wherein the increase in the number of megakaryocytes, proplatelets, and/or platelets is relative to the population of progenitor cells prior to contacting with the at least one perturbagen.
Embodiment 245: The method of Embodiment 243 or Embodiment 244, wherein the change in cell state provides an increase in the number of megakaryocytes, proplatelets, and/or platelets.
Embodiment 246: The method of Embodiment 245, wherein the change in cell state provides an increase in the number of other committed blood cells, optionally erythrocytes.
Embodiment 247: The method of Embodiment 245, wherein the change in cell state does not provide a substantial increase in the number of other committed blood cells, optionally erythrocytes and/or provides a decrease in the number of other committed blood cells, optionally erythrocytes.
Embodiment 248: The method of any one of Embodiments 245 to 247, wherein the ratio of the number of megakaryocytes, proplatelets, and/or platelets to the number of other committed blood cells, optionally erythrocytes is increased relative to the ratio obtained from a population of progenitor cells that is not contacted with the at least one perturbagen.
Embodiment 249: The method of any one of Embodiments 245 to 247, wherein the ratio of the number of megakaryocytes, proplatelets, and/or platelets to the number of other committed blood cells, optionally erythrocytes is increased relative to the ratio in the population of progenitor cells prior to contacting with the at least one perturbagen.
Embodiment 250: The method of Embodiment 242, wherein the ratio of the number of other committed blood cells, optionally erythrocytes to the number of progenitor cells is increased relative to the ratio obtained from a population of progenitor cells that is not contacted with the at least one perturbagen.
Embodiment 251: The method of Embodiment 242, wherein the ratio of the number of other committed blood cells, optionally erythrocytes to the number of progenitor cells is increased relative to the ratio in the population of progenitor cells prior to contacting with the at least one perturbagen.
Embodiment 252: The method of Embodiment 242, wherein the ratio of the number of megakaryocytes, proplatelets, and/or platelets to the number of progenitor cells is increased relative to the ratio obtained from a population of progenitor cells that is not contacted with the at least one perturbagen.
Embodiment 253: The method of Embodiment 242, wherein the ratio of the number of megakaryocytes, proplatelets, and/or platelets to the number of progenitor cells is increased relative to the ratio in the population of progenitor cells prior to contacting with the at least one perturbagen.
Embodiment 254: The method of any one of Embodiments 242 to 253, wherein the increase in the number of megakaryocytes, proplatelets, platelets and/or the number of other committed blood cells, optionally erythrocytes is due in part to increased cell proliferation of the megakaryocytes, proplatelets, platelets and/or the other committed blood cells, optionally erythrocytes.
Embodiment 255: The method of any one of Embodiments 242 to 254, wherein the increase in the number of megakaryocytes, proplatelets, platelets and/or the number of other committed blood cells, optionally erythrocytes is due in part to an increased lifespan of the megakaryocytes, proplatelets, platelets and/or the other committed blood cells, optionally erythrocytes.
Embodiment 256: The method of any one of Embodiments 242 to 255, wherein the increase in the number of megakaryocytes, proplatelets, platelets and/or the number of other committed blood cells, optionally erythrocytes is due in part to reduced cell death among the megakaryocytes, proplatelets, platelets and/or the other committed blood cells, optionally erythrocytes.
Embodiment 257: The method of any one of Embodiments 242 to 256, wherein the increase in the number of megakaryocytes, proplatelets, platelets and/or the number of other committed blood cells, optionally erythrocytes is due in part to a change of cell state from progenitor cells into the megakaryocyte and/or erythrocyte lineage.
Embodiment 258: The method of any one of Embodiments 227 to 258, wherein the number of progenitor cells is decreased.
Embodiment 259: The method of Embodiment 258, wherein the decrease in the number of progenitor cells is due in part to decreased cell proliferation of the progenitor cells.
Embodiment 260: The method of Embodiment 258 or Embodiment 259, wherein the decrease in the number of progenitor cells is due in part to a decreased lifespan of the progenitor cells.
Embodiment 261: The method of any one of Embodiments 258 to 260, wherein the decrease in the number of progenitor cells is due in part to increased cell death among the progenitor cells.
Embodiment 262: The method of any one of Embodiments 258 to 261, wherein the decrease in the number of progenitor cells is relative to the number of progenitor cells in a population of progenitor cells that is not contacted with the at least one perturbagen.
Embodiment 263: The method of any one of Embodiments 258 to 262, wherein the decrease in the number of progenitor cells is relative to the number of progenitor cells in the population prior to contacting with the at least one perturbagen.
Embodiment 264: The method of any one of Embodiments 258 to 263, wherein the decrease in the number of progenitor cells is due to a change of cell state from a progenitor cell into the megakaryocyte lineage and/or erythrocyte lineage.
Embodiment 265: The method of any one of Embodiments 227 to 257, wherein the number of progenitor cells is increased.
Embodiment 266: The method of Embodiment 265, wherein the increase in the number of progenitor cells is due in part to increased cell proliferation of the progenitor cells.
Embodiment 267: The method of Embodiment 265 or Embodiment 266, wherein the increase in the number of progenitor cells is due in part to an increased lifespan of the progenitor cells.
Embodiment 268: The method of any one of Embodiments 265 to 267, wherein the increase in the number of progenitor cells is due in part to decreased cell death among the progenitor cells.
Embodiment 269: The method of any one of Embodiments 265 to 268, wherein the increase in the number of progenitor cells is relative to the number of progenitor cells in a population of progenitor cells that is not contacted with the at least one perturbagen.
Embodiment 270: The method of any one of Embodiments 265 to 268, wherein the increase in the number of progenitor cells is relative to the number of progenitor cells in the population prior to contacting with the at least one perturbagen.
Embodiment 271: The method of any one of Embodiments 227 to 257, wherein the number of MEP cells, committed megakaryocyte progenitor cells, promegakaryocytes, megakaryocytes, proplatelets, and/or platelets is increased after contacting the population of cells comprising a CD34+ cell with the at least one perturbagen.
Embodiment 272: The method of any one of Embodiments 227 to 257, wherein the number of MEP cells and megakaryocytes, proplatelets, and/or platelets is increased after contacting the population of cells comprising a CD34+ cell with the at least one perturbagen.
Embodiment 273: The method of any one of Embodiments 227 to 257, wherein the number of promegakaryocytes and megakaryocytes, proplatelets, and/or platelets is increased after contacting the population of cells comprising a CD34+ cell with the at least one perturbagen.
Embodiment 274: The method of Embodiment 271, wherein the ratio of the number of MEP cells, committed megakaryocyte progenitor cells, promegakaryocyte cells, megakaryocytes, proplatelets, and/or platelets to the number of progenitor cells is increased relative to the ratio obtained from a population of progenitor cells that is not contacted with the at least one perturbagen.
Embodiment 275: The method of Embodiment 271, wherein the ratio of the number MEP cells, committed megakaryocyte progenitor cells, promegakaryocytes, megakaryocytes, proplatelets, and/or platelets to the number of progenitor cells is increased relative to the ratio in the population of progenitor cells prior to contacting with the at least one perturbagen.
Embodiment 276: The method of any one of Embodiments 227 to 257, wherein the ratio of the number of committed megakaryocyte progenitor cells to the number of MEP cells is increased relative to the ratio obtained from a population of progenitor cells that is not contacted with the at least one perturbagen.
Embodiment 277: The method of Embodiment 276, wherein the ratio of the number of committed megakaryocyte progenitor cells to the number of MEP cells is increased relative to the ratio in the population of progenitor cells prior to contacting with the at least one perturbagen.
Embodiment 278: The method of any one of Embodiments 227 to 257, wherein the ratio of the number of promegakaryocytes to the number of committed megakaryocyte progenitor cells is increased relative to the ratio obtained from a population of progenitor cells that is not contacted with the at least one perturbagen.
Embodiment 279: The method of Embodiment 278, wherein the ratio of the number of promegakaryocytes to the number of committed megakaryocyte progenitor cells is increased relative to the ratio in the population of progenitor cells prior to contacting with the at least one perturbagen.
Embodiment 280: The method of any one of Embodiments 227 to 257, wherein the ratio of the number of megakaryocytes, proplatelets, and/or platelets to promegakaryocytes is increased relative to the ratio obtained from a population of progenitor cells that is not contacted with the at least one perturbagen.
Embodiment 281: The method of Embodiment 280, wherein the ratio of the number of megakaryocytes, proplatelets, and/or platelets to promegakaryocytes is increased relative to the ratio in the population of progenitor cells prior to contacting with the at least one perturbagen.
Embodiment 282: The method of any one of Embodiments 227 to 257, wherein the ratio of the number of megakaryocytes, proplatelets, and/or platelets to committed megakaryocyte progenitor cells is increased relative to the ratio obtained from a population of progenitor cells that is not contacted with the at least one perturbagen.
Embodiment 283: The method of Embodiment 282, wherein the ratio of the number of megakaryocytes, proplatelets, and/or platelets to committed megakaryocyte progenitor cells is increased relative to the ratio in the population of progenitor cells prior to contacting with the at least one perturbagen.
Embodiment 284: The method of any one of Embodiments 227 to 257, wherein the ratio of the number of megakaryocytes, proplatelets, and/or platelets to MEP cells is increased relative to the ratio obtained from a population of progenitor cells that is not contacted with the at least one perturbagen.
Embodiment 285: The method of Embodiment 284, wherein the ratio of the number of megakaryocytes, proplatelets, and/or platelets to MEP cells is increased relative to the ratio in the population of progenitor cells prior to contacting with the at least one perturbagen.
Embodiment 286: The method of any of Embodiments 227 to 270, wherein the number of MEP cells is decreased.
Embodiment 287: The method of any of Embodiments 227 to 270, wherein the number of committed megakaryocyte progenitor cells is decreased.
Embodiment 288: The method of any of Embodiments 227 to 270, wherein the number of promegakaryocytes is decreased.
Embodiment 289: The method of any of Embodiments 227 to 270, wherein the number of MEP cells is increased.
Embodiment 290: The method of any of Embodiments 227 to 270, wherein the number of committed megakaryocyte progenitor cells is increased.
Embodiment 291: The method of any of Embodiments 227 to 270, wherein the number of promegakaryocytes is increased.
Embodiment 292: The method of any one of Embodiments 227 to 291, wherein the at least one perturbagen selected from Table 6, or a variant thereof, comprises at least 2, at least 3, at least 4, or at least 5 perturbagens selected from Table 6, or variants thereof.
Embodiment 293: The method of Embodiment 228, wherein the one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or 23 or more genes designated as an “up” gene in the gene directionality column of Table 3.
Embodiment 294: The method of Embodiment 293, wherein the one or more genes selected from Table 3 comprises at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK.
Embodiment 295: The method of Embodiment 228, wherein the one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3.
Embodiment 296: The method of Embodiment 295, wherein the one or more genes selected from Table 3 comprises at leastone of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.
Embodiment 297: The method of any one of Embodiments 227 to 296, wherein contacting the population of progenitor cells occurs in vitro or ex vivo.
Embodiment 298: The method of any one of Embodiments 227 to 297, wherein contacting the population of progenitor cells occurs in vivo in a subject.
Embodiment 299: The method of Embodiment 298, wherein the subject is a human.
Embodiment 300: The method of Embodiment 299, wherein the human is an adult human.
Embodiment 301: A perturbagen for use in the method of any one of Embodiments 227 to 300.
Embodiment 302: A pharmaceutical composition comprising the perturbagen of Embodiment 301.
Embodiment 303: A method for promoting the formation of a megakaryocyte cell, or an immediate progenitor thereof, comprising: exposing a starting population of stem/progenitor cells comprising a non-lineage committed CD34+ cell to a perturbation having a perturbation signature that promotes the transition of the starting population of stem/progenitor cells into a MEP cell, committed megakaryocyte progenitor cell, or a promegakaryocyte, wherein the perturbation signature comprises increased expression and/or activity of one or more of genes selected from Table 3 designated as an “up” gene in the gene directionality column of Table 3 and/or a decreased expression and/or activity in the non-lineage committed CD34+ cell of one or more genes selected from Table 3 designated as a “down” gene in the gene directionality column of Table 3.
Embodiment 304: The method of Embodiment 303, wherein the perturbation signature comprises an activation of a network module designated in the network module column of Table 3.
Embodiment 305: The method of Embodiment 304, wherein the activation of the network module designated in the network module column of Table 3 comprises modulating expression and/or activity of 2 or more genes within a network module.
Embodiment 306: The method of Embodiment 305, wherein the activation of the network module designated in the network module column of Table 3 comprises modulating expression and/or activity of all of the genes within a network module.
Embodiment 307: The method of Embodiment 303, wherein the activation of the network module designated in the network module column of Table 3 comprises modulating expression and/or activity of 2 or more genes within 2 or more network modules.
Embodiment 308: The method of Embodiment 303, wherein the perturbation signature comprises an increase in expression and/or activity in the progenitor cell of two or more genes designated as an “up” gene in the gene directionality column of Table 3.
Embodiment 309: The method of Embodiment 303, wherein the perturbation signature comprises a decrease in expression and/or activity in the progenitor cell of two or more genes designated as a “down” gene in the gene directionality column of Table 3.
Embodiment 310: A method of increasing a quantity of megakaryocyte cell, or immediate progenitors thereof, comprising: exposing a starting population of stem/progenitor cells comprising a non-lineage committed CD34⁺ cell to a pharmaceutical composition that promotes the formation of lineage specific progenitor population selected from MEP cell, committed megakaryocyte progenitor cell, or a promegakaryocyte, the pharmaceutical composition promoting the transition of a primitive stem/progenitor population into the lineage specific progenitor population that has the capacity to differentiate into megakaryocytes, proplatelets, and/or platelets or immediate progenitors thereof, wherein the pharmaceutical composition comprises at least one perturbagen selected from Table 6, or a variant thereof.
Embodiment 311: A method for treating a disease or disorder disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, the method comprising: (a) administering to a patient in need thereof a therapeutically effective amount of at least one perturbagen selected from Table 6, or a variant thereof, wherein the at least one perturbagen is capable of changing a gene signature in a progenitor cell, or (b) administering to a patient in need thereof a cell, the cell having been contacted with at least one perturbagen selected from Table 6, or a variant thereof, wherein the at least one perturbagen is capable of changing a gene signature in a progenitor cell.
Embodiment 312: A method for treating a disease or disorder characterized by an abnormal number of megakaryocytes, proplatelets, and/or platelets, comprising: (a) administering to a patient in need thereof a therapeutically effective amount of at least one perturbagen selected from Table 6, or a variant thereof, wherein the at least one perturbagen is capable of changing a gene signature in a progenitor cell, or (b) administering to a patient in need thereof a cell, the cell having been contacted with at least one perturbagen selected from Table 6, or a variant thereof, wherein the at least one perturbagen is capable of changing a gene signature in a progenitor cell.
Embodiment 313: The method of Embodiment 312, wherein the abnormal number of megakaryocytes, proplatelets, and/or platelets is a megakaryocyte deficiency.
Embodiment 314: The method of any one of Embodiments 311 to 313, wherein the administering is directed to the bone marrow of the patient.
Embodiment 315: The method of Embodiment 314, wherein the administering is via intraosseous injection or intraosseous infusion.
Embodiment 316: The method of any one of Embodiments 311 to 315, wherein the administering the cell is via intravenous injection or intravenous infusion.
Embodiment 317: The method of any one of Embodiments 311 to 316, wherein the administering is simultaneously or sequentially to one or more mobilization agents.
Embodiment 318: The method of any one of Embodiments 311 to 317, wherein the disease or disorder characterized by an abnormal number of megakaryocytes, proplatelets, and/or platelets is a thrombocytopenia.
Embodiment 319: The method of any one of Embodiments 311 to 318, wherein the disease or disorder characterized by impaired megakaryopoiesis.
Embodiment 320: The method of any one of Embodiments 311 to 319, wherein the disease or disorder characterized by an abnormal number of megakaryocytes, proplatelets, and/or platelets is a bleeding disorder.
Embodiment 321: The method of any one of Embodiments 313 to 320, wherein the disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia.
Embodiment 322: The method of any one of Embodiments 311 to 321, wherein at least one perturbagen is administered on the basis of previously determining the patient exhibits an abnormal number of one or more of megakaryocytes, protoplatelets, or platelets, or a disease or disorder characterized thereby.
Embodiment 323: A method for treating a disease or disorder characterized by an abnormal ratio of megakaryocytes, proplatelets, and/or platelets to non-lineage committed CD34+ cells, MEP cells, committed megakaryocyte progenitor cells, and/or promegakaryocytes, comprising: (a) administering to a patient in need thereof at least one perturbagen selected from Table 6, or a variant thereof, wherein the at least one perturbagen is capable of changing a gene signature in a progenitor cell, or (b) administering to a patient in need thereof a cell, the cell having been contacted with at least one perturbagen selected from Table 6, or a variant thereof, wherein the at least one perturbagen is capable of changing a gene signature in a progenitor cell.
Embodiment 324: The method of Embodiment 323, wherein the abnormal ratio comprises a decreased number of megakaryocytes, proplatelets, and/or platelets and/or an increased number of progenitor cells.
Embodiment 325: The method of Embodiment 324, wherein the abnormal ratio comprises an increased number of progenitor cells.
Embodiment 326: The method of Embodiment 325, wherein the abnormal ratio comprises an increased number of non-lineage committed CD34+ cells, MEP cells, committed megakaryocyte progenitor cells, and/or promegakaryocytes.
Embodiment 327: The method of any one of Embodiments 322 to 326, wherein the administering is directed to the bone marrow of the patient.
Embodiment 328: The method of Embodiment 327, wherein the administering is via intraosseous injection or intraosseous infusion.
Embodiment 329: The method of any one of Embodiments 322 to 328, wherein the administering the cell is via intravenous injection or intravenous infusion.
Embodiment 330: The method of any one of Embodiments 322 to 329, wherein the administering is simultaneously or sequentially to one or more mobilization agents.
Embodiment 331: The method of any one of Embodiments 322 to 330, wherein the disease or disorder characterized by an abnormal ratio of megakaryocytes, proplatelets, and/or platelets to non-lineage committed CD34+ cells, MEP cells, committed megakaryocyte progenitor cells, and/or promegakaryocytes is a thrombocytopenia.
Embodiment 332: The method of any one of Embodiments 322 to 331, wherein the disease or disorder characterized by an abnormal ratio of megakaryocytes, proplatelets, and/or platelets to non-lineage committed CD34+ cells, MEP cells, committed megakaryocyte progenitor cells, and/or promegakaryocytes is a characterized by impaired megakaryopoiesis.
Embodiment 333: The method of any one of Embodiments 322 to 332, wherein the disease or disorder characterized by an abnormal ratio of megakaryocytes, proplatelets, and/or platelets to non-lineage committed CD34+ cells, MEP cells, committed megakaryocyte progenitor cells, and/or promegakaryocytes is a bleeding disorder.
Embodiment 334: The method of any one of Embodiments 322 to 333, wherein the disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia.
Embodiment 335: The method of any one of Embodiments 311 to 334, wherein the at least one perturbagen is capable of changing a gene signature in a progenitor cell.
Embodiment 336: The method of any one of Embodiments 311 to 335, wherein the patient is selected by steps comprising: obtaining from the patient having the disease or disorder a sample of cells comprising a non-lineage committed CD34+ cell; and contacting the sample of cells with least one perturbagen selected from Table 6, or a variant thereof, wherein the at least one perturbagen alters a gene signature in the sample of cells.
Embodiment 337: The method of any one of Embodiments 311 to 335, wherein the patient is selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3.
Embodiment 338: The method of Embodiment 337, wherein altering the gene signature comprises an activation of a network module designated in the network module column of Table 3.
Embodiment 339: The method of Embodiment 338, wherein the activation of the network module designated in the network module column of Table 3 comprises modulating expression and/or activity of 2 or more genes within a network module.
Embodiment 340: The method of Embodiment 339, wherein the activation of the network module designated in the network module column of Table 3 comprises modulating expression and/or activity of all of the genes within a network module.
Embodiment 341: The method of Embodiment 337, wherein the activation of the network module designated in the network module column of Table 3 comprises modulating expression and/or activity of 2 or more genes within 2 or more network modules.
Embodiment 342: The method of Embodiment 339, wherein altering the gene signature comprises an increase in expression and/or activity in the progenitor cell of two or more genes designated as an “up” gene in the gene directionality column of Table 3.
Embodiment 343: The method of Embodiment 339, wherein altering the gene signature comprises a decrease in expression and/or activity in the progenitor cell of two or more genes designated as a “down” gene in the gene directionality column of Table 3.
Embodiment 344: The method of any one of Embodiments 311 to 335, wherein the patient is selected by steps comprising: obtaining from a subject having the disease or disorder a sample of cells comprising a non-lineage committed CD34+ cell; and contacting the sample of cells with at least one perturbagen selected from Table 6, or a variant thereof; wherein the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3.
Embodiment 345: The method of Embodiment 344, wherein altering the gene signature comprises an activation of a network module designated in the network module column of Table 3.
Embodiment 346: The method of Embodiment 345, wherein the activation of the network module designated in the network module column of Table 3 comprises modulating expression and/or activity of 2 or more genes within a network module.
Embodiment 347: The method of Embodiment 346, wherein the activation of the network module designated in the network module column of Table 3 comprises modulating expression and/or activity of all of the genes within a network module.
Embodiment 348: The method of Embodiment 346, wherein the activation of the network module designated in the network module column of Table 3 comprises modulating expression and/or activity of 2 or more genes within 2 or more network modules.
Embodiment 349: The method of Embodiment 346, wherein altering the gene signature comprises an increase in expression and/or activity in the progenitor cell of two or more genes designated as an “up” gene in the gene directionality column of Table 3.
Embodiment 350: The method of Embodiment 346, wherein altering the gene signature comprises a decrease in expression and/or activity in the progenitor cell of two or more genes designated as a “down” gene in the gene directionality column of Table 3.
Embodiment 351: A method for selecting the patient of any one of Embodiments 311 to 335, comprising: obtaining from a subject having the disease or disorder a sample of cells comprising a non-lineage committed CD34+ cell; and contacting the sample of cells with least one perturbagen selected from Table 6, or a variant thereof, wherein when the at least one perturbagen alters a gene signature in the sample of cells, the subject is selected as a patient.
Embodiment 352: A method for selecting the patient of any one of Embodiments 311 to 335, comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3, the subject is selected as a patient.
Embodiment 353: The method of Embodiment 352, wherein the method alters a gene signature in the sample of cells, comprising activation of a network module designated in the network module column of Table 3.
Embodiment 354: The method of Embodiment 353, wherein the activation of the network module designated in the network module column of Table 3 comprises modulating expression and/or activity of 2 or more genes within a network module.
Embodiment 355: The method of Embodiment 354, wherein the activation of the network module designated in the network module column of Table 3 comprises modulating expression and/or activity of all of the genes within a network module.
Embodiment 356: The method of Embodiment 352, wherein the activation of the network module designated in the network module column of Table 3 comprises modulating expression and/or activity of 2 or more genes within 2 or more network modules.
Embodiment 357: The method of Embodiment 352 wherein the method causes an increase in expression and/or activity in the progenitor cell of two or more genes designated as an “up” gene in the gene directionality column of Table 3.
Embodiment 358: The method of Embodiment 352, wherein the method causes a decrease in expression and/or activity in the progenitor cell of two or more genes designated as a “down” gene in the gene directionality column of Table 3.
Embodiment 359: A method for selecting the patient of any one of Embodiments 311 to 335, comprising: obtaining from a subject having the disease or disorder a sample of cells comprising a non-lineage committed CD34+ cell; and contacting the sample of cells with at least one perturbagen selected from Table 6, or a variant thereof; wherein when the at least one perturbagen increases in the sample of cells the expression and/or activity of one or more genes selected from Table 3 designated as an “up” gene in the gene directionality column of Table 3 and/or decreases in the sample of cells the expression and/or activity of one or more genes selected from Table 3 designated as a “down” gene in the gene directionality column of Table 3, the subject is selected as a patient.
Embodiment 360: The method of Embodiment 359, wherein the method alters a gene signature in the sample of cells, comprising an activation of a network module designated in the network module column of Table 3.
Embodiment 361: The method of Embodiment 360, wherein the activation of the network module designated in the network module column of Table 3 comprises modulating expression and/or activity of 2 or more genes within a network module.
Embodiment 362: The method of Embodiment 362, wherein the activation of the network module designated in the network module column of Table 3 comprises modulating expression and/or activity of all of the genes within a network module.
Embodiment 363: The method of Embodiment 359, wherein the activation of the network module designated in the network module column of Table 3 comprises modulating expression and/or activity of 2 or more genes within 2 or more network modules.
Embodiment 364: The method of Embodiment 359, wherein the method causes an increase in expression and/or activity in the progenitor cell of two or more genes designated as an “up” gene in the gene directionality column of Table 3.
Embodiment 365: The method of Embodiment 359, wherein the method causes a decrease in expression and/or activity in the progenitor cell of two or more genes designated as a “down” gene in the gene directionality column of Table 3.
Embodiment 366: Use of the perturbagen of Table 6, or a variant thereof in the manufacture of a medicament for treating a disease or disorder characterized by an abnormal ratio of megakaryocytes, proplatelets, and/or platelets to progenitor cells.
Embodiment 367: Use of the perturbagen of Table 6, or a variant thereof in the manufacture of a medicament for treating a disease or disorder characterized by an abnormal ratio of megakaryocytes, proplatelets, and/or platelets to other committed blood cells, optionally erythrocytes.
Embodiment 368: A method of identifying a candidate perturbation for promoting the transition of a starting population of progenitor cells into megakaryocytes, proplatelets, and/or platelets or immediate progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into megakaryocytes, proplatelets, and/or platelets or immediate progenitors thereof following exposure of the population of cells to the perturbation; and identifying the perturbation as a candidate perturbation for promoting the transition of a population of progenitor cells into megakaryocytes, proplatelets, and/or platelets or immediate progenitors thereof based on the perturbation signature, wherein the perturbation signature is an increase in expression and/or activity in the progenitor cell of one or more genes selected from Table 3 designated as an “up” gene in the gene directionality column of Table 3, and/or a decrease in expression and/or activity in the progenitor cell of one or more genes selected from Table 3 designated as a “down” gene in the gene directionality column of Table 3.
Embodiment 369: The method of Embodiment 368, wherein the perturbation signature comprises an activation of a network module designated in the network module column of Table 3.
Embodiment 370: The method of Embodiment 369, wherein the activation of the network module designated in the network module column of Table 3 comprises modulating expression and/or activity of 2 or more genes within a network module.
Embodiment 371: The method of Embodiment 370, wherein the activation of the network module designated in the network module column of Table 3 comprises modulating expression and/or activity of all of the genes within a network module.
Embodiment 372: The method of Embodiment 369, wherein the activation of the network module designated in the network module column of Table 3 comprises modulating expression and/or activity of 2 or more genes within 2 or more network modules.
Embodiment 373: The method of Embodiment 369, wherein the perturbation signature comprises an increase in expression and/or activity in the progenitor cell of two or more genes designated as an “up” gene in the gene directionality column of Table 3.
Embodiment 374: The method of Embodiment 369, wherein the perturbation signature comprises a decrease in expression and/or activity in the progenitor cell of two or more genes designated as a “down” gene in the gene directionality column of Table 3.
Embodiment 375: A method for making a therapeutic agent for a disease or disorder selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, comprising: (a) identifying a candidate perturbation according to the method of Embodiment 367, and (b) formulating the candidate perturbation as a therapeutic agent for the treatment of the disease or disorder.

In aspects, the disclosure also provides a method for directing a change in cell state of a plurality of progenitor cells comprising: contacting a population of cells comprising a plurality of progenitor cells with at least one perturbagen selected from Table 4, Table 5, and/or Table 6, or a variant thereof, including combinations of the foregoing, wherein the at least one perturbagen is capable of altering one or more gene signatures in the plurality of progenitor cells; and wherein the plurality of progenitor cells are non-lineage committed CD34+ cells. In embodiments, the method provides the combinatorial use of perturbagens and/or gene signatures to simultaneously drive and/or direct different cell states.

In a non-limiting example, the method includes contacting a plurality of progenitor cells with at least one perturbagen (e.g. at least about one, about 2, about 3, about 4, about 5, about 6, about 7, about 8, about 9, about 10, or about 10 or more perturbagens) selected from Table 4, Table 5, and/or Table 6, or a variant thereof, including combinations of the foregoing (e.g. a combination of perturbagens) wherein the at least one perturbagen (e.g. combination of perturbagens) provides alteration of one or more gene signatures, for example one or more gene signatures (e.g. about 2 or more, about 3 or more, about 4 or more, about 5 or more, about 6 or more, about 7 or more, about 8 or more, about 9 or more, or about 10 or more gene signatures) selected from Table 1, Table 2, and/or Table 3, including combinations of the foregoing, and any combination thereof. In embodiments, the alteration of one or more gene signatures provides an increase in the number of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets, and any combination thereof, of the plurality of progenitor cells, optionally wherein one or more of the proerythroblasts, early erythroblasts, intermediate erythroblasts, late erythroblasts, reticulocytes, and erythrocytes expresses fetal hemoglobin (HbF). As would be understood by one of ordinary skill in the art, any of the perturbagens and/or methods and/or compositions described herein can be used for directing a change in cell state of a plurality of progenitor cells.

The instant disclosure also provides certain embodiments as follows:

Embodiment 376: A method for directing a change in cell state of a plurality of progenitor cells comprising: contacting a population of cells comprising a plurality of progenitor cells with at least one perturbagen selected from Table 4, Table 5, and/or Table 6, or a variant thereof, including combinations of the foregoing, wherein the at least one perturbagen is capable of altering one or more gene signatures in the plurality of progenitor cells; and wherein the plurality of progenitor cells are non-lineage committed CD34+ cells.
Embodiment 377: The method of Embodiment 376, wherein the change in cell state provides an increase in the number of one or more of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets, and any combination thereof, optionally wherein one or more of the proerythroblasts, early erythroblasts, intermediate erythroblasts, late erythroblasts, reticulocytes, and erythrocytes expresses fetal hemoglobin (HbF).
Embodiment 378: The method of Embodiment 377, wherein the number of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets is increased after contacting the population of cells comprising a CD34+ cell with the at least one perturbagen.
Embodiment 379: The method of any one of Embodiments 376-378, wherein the at least one perturbagen selected from Table 4, Table 5, and/or Table 6, or a variant thereof, including combinations of the foregoing, comprises at least 2, at least 3, at least 4, or at least 5 perturbagens selected from Table 4, Table 5, and/or Table 6, or variants thereof, including combinations of the foregoing.
Embodiment 380: The method of any one of Embodiments 376-379, wherein the one or more gene signatures are selected from:
a) one or more genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, genes selected from the genes designated as an “up” gene in the gene directionality column of Table 1;
b) one or more genes selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1;
c) one or more genes selected from the genes designated as an “up” gene in the gene directionality column of Table 2, comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2:
d) one or more genes selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2;
e) one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or 23 or more genes designated as an “up” gene in the gene directionality column of Table 3; and
f) one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3.
   Embodiment 381: The method of any one of Embodiments 376-380, wherein the one or more genes comprise one or more of a)-f):
a) the one or more genes selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1;
b) the one or more genes selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2;
c) the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39;
d) the one or more genes selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNA/2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1;
e) the one or more genes selected from Table 3 comprises at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK; and/or
f) the one or more genes selected from Table 3 comprises at least one of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.

### Methods of Culturing Cells In Vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue or from a blood product. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq measure protein expression in the cell. CITE-seq simultaneously measures both gene expression and protein expression in the cell. And scATAC-seq measures chromatin conformation in the cell. Table 7 below provides links to example protocols for performing each of the single-cell cellular-component expression measurement techniques described herein.

TABLE 7
Example Measurement Protocols

| Technique | Protocol |
|---|---|
| RNA-seq | Olsen and Baryawno “Introduction to Single-Cell RNA Sequencing” Current |
| | Protocols in Molecular Biology. Volume122, Issue1, April 2018, e57 |
| Tag-seq | Rozenberg et al., “Digital gene expression analysis with sample multiplexing and |
| | PCR duplicate detection: A straightforward protocol”, BioTechniques, vol. 61, |
| | No. 1, March 2018 |
| ATAC-seq | Buenrostro et al., “ATAC-seq: A Method for Assaying Chromatin Accessibility |
| | Genome-Wide”, Curr Protoc Mol Biol. 2015; 109: 21.29.1-21.29.9 |
| miRNA-seq | Faridani et al., “Single-cell sequencing of the small-RNA transcriptome” Nature |
| | Biotechnology volume 34, pages1264-1266 (2016) |
| CyTOF/SCOPE-MS/Abseq | Bandura et al., “Mass Cytometry: Technique for Real Time Single Cell |
| | Multitarget Immunoassay Based on Inductively Coupled Plasma Time-of-Flight |
| | Mass Spectrometry”, Anal Chem. 2009 Aug 15; 81(16): 6813-22 |
| | Shahi et al., “Abseq: Ultrahigh-throughput single cell protein profiling with droplet |
| | microfluidic barcoding”, Scientific Reports volume 7, Article number: 44447 |
| | (2017) |
| | Budnik et al., “SCOPE-MS: mass spectrometry of single mammalian cells |
| | quantifies proteome heterogeneity during cell differentiation”, Genome Biology |
| | 2018 19:161 |
| CITE-seq | Stoeckius et al., “Simultaneous epitope and transcriptome measurement in |
| | single cells”, Nature Methods, vol 14, pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same “progenitor” cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “progenitor” cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same “progenitor” cell. The term pseudo-time is used in two respects, first, in that cell state transition is not necessarily the same from cell to cell, and thus the population of cell provides a distribution of what transition processes a cell of that “progenitor” type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a “progenitor” cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, clustered regularly interspaced short palindromic repeats (CRISPR) may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:
Day 0: Thaw cells in the first cell state into a plate in a media suitable for growth of the cells.
Day 1: Seed cells in the first cell state into a multi-well plate. If applicable, perform additional steps to affect gene expression by cells. For example, simultaneously infect with one or more viruses to activate or knock out genes of interest.
   Perform gene expression measurement iteration t₁ for cells in the wells.
Day 1+I: Change media as needed if any additional processes are performed.
   If applicable, perform gene expression measurement iteration t, for cells in the wells.
Day 1+m: Change media to media appropriate to support growth of cells in the second cell state.
   If applicable, perform gene expression measurement iteration tₘ for cells in the wells.
Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.
   If applicable, perform gene expression measurement iterations tₙ, tₒ, tₚ, etc., for cells in the wells.
Day q: Perform gene expression measurement iteration t_{q} for cells in the wells and in the second state.
Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be 5 within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

As used herein, the terms “cell fate” and “cell state” are interchangeable and synonymous.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “ex vivo” refers to a medical procedure in which an organ, cells, or tissue are taken from a living body for a treatment or procedure, and then returned to the living body (See NCI Dictionary of Cancer Terms, https://www.cancer.gov/publications/dictionaries/cancer-terms/def/ex-vivo).

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “perturbation” in reference to a cell (e.g., a perturbation of a cell or a cellular perturbation) refers to any treatment of the cell with one or more active agents capable of causing a change in the cell's lineage or cell state (or in the lineage or cell state of the cell's progeny). These active agents can be referred to as “perturbagens.” In some embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system, Cas9-gRNA complex, or other gene editing system), or any combination of any of the foregoing. As used herein, a perturbagen classified as a“compound” may be a small molecule or a biologic. Also, a perturbagen classified as “overexpression of gene” may be cDNA over-expressing a wild-type gene or an mRNA encoding a wild-type gene.

In some embodiments, an mRNA may comprise a modified nucleotide that promotes stability of the mRNA and/or reduces toxicity to a subject. Examples of modified nucleotides useful in the present disclosure include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about: 1%, 2%, 3%, 4%, 5%, 6%, 7%, 8%, 9%, 10%, 11%, 12%, 13%, 14%, 15%, or more, variation, i.e., having about: 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

The term “progenitor” in reference to a cell (e.g., a progenitor cell) refers to any cell that is capable of transitioning from one cell state to at least one other cell state. Thus, a progenitor can differentiate into one or more cell types and/or can expand into one or more types of cell populations.

As used herein, the terms “cell fate” and “cell state” are interchangeable and synonymous.

As used herein, the term “red blood cells” refers to both reticulocytes and erythrocytes.

As used herein, the terms “treat,” “treatment,” and/or “treating” may refer to the management of a disease, disorder, or pathological condition, or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the term “preventing” (also prophylaxis) refers to action taken to decrease the chance of getting a disease or condition.

The term “subject,” refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human (e.g., a human patient). In some embodiments, the subject is a rodent, a mouse, a rat, a hamster, a rabbit, a dog, a cat, a cow, a goat, a sheep, or a pig.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The disclosure will be further described in the following examples, which do not limit the scope of the disclosure described in the claims.

### EXAMPLES

### Example 1: Single Cell Gene Expression Profiling of In Vitro Hematopoietic Differentiation

To develop a model of early hematopoietic differentiation, mobilized peripheral blood (mPB) CD34+ cells were thawed, cultured and subjected to single cell gene expression profiling (GEP) at four (4) timepoints throughout the differentiation process. Briefly, three (3) cryopreserved vials of mPB CD34+ donors were thawed. Approximately 1×10⁵ cells from each donor were aliquoted for single cell and bulk RNA sequencing, as described below. Donors 1 and 2 and Donors 2 and 3 were pooled and cultured for five days in expansion media comprising StemSpan Serum Free media (Stem Cell Technologies Cat #09650) and CC100 cocktail (Stem Cell Technologies Cat #02690), which contains SCF, Flt3L, IL-3, and IL-6, which promotes expansion and non-directed lineage differentiation. Following five days in expansion media, cells were transferred to differentiation media, containing EPO, to drive erythroid differentiation. Two parallel differentiation runs were performed: donor 1+2 and donor 2+3, which allowed, following donor deconvolution post-sequencing, detection of differentiation run or donor specific differences throughout the differentiation process. Cells were collected for counts, single cell and bulk RNA sequencing at four time points: E0 (freshly thawed), E3 (Expansion phase day 3), E5 and D1 (Differentiation phase day 1).

Bulk mRNA sequencing was performed separately on three donors at E0 (time of thaw). This was used to deconvolute the single cell samples using donor specific Single-nucleotide polymorphisms (SNPs). Deconvolution of donor contribution using donor specific SNPs was performed as part of the downstream analysis.

For the subsequent time points, the two pooled donor differentiation runs were sequenced independently for bulk RNA sequencing (two sequencing runs: 1+2, 2+3) and both differentiation runs were pooled for single cell mRNA sequencing (one pool: 1+2+2+3).

RNA isolation was performed for bulk total RNA sequencing using the miRNeasy Micro Kit (50) QIAGEN 217084 kit. RNA was quantified using the Qbit and Bioanalyzer.

Bulk total RNAseq NGS libraries were prepared using the KAPA Stranded RNA-Seq with RiboErase kit (Kapa BioSystems).

Paired end (2×125) total bulk RNA sequencing (mRNA, isoforms, SNPs, non-coding RNA) was performed on the Illumina HiSeq 2500 using the High Output v4 single lane flow cells.

For single cell sequencing at each time point, all donors were pooled into a single tube prior to running through the 10× Genomics Single Cell 3′ v2 workflow steps. Cells were washed, counted, and run within one hour using 10× partitioning and cDNA amplification (10× Genomics, Single Cell 3′ v2). The rest of the library prep was completed as per the 10× Genomics Single Cell 3′ v2 library prep protocol. 10× samples were sequenced using three NextSeq High 1×75 flow cell runs.

Count matrices were obtained by running Cell Ranger (10× Genomics) with default settings. Low quality cells with less than 200 genes expressed were filtered, genes expressed in less than three cells were filtered, and data was CPM normalized and log-transformed. Highly variable genes were detected, and the count matrices were corrected for mitochondrial gene expression. A dimensionality-reduced representation of the count matrix was embedded using UMAP, clustered using Louvain clustering and annotated using differential expression testing (Mann Whitney U test) and comparisons to established marker genes. Proxies for cellular states are the annotated clusters, as shown in FIG. 1A. Differential signatures for the 11 to 0 transitions from a committed erythroid progenitor cell state to cells of an erythroid cell state expressing HBG1 (fetal hemoglobin), were then used to predict perturbations that would promote fetal hemoglobin induction.

### Example 2: Testing Compounds In Vitro Culture

Briefly, mobilized peripheral blood (mPB), or bone marrow (BM) derived CD34+ hematopoietic stem and progenitor cells (HSPCs) were thawed and differentiated towards the erythroid lineage using a 3-stage in vitro erythroid differentiation protocol (TABLE 8). Specifically, cells were cultured in erythroid expansion media (phase 1) from Day 0-6, followed by differentiation media (phase 2) between Day 7-10, and culminating with incubation in erythroid maturation media (phase 3) from Day 10-18. Small molecules (perturbagens) reconstituted in DMSO (or appropriate solvent) were added starting at day 7 (corresponding to a stage encompassing early erythroid progenitors marked by CD71+CD235a+. Day 7 erythroid progenitors were incubated with perturbagens for the remaining time course of the erythroid differentiation (Day 7-18) with addition of fresh compound at each cell passaging. As part of our analysis, the expansion (fold growth), viability, and erythroid maturation was measured throughout the course of differentiation (Day 7-18). Erythroid maturation was determined by flow cytometry using a four-antibody panel (TABLE 9) (CD71, CD235a, CD233, CD49d) tracking increased CD233 expression, with a concomitant loss of CD49d expression, and a shift in CD71^{Hi} to CD71^{low} erythroid population (CD235a+) over 18 days. The testing results are summarized in FIG. 1C.

FIG. 1C demonstrates that we can efficiently generate mature erythrocytes from human CD34+ progenitors using described erythroid differentiatin protocol in FIG. 1B.

To measure the efficacy of the perturbagens at promoting a fetal hemoglobin (HbF) cell state, a flow cytometry assay was used measure the percentage of HbF expressing cells (F-cells) compared to vehicle control and hydroxyurea (50 uM) an FDA approved compound shown to increase HbF in humans. In brief, day 18 in vitro derived erythrocytes were fixed and stained with anti-HbF (gamma globin subunit) and anti-carbonic anhydrase 1 (CA1), an enzyme expressed predominantly in adult red blood cells. Furthermore, to accurately gate F-cells using flow cytometry, cord blood derived erythrocytes and Fetaltrol (Thermo Fisher) were used as positive controls. In addition to flow cytometry, ion-exchange chromatography will be used to measure the percentage HbF relative to all other hemoglobin (HbF/HbA+HbF) in samples tested.

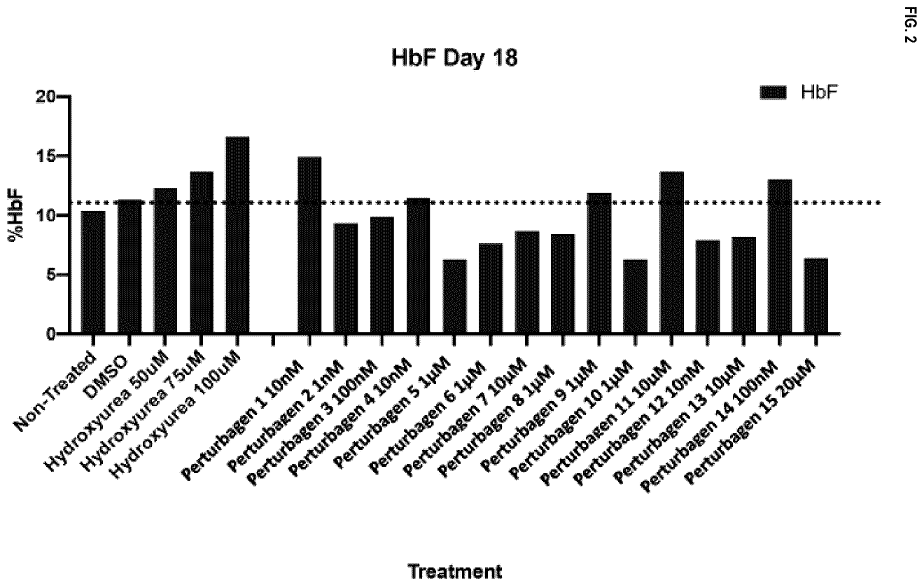

A panel of 15 compounds as listed in Table 3 are profiled HbF induction activities used the assay protocol described above. The testing results are summarized in FIG. 2.

TABLE 8
Erythroid Differentiation Media Composition

| Phase 1 | Phase2 | Phase 3 | | |
|---|---|---|---|---|
| (Expansion) | (Differentiation) | (maturation) | Vendor | Cat# |
| StemSpan SFEM | StemSpan SFEM | StemSpan SFEM | StemCellTech | 09650 |
| GlutaMax (100x) | GlutaMax (100x) | GlutaMax (100x) | ThermoFisher | 35050061 |
| Holo-transferrin | Holo-transferrin | Holo-transferrin | Sigma | T0665 |
| (500 ug/ml) | (500 ug/ml) | (500 ug/ml) | | |
| Lipid Mixture (5 ul/ml) | Lipid Mixture (5 ul/ml) | Lipid Mixture (5 ul/ml) | Sigma | L0288 |
| Insulin (10 ng/ml) | Insulin (10 ng/ml) | Insulin (10 ng/ml) | Sigma | 19278 |
| Epo (2.5 U/ml) | Epo (2.5 U/ml) | Epo (2.5 U/ml) | R&D | 287-TC |
| SCF (50 ng/ml) | SCF (50 ng/ml) | | R&D | 255-SC050 |
| IL3 (10 ng/ml) | | | R&D | 203-IL-010 |

TABLE 9
Antibodies for Erythroid Maturation and Fetal Hemoglobin Detection

| Antibody | Fluorophore | Vendor | Clone | Cat # |
|---|---|---|---|---|
| CD71 | PE | eBiosciences | OKT9 | 12-0719-42 |
| CD235a | PE-Cy7 | BD | HIR2 | 563666 |
| CD49d | BV421 | BD | 9F10 | 565277 |
| CD233 (Band3) | FITC | IBGRL | BRIC6 | 9439 |
| HbF | APC | ThermoFisher | HBF-1 | MHFH05 |
| HbF | PE | IQProducts | N/A | IQP363 |
| CA | FITC | IQProducts | N/A | IQP363 |

### Example 3: Testing Functional Impact of Compounds In Vitro (Disease Tissue)

Hereditary persistence of fetal hemoglobin (HPFH) is the name given to a cohort of genetic mutations resulting in reactivation of HbF in adult red blood cells. Genetic studies of beta-thalassemia and sickle cell disease patients have demonstrated that reactivation of HbF can reduced disease burden in both hemoglobinopathies in a concentration dependent manner. To further evaluate the efficacy of perturbagens in a disease state, peripheral blood (PB), BM, or mPB from sickle cell patients is used as a source of CD34⁺ HSPCs for in vitro erythroid differentiation. In brief, in vitro derived erythrocytes from sickle patients are treated with validated perturbagens as described in Example 2, followed by readouts of HbF induction by both flow cytometry and HPLC. These two readouts address pancellularity of HbF induction (% F-Cells), and the ratio anti-sickling hemoglobin (HbF) to sickling hemoglobin (HbS). To further evaluate the impact of the perturbagens in reducing disease burden, in vitro sickling assays are performed on sickle derived erythrocytes cells by enrichment of enucleated erythrocytes followed by incubation of cells at low oxygen or incubation in 2% sodium metabisulfite. Cell sickling is monitored using time lapse imaging.

### Example 4: Single Cell Gene Expression Profiling of In Vitro Hematopoietic Differentiation

To develop a model of early hematopoietic differentiation, mobilized peripheral blood (mPB) CD34+ cells were thawed, cultured and subjected to single cell gene expression profiling (GEP) at four (4) timepoints throughout the differentiation process. Briefly, three (3) cryopreserved vials of mPB CD34+ donors were thawed. Approximately 1×10⁵ cells from each donor were aliquoted for single cell and bulk RNA sequencing, as described below. Donors 1 and 2 and Donors 2 and 3 were pooled and cultured for five days in expansion media comprising StemSpan Serum Free media (Stem Cell Technologies Cat #09650) and CC100 cocktail (Stem Cell Technologies Cat #02690), which contains SCF, Flt3L, IL-3, and IL-6, which promotes expansion and non-directed lineage differentiation. Following five days in expansion media, cells were transferred to differentiation media, containing EPO, to drive erythroid differentiation. Two parallel differentiation runs were performed: donor 1+2 and donor 2+3, which allowed, following donor deconvolution post-sequencing, detection of differentiation run or donor specific differences throughout the differentiation process. Cells were collected for counts, single cell and bulk RNA sequencing at four time points: E0 (freshly thawed), E3 (Expansion phase day 3), E5 and D1 (Differentiation phase day 1).

Bulk mRNA sequencing was performed separately on three donors at E0 (time of thaw). This was used to deconvolute the single cell samples using donor specific Single-nucleotide polymorphisms (SNPs). Deconvolution of donor contribution using donor specific SNPs was performed as part of the downstream analysis.

For the subsequent time points, the two pooled donor differentiation runs were sequenced independently for bulk RNA sequencing (two sequencing runs: 1+2, 2+3) and both differentiation runs were pooled for single cell mRNA sequencing (one pool: 1+2+2+3).

RNA isolation was performed for bulk total RNA sequencing using the miRNeasy Micro Kit (50) QIAGEN 217084 kit. RNA was quantified using the Qbit and Bioanalyzer.

Bulk total RNAseq NGS libraries were prepared using the KAPA Stranded RNA-Seq with RiboErase kit (Kapa BioSystems).

Paired end (2×125) total bulk RNA sequencing (mRNA, isoforms, SNPs, non-coding RNA) was performed on the Illumina HiSeq 2500 using the High Output v4 single lane flow cells.

For single cell sequencing at each time point, all donors were pooled into a single tube prior to running through the 10× Genomics Single Cell 3′ v2 workflow steps. Cells were washed, counted, and run within one hour using 10× partitioning and cDNA amplification (10× Genomics, Single Cell 3′ v2). The rest of the library prep was completed as per the 10× Genomics Single Cell 3′ v2 library prep protocol. 10× samples were sequenced using three NextSeq High 1×75 flow cell runs.

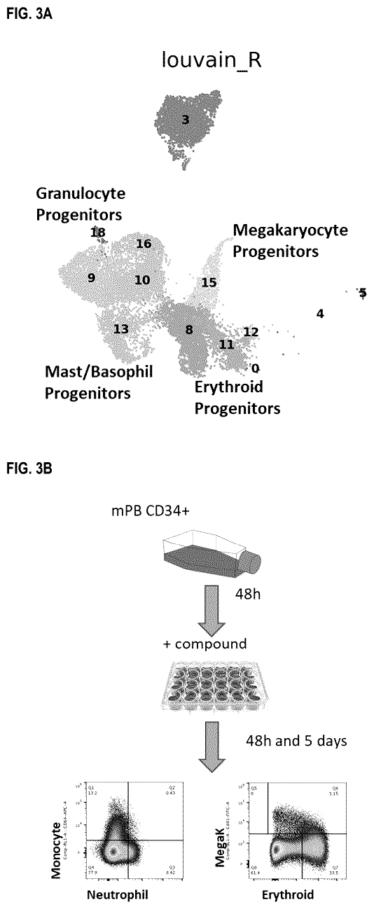

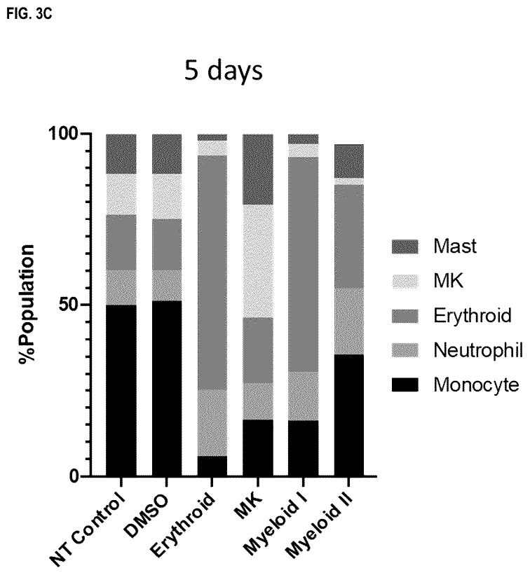

Count matrices were obtained by running Cell Ranger (10× Genomics) with default settings. Low quality cells with less than 200 genes expressed were filtered, genes expressed in less than three cells were filtered, and data was CPM normalized and log-transformed. Highly variable genes were detected, and the count matrices were corrected for mitochondrial gene expression. A dimensionality-reduced representation of the count matrix was embedded using UMAP, clustered using Louvain clustering and annotated using differential expression testing (Mann Whitney U test) and comparisons to established marker genes. These analyses led to generation of predictions that drive the transition of cells from CD34+ erythroid stem cells to the indicated cell types, including the cells of erythroid lineage, as shown in FIG. 3A. Differential signatures for the 8 to 11 transitions, i.e., from a non-lineage committed CD34+ progenitor cell to cells of the erythroid lineage, were then used to predict perturbations that would promote the transition.

### Example 5: Testing Compounds In Vitro Culture

Briefly, mobilized peripheral blood (mPB), cord blood (CB) or bone marrow (BM) derived CD34⁺ hematopoietic stem and progenitor cells were thawed and allowed to recover in StemSpan SFEM (Stem Cell Technologies) supplemented with TPO and CC100 cytokine supplement (Stem Cell Technologies) that contains SCF, Flt3L, IL-6 and IL-3 (Expansion media) for 48 hrs prior to delivery of the perturbagens. Perturbagens reconstituted in DMSO were added to media at different concentrations. Cells were cultured for 2 and 5 days in the presence of small molecules prior to collection for cell counts, viability and flow-cytometric analysis to assess the impact of the small molecules on the culture composition. Perturbagens are listed in Table 5.

Expansion of committed erythroid progenitors was measured by positive expression of the surface marker CD235a+(GYPA) with co-expression of CD71+(Transferrin receptor) within the CD34⁺CD38^{+/-} gate (encompassing hematopoietic stem cells progenitors) relative to vehicle control conditions (DMSO) at 2 days post-treatment. At 5 days post-treatment, cells were gated for expression of CD235a⁺ and CD71⁺ with the CD34-CD38^{+/-} gate (encompassing lineage positive population). Conditions promoting erythroid differentiation (StemSpan+SCF, EPO, IL3) were included to demonstrate that lineage specific changes could be measured following 2 and 5 days of treatment.

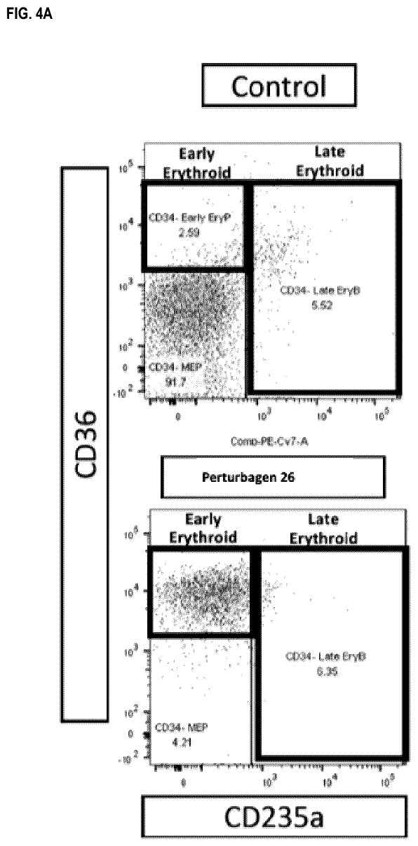

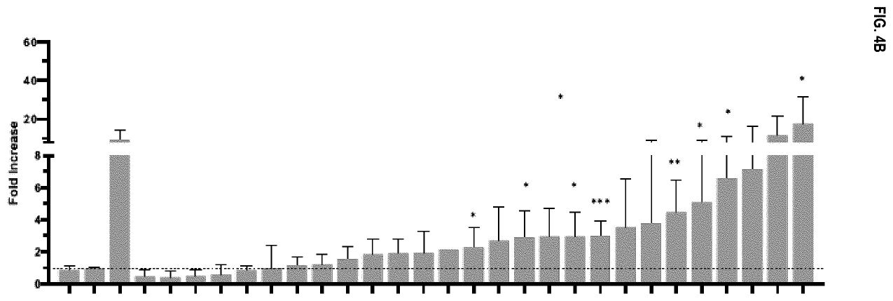

To further characterize erythrocyte lineage commitment and differentiation, a modified flow cytometry panel was used to identify megakaryocyte/erythroid progenitors/MEPs (CD71⁺, CD41a⁻, CD36⁻, CD235a⁻), early erythroid progenitors (CD71^{high}CD441a⁻, CD36⁺, CD235a⁻), late erythroid progenitors (CD71^{high}, CD41a⁻, CD36⁺, CD235a⁺), and megakaryocyte progenitors (CD71⁻CD41a⁺, CD42b^{+/-}, CD235a⁻) allowing to identify define states where are perturbagens are acting on erythrocyte lineage commitment and differentiation. See FIG. 4A and FIG. 4B.

In this and the above examples, antibodies directed against hematopoietic surface proteins and conjugated fluorophores are listed below in Table 10.

TABLE 10
Antibodies Against Hematopoietic Surface Proteins and
Conjugated Fluorophore

| Antibody | Fluorophore | Vendor | Clone | Cat # |
|---|---|---|---|---|
| CD41a | FITC | eBiosciences | HIP8 | 11-0419-42 |
| CD66b | Percp5.5 | Biolegend | G10F5 | 305108 |
| CD71 | PE | eBiosciences | OKT9 | 12-0719-42 |
| FcεRla+ | PE-Cy7 | eBiosciences | AER-37 | 25-5899-42 |
| CD64 | APC | Biolegend | 10.1 | 305014 |
| CD38 | AF700 | eBiosciences | HIT2 | 56-0389-42 |
| CD34 | APC-eFlur780 | eBiosciences | 4H11 | 47-0349-42 |
| CD123 | BV421 | Biolegend | 6H6 | 306018 |
| CD135 | BV421 | BD | 4G8 | 564708 |
| CD45RA | BV785 | BD | Hi100 | 563870 |
| CD235a | PE-Cy7 | BD | HIR2 | 563666 |
| CD42b | APC | eBiosciences | HIP1 | 17-0429-41 |
| CD36 | BV605 | BD | CB38 | 563518 |

In this and the above examples, flow cytometry panel comparisons with either CD135 or CD123 to identify myeloid progenitor populations and/or to identify cell states are listed below in Table 11.

TABLE 11
Flow Cytometry Panel Comparison with CD135 or CD123 to Identify Myeloid Progenitor Populations

| | Immunophenotype | Immunophenotyep |
|---|---|---|
| Cell Type | (Lineage−) CD135 | (Lin−) CD123 |
| Megakaryocyte- | CD34⁺CD38⁺CD45RA⁻CD135⁻CD71⁺ | CD34⁺CD38⁺CD45RA⁻CD123⁻CD71⁺ |
| erythroid | | |
| progenitor MEP′ | | |
| (Meg/Eryth Pro; | | |
| MkP) | | |
| Erythroid | CD34⁺CD38⁺CD45RA⁻CD135⁻CD71⁺ | CD34⁺CD38⁺CD45RA⁻CD123⁻CD71⁺ |
| Progenitor | CD41⁻CD235a⁺ | CD41⁻CD235⁺ |
| Early Erythroid | CD34+CD38+CD45RA−CD135− | CD34+CD38+CD45RA−CD123− |
| Progenitor | CD71+CD41−CD36+CD235a− | CD71+CD41−CD36+CD235a− |
| Late Erythroid | CD34+CD38+CD45RA−CD135− | CD34+CD38+CD45RA−CD123− |
| Progenitor | CD71+CD41−CD36+CD235a+ | CD71+CD41−CD36+CD235a+ |
| MkP Progenitor | CD34⁺CD38⁺CD45RA⁻CD135⁻CD71⁺ | CD34⁺CD38⁺CD45RA⁻CD123⁻CD71⁺ |
| | CD41⁺CD235a⁻ | CD41⁺CD235a⁻ |
| Common myeloid | CD34⁺CD38⁺CD45RA⁻CD135⁺ | CD34⁺CD38⁺CD45RA⁻CD123⁺ |
| progenitor (CMP; | | |
| immature | | |
| populations - | | |
| myeloid and | | |
| early stage | | |
| Neutrophil) | | |
| Eosinophil/ | CD34⁺CD38⁺CD45RA⁻CD135mid | CD34⁺CD38⁺CD45RA⁻CD123⁺ |
| basophil/mast | | |
| cell progenitor | | |
| Mast Cell/ | CD34⁺CD38⁺CD45RA⁻ | CD34⁺CD38⁺CD45RA⁻CD123⁺FcεRla⁺ |
| Basophil | CD135midFcεRla⁺ | |
| GMP | CD34⁺CD38⁺CD45RA⁺CD135⁺ | CD34⁺CD38⁺CD45RA⁺CD123⁺ |
| (Neutrophil | | |
| progenitor; | | |
| Monocyte/ | | |
| DC progenitor) | | |
| Neutrophil | CD34⁺CD38⁺CD45RA⁺CD135⁺CD66b⁺ | CD34⁺CD38⁺CD45RA⁺CD123⁺CD66b⁺CD64⁻ |
| Progenitor | | |
| Monocyte | CD34⁺CD38⁺CD45RA⁺CD135⁺CD64⁺ | CD34⁺CD38⁺CD45RA⁺CD123⁺CD66b⁻CD64⁺ |
| Progenitor | | |

NOTE:
Lineage: CD4⁻CD8⁻CD11b⁻CD14⁻CD19⁻CD20⁻CD56⁻CD10⁻ (this stain is optional if working with purified CD34⁺ cells)

### Example 6: Testing Functional Impact of Compounds in In Vitro Colony Forming Culture Assay

Methocult colony forming unit (CFU) assay is a semi-solid methylcellulose culture system containing defined human cytokines (rh SCF, rh GM-CSF, rh IL-3, rh G-CSF, rh EPO), which allows for the detection of hematopoietic progenitors, including the multipotent granulocyte/erythroid/monocyte/megakaryocyte progenitors (CFU-GEMM), erythroid progenitors (BFU-E and CFU-E) and granulocyte/monocyte progenitors (CFU-GM, CFU-G, CFU-M) in BM, CB, PB, mPB following 14-16 days of culture. The CFU assay provides an in vitro functional readout that complements the phenotypic readout described in Example 4. Briefly, mPB, cord blood (CB) or bone marrow (BM) derived CD34+ hematopoietic stem and progenitor cells will be thawed and allowed to recover in StemSpan SFEM (Stem Cell Technologies) supplemented with TPO and CC100 cytokine supplement (Stem Cell Technologies) that contains SCF, Flt3L, IL-6 and IL-3 (Expansion media) for 48 h prior to exposure to perturbagens for 2 days followed by resuspension of treated cells in MethoCult™ H4034 Optimum (Stem Cell Technologies) and culture for 14 days. At the end of culture period, changes in number and/or size of colony will be quantified using a STEMvision (Stem Cell Technologies) or similar imaging unit. Specifically, an increase in the total number of BFU-E and/or CFU-E or in the ratio of BFU-E/CFU-E to other colony types will be indicative of lineage commitment providing a functional assay to validate the ability of the predicted perturbagen to direct hematopoietic differentiation towards the erythroid lineage.

### Example 7: Testing Compounds In Vivo

Following in vitro validation of compounds that promoted erythroid progenitors, molecules will be tested in C57BL/6 mice to evaluate the compounds ability to promote erythroid expansion and an increase in circulating red blood cells (RBCs) number in vivo during steady state hematopoiesis or following sub-lethal myeloablation. Sublethal myeloablation was induced by sublethal irradiation or busulfan treatment. Busulfan is DNA alkylating reagent used in the clinic for bone marrow conditioning with reduced toxicity. Similarly, busulfan dosing has been previously established in murine models as a less toxic conditioning method and an alternative to whole body irradiation.

Briefly, in the setting of normal steady state hematopoiesis or in the setting of sub-lethal myeloablation, a single dose or repeated doses (daily dose) of compounds will be delivered via intraperitoneal, subcutaneous, or intravenous injection into 8-15 week old female C57/Bl6 mice. For repeat dosing, compounds will be injected once every 24 hrs or every other day for up to 3 weeks. As part of the experimental readout, weight and health of each mouse was monitored for overt signs of compound toxicity. As a primary readout of efficacy, peripheral blood will be collected every 2 days post-injection for 2-4 weeks. Blood will be analyzed using a complete blood count (CBC) analyzer (Heska) and by flow cytometry using a multiparameter in-house assay (Table 12). Using these two methods, the frequency of RBCs within the peripheral blood and other major changes in other blood populations (T-cells, B-cells, monocyte, neutrophils, and platelets) can be accurately monitored during dosing with test compounds. At the study termination, mice will be sacrificed and analysis of the bone marrow, in addition to peripheral blood, will be performed using flow cytometry to measure changes in erythroid progenitor and all other major blood lineages present the bone marrow. Furthermore, bone marrow analysis can be expanded to measure stem cells and progenitor populations relevant to erythroid development.

TABLE 12
Antibodies Against Murine Hematopoietic Surface Proteins and
Conjugated Fluorophore

| Antibody | Fluorophore | Vendor | Clone | Catalog# |
|---|---|---|---|---|
| CD3 | BV510 | Biolegend | 17A2 | 100233 |
| CD19 | BV605 | Biolegend | 6D5 | 115539 |
| Ter119 | APC-Cy7 | Biolegend | Ter119 | 116223 |
| CD71 | PE | Biolegend | RI7217 | 113808 |
| CD11b | BB515 | BD | M1/70 | 564455 |
| Ly6G | BV786 | BD | 1A8 | 740953 |
| Ly6C | AF 700 | Biolegend | A7R34 | 128023 |
| CD41 | BV421 | Biolegend | MWewg30 | 133911 |
| CD45 | PerCPCy5.5 | Biolegend | 30-F11 | 103131 |
| SytoxRed | Alexa 647 | Thermo Fisher | — | S34859 |

TABLE 13
Immunophenotype for Murine Blood Lineages

| Cell Type | Immunophenotype |
|---|---|
| Erythroid Cells | CD45⁻Ter119⁺ |
| Erythroid Progenitors (bone marrow) | CD71⁺Ter119⁺ |
| T-cells | CD45⁺CD11b⁻CD3⁺ |
| B-cells | CD45⁺CD11b⁻CD19⁺ |
| Neutrophils | CD45⁺CD11b⁺ly6-C_{low}Ly6-G⁺ |
| Monocytes | CD45⁺CD11b⁺Ly6-G⁻ |
| Megakaryocytes (bone marrow) | CD41⁺FSC_{med-high} |
| Platelets (periphery) | CD41⁺FSC_{low} |

### Example 8: Single Cell Gene Expression Profiling of In Vitro Hematopoietic Differentiation

To develop a model of early hematopoietic differentiation, mobilized peripheral blood (mPB) CD34+ cells were thawed, cultured and subjected to single cell gene expression profiling (GEP) at four (4) time points throughout the differentiation process. Briefly, cryopreserved vials of three (3) mPB CD34+ donors were thawed. Approximately 1×10⁵ cells from each donor were aliquoted for single cell and bulk RNA sequencing, as described below. Donors 1 and 2 and Donors 2 and 3 were pooled and cultured for five days in expansion media comprising StemSpan Serum Free media (Stem Cell Technologies Cat #09650) and CC100 cocktail (Stem Cell Technologies Cat #02690), which contains SCF, Flt3L, IL-3, and IL-6, which promotes CD34⁺ expansion and non-directed lineage differentiation. Following five days in expansion media, cells were transferred to differentiation media, containing EPO, to drive erythroid differentiation. Two parallel differentiation runs were performed: donor 1+2 and donor 2+3, which allowed, following donor deconvolution post-sequencing, detection of differentiation run or donor specific differences throughout the differentiation process. Cells were collected for counts, single cell and bulk RNA sequencing at four time points: E0 (freshly thawed), E3 (Expansion phase day 3), E5 and D1 (Differentiation phase day 1).

Bulk mRNA sequencing was performed separately on three donors at E0 (time of thaw). This was used to deconvolute the single cell samples using donor specific Single-nucleotide polymorphisms (SNPs). Deconvolution of donor contribution using donor specific SNPs was performed as part of the downstream analysis.

For the subsequent time points, the two pooled donor differentiation runs were sequenced independently for bulk RNA sequencing (two sequencing runs: 1+2, 2+3) and both differentiation runs were pooled for single cell mRNA sequencing (one pool: 1+2+2+3).

RNA isolation was performed for bulk total RNA sequencing using the miRNeasy Micro Kit (50) QIAGEN 217084 kit. RNA was quantified using the Qbit and Bioanalyzer.

Bulk total RNAseq NGS libraries were prepared using the KAPA Stranded RNA-Seq with RiboErase kit (Kapa BioSystems).

Paired end (2×125) total bulk RNA sequencing (mRNA, isoforms, SNPs, non-coding RNA) was performed on the Illumina HiSeq 2500 using the High Output v4 single lane flow cells.

For single cell sequencing at each time point, all donors were pooled into a single tube prior to running through the 10× Genomics Single Cell 3′ v2 workflow steps. Cells were washed, counted, and run within one hour using 10× partitioning and cDNA amplification (10× Genomics, Single Cell 3′ v2). The rest of the library prep was completed as per the 10× Genomics Single Cell 3′ v2 library prep protocol. 10× samples were sequenced using three NextSeq High 1×75 flow cell runs.

Count matrices were obtained by running Cell Ranger (10× Genomics) with default settings. Low quality cells with less than 200 genes expressed were filtered, genes expressed in less than three cells were filtered, and data was CPM normalized and log-transformed. Highly variable genes were detected, and the count matrices were corrected for mitochondrial gene expression. A dimensionality-reduced representation of the count matrix was embedded using UMAP, clustered using Louvain clustering and annotated using differential expression testing (Mann Whitney U test) and comparisons to established marker genes. Proxies for cellular states are the annotated clusters, as shown in FIG. 5A. Differential signatures for the 8 to 15 transitions, i.e., from a non-lineage committed CD34⁺ progenitor cell to cells of the megakaryocyte lineage, were then used to predict perturbations that would promote the transition.

### Example 9: Testing Compounds In Vitro Culture

Briefly, mobilized peripheral blood (mPB), cord blood (CB) or bone marrow (BM) derived CD34+ hematopoietic stem and progenitor cells were thawed and allowed to recover in StemSpan SFEM (Stem Cell Technologies) supplemented with TPO and CC100 cytokine supplement (Stem Cell Technologies) that contains SCF, Flt3L, IL-6 and IL-3 (Expansion media) for 48 hrs prior to delivery of the perturbagens. Perturbagens reconstituted in DMSO were added to media at different concentrations. Cells were cultured for 2 and 5 days in the presence of perturbagens prior to collection for cell counts, viability and flow-cytometric analysis to assess the impact of the perturbagens on the culture composition. Perturbagens are listed in Table 6.

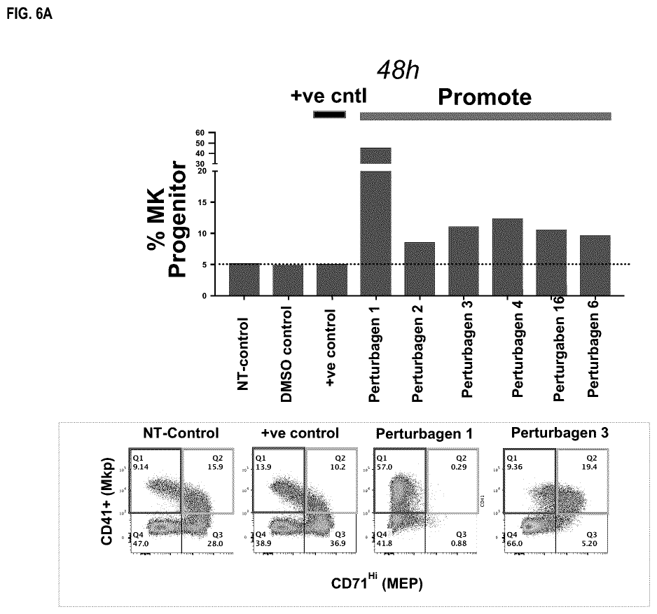

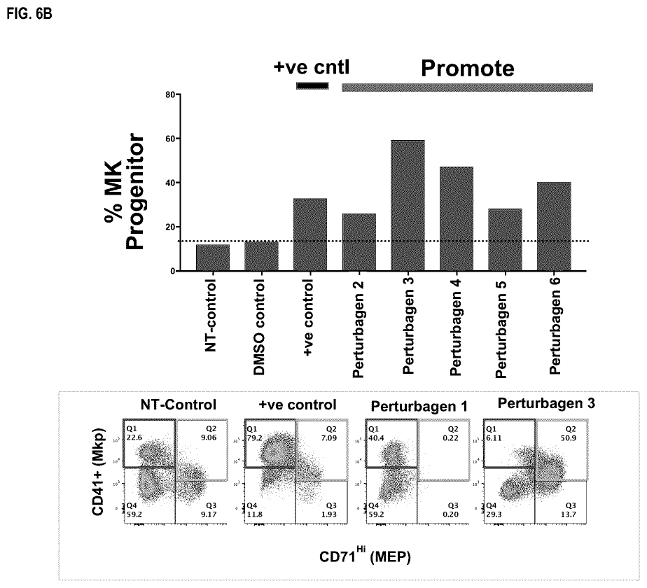

Expansion of megakaryocyte progenitors was measured by positive expression of the surface marker CD41a+(ITGA2B) with co-expression of CD71+(Transferrin receptor) within the CD34⁺CD38^{+/-} gate (encompassing hematopoietic stem cells progenitors) relative to vehicle control conditions (DMSO) at 2 days post-treatment. At 5 days post-treatment, cells were gated for expression of CD41a+ and CD71+ with the CD34⁻CD38^{+/-} gate (encompassing lineage positive population). Conditions promoting megakaryocyte differentiation (StemSpan+SCF, IL-6, IL-9, and TPO) were included to demonstrate that lineage specific changes could be measured following 2 and 5 days of treatment. After 5 days of treatment it was observed that 3/4 of predictions resulted in promotion of MkP lineage direction, shown by increased expression of CD41a (FIG. 6A-6B). Interestingly, one of the predicted compounds, Perturbagen 3, resulted in a 60% increase in CD41a+ positive progenitors in a 5-day treatment window.

To further characterize megakaryocyte lineage commitment and differentiation, a modified flow cytometry panel will be used to identify megakaryocyte/erythroid progenitors (CD71^{high}, CD41⁻CD235a⁻), Erythroid progenitors (CD71^{high}, CD235a⁺), early megakaryocyte progenitors (CD71⁺CD41⁺CD235a⁻), and late megakaryocyte progenitors (CD71^{low}CD41a⁺CD42b⁺CD235a⁻) allowing to identify define states where are perturbagens are acting on during megakaryocyte lineage commitment and differentiation.

In this and the above examples, antibodies directed against hematopoietic surface proteins and conjugated fluorophores are listed below in Table 14.

TABLE 14
Antibodies against Hematopoietic Surface Proteins and
Conjugated Fluorophore

| Antibody | Fluorophore | Vendor | Clone | Cat # |
|---|---|---|---|---|
| CD41a | FITC | eBiosciences | HIP8 | 11-0419-42 |
| CD66b | Percp5.5 | Biolegend | G10F5 | 305108 |
| CD71 | PE | eBiosciences | OKT9 | 12-0719-42 |
| FcεRla+ | PE-Cy7 | eBiosciences | AER-37 | 25-5899-42 |
| CD64 | APC | Biolegend | 10.1 | 305014 |
| CD38 | AF700 | eBiosciences | HIT2 | 56-0389-42 |
| CD34 | APC-eFlur780 | eBiosciences | 4H11 | 47-0349-42 |
| CD123 | BV421 | Biolegend | 6H6 | 306018 |
| CD135 | BV421 | BD | 4G8 | 564708 |
| CD45RA | BV785 | BD | Hi100 | 563870 |
| CD235a | PE-Cy7 | BD | HIR2 | 563666 |
| CD42b | APC | eBiosciences | HIP1 | 17-0429-41 |

In this and the above examples, flow cytometry panel comparisons with either CD135 or CD123 to identify myeloid progenitor populations and/or to identify cell states are listed below in Table 15.

TABLE 15
Flow Cytometry Panel Comparison with CD135 or CD123 to Identify Human Myeloid
Progenitor Populations

| | Immunophenotype | Immunophenotype |
|---|---|---|
| Cell Type | (Lineage−) CD135 | (Lin−) CD123 |
| Megakaryocyte- | CD34+CD38+CD45RA−CD135−CD71+ | CD34+CD38+CD45RA−CD123− |
| erythroid | | CD71Low |
| progenitor MEP′ | | |
| (Meg/Eryth | | |
| Pro; MkP) | | |
| Erythroid | CD34+CD38+CD45RA−CD135− | CD34+CD38+CD45RA−CD123− |
| Progenitor | CD71+CD41−CD235a+ | CD71+CD41−CD235+ |
| MkP Progenitor | CD34+CD38+CD45RA−CD135− | CD34+CD38+CD45RA−CD123− |
| | CD71+CD41+CD235a− | CD71+CD41+CD235a− |
| Common myeloid | CD34+CD38+CD45RA−CD135+ | CD34+CD38+CD45RA−CD123+ |
| progenitor (CMP; | | |
| immature | | |
| populations - | | |
| myeloid and | | |
| early stage | | |
| Neutrophil) | | |
| Eosinophil/ | CD34+CD38+CD45RA−CD135mid | CD34+CD38+CD45RA−CD123+ |
| basophil/mast | | |
| cell progenitor | | |
| Mast Cell/ | CD34+CD38+CD45RA− | CD34+CD38+CD45RA− |
| Basophil | CD135midFcεRla+ | CD123+FcεRla+ |
| GMP | CD34+CD38+CD45RA+CD135+ | CD34+CD38+CD45RA+CD123+ |
| (Neutrophil | | |
| progenitor; | | |
| Monocyte/ | | |
| DC progenitor) | | |
| Neutrophil | CD34+CD38+CD45RA+CD135+ | CD34+CD38+CD45RA+CD123+ |
| Progenitor | CD66b+ | CD66b+CD64− |
| Monocyte | CD34+CD38+CD45RA+CD135+ | CD34+CD38+CD45RA+CD123+ |
| Progenitor | CD64+ | CD66b−CD64+ |

NOTE:
Lineage: CD4−CD8−CD11b−CD14−CD19−CD20−CD56−CD10− (this stain is optional if working with purified CD34+ cells)

### Example 10: Testing Functional Impact of Compounds In Vitro

To further evaluate megakaryocyte differentiation beyond lineage commitment, hematopoietic CD34+ progenitors will be cultured in vitro for extended period of time (7-14 days). The extended culture conditions will allow for the quantification of other important functional characteristic of in vitro generated megakaryocytes, including ploidy, size, and granularity. For ploidy analysis CD42b+ cells will be co-stained with Vybrant DyeCycle violet (ThermoFisher) and ploidy will be measured using flow cytometry at define time points (10-14 days). In addition, changes in size and granularity will be assessed by measuring changes in forward scatter (size) and side scatter (granularity) gating on CD42b+ positive cells (megakaryocytes). To complement flow cytometry readouts, morphological features of megakaryocytes (size, granularity, and ploidy) will be studied through histology. Briefly, cells will be immobilized on glass slides using a cytospin, followed by hematoxylin and eosin (H&E) and May-Grunwald/Giemsa (MGG) staining. These additional readouts will allow to further validate perturbagens tested during our in vitro validation.

### Example 11: Testing Compounds In Vivo

Following in vitro validation of compounds that promoted MkP expansion, molecules were tested in C57BL/6 mice to evaluate the compounds ability to promote megakaryocyte expansion and an increase in platelet number in vivo during steady state hematopoiesis or following sub-lethal myeloablation.

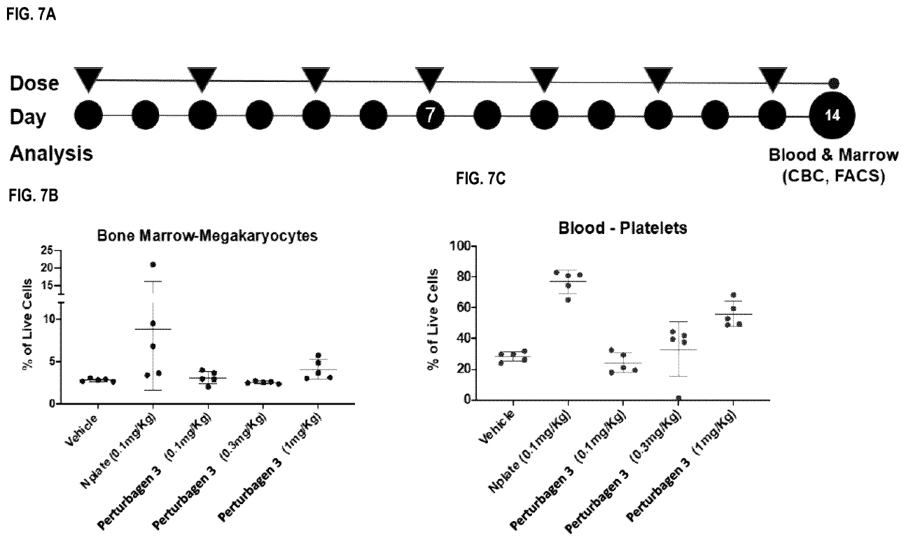

FIGS. 7A-7C show results obtained when animals were dosed daily i.p. for 14 days with two concentrations of Perturbagen 3 or the FDA approved thrombopoietin mimetic Romiplostim (Nplate). Changes in bone marrow Mkps (FIG. 7B) and blood platelets (FIG. 7C) demonstrate that Perturbagen 3 can increase Mkp lineage in vivo.

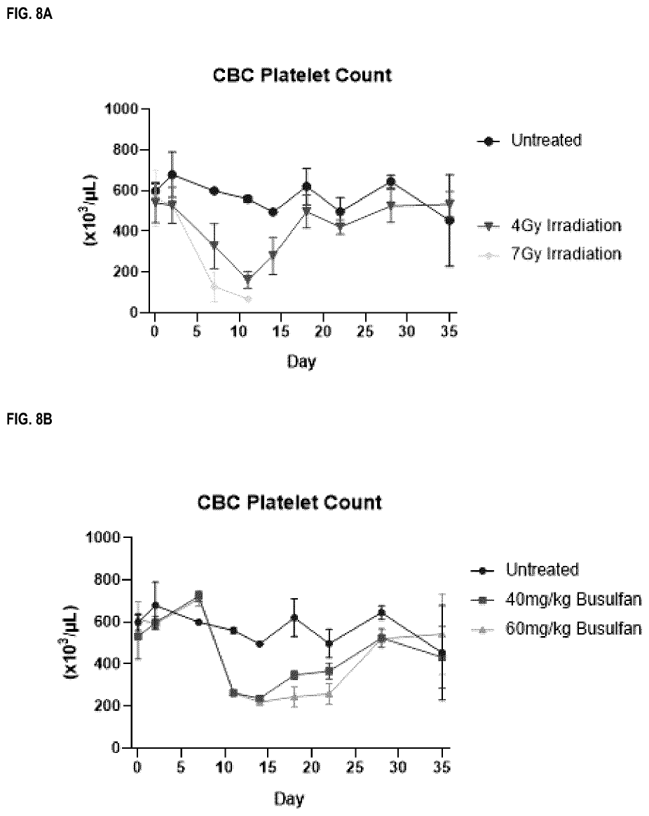

Sublethal myeloablation was induced by sublethal irradiation or busulfan treatment. Busulfan is DNA alkylating reagent used in the clinic for bone marrow conditioning with reduced toxicity. Similarly, busulfan dosing has been previously established in murine models as a less toxic conditioning method and an alternative to whole body irradiation. See FIGS. 8A-8B.

Briefly, in the setting of normal steady state hematopoiesis or in the setting of busulfan mediated conditioning (sub-lethal myeloablation), a single dose or repeated doses (dosed daily or every other day) of compounds were delivered via intraperitoneal, subcutaneous, or intravenous injection into 8-15 week old female C57/Bl6 mice. For repeat dosing, compounds were injected once every 24 hrs or every other day for up to 2 weeks. As part of the experimental readout, weight and health of each mouse was monitored for overt signs of compound toxicity. As a primary readout of efficacy, peripheral blood was collected every 2 days post-injection for 2-4 weeks. Blood was analyzed using a complete blood count (CBC) analyzer and by flow cytometry using a multiparameter in-house assay (Table 16). Using these two methods, the frequency of platelets within the peripheral blood and other major changes in other blood populations (T-cells, B-cells, monocyte, neutrophils, and RBCs) can be accurately monitored during dosing with test compounds. At the study termination, mice were sacrificed and analysis of the bone marrow, in addition to peripheral blood, was performed using flow cytometry to measure changes in megakaryocytes and all other major blood lineages present the bone marrow. Furthermore, bone marrow analysis can be expanded to measure stem cells and progenitor populations (MEP) relevant to megakaryocyte development. As part of in vivo experiments to evaluate compounds that increase Mkp/platelets, animals were dosed with the thrombopoietin mimetic romiplostim (Nplate) which has been FDA approved to treat thrombocytopenia.

TABLE 16
Antibodies against Murine Hematopoietic Surface Proteins and
Conjugated Fluorophore

| Antibody | Fluorophore | Vendor | Clone | Catalog# |
|---|---|---|---|---|
| CD3 | BV510 | Biolegend | 17A2 | 100233 |
| CD19 | BV605 | Biolegend | 6D5 | 115539 |
| Ter119 | APC-Cy7 | Biolegend | Ter119 | 116223 |
| CD11b | BB515 | BD | M1/70 | 564455 |
| Ly6G | BV786 | BD | 1A8 | 740953 |
| Ly6C | AF 700 | Biolegend | A7R34 | 128023 |
| CD41 | BV421 | Biolegend | MWewg30 | 133911 |
| CD45 | PerCPCy5.5 | Biolegend | 30-F11 | 103131 |
| SytoxRed | Alexa 647 | Thermo Fisher | — | S34859 |

TABLE 17
Immunophenotype for Murine Blood Lineages

| Cell Type | Immunophenotype |
|---|---|
| Erythroid Cells | CD45−Ter119+ |
| T-cells | CD45+CD11b−CD3+ |
| B-cells | CD45+CD11b−CD19+ |
| Neutrophils | CD45+CD11b+ly6-C_{low}Ly6-G+ |
| Monocytes | CD45+CD11b+Ly6-G− |
| Megakaryocytes (bone marrow) | CD41+FSC_{med-high} |
| Platelets (periphery) | CD41+FSC_{low} |

### Example 12: In Vitro Testing of Additional Compounds

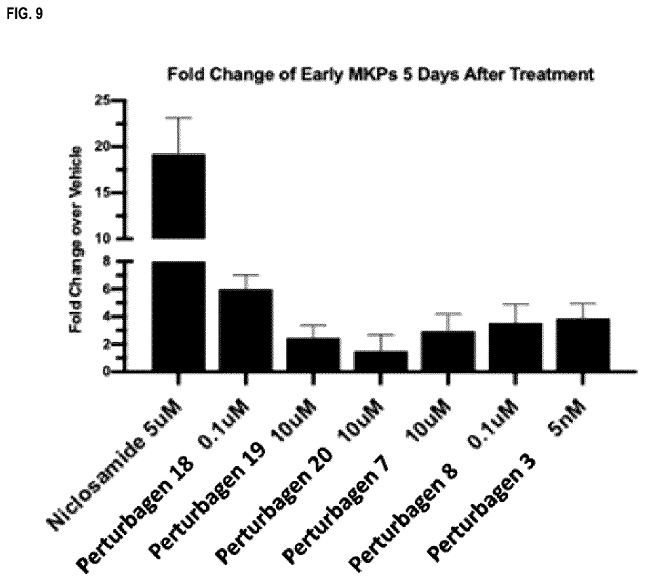

Based on initial compounds tested and shown to have activity, additional molecules were tested in the assay of Example 9. Briefly, molecules matching the predicted signatures were evaluated for their ability to increase megakaryocyte progenitors. Consistent with the predicted signatures a number of compounds tested had activity greater than Perturbagen 3 at inducing CD34−CD41+ committed megakaryocyte progenitors. These assays were carried out as discussed in Example 9. A bar graph of the fold change of early megakaryocytes progenitors was plotted after five days of treatment. Early MkP were defined at day 5 were defined as CD71+CD41+CD235a−. As shown in FIG. 9, several compounds were active at increasing megakaryocyte progenitors in the in vitro human lineage differentiation assay. Plot represent at least 3 biological replicates.

These results demonstrate that the compounds disclosed herein, including Perturbagen 6, can direct a change in cell state of a progenitor cells to increase early megakaryocyte progenitors.

### Example 13: Perturbagen 6 and Related Compounds

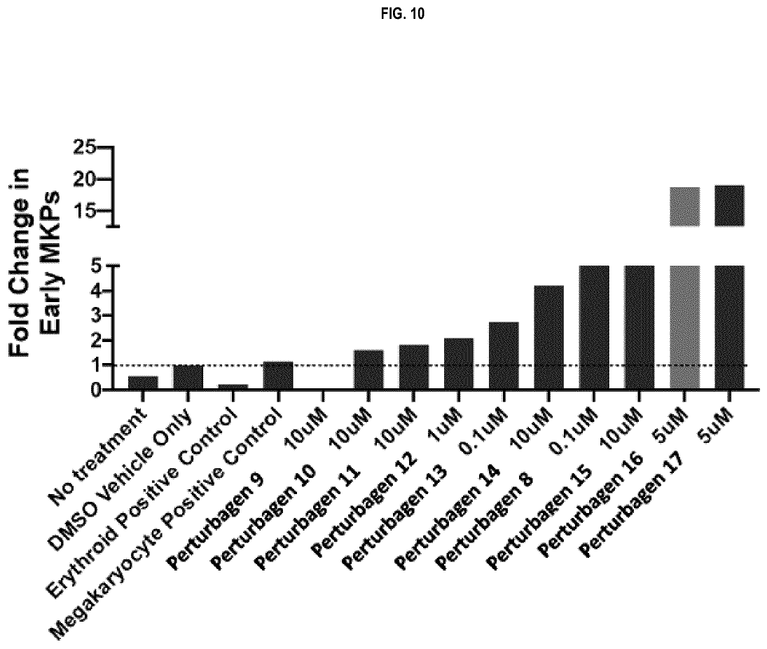

Perturbagen 6 was identified to significantly increase early megakaryocyte progenitors in the in vitro lineage differentiation assay of Example 9. Therefore, similar molecules were that were expected target similar cell behaviors were tested. These compounds were tested in the cell-based assay out as discussed in Example 9. A bar graph of the fold change of early megakaryocytes progenitors was plotted after five days of treatment. Early MkP were defined at day 5 were defined as CD71+CD41+CD235a−. As shown in FIG. 10B, several of the evaluated compounds confirmed to be active at increasing megakaryocyte progenitors. Perturbagen 7 increased megakaryocyte progenitors to the same magnitude as Perturbagen 6 (FIG. 10B). Perturbagens are listed in Table 6.

These results demonstrate that the compounds disclosed herein, including Perturbagen 7, can direct a change in cell state of a progenitor cells to increase early megakaryocyte progenitors.

### Example 14: In Vivo Model (Healthy) to Monitor Changes in Megakaryocyte Progenitors and Mature Platelets

One of the objectives of this study was to expand the analysis of Example 11 to the hematopoietic progenitor compartment.

### Study Design:

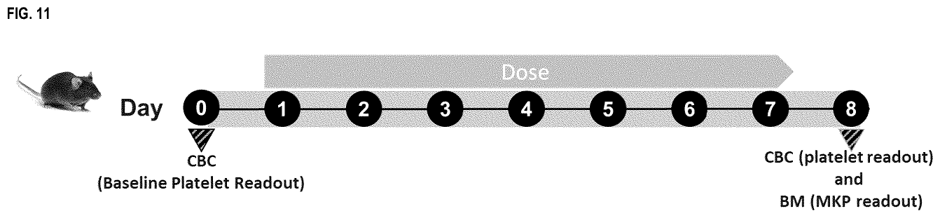

To determine whether in vitro validated compounds translate in vivo, a 7-day and 14-day healthy mouse model was developed to measure changes in platelets in the blood and megakaryocytes/megakaryocyte progenitors in the bone marrow. A diagram of the assay design is provided in FIG. 11. In contrast to the 14-day study shown in FIG. 7A, this study was shorter (7 days). To measure blood platelets, animals were bled and complete blood counts (CBC) were measured on a blood analyzer (HESKA HT5 ANALYZE) at baseline (Day 0) and at the end of the study (Day 7 or Day 14 respectively). For analysis of megakaryocyte and megakaryocyte progenitors, bone marrow was harvested from the femur bone and cells were analyzed by flow cytometry using a 14-antibody panel. The designed flow cytometry panel allows for resolution of different populations within the bone marrow. Specifically, the stem/progenitor compartment (Lin−Sca+ckit+), the progenitor compartment (Lin−Sca−ckit+), committed early megakaryocyte progenitors (Lin−CD41+CD42−), late megakaryocyte progenitors (Lin−CD41+CD42+, FSC^{low}) and mature megakaryocytes (Lin-CD41+CD42+, FSC^{high}) were measured.

### Bone Marrow Harvest and Analysis of Hematopoietic Stem Cell and Progenitor Compartment:

Animals were euthanized using CO₂ asphyxiation, with cervical dislocation for confirmation according to IACUC protocol. Animals were sprayed with 70% EtOH, and both femurs were dissected out of the animal using scissors and forceps. The femurs were gently scraped of all tissue with scalpel while being kept cold at all times. The epiphysis of each femur were cut using scalpel and forceps to flush bone marrow. Femurs were flushed using 1 ml syringes (with 25 gauge needles) using 1 ml of disassociation buffer (4% Grifols human Albumin, NDC-68516-1 in Dulbecco's Phosphate-Buffered Saline (dPBS) with 2 mM EDTA. Slowly femurs were flushed into 1.5 ml Eppendorf tubes on ice, alternating entry side using the 1 ml syringe. After flushing the bones, bone marrow cells were pipetted up and down gently and filtered using a 35 μm cell strainer (Corning 352235) to break up bone marrow suspension into single cells. For counting bone marrow cells, 20 μl of the filtered cell suspension was diluted 1:3 with 40 μl of dPBS. Diluted cell samples were mixed 1:1 with AO/PI dye and cell number and viability was measured using a NEXCELOM cell counter. Using this method for flushing bone marrow, 6-10×10⁶ total cells can be harvested from a single femur.

For cell surface staining, 2.5×10⁶ cells/well were stained in duplicate and placed in separate U bottom 96 well plates (Corning, 3799). Cells were spun down using plate adaptors for 8 minutes at 300 g (SORVAL×PRO Series centrifuge). After spinning, supernatant was decanted by flicking plate on to an absorbent pad. Cells were washed and spun by adding 250 μl of FACS buffer (1% GRIFOLS human albumin diluted in dPBS) prior to staining. Staining was performed by adding 250 μl of flow panel cocktail (Table 18) diluted in ice cold BD Brilliant stain buffer (BD Biosciences, 566349) to cell pellets and resuspended by gentle pipetting. The stained sample plate was incubated at 4° C. for 1 hour in the dark. After incubation, cells were washed twice with 250 μl of FACS buffer and spun at 300 g for 8 minutes. After the last wash, cells were resuspended cells in 250 μl of FACS buffer with live/dead SYTOX AADVANCED (1:1000). Samples were incubated at room temperature for 5 minutes in the dark and filtered through 35 μm strainer prior to acquisition.

Compensation was performed using ULTRACOMP beads (Thermo Fisher, 01-2222-42) following manufacturers staining protocol. In brief, 5 μl of each antibody was incubated with for 15 minutes at 4° C. After incubation, compensation samples were ready for acquisition. Live/Dead compensation was done with cells. Briefly, ˜3×10⁵ bone marrow cells were incubated in 0.2 ml of 70% ethanol/PBS for 2 minutes at room temperature. Treated cells were diluted with 1.3 mls of FACS buffer, spun down and resuspended in FACS buffer containing Live/Dead SytoxAADvanced and 1.5×10⁵ live cells.

TABLE 18
Antibody panel for bone marrow progenitor compartment and megakaryocyte progenitors. 14
antibodies, 11 colors (FITC antibodies were used in combination to create a dump channel)

| | | | | | Microliter |
|---|---|---|---|---|---|
| | | | | | per 2.5 |
| Antibody | Fluorophore | Vendor | Clone | Cat # | million cells |
| CD4 | FITC | Biolegend | GK1.5 | 100406 | 1.25 |
| CD48 | BV785 | Biolegend | HM48-1 | 103449 | 1.25 |
| CD127 | FITC | Biolenged | A7R34 | 135008 | 1.25 |
| CD42d PE | PE | Biolenged | IC2 | 148504 | 1.25 |
| CD34 | AF700 | BD Biosciences | RAM34 | 305014 | 3.125 |
| CD16/32 | APC-Cy7 | BD Biosciences | 2.4G2 | 560541 | 1.25 |
| CD135 BV421 | BV421 | Biolegend, | A2F10 | 135315 | 1.25 |
| CD41 BV510 | BV510 | Biolegend, | MWREG30 | 133923 | 1.25 |
| CD150 BV650 | BV650 | Biolegend, | TC15-12F12-2 | 115932 | 1.25 |
| CD117 APC | APC | Biolegend, | 104D2 | 313216 | 1.25 |
| CD3/Gr-1/CD11b/CD45R | FITC | Biolegend | 145-2C11, RB6-8C5, | 13002 | 25 |
| (B220/TER-119) | | | M1/70, RA36B2, Ter-119 | | |
| Brilliant Stain Buffer | | BD Biosciences | N/A | 566349 | 208.125 |
| Live/Dead Sytox | 7-AAD | Thermo Fisher | N/A | S10374 | 1:1000 |
| AADvanced | | | | | dilution |

TABLE 19
Murine Bone marrow defined cell types and associated flow
cytometry markers

| | Cell Type | Marker Selection |
|---|---|---|
| | Lineage + | CD11b(Mac1), Gr1, Ter119, |
| | | CD45R(B220), CD3e, CD4, CD8 |
| LSK | Long term | Lin⁻c-Kit+Sca-1+CD34⁻CD135(Flk2)⁻ |
| Compart- | HSCs | |
| ment | Short term | Lin⁻c-Kit+Sca-1+CD34⁺CD135(Flk2)⁻ |
| | HSCs | |
| | MPP (Flk2⁺) | Lin⁻c-Kit+Sca-1+CD34⁺CD135(Flk2)⁺ |
| Oligo/ | CMP | Lin⁻c-Kit+Sca1⁻CD34⁺CD16/32(FcgR)^{low} |
| bipotent | GMP | Lin⁻c-Kit+Sca-1⁻CD34⁺CD16/32(FcgR)⁺ |
| progenitor | MEP | Lin⁻c-Kit+Sca-1⁻CD34⁻CD16/32(FcgR)⁻ |
| compartment | | |
| Committed | Early MKP | Lin⁻CD41+CD42⁻ |
| progenitor | Late MKP | Lin⁻CD41+CD42⁺low FSC |
| compartment | MK | Lin⁻CD41+CD42⁺High FSC |

### Gating Strategy:

For all the populations listed in Table 19, debris gating was the same. Briefly, FSC (size) vs SSC (granularity) was used to gate out debris. Next, doublets were removed by plotting FSC-H vs FSC-W and gating the linear population. Finally, SSC-H was plotted against SSC-W and doublets were gated out to obtain a population of single cells. For Lin-Sca+cKit+(LSK) and Oligo/bipotent progenitor compartments, doublet exclusion was performed by plotting the height and width for both forward scatter (FSC) and side scatter (SSC).

The cells in the LSK compartment could be expanded to make MKs, and cells in this compartment were the cells that repopulate specially after CIT. These cell populations were partially equivalent to the CD34+ cells in humans that give rise to MKs. Doublets had double the area and width values of single cells while the height was roughly the same.

### Identifying Live Cells and Lineage Negative Cells:

Approximately 95% of cells isolated from bone marrow using flushing method were live (CytoxAAD negative). Lineage negative gate were consequently negative for all the lineage markers used for dump channel for exclusion (FITC).

Identifying HSCs within the LSK Population:

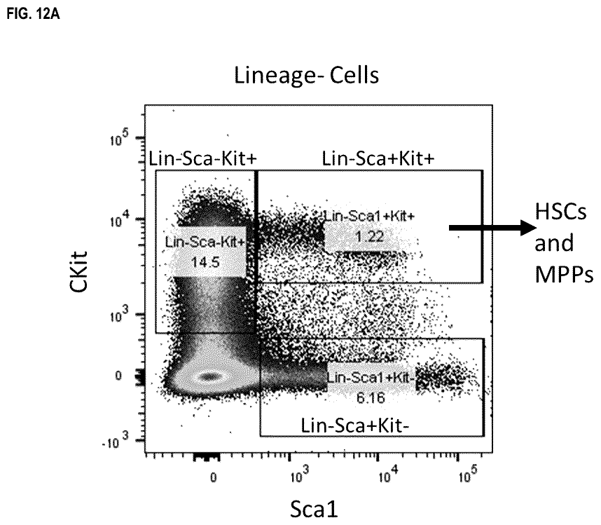

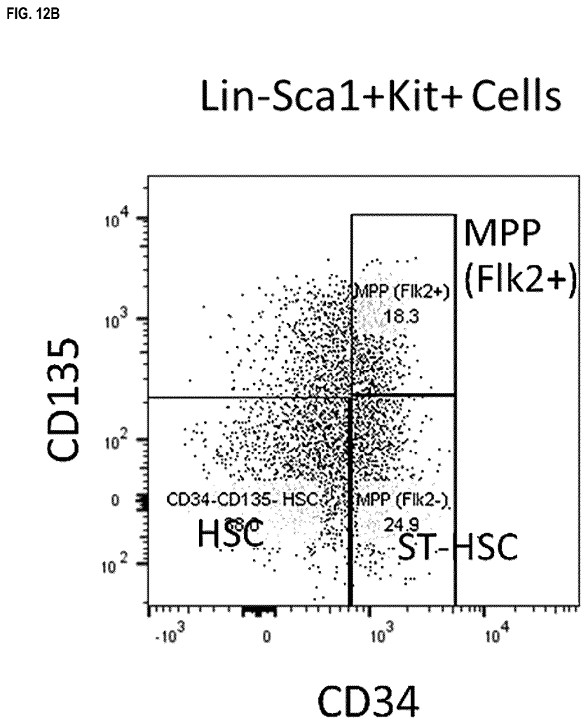

As shown in FIG. 12A, LSK cells were defined as Lin-Sca1+cKit+. A subset of the Lin-Sca1+cKit+ gate were long term HSCs, denoted by CD34−CD135−, short term HSC were were CD34+CD135−, and MPP population were CD34+CD135+(FIG. 12B).

### Identifying Oligopotent and Bipotent Progenitors:

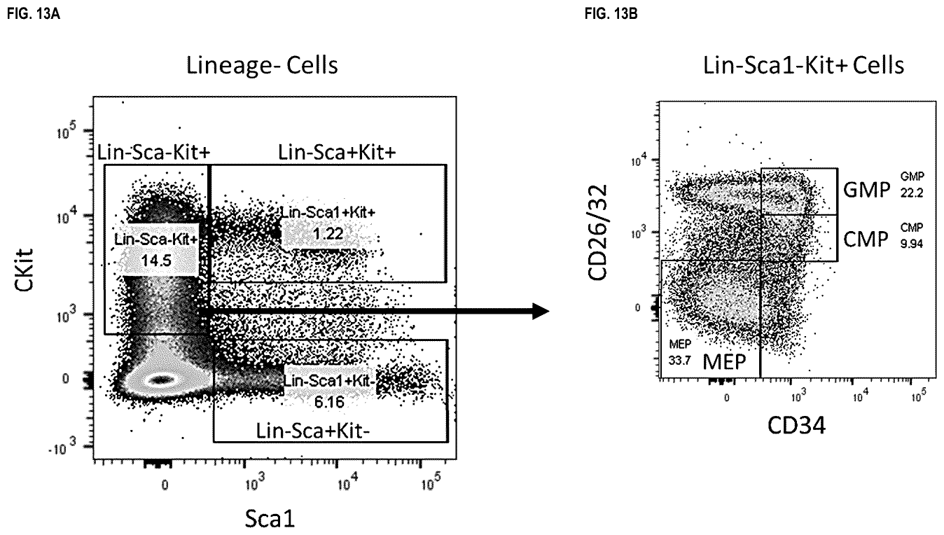

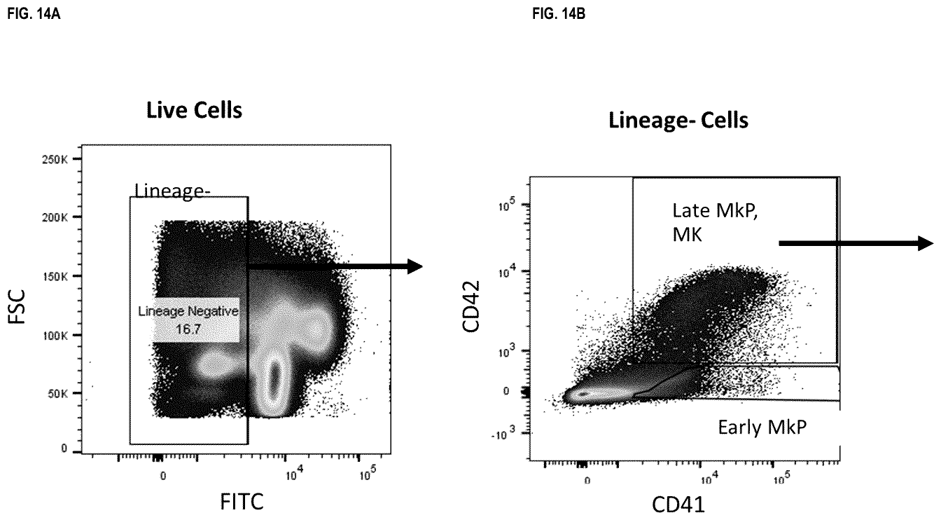

Oligo and bipotent progenitors refer to differentiation potential of cell types found within the lin-Sca1-cKit+ gate (FIG. 13A). Common myeloid progenitor (CMP) can give rise to granulocyte myeloid progenitors (GMP) and megakaryocyte erythroid progenitors (MEP). Cell surface phenotyping of these different populations was based on levels of CD34 and CD16. MEP population was defined by CD34−CD16/32−. CMP population was defined by CD34+CD16/32 low and GMP was defined CD34+CD16/32 high (FIG. 13B)

### Identifying Megakaryocytes and Megakaryocyte Progenitors:

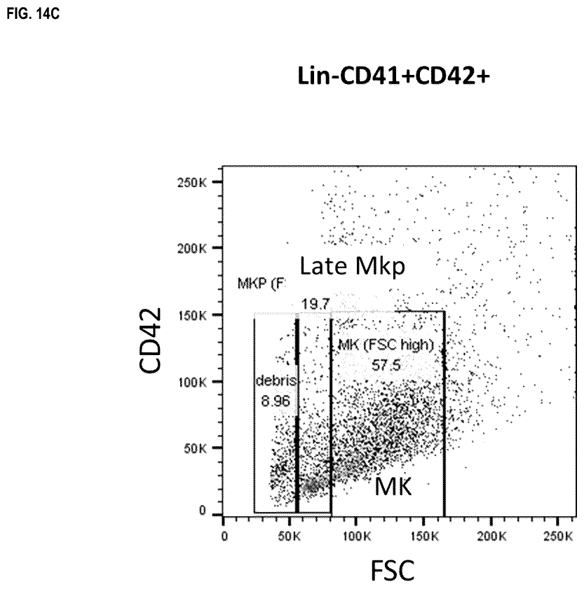

Megakaryocyte progenitors (MkPs) and mature megakaryocyte (MKs) were assessed using an alternative gating strategy. In this strategy MkPs and mature MKs were captured in the lineage negative gate (Lin−). Due to the large size of megakaryocytes there was no doublet exclusion gate prior to gating for this population. To identify these cellular populations debris gating followed by live cell identification was completed prior to defining lineage negative compartment, respectively. Early MkP's were defined as Lin−CD41+CD42d− (FIG. 14A and FIG. 14B). Late MkP and mature MK were defined by Lin−CD41+CD42d+. Resolution of late MkP and mature MKs was attained by using size discrimination using FSC and SCC paramaters (FIG. 14C). In this gating strategy, small cells (<50 k) were labeled debris (platelets aggregates), cells with low FSC were defined as late MkPs and cell with high FSC were defined as megakaryocytes (FIG. 14C).

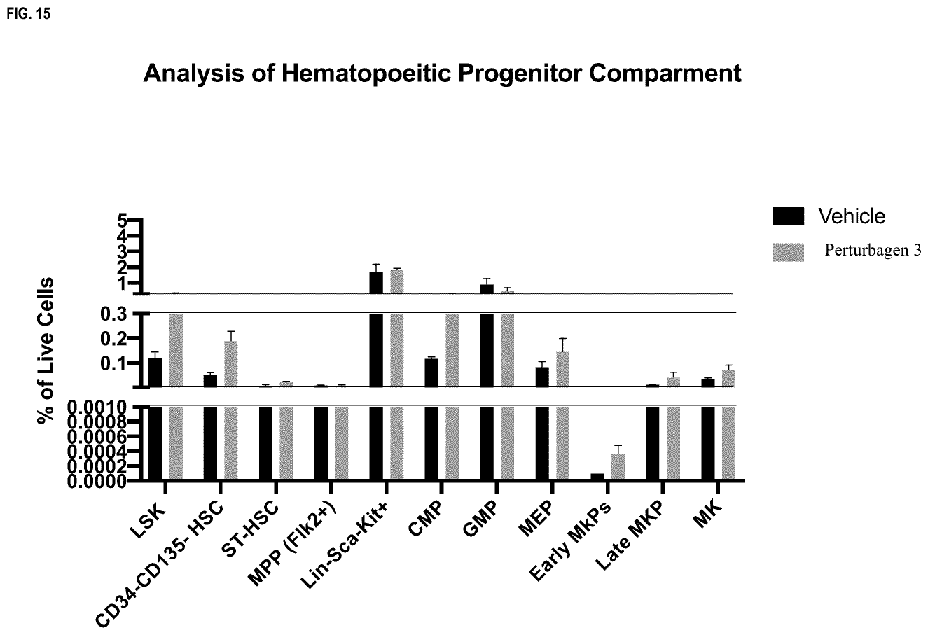

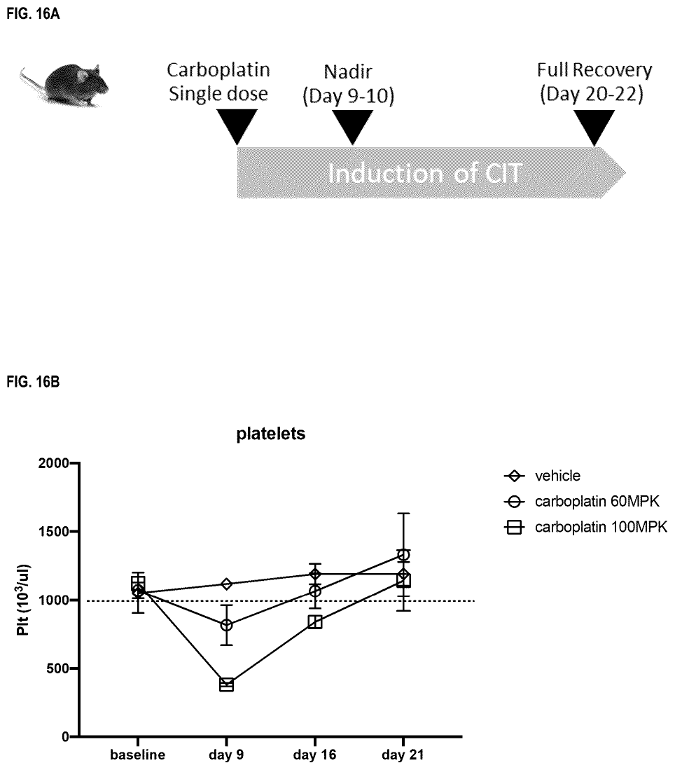

Next, the differences between different cell populations were compared in mice treated with vehicle only or Perturbagen 3. Briefly, mice were treated with vehicle only (N=2) or 1 mg/kg Perturbagen 3 administered every-other-day (EOD) for 7 days (N=3). Following the treatment, analysis of bone marrow was performed for changes in megakaryocyte progenitors and hematopoietic compartments. FIG. 15 shows a bar graph summarizing the results of bone marrow analysis of vehicle- and Perturbagen 3-treated animals. As shown in FIG. 15, the LSK compartment and the CD34-CD135-HSC (long term HSC) compartments increased in mice treated with Perturbagen 3 compared to the vehicle-treated mice. CMP, MEP, and early MkPs compartments increased in mice treated with Perturbagen 3 compared to the vehicle-treated mice.

These results demonstrate that the treatment with Perturbagen 3 causes an increase in LSK, early MkP, early MK, and late MK compartments. Accordingly, the perturbogens disclosed herein, including Perturbagen 3, can direct a change in cell state of a progenitor cells to increase early megakaryocyte progenitors. Perturbagens are listed in Table 6.

### Example 15: In Vivo Model of Chemotherapy Induced Thrombocytopenia

Chemotherapy induced thrombocytopenia (CIT) was a major disease burden in cancer patients undergoing chemotherapy. CIT increases the risk of bleeding resulting in delay of chemotherapy and/or reduction in relative dose intensity (RDI), which has a major impact in overall outcomes to cancer treatment. Currently, there were no approved treatments besides platelet transfusions in severe CIT cases. Thrombopoietin (TPO) mimetics, such romiplostim, were being investigated as a possible new treatment to address CIT. Based on the ability to modulate megakaryocyte progenitors in vitro and healthy animals, murine models that replicate CIT were developed (Table 19.) To this end, the following chemotherapeutic compounds known to induced myelosuppression and thrombocytopenia in mouse and humans were selected to develop CIT models:

Carboplatin a cisplatin analog commonly used in the clinic to treat a diverse type of malignant tumors. Carboplatin works by cross-linking to DNA resulting in DNA damage and decreased tumor burden. In addition, carboplatin also leads to bone marrow myelosuppression including transient thrombocytopenia in both human and mice.

Gemcitabine was a deoxycytidine analogue acting as DNA synthesis (S-phase) inhibitor widely used as a first line anticancer drug. Along with its anti-cancer properties, gemcitabine has myelosuppressive properties resulting in transient thrombocytopenias in both human and mice.

Cyclophosphamide was a prodrug used widely as an antitumor agent. Once in the body cyclophosphamide was metabolized into phosphoramide mustard which acts a DNA crosslinking agent resulting in DNA damage. Interestingly, cells with high aldehyde dehydrogenase (ALDH) activity were spared from cyclophosphamide toxicity due to their ability to metabolize phosphoramide mustard to carboxyphosphamide. Similar to the other compounds listed above cyclophosphamide results in bone marrow myelosuppression in both human and mice.

Fluorouacil (5-FU) was an antimetabolite used in the clinic to treat a diverse number of solid tumors. 5-FU was a pyrimidine analog acting through inhibition of thymidylate synthase resulting in blocked DNA synthesis. Similar to the compounds above one of the cytotoxic effects results in bone marrow myelosuppression in both human and mice.

TABLE 20
Table depicting dose, route of administration, and frequency of
chemotherapy drugs to induce thrombocytopenia in mice

| Compound/Treatment | Dose | | ROA | Regimen |
|---|---|---|---|---|
| Carboplatin | 100 | mg/kg | i.p. | Single dose |
| Gemcitabine | 120 | mg/kg | i.p. | 1-2 doses in |
| | | | | 48 hrs |
| Cyclophosphamide | 150 | mg/kg | i.p. | 1-2 doses in |
| | | | | 48 hrs |
| Fluorouacil | 50-150 | mg/kg | i.p. | Single dose |

### Carboplatin Induced Thrombocytopenia

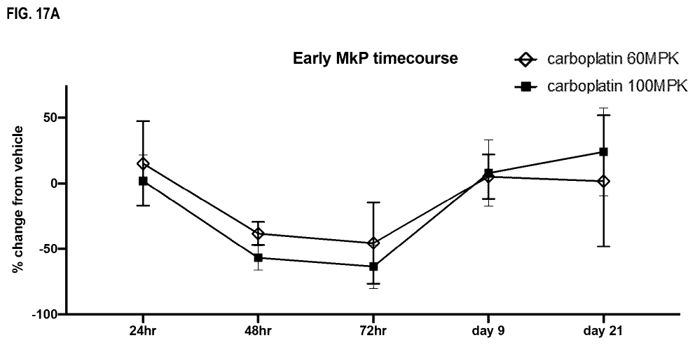

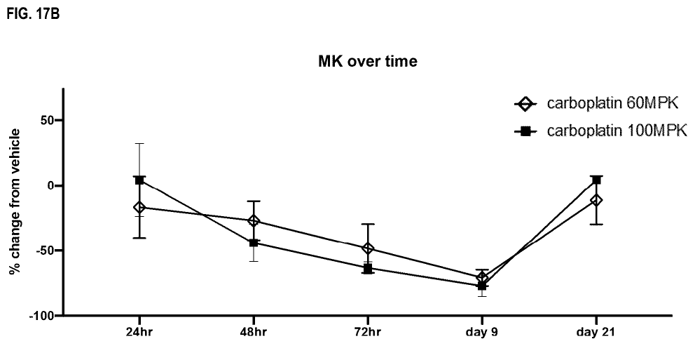

Healthy C57Bl/6J mice were treated with a single dose of carboplatin via intraperitoneal injection (60 or 100 mg/kg) (FIG. 16A). Complete blood count (CBC), specifically changes in platelet number were measured using a blood analyzer (Heska HT5) at baseline Day 0 (baseline), Day 9, Day 16 and Day 21. In addition, bone marrowwas harvested at 24 hrs, 48 hrs, 9 days and 21 days post-carboplatin injection to characterize changes in the hematopoietic stem cells compartment, and changes in megakaryocyte compartment. As shown in FIG. 16B, the treatment with carboplatin resulted in a decrease of blood platelets in a dose dependent manner with the nadir at day 9 post-injection. Animals tolerated the treatment and their platelet number fully recovered by day 21 (FIG. 16B). Analysis of the bone marrow hematopoietic compartments was performed using flow cytometry as described in Example 14. This analysis revealed a dynamic loss and recovery of distinct populations which corresponded to the observed nadir of platelet number in the periphery (FIG. 17A and FIG. 17B).

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1. A method for directing a change in cell state of a progenitor cell comprising:
contacting a population of cells comprising a progenitor cell with at least one perturbagen selected from Table 4, Table 5, and/or Table 6, or a variant thereof, including combinations of the foregoing,
wherein the at least one perturbagen is capable of altering a gene signature in the progenitor cell; and
wherein the progenitor cell is a non-lineage committed CD34+ cell.

2. A method for directing a change in cell state of a progenitor cell, comprising:
contacting a population of cells comprising a progenitor cell with at least one perturbagen capable of altering a gene signature in the progenitor cell,
wherein altering the gene signature comprises an increase in expression and/or activity in the progenitor cell of one or more genes selected from the genes designated as an “up” gene in the gene directionality column of Table 1, Table 2, and/or Table 3, including combinations of the foregoing, and/or a decrease in expression and/or activity in the progenitor cell of one or more genes selected from the genes designated as a “down” gene in the gene directionality column of Table 1, Table 2, and/or Table 3, including combinations of the foregoing, and
wherein the progenitor cell is a non-lineage committed CD34+ cell.

3. A method for directing a change in cell state of a progenitor cell, comprising:
contacting a population of cells comprising a progenitor cell with at least one perturbagen selected from Table 4, Table 5, Table 6, or a variant thereof, including combinations of the foregoing, and capable of altering a gene signature in the progenitor cell,
wherein altering the gene signature comprises an increase in expression and/or activity in the progenitor cell of one or more genes selected from the genes designated as an “up” gene in the gene directionality column of Table 1, Table 2, and/or Table 3, including combinations of the foregoing, and/or a decrease in expression and/or activity in the progenitor cell of one or more genes selected from the genes designated as a “down” gene in the gene directionality column of Table 1, Table 2, and/or Table 3, including combinations of the foregoing, and
wherein the progenitor cell is a non-lineage committed CD34+ cell.

4. The method of claim 2 or 3, wherein altering the gene signature comprises an increase in expression and/or activity in the progenitor cell of a network module designated in the network module column of Table 1, Table 2, and/or Table 3, including combinations of the foregoing.

5. The method of any one of claims 1 to 4, wherein the change in cell state provides an increase in the number of one or more of proerythroblasts, early erythroblasts, intermediate erythroblasts, late erythroblasts, reticulocytes, and erythrocytes, wherein one or more of the proerythroblasts, early erythroblasts, intermediate erythroblasts, late erythroblasts, reticulocytes, and erythrocytes expresses fetal hemoglobin (HbF).

6. The method of any one of claims 1 to 5, wherein the change in cell state provides an increase in F cells.

7. The method of claim 5 or 6, wherein one or more of the proerythroblasts, early erythroblasts, intermediate erythroblasts, late erythroblasts, reticulocytes, and erythrocytes expresses HBG1 and/or HBG2.

8. The method of claim 7, wherein the increase in the number of erythrocytes comprising HbF is relative to the number of erythrocytes obtained from a population of progenitor cells that is not contacted with the at least one perturbagen, or relative to the population of progenitor cells prior to contacting with the at least one perturbagen.

9. The method of claim 8, wherein the increase in the ratio of the number of F cells to non-F cells is increased relative to the ratio obtained from a population of progenitor cells that is not contacted with the at least one perturbagen or relative to the ratio in the population of progenitor cells prior to contacting with the at least one perturbagen.

10. The method of any one of claims 1 to 4, wherein the change in cell state provides an increase in the number of one or more of proerythroblasts, early erythroblasts, intermediate erythroblasts, late erythroblasts, reticulocytes, and/or erythrocytes.

11. The method of claim 10, wherein the increase in the number of erythrocytes is relative to the number of erythrocytes obtained from a population of progenitor cells that is not contacted with the at least one perturbagen or relative to the population of progenitor cells prior to contacting with the at least one perturbagen.

12. The method of any one of claims 1 to 4, wherein the change in cell state provides an increase in the number of one or more of megakaryocyte/erythroid progenitor cells (MEP), committed megakaryocyte progenitor cells, promegakaryocytes, megakaryocytes, proplatelets, and platelets.

13. The method of claim 12, wherein the change in cell state provides an increase in the number of megakaryocytes, proplatelets, and/or platelets relative to the number of megakaryocytes, proplatelets, and/or platelets obtained from a population of progenitor cells that is not contacted with the at least one perturbagen or relative to the population of progenitor cells prior to contacting with the at least one perturbagen.

14. The method of any one of claims 1 to 13, wherein the number of progenitor cells is decreased.

15. The method of any one of claims 1 to 13, wherein the number of progenitor cells is increased.

16. The method of any one of claims 1 to 9, 14, or 15, wherein the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.

17. The method of any one of claims 1 to 4, 10, or 11, wherein the number of proerythroblasts, early erythroblasts, intermediate erythroblasts, late erythroblasts, reticulocytes, and/or erythrocytes is increased after contacting the population of cells comprising a CD34+ cell with the at least one perturbagen.

18. The method of any one of claims 1 to 4, 12, or 13, wherein the number of MEP cells, committed megakaryocyte progenitor cells, promegakaryocytes, megakaryocytes, proplatelets, and/or platelets is increased after contacting the population of cells comprising a CD34+ cell with the at least one perturbagen.

19. The method of any one of claims 1 to 18, wherein the at least one perturbagen selected from Table 4, Table 5, and/or Table 6, or a variant thereof, including combinations of the foregoing, comprises at least 2, at least 3, at least 4, or at least 5 perturbagens selected from Table 4, Table 5, and/or Table 6, or variants thereof, including combinations of the foregoing.

20. The method of claim 2 or 3, wherein the one or more genes selected from the genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, genes selected from the genes designated as an “up” gene in the gene directionality column of Table 1.

21. The method of claim 20, wherein the one or more genes selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1.

22. The method of claim 2 or 3, wherein the one or more genes selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1.

23. The method of claim 22, wherein the one or more genes selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2.

24. The method of claim 2 or 3, wherein the one or more genes selected from the genes designated as an “up” gene in the gene directionality column of Table 2, comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2.

25. The method of claim 24, wherein the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39.

26. The method of claim 2 or 3, wherein the one or more genes selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2.

27. The method of claim 26, wherein the one or more genes selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNAI2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1.

28. The method of claim 2 or 3, wherein the one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or 23 or more genes designated as an “up” gene in the gene directionality column of Table 3.

29. The method of claim 28, wherein the one or more genes selected from Table 3 comprises at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK.

30. The method of claim 2 or 3, wherein the one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3.

31. The method of claim 30, wherein the one or more genes selected from Table 3 comprises at least one of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.

32. The method of any one of claims 1 to 31, wherein contacting the population of progenitor cells occurs in vitro or ex vivo.

33. A perturbagen for use in the method of any one of claims 1 to 32.

34. A pharmaceutical composition comprising the perturbagen of claim 33.

35. A method for promoting the formation of a megakaryocyte cell, or an immediate progenitor thereof, comprising:
exposing a starting population of stem/progenitor cells comprising a non-lineage committed CD34+ cell to a perturbation having a perturbation signature that promotes the transition of the starting population of stem/progenitor cells into a MEP cell, committed megakaryocyte progenitor cell, or a promegakaryocyte,
wherein the perturbation signature comprises increased expression and/or activity of one or more of genes selected from Table 3 designated as an “up” gene in the gene directionality column of Table 3 and/or a decreased expression and/or activity in the non-lineage committed CD34+ cell of one or more genes selected from Table 3 designated as a “down” gene in the gene directionality column of Table 3.

36. The method of claim 35, wherein the perturbation signature comprises an activation of a network module designated in the network module column of Table 3.

37. A method of increasing a quantity of megakaryocyte cell, or immediate progenitors thereof, comprising:
exposing a starting population of stem/progenitor cells comprising a non-lineage committed CD34+ cell to a pharmaceutical composition that promotes the formation of lineage specific progenitor population selected from MEP cell, committed megakaryocyte progenitor cell, or a promegakaryocyte, the pharmaceutical composition promoting the transition of a primitive stem/progenitor population into the lineage specific progenitor population that has the capacity to differentiate into megakaryocytes, proplatelets, and/or platelets or immediate progenitors thereof,
wherein the pharmaceutical composition comprises at least one perturbagen selected from Table 6, or a variant thereof.

38. A method for treating a disease or disorder disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, the method comprising:
(a) administering to a patient in need thereof a therapeutically effective amount of at least one perturbagen selected from Table 6, or a variant thereof, wherein the at least one perturbagen is capable of changing a gene signature in a progenitor cell, or
(b) administering to a patient in need thereof a cell, the cell having been contacted with at least one perturbagen selected from Table 6, or a variant thereof, wherein the at least one perturbagen is capable of changing a gene signature in a progenitor cell.

39. A method for treating a disease or disorder characterized by an abnormal number of megakaryocytes, proplatelets, and/or platelets, comprising:
(a) administering to a patient in need thereof a therapeutically effective amount of at least one perturbagen selected from Table 6, or a variant thereof, wherein the at least one perturbagen is capable of changing a gene signature in a progenitor cell, or
(b) administering to a patient in need thereof a cell, the cell having been contacted with at least one perturbagen selected from Table 6, or a variant thereof, wherein the at least one perturbagen is capable of changing a gene signature in a progenitor cell.

40. The method of claim 39, wherein the abnormal ratio comprises a decreased number of megakaryocytes, proplatelets, and/or platelets and/or an increased number of progenitor cells.

41. A method for treating a disease or disorder characterized by an abnormal oxygen delivery, comprising:
(a) administering to a subject in need thereof a therapeutically effective amount of at least one perturbagen selected from Table 4, or a variant thereof, wherein the at least one perturbagen is capable of changing a gene signature in a progenitor cell or
(b) administering to a subject in need thereof a cell, the cell having been contacted with at least one perturbagen selected from Table 4, or a variant thereof, wherein the at least one perturbagen is capable of changing a gene signature in a progenitor cell.

42. A method for treating a disease or disorder characterized by a hemoglobin deficiency, comprising:
(a) administering to a subject in need thereof a therapeutically effective amount of at least one perturbagen selected from Table 4, or a variant thereof, wherein the at least one perturbagen is capable of changing a gene signature in a progenitor cell or
(b) administering to a subject in need thereof a cell, the cell having been contacted with at least one perturbagen selected from Table 4, or a variant thereof, wherein the at least one perturbagen is capable of changing a gene signature in a progenitor cell.

43. A method for treating or preventing an sickle cell disease or a thalassemia, comprising:
(a) administering to a subject in need thereof a therapeutically effective amount of at least one perturbagen selected from Table 4, or a variant thereof, wherein the at least one perturbagen is capable of changing a gene signature in a progenitor cell or
(b) administering to a subject in need thereof a cell, the cell having been contacted with at least one perturbagen selected from Table 4, or a variant thereof, wherein the at least one perturbagen is capable of changing a gene signature in a progenitor cell.

44. A method for treating a disease or disorder characterized by an abnormal erythron distribution and/or physiology, comprising:
(a) administering to a subject in need thereof a therapeutically effective amount of at least one perturbagen selected from Table 5, or a variant thereof, wherein the at least one perturbagen is capable of changing a gene signature in a progenitor cell or
(b) administering to a subject in need thereof a cell, the cell having been contacted with at least one perturbagen selected from Table 5, or a variant thereof, wherein the at least one perturbagen is capable of changing a gene signature in a progenitor cell.

45. A method for treating a disease or disorder characterized by an erythrocyte deficiency, comprising:
(a) administering to a subject in need thereof a therapeutically effective amount of at least one perturbagen selected from Table 5, or a variant thereof, wherein the at least one perturbagen is capable of changing a gene signature in a progenitor cell or
(b) administering to a subject in need thereof a cell, the cell having been contacted with at least one perturbagen selected from Table 5, or a variant thereof, wherein the at least one perturbagen is capable of changing a gene signature in a progenitor cell.

46. A method for treating or preventing an anemia, comprising:
(a) administering to a subject in need thereof a therapeutically effective amount of at least one perturbagen selected from Table 5, or a variant thereof, wherein the at least one perturbagen is capable of changing a gene signature in a progenitor cell or
(b) administering to a subject in need thereof a cell, the cell having been contacted with at least one perturbagen selected from Table 5, or a variant thereof, wherein the at least one perturbagen is capable of changing a gene signature in a progenitor cell.

47. The method of claim 41, wherein the disease is sickle cell disease or a thalassemia is sickle cell anemia (SS), sickle hemoglobin-C disease (SC), sickle beta-plus thalassemia and sickle beta-zero thalassemia.

48. The method of claim 44 to 46, wherein the anemia is selected from aplastic anemia, iron deficiency anemia, sickle cell anemia, thalassemia, vitamin deficiency anemia, chemotherapy induced anemia, erythropoietin (EPO) refractory anemia, aplastic anemia, and Diamond-Blackfan anemia.

49. The method of any one of claims 38 to 40, wherein the disease or disorder is selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia.

50. The method of any one of claims 35 to 49, wherein the subject is selected by steps comprising:
obtaining from the subject having the disease or disorder a sample of cells comprising a non-lineage committed CD34+ cell; and
contacting the sample of cells with at least one perturbagen selected from Table 4, Table 5, and/or Table 6, or a variant thereof, including combinations of the foregoing,
wherein the at least one perturbagen alters a gene signature in the sample of cells.

51. The method of any one of claims 35 to 49, wherein the subject is selected by steps comprising:
obtaining from a subject having the disease or disorder a sample of cells comprising a non-lineage committed CD34+ cell; and
contacting the sample of cells with at least one perturbagen capable of altering a gene signature in a non-lineage committed CD34+ cell,
wherein the at least one perturbagen increases in the sample of cells the expression and/or activity of one or more genes selected from the genes designated as an “up” gene in the gene directionality column of Table 1, Table 2, and/or Table 3, including combinations of the foregoing, and/or decreases in the sample of cells the expression and/or activity of one or more genes selected from the genes designated as a “down” gene in the gene directionality column of Table 1, Table 2, and/or Table 3, including combinations of the foregoing.

52. The method of claim 51, wherein altering the gene signature comprises an activation of a network module designated in the network module column of Table 3.

53. The method of any one of claims 35 to 49, wherein the subject is selected by steps comprising:
obtaining from a subject having the disease or disorder a sample of cells comprising a non-lineage committed CD34+ cell; and
contacting the sample of cells with at least one perturbagen selected from Table 4, Table 5, and/or Table 6, or a variant thereof, including combinations of the foregoing;
wherein the at least one perturbagen increases in the sample of cells the expression and/or activity of one or more genes selected from the genes designated as an “up” gene in the gene directionality column of Table 1, Table 2, and/or Table 3, including combinations of the foregoing, and/or decreases in the sample of cells the expression and/or activity of one or more genes selected from the genes designated as a “down” gene in the gene directionality column of Table 1, Table 2, and/or Table 3, including combinations of the foregoing.

54. The method of claim 53, wherein altering the gene signature comprises an activation of a network module designated in the network module column of Table 3.

55. Use of the perturbagen of Table 4, or a variant thereof in the manufacture of a medicament for treating a disease or disorder characterized an abnormal oxygen delivery or a hemoglobin deficiency.

56. Use of the perturbagen of Table 4, or a variant thereof in the manufacture of a medicament for treating sickle cell disease or a thalassemia.

57. Use of the perturbagen of Table 5, or a variant thereof in the manufacture of a medicament for treating a disease or disorder characterized by an abnormal ratio of erythrocytes to progenitor cells.

58. Use of the perturbagen of Table 6, or a variant thereof in the manufacture of a medicament for treating a disease or disorder characterized by an abnormal ratio of megakaryocytes, proplatelets, and/or platelets to progenitor cells.

59. Use of the perturbagen of Table 6, or a variant thereof in the manufacture of a medicament for treating a disease or disorder characterized by an abnormal ratio of megakaryocytes, proplatelets, and/or platelets to other committed blood cells, optionally erythrocytes.

60. A method of identifying a candidate perturbation for promoting the transition of a starting population of progenitor cells into erythrocytes comprising HbF or HbF-expressing progenitors thereof, the method comprising:
exposing the starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation; and
identifying the perturbation as a candidate perturbation for promoting the transition of a population of progenitor cells into erythrocytes comprising HbF or HbF-expressing progenitors thereof based on the perturbation signature,
wherein the perturbation signature is an increase in expression and/or activity in the progenitor cell of one or more genes selected from the genes designated as an “up” gene in the gene directionality column of Table 1, and/or a decrease in expression and/or activity in the progenitor cell of one or more genes selected from the genes designated as a “down” gene in the gene directionality column of Table 1.

61. A method of identifying a candidate perturbation for promoting the transition of a starting population of progenitor cells into erythrocytes or immediate progenitors thereof, the method comprising:
exposing the starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes or immediate progenitors thereof following exposure of the population of cells to the perturbation; and
identifying the perturbation as a candidate perturbation for promoting the transition of a population of progenitor cells into erythrocytes or immediate progenitors thereof based on the perturbation signature,
wherein the perturbation signature is an increase in expression and/or activity in the progenitor cell of one or more genes selected from the genes designated as an “up” gene in the gene directionality column of Table 2, and/or a decrease in expression and/or activity in the progenitor cell of one or more genes selected from the genes designated as an “down” gene in the gene directionality column of Table 2.

62. A method of identifying a candidate perturbation for promoting the transition of a starting population of progenitor cells into megakaryocytes, proplatelets, and/or platelets or immediate progenitors thereof, the method comprising:
exposing the starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into megakaryocytes, proplatelets, and/or platelets or immediate progenitors thereof following exposure of the population of cells to the perturbation; and
identifying the perturbation as a candidate perturbation for promoting the transition of a population of progenitor cells into megakaryocytes, proplatelets, and/or platelets or immediate progenitors thereof based on the perturbation signature,
wherein the perturbation signature is an increase in expression and/or activity in the progenitor cell of one or more genes selected from Table 3 designated as an “up” gene in the gene directionality column of Table 3, and/or a decrease in expression and/or activity in the progenitor cell of one or more genes selected from Table 3 designated as a “down” gene in the gene directionality column of Table 3.

63. The method of any one of claims 60 to 62, wherein the perturbation signature is an increase in expression and/or activity in the progenitor cell of a network module designated in the network module column of Table 1, Table 2, and/or Table 3, including combinations of the foregoing.

64. A method for making a therapeutic agent for a disease or disorder selected from a sickle cell disease or a thalassemia or a disease or disorder characterized by an abnormal oxygen delivery or a hemoglobin deficiency, comprising:
(a) identifying a candidate perturbation for therapy according to the method of claim 60 and
(b) formulating the candidate perturbation as a therapeutic agent for the treatment of the disease or disorder.

65. A method for making a therapeutic agent for a disease or disorder selected from a disease or disorder characterized by an abnormal erythron distribution and/or physiology or erythrocyte deficiency, comprising:
(a) identifying a candidate perturbation for therapy according to the method of claim 61 and
(b) formulating the candidate perturbation as a therapeutic agent for the treatment of the disease or disorder.

66. A method for making a therapeutic agent for a disease or disorder selected from congenital amegakaryocytic thrombocytopenia, thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), myeloablation/chemotherapy induced thrombocytopenia, thrombocytopenia resulting from liver disease, thrombocytosis, myelofribrosis, and radiation-induced thrombocytopenia, comprising:
(a) identifying a candidate perturbation according to the method of claim 62, and
(b) formulating the candidate perturbation as a therapeutic agent for the treatment of the disease or disorder.

67. A method for directing a change in cell state of a plurality of progenitor cells comprising:
contacting a population of cells comprising a plurality of progenitor cells with at least one perturbagen selected from Table 4, Table 5, and/or Table 6, or a variant thereof, including combinations of the foregoing,
wherein the at least one perturbagen is capable of altering one or more gene signatures in the plurality of progenitor cells; and
wherein the plurality of progenitor cells are non-lineage committed CD34+ cells.

68. The method of claim 67, wherein the change in cell state provides an increase in the number of one or more of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets, and any combination thereof, optionally wherein one or more of the proerythroblasts, early erythroblasts, intermediate erythroblasts, late erythroblasts, reticulocytes, and erythrocytes expresses fetal hemoglobin (HbF).

69. The method of claim 68, wherein the number of proerythroblasts, early erythroblasts, intermediate erythroblasts, late erythroblasts, reticulocytes, erythrocytes, megakaryocyte/erythroid progenitor cells (MEP), committed megakaryocyte progenitor cells, promegakaryocytes, megakaryocytes, proplatelets, and/or platelets is increased after contacting the population of cells comprising a CD34+ cell with the at least one perturbagen.

70. The method of any one of claims 67-69, wherein the at least one perturbagen selected from Table 4, Table 5, and/or Table 6, or a variant thereof, including combinations of the foregoing, comprises at least 2, at least 3, at least 4, or at least 5 perturbagens selected from Table 4, Table 5, and/or Table 6, or variants thereof, including combinations of the foregoing.

71. The method of any one of claims 67-70, wherein the one or more gene signatures are selected from:
a) one or more genes designated as an “up” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, genes selected from the genes designated as an “up” gene in the gene directionality column of Table 1;
b) one or more genes selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, genes selected from the genes designated as a “down” gene in the gene directionality column of Table 1;
c) one or more genes selected from the genes designated as an “up” gene in the gene directionality column of Table 2, comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 genes selected from the genes designated as an “up” gene in the gene directionality column of Table 2:
d) one or more genes selected from the genes designated as an “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, or 79 genes selected from the genes designated as an “down” gene in the gene directionality column of Table 2;
e) one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, or 23 or more genes designated as an “up” gene in the gene directionality column of Table 3; and
f) one or more genes selected from Table 3 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more or 23 or more, 24 or more, or 25 or more genes designated as a “down” gene in the gene directionality column of Table 3.

72. The method of claim 71, wherein the one or more genes comprise one or more of a)-f):
a) the one or more genes selected from the genes designated as an “up” gene in the gene directionality column of Table 1 comprises at least one of KIT, APOE, RNH1, ID2, BLVRA, TSKU, HEBP1, TRAK2, HK1, GAPDH, MPC2, CTNNAL1, CAST, CALM3, RPA3, ELOVL6, BNIP3, SPAG4, S100A4, RALB, RAP1GAP, DENND2D, CTSL, DDIT4, BNIP3L, and VAT1;
b) the one or more genes selected from the genes designated as a “down” gene in the gene directionality column of Table 1 comprises at least one of CDK6, PLP2, MAP7, TRAPPC6A, BID, SYK, FAIM, BTK, TBXA2R, LYPLA1, MAPKAPK3, SLC35F2, ANXA7, ATP6VOB, SYPL1, BCL7B, INPP1, ADI1, MACF1, MLLT11, FHL2, RNPS1, TPM1, THAP11, DUSP14, PSMB8, EIF4EBP1, MFSD10, PSMD2, SPTLC2, CORO1A, PDLIM1, CCDC85B, ITGAE, CCDC86, SLC5A6, GRWD1, SNCA, IL1B, MEST, DAXX, UBE2L6, PTPRC, GADD45A, NENF, PTPN6, RHOA, EVL, VDAC1, TIMM17B, MTHFD2, XBP1, EBNA1BP2, CYCS, TCEAL4, TMEM109, MLEC, HDAC2, SKP1, MEF2C, SPAG7, ICAM3, RPL39L, SOX4, MYC, IL4R, TES, CASP3, PHGDH, DRAP1, RPS6, RNF167, and PSME2;
c) the one or more genes selected from the genes designated as an “up” gene in the gene directionality column of Table 2 comprises at least one of TSC22D3, DDIT4, TNIP1, FHL2, HMGCS1, CYCS, HK1, ACLY, JADE2, P/H1D1, BAX, RPA2, CCND3, KIT, CYB561, S100A4, PIN1, NT5DC2, CD320, APOE, ID2, DAXX, CTTN, IFRD2, and CAB39;
d) the one or more genes selected from the genes designated as an “down” gene in the gene directionality column of Table 2 comprises at least one of DNAJC15, SNCA, CEP57, BZW2, BID, SMC3, VDAC1, RNPS1, PSMB8, MLEC, SNX6, SMARCA4, HSPD1, NUCB2, PHGDH, GABPB1, CCNH, RBM6, MAT2A, RAB4A, HEBP1, CORO1A, ACAA1, PPOX, MEST, STX4, FKBP4, UBE2A, DERA, ATG3, NUSAP1, NUP88, H2AFV, PLP2, UBE2L6, HLA-DRA, MLLT11, SCP2, OXA1L, KTN1, GNA/2, DECR1, LSM6, HADH, WDR61, DCK, KLHDC2, CAT, CBR3, DHRS7, BAD, GAPDH, CDK4, MAPKAPK3, PSIP1, PCM1, PSMD4, HSPA8, SPTLC2, SOX4, HLA-DMA, SCCPDH, LAGE3, PDLIM1, EAPP, MRPS16, VPS28, FAH, PSMB10, ICAM3, HSD17B11, MIF, NENF, RPA3, ADI1, AKR7A2, KDELR2, PGAM1, and CREG1;
e) the one or more genes selected from Table 3 comprises at least one of CCND3, RSU1, PDLIM1, DNM1L, PTPN12, GADD45A, SH3BP5, TSC22D3, CXCL2, TPM1, PTPN6, ABHD4, SNCA, INSIG1, STXBP2, LRRC16A, ZFP36, NFKBIA, CXCR4, BTK, GNB5, PROS1, HSPB1, and MYLK; and/or
f) the one or more genes selected from Table 3 comprises at least one of CD320, PAFAH1B3, TRAP1, RRP1B, HLA-DRA, EIF4EBP1, TFDP1, CDK6, CDK4, MIF, MYC, RPL39L, PAICS, FBXO7, IFRD2, CD44, APOE, MAT2A, MPC2, RPS5, ICAM3, RPS6, CISD1, GAPDH, HSPA8, and HSPD1.
